(19) United States
(12) Patent Application Publication
(10) Pub. No.: US 2013/0261130 A1
(43) Pub. Date: Oct. 3, 2013

(54) NEUROTRYPSIN INHIBITORS

(71) Applicants: Shaheen Ahmed, Zurich (CH); Stefan Hettwer, Rupperwil (CH); Jan Willem Vrijbloed, Mohlin (CH); Carlo Farina, Valsolda (IT); Hans Allgeier, Lorrach-Haagen (DE)

(72) Inventors: Shaheen Ahmed, Zurich (CH); Stefan Hettwer, Rupperwil (CH); Jan Willem Vrijbloed, Mohlin (CH); Carlo Farina, Valsolda (IT); Hans Allgeier, Lorrach-Haagen (DE)

(21) Appl. No.: 13/882,351

(22) PCT Filed: Oct. 31, 2011
(86) PCT No.: PCT/EP2011/069074

§ 371 Date: Jun. 5, 2013

(30) Foreign Application Priority Data
Nov. 11, 2010 (EP) 10189540.7

(51) Int. Cl. C07D 409/12; C07D 401/12; C07D 413/12; C07D 333/38; C07D 409/14; C07D 417/12; C07D 333/70; C07D 209/42

(52) U.S. Cl. CPC C07D 409/12; C07D 333/70; C07D 401/12; C07D 209/42; C07D 333/38; C07D 409/14; C07D 417/12; C07D 413/12
USPC 514/252.13; 549/57; 514/443; 546/199; 514/322; 546/281.1; 514/337; 544/327; 514/256; 546/202; 514/324; 544/376; 548/492; 514/419; 549/72; 514/448; 548/253; 514/382; 548/195; 514/371; 548/255; 514/359; 548/229; 514/376

(57) ABSTRACT

The invention relates to acylamino-phthalic acid amides and related compounds of formula (I) wherein A is —CON—R³R⁴, —NR⁵COR⁶, —NHR⁷, —OR⁸, —SR⁹, —CH₂NR¹⁰R¹¹, —(CH2)2-R¹², —CH═CH—R¹², —C≡C—R¹², optionally substituted phenyl, optionally substituted thiophenyl, or optionally substituted 1,2,3-triazol-4-yl, W is hydrogen, hydroxy or carboxymethoxy, Y is carboxy, methoxycarbonyl or 2H-tetrazol-5-yl, and the various substituents R have the meanings indicated in the description. These compounds are useful for the treatment and/or prophylaxis of skeletal muscle atrophy, schizophrenia and Alzheimer's disease, and as cognitive enhancers.

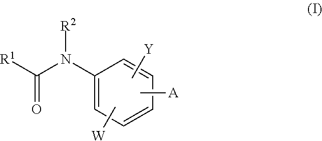

### FIELD OF THE INVENTION

The invention relates to acylamino-phthalic acid amides and related compounds, and the use of such compounds for the treatment and/or prophylaxis of skeletal muscle atrophy and schizophrenia, Alzheimer's disease, and as cognitive enhancers.

### BACKGROUND OF THE INVENTION

The enzyme neurotrypsin (WO 98/49322) belongs to the chymotrypsin family, whose members are almost entirely confined to animals. The amino acid sequence of neurotrypsin defines a mosaic protein of 875 amino acids consisting of a Kringle domain, followed by four scavenger receptor cysteine-rich repeats (three in the mouse), and the serine protease domain. Neurotrypsin contains, like thrombin, tPA, trypsin and some other enzymes, an aspartate residue in the bottom of its S1 pocket, therefore showing specificity for basic amino acids at this binding site. The structural similarity of neurotrypsin to the proteases of the blood coagulation cascade and the fibrinolytic system, such as factor X, factor IX, thrombin, tissue plasminogen activator, and plasmin suggests that it may be an element of a protease-driven extracellular signaling mechanism in the nervous system.

As was shown in WO 2006/103261, neurotrypsin is located at the presynaptic nerve terminal of synapses of the central nervous system (CNS) and at the neuromuscular junction (NMJ). The synapse is the connection between nerve cells (neurons) where messages are communicated in the form of chemical substances, termed neurotransmitters. The synapse is composed of a presynaptic terminal formed by the signal-emitting cell and the postsynaptic specialization of the signal-receiving cell. Neurotransmitters released from the presynaptic terminal cross the synaptic cleft and bind to the neurotransmitter receptors in the postsynaptic specialization. Upon binding of the neurotransmitter the receptor induces the generation of an electrical pulse in the postsynaptic cell. Signal transmission between two neurons is the basis of neuronal function. Brain functions are the result of the specific assembly of an enormous number of neurons to information-processing networks.

The majority of synapses is found in the central nervous system (CNS, brain and spine), where every synapse connects two neurons. By such bilateral point-to-point connections, every neuron may connect to thousands of other neurons. However, synapses also connect a neuron to a gland or a muscle cell. The neuromuscular junction (NMJ, muscle end-plate) is the synapse that connects a nerve cell with a striated muscle cell. Synapses located outside of the brain, the brain stem and the spinal cord are termed peripheral nervous system (PNS) synapses. CNS synapses and PNS synapses exhibit many structural and functional commonalities and share many of their molecular components (synaptic molecules). Therefore, synaptic target molecules may be useful for targeting synaptic functions of both the CNS and the PNS.

Skeletal muscle atrophy (sarcopenia), defined as the loss of muscle mass and strength, plays a major role in the pathogenesis of frailty and functional impairment that occurs with old age. It plays a major role in the loss of muscular strength, decreased metabolic rate, gradual reduction of bone density and decreased aerobic capacity (Doherty, T. J., J. Appl. Physiol. 95: 1717-1727, 2003). The loss of muscle mass manifests as a decrease in the cross-sectional area of the muscle with age, which has been determined to result from a combined effect of a reduction in both the number of muscle fibers and the thickness of the individual remaining fibers.

Over the past years, considerable progress has been made in the identification and characterization of factors contributing to the degradation of muscle mass. Important genes associated with these processes encode ubiquitin protein ligases that were found increased in atrophic muscle. Among the factors that have a hypertrophic activity and, as such, block atrophy, insulin-like growth factor 1 (IGF-1) has been found to play an essential role. This and several other regulatory pathways controlling skeletal muscle mass have been investigated intensively (for a review see: Glass, D. J., Nature Cell Biol. 5: 87-90, 2003). In spite of important progress in both the characterization of the molecular mechanisms that control muscle degradation leading to atrophy and the hypertrophic effects of insulin-like growth factor, and in spite of the fact that several companies work on the development of drugs capable of stimulating the increase of muscle mass, no drugs have been approved up to now.

A morphological hallmark of the skeletal muscle atrophy found at old age (sarcopenia) is a considerable reduction of the number of muscle fibers. Ample evidence from numerous independent studies supports that neural input to a fraction of the muscle fibers is disrupted with age, resulting in subsequent atrophy and eventually the disappearance of the denervated fibers (Kamal, H. K., Nutrition Reviews 61: 157-167, 2003). Another characteristic feature of the skeletal muscle atrophy found at old age is a coincidence of the muscular atrophy with a considerable reduction of the number of motoneurons (Welle, S., Can. J. Appl. Physiol. 27: 19-41, 2002) and a marked structural alteration of the neuromuscular junction (Tapia, J. C. et al., Abstract Viewer/Itinerary Planner, Washington D.C.: Society for Neuroscience). These characteristics indicate that a significant age-related deterioration of the structure and the function of the neuromuscular junction is a major contributing factor to a process that ultimately results in a structural and functional denervation. Denervated muscle fibers that do not receive compensatory reinnervation within weeks become progressively atrophic and eventually disappear.

Schizophrenia is a chronic, severe, and disabling brain disease. Approximately 1% of the world population develops schizophrenia during their lifetime. Individuals who develop schizophrenia experience severe suffering. Approximately 10% commit suicide. Although schizophrenia affects men and women with equal frequency, the disorder often appears earlier in men, usually in the late teens or early twenties, than in women, who are generally affected in the twenties to early thirties. People with schizophrenia often suffer terrifying symptoms such as hearing internal voices not heard by others, or believing that other people are reading their minds, controlling their thoughts, or plotting to harm them. These symptoms may leave them fearful and withdrawn. Their speech and behavior can be so disorganized that they may be incomprehensible or frightening to others. The currently available treatments of schizophrenia reduce suffering considerably, but approximately ⅔ of the people affected by schizophrenia require public assistance within a few years after onset. The majority of them are unable to return to work or school and have relatively little or no social interactions, and most people with schizophrenia continue to suffer some symptoms throughout their lives. It has been estimated that no more than one in five individuals recovers completely. Therefore schizophrenia is one of the most important public health problems world-wide, and the costs to society are counted in billions of dollars.

The currently most consistent neuropathological finding in brains of schizophrenic patients is a reduction of the number of synapses in the gray matter of the central nervous system, which is reflected by a decrease in the volume of the neuropil (the synaptic area). No evidence for neuronal degeneration is observed. Typically, the number of neurons counted per area of tissue is rather increased, an observation explained by a selective decrease in the number of synapses in the neuropil area between the neurons while the number of neuronal cell somas remained constant. The phenomenon has been reported over the past two decades by several independent studies on post mortem material and has been found most extensive in the prefrontal cortex. The literature documenting this observation has been carefully reviewed by Selemon, L. D. and Goldman-Rakic, P.S. (Psychiatry 45: 17-25, 1999). McGlashan, T. H. and Hoffman, R. E. (Arch. Gen. Psychiatry 57: 637-648, 2000) summarized the essential morphological, developmental, electrophysiological, and metabolic observations in schizophrenia in the light of the “excessive synaptic pruning” hypothesis and came to the conclusion that “excessive synaptic pruning” or “developmentally reduced synaptic connectivity” is an increasingly attractive pathophysiological model of schizophrenia. Based on this model, schizophrenia arises from critically reduced synaptic connectedness as a result of developmental disturbances of synaptogenesis during gestation and early childhood and/or excessive synaptic pruning during adolescence. The model accounts for the phenomenology of the disorder, the symptomatic states, the onset, neurodevelopmental deficits, window of deterioration, sex differences in clinical presentation, course determined by age of onset, and preservation of the schizophrenic genotype in the population despite diminished phenotypic fecundity.

Alzheimer's disease (AD) is the most common form of dementia whose most common symptoms include inability to acquire new memories, difficulty in recalling recently observed facts, confusion, irritability and aggression, mood swings, language breakdown and long-term memory loss. The cause and progression of Alzheimer's disease are not well understood. Research indicates that the disease is associated with plaques and tangles in the brain. Recently, it has been shown that loss of lean mass is accelerated in AD and is associated with brain atrophy and cognitive performance, perhaps as a direct or indirect consequence of AD pathophysiology or through shared mechanisms common to both AD and sarcopenia. (Burns et al., Arch Neurol. 67: 428-33, 2010). Therefore, inhibitors of neurotrypsin could also be effective in treating Alzheimer's disease or in reducing its symptoms.

Cognitive enhancers are drugs aimed at preventing, improving, or treating cognitive deficits at both the clinical and subclinical level. Such drugs are beneficial for the treatment of memory difficulties of elderly people who have not progressed to Alzheimer's disease (mild cognitive impairment). However, such drugs are also beneficial for the improvement of cognitive functions in patients with the established diagnosis of Alzheimer's disease or other diseases associated with dementia or for the improvement of cognitive functions in posttraumatic cognitive dysfunction, as well as for the improvement of the age-related impairment of cognitive functions that are considered as a normal feature of the ageing process.

Mild cognitive impairment is a widely cited concept in clinical research on ageing-related cognitive disorders (Ritchie, K. and Touchon, J., The Lancet 355: 225-228, 2000). It refers generally to subclinical complaints of memory functioning in elderly people, which are judged to have a high probability of evolving towards Alzheimer's disease. The identification of people at potential risk for dementia with a view to early therapeutic intervention is important, because it may lessen distress for both patient and family, minimize the risk of accidents, prolong autonomy, and perhaps even ultimately prevent the onset of the process leading to dementia itself.

The impairment of cognitive functions without dementia is so common among elderly people that it is considered by many as an inevitable feature of the ageing process. Nonetheless, it has acquired clinical significance because of the difficulties patients may have with carrying out everyday activities. Although the range of impairments seen in populations without dementia is extremely broad, several clinical labels have been proposed to describe this tail-end of the normal cognitive range. One of the earliest was benign senescent forgetfulness. Its clinical features include an inability to recall minor detail, the forgetting of remote as opposed to recent events, and awareness of memory problems. The term ageing-associated cognitive decline refers to a wider range of cognitive functions (attention, memory, learning, thinking, language, and visuospatial function), and is diagnosed by reference to norms for elderly people. Prescription of cognitive enhancers may prolong the capacity of the affected individuals to carry out their daily activities and, thus, prolong their autonomy. Other disorders associated at least in part of the affected individuals with cognitive impairments that may eventually lead to dementia include Parkinson's disease, multiple sclerosis, stroke, and head trauma. The prescription of cognitive enhancer drugs may also improve cognitive functions in these patients.

### SUMMARY OF THE INVENTION

The invention relates to compounds of formula (I)

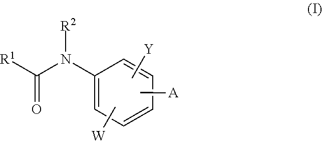

wherein
A is —CONR³R⁴, —NR⁵COR⁶, —NHR⁷, —OR⁸, —SR⁹, —CH₂NR¹⁰R¹¹, —(CH₂)₂—R¹², —CH═CH—R¹², —C≡C—R¹², optionally substituted phenyl, optionally substituted thiophenyl, or optionally substituted 1,2,3-triazol-4-yl;
W is hydrogen, hydroxy or carboxymethoxy;
Y is carboxy, methoxycarbonyl or 2H-tetrazol-5-yl;
R¹ is cycloalkyl, cycloalkenyl, aryl, arylmethyl, heteroaryl, or heteroarylmethyl;
R² is hydrogen or methyl;
R³ is alkyl, optionally substituted amino-, hydroxy-, carbamimidoyl-, or cycloalkyl-lower alkyl; aryl-lower alkyl, heteroaryl-lower alkyl, cycloalkyl, bicycloalkyl, tricycloalkyl, heterocyclyl, aryl, or heteroaryl;
R⁴ is hydrogen, lower alkyl, carboxy-, lower alkoxycarbonyl-, dimethylcarbamoyl-, hydroxy- or lower alkoxy-lower alkyl; or R³ and R⁴ together with the nitrogen atom, to which they are bound, are optionally substituted pyrrolidino, optionally substituted piperidino, morpholino, or optionally substituted piperazino;
R⁵ is hydrogen or methyl;
R⁶ is aryl, heteroaryl, optionally substituted alkylamino, arylamino, optionally substituted pyrrolidino, optionally substituted piperidino, morpholino, or optionally substituted piperazino, with the proviso that R⁶ cannot be 2-thiophenyl if R¹ is 2-thiophenyl;
or R⁵ and R⁶ together with the nitrogen atom and the carbonyl group, to which they are bound, are optionally substituted 2-oxopyrrolidino, optionally substituted 2-oxopiperidino, or optionally substituted 2-oxo-oxazolidin-3-yl;
R⁷ is cycloalkyl, cycloalkenyl, aryl, aryl-lower alkyl, optionally substituted alkylsulfonyl, or arylsulfonyl;
R⁸ is phenyl if Y is carboxy and R¹ is optionally substituted benzimidazolyl-phenyl or chloro-substituted benzothiophenyl, aryl-lower alkyl with the exclusion of ortho-methoxybenzyl, optionally substituted benzocycloalkyl or benzocycloalkenyl, optionally substituted alkyl-, dialkyl- or aryl-carbamoylmethyl, or heterocyclylcarbonylmethyl, wherein heterocyclyl is bound to carbonyl through a nitrogen atom;
R⁹ is aryl;
R¹⁰ is arylcarbonyl, heteroarylcarbonyl or optionally substituted alkylcarbonyl;
R¹¹ is hydrogen or methyl;
or R¹⁰ and R¹¹ together with the nitrogen atom, to which they are bound, are optionally substituted 2-oxopyrrolidino, optionally substituted 2-oxopiperidino, or optionally substituted 2-oxo-oxazolidin-3-yl; and
R¹² is aryl or aryl-lower alkyl if R¹ is optionally substituted benzimidazolyl-phenyl or chloro-substituted benzothiophenyl;
and salts thereof.

A bond pointing to the centre of the phenyl ring in formula (I) means that the corresponding substituent can take any place. For example, if W is hydrogen, A and Y may be in position 2 and 3, 2 and 4, 2 and 5, 2 and 6, 3 and 2, 3 and 4, 3 and 5, 3 and 6, 4 and 2, and 4 and 3, respectively. If W is different from hydrogen, this substituent may then occupy any of the remaining positions.

The invention further relates to compounds as defined hereinbefore for use as medicaments, in particular for use in the treatment and/or prophylaxis of diseases caused by deficiency of synapses, for example skeletal muscle atrophy, schizophrenia, Alzheimer's disease and cognitive disturbance, pharmaceutical preparations containing these compounds, and a method of treatment and/or prophylaxis of diseases caused by deficiency of synapses, for example skeletal muscle atrophy, schizophrenia, Alzheimer's disease and cognitive disturbance.

### DETAILED DESCRIPTION OF THE INVENTION

The invention is based on the fact that inhibition of neurotrypsin allows enhancing pro-synaptic (synapse-forming, synapse-differentiating, synapse-organizing, synapse-protecting, synapse-strengthening) activities. The neurotrypsin gene is expressed in many neurons of the central nervous system, including the motoneurons of the spinal cord, and the neurotrypsin protein is found in many CNS synapses, as well as at the neuromuscular junction. Neurotrypsin plays a substantial role in the development and/or the maintenance of a well balanced synaptic function. Too much neurotrypsin (overexpression) correlates with too few synaptic connections. Transgenic mice overexpressing neurotrypsin in CNS neurons show a reduced number of synapses in the cerebral cortex and the hippocampus, two brain structures that are highly important for cognitive functions, such as memory and learning. Likewise, transgenic mice overexpressing neurotrypsin in spinal motoneurons show a reduction of the neuromuscular junctions, the synapses that mediate the neural control of muscular activity (WO 2006/103261).

The pharmaceutical tuning of neurotrypsin activity provides an unprecedented access to the regulatory machinery of synaptic function. Inhibiting proteolytic activity of neurotrypsin shifts the synaptic balance towards strengthening the pro-synaptic activities at the expense of the anti-synaptic activities and thus towards increasing the number and/or the size and/or the strength of synapses.

The invention relates to compounds of formula (I)

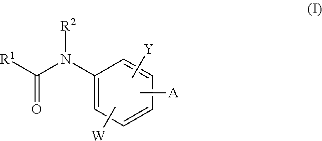

wherein
A is —CONR³R⁴, —NR⁵COR⁸, —NHR⁷, —OR⁸, —SR⁹, —CH₂NR¹⁰R¹¹, or optionally substituted 1,2,3-triazol-4-yl;
W is hydrogen, hydroxy or carboxymethoxy;
Y is carboxy, methoxycarbonyl or 2H-tetrazol-5-yl;
R¹ is cycloalkyl, cycloalkenyl, aryl, arylmethyl, heteroaryl, or heteroarylmethyl;
R² is hydrogen or methyl;
R³ is alkyl, optionally substituted amino-, hydroxy-, carbamimidoyl-, or cycloalkyl-lower alkyl; aryl-lower alkyl, heteroaryl-lower alkyl, cycloalkyl, cycloalkenyl, bicycloalkyl, tricycloalkyl, heterocyclyl, aryl, or heteroaryl;
R⁴ is hydrogen, lower alkyl, carboxy-, lower alkoxycarbonyl-, dimethylcarbamoyl-, hydroxy- or lower alkoxy-lower alkyl;
or R³ and R⁴ together with the nitrogen atom, to which they are bound, are optionally substituted pyrrolidino, optionally substituted piperidino, morpholino, or optionally substituted piperazino;
R⁵ is hydrogen or methyl;
R⁶ is aryl, heteroaryl, optionally substituted alkylamino, arylamino, optionally substituted pyrrolidino, optionally substituted piperidino, morpholino, or optionally substituted piperazino; with the proviso that R⁶ cannot be 2-thiophenyl if R¹ is 2-thiophenyl;
or R⁵ and R⁶ together with the nitrogen atom and the carbonyl group, to which they are bound, are optionally substituted 2-oxopyrrolidino, optionally substituted 2-oxopiperidino, or optionally substituted 2-oxo-oxazolidin-3-yl;
R⁷ is cycloalkyl, cycloalkenyl, aryl, aryl-lower alkyl, optionally substituted alkylsulfonyl, or arylsulfonyl;
R⁸ is aryl-lower alkyl with the exclusion of ortho-methoxybenzyl, optionally substituted benzocycloalkyl or benzocycloalkenyl, optionally substituted alkyl-, dialkyl- or aryl-carbamoylmethyl, or heterocyclylcarbonyl wherein heterocyclyl is bound to carbonyl through a nitrogen atom;
R⁹ is aryl;
R¹⁰ is arylcarbonyl, heteroarylcarbonyl, or optionally substituted alkylcarbonyl; and
R¹¹ is hydrogen or methyl;
or R¹⁰ and R¹¹ together with the nitrogen atom, to which they are bound, are optionally substituted 2-oxopyrrolidino, optionally substituted 2-oxopiperidino, or optionally substituted 2-oxo-oxazolidin-3-yl;
or A is —OR⁸; —(CH₂)₂—R¹², —CH═CH—R¹², —C≡C—R¹²; optionally substituted phenyl, or optionally substituted thiophenyl, W is hydrogen; Y is carboxy, methoxycarbonyl or 2H-tetrazol-5-yl; R¹ is optionally substituted benzimidazolyl-phenyl or chloro-substituted benzothiophenyl; R² is hydrogen; R⁸ is phenyl if Y is carboxy; and R¹² is aryl or aryl-lower alkyl;
and pharmaceutically acceptable salts thereof.

A bond pointing to the centre of the phenyl ring in formula (I) means that the corresponding substituent can take any place. For example, if W is hydrogen, A and Y may be in position 2 and 3, 2 and 4, 2 and 5, 2 and 6, 3 and 2, 3 and 4, 3 and 5, 3 and 6, 4 and 2, and 4 and 3, respectively. If W is different from hydrogen, this substituent may then occupy any of the remaining positions.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Double bonds in principle can have E- or Z-configuration. The compounds of this invention may therefore exist as isomeric mixtures or single isomers. If not specified both isomeric forms are intended.

Any asymmetric carbon atoms may be present in the (R)—, (S)— or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula (I).

Alkyl has from 1 to 12, preferably from 1 to 7 carbon atoms, and is linear or branched. Alkyl is preferably lower alkyl.

Lower alkyl has 1 to 7, preferably 1 to 4 carbon atoms and is butyl, such as n-butyl, sec-butyl, isobutyl, tert-butyl, propyl, such as n-propyl or iso-propyl, ethyl or methyl. Preferably lower alkyl is methyl or ethyl. Lower alkyl may also be designated as C₁-C₇-alkyl, preferably C₁-C₄-alkyl.

Cycloalkyl has preferably 3 to 7 ring carbon atoms, and may be unsubstituted or substituted, e.g. by lower alkyl or lower alkoxy. Cycloalkyl is, for example, cyclohexyl, cyclopentyl, methylcyclopentyl, cycloheptyl or cyclopropyl.

Cycloalkenyl has preferably 5 to 7 ring carbon atoms, and may be unsubstituted or substituted, e.g. by lower alkyl. Cycloalkenyl is, for example, 1-cyclohexenyl, 3-cyclohexenyl, 1-cyclopentenyl, or 1-cycloheptenyl.

Aryl stands for a mono- or bicyclic fused ring aromatic group with 5 to 10 carbon atoms, such as phenyl, 1-naphthyl or 2-naphthyl, or also a partially saturated bicyclic fused ring comprising a phenyl group, for example benzo-C₅- or -C₆-cycloalkyl or -cycloalkenyl, such as indanyl, indenyl, dihydro- or tetrahydronaphthyl. Preferably, aryl is phenyl or benzo-C₅- or -C₆-cycloalkyl, in particular phenyl.

Aryl is unsubstituted or substituted. Aryl may be substituted by up to four substituents independently selected from lower alkyl, halo-lower alkyl, cycloalkyl-lower alkyl, carboxy-lower alkyl, lower alkoxycarbonyl-lower alkyl; arylalkyl or heteroarylalkyl, wherein aryl or heteroaryl are unsubstituted or substituted by up to three substituents selected from lower alkyl, halo-lower alkyl, lower alkoxy, halogen, amino, cyano and nitro; hydroxy-lower alkyl, lower alkoxy-lower alkyl, aryloxy-lower alkyl, heteroaryloxy-lower alkyl, aryl-lower alkoxy-lower alkyl, heteroaryl-lower alkoxy-lower alkyl, lower alkoxy-lower alkoxy-lower alkyl; aminoalkyl wherein amino is unsubstituted or substituted by one or two substituents selected from lower alkyl, hydroxy-lower alkyl, alkoxy-lower alkyl, amino-lower alkyl, alkylcarbonyl, alkoxycarbonyl, amino-lower alkoxycarbonyl, lower alkoxy-lower alkoxy-carbonyl and carbamoyl (i.e. aminocarbonyl), or wherein the two substituents on nitrogen form together with the nitrogen heterocyclyl; optionally substituted alkenyl, optionally substituted alkinyl, cycloalkyl, aryl, heteroaryl, heterocyclyl, hydroxy, lower alkoxy, halo-lower alkoxy, lower alkoxy-lower alkoxy, cycloalkyl-lower alkoxy, aryloxy, aryl-lower alkoxy, aryloxy-lower alkoxy, heteroaryloxy, heteroaryl-lower alkoxy, heteroaryloxy-lower alkoxy, optionally substituted alkenyloxy, optionally substituted alkinyloxy, cycloalkyloxy, heterocyclyloxy, alkylmercapto, alkylsulfinyl, halo-lower alkylsulfinyl, alkylsulfonyl, arylsulfonyl, heteroarylsulfonyl, guanidinosulfonyl; sulfamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, optionally substituted phenyl, optionally substituted phenyl-lower alkyl, optionally substituted heteroaryl, optionally substituted heteroaryl-lower alkyl, alkylcarbonyl, alkoxycarbonyl or carbamoyl, and wherein alkyl or lower alkyl in each case may be substituted by halogen, lower alkoxy, aryl, heteroaryl or optionally substituted amino, or wherein the two substituents on nitrogen form together with the nitrogen heterocyclyl; lower alkylcarbonyl, halo-lower alkylcarbonyl, optionally substituted phenylcarbonyl, optionally substituted heteroarylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; cyano, halogen, and nitro; and wherein two substituents in ortho-position to each other can form a 5-, 6- or 7-membered carbocyclic or heterocyclic ring containing one, two or three oxygen atoms, one or two nitrogen atoms and/or one sulfur atom.

In particular, the substituents may be independently selected from lower alkyl, halo-lower alkyl, hydroxy-lower alkyl, lower alkoxy-lower alkyl, optionally substituted alkenyl, optionally substituted alkinyl, cyclohexyl, aryl, heteroaryl, heterocyclyl, hydroxy, lower alkoxy, halo-lower alkoxy, lower alkoxy-lower alkoxy, cycloalkyloxy, optionally substituted phenyloxy, optionally substituted phenyl-lower alkoxy; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, optionally substituted heteroaryl, alkylcarbonyl, alkoxycarbonyl or carbamoyl, or wherein the two substituents on nitrogen form together with the nitrogen heterocyclyl; lower alkylcarbonyl, halo-lower alkylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, hydroxy-lower alkyl, lower alkoxy-lower alkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; arylsulfonyl, heteroarylsulfonyl, guanidinosulfonyl, sulfamoyl, cyano, halogen, and nitro; and wherein two substituents in ortho-position to each other can form a 5- or 6-membered heterocyclic ring containing one or two oxygen atoms and/or one nitrogen atom.

In optionally substituted phenyl, phenoxy or phenylamino, substituents are preferably lower alkyl, halo-lower alkyl, lower alkoxy-lower alkyl, hydroxy, lower alkoxy, halo-lower alkoxy, lower alkoxy-lower alkoxy, phenyloxy, methylenedioxy or ethylenedioxy, sulfamoyl, lower alkyl- or di-lower alkyl-sulfamoyl, guanidinosulfonyl, halo, carboxy, cyano or nitro.

Heteroaryl represents an aromatic group containing at least one heteroatom selected from nitrogen, oxygen and sulfur, and is mono- or bicyclic. Monocyclic heteroaryl includes 5 or 6 membered heteroaryl groups containing 1, 2, 3 or 4 heteroatoms selected from nitrogen, sulfur and oxygen. Bicyclic heteroaryl includes 9 or 10 membered fused-ring heteroaryl groups. Examples of heteroaryl include pyrrolyl, thiophenyl (i.e. thienyl), furyl, pyrazolyl, imidazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, and benzo fused derivatives of such monocyclic heteroaryl groups, such as indolyl, benzimidazolyl, benzothiophenyl or benzothiazolyl, quinolinyl, isoquinolinyl, quinazolinyl, or purinyl. Preferably, heteroaryl is pyrrolyl, thiophenyl, pyrazolyl, imidazolyl, triazolyl, tetrazolyl, oxazolyl, isoxazolyl, thiazolyl, pyridyl, pyridazinyl, pyrimidinyl, or pyrazinyl, in particular thiophenyl, benzothiophenyl, pyrazolyl, imidazolyl, benzimidazolyl, triazolyl, tetrazolyl, isoxazolyl, thiazolyl, benzothiazolyl, pyridyl, or pyridazinyl.

Heteroaryl is unsubstituted or substituted. Heteroaryl may be substituted by up to three substituents independently selected from lower alkyl, halo-lower alkyl, cycloalkyl-lower alkyl, hydroxy-lower alkyl, lower alkoxy-lower alkyl, aryloxy-lower alkyl, heteroaryloxy-lower alkyl, lower alkoxy-lower alkoxy-lower alkyl; aminoalkyl, wherein amino is unsubstituted or substituted by one or two substituents selected from lower alkyl, hydroxy-lower alkyl, alkoxy-lower alkyl, amino-lower alkyl, alkylcarbonyl, alkoxycarbonyl, amino-lower alkoxycarbonyl, lower alkoxy-lower alkoxycarbonyl and carbamoyl; optionally substituted alkenyl, optionally substituted alkinyl, cycloalkyl; aryl, heteroaryl, arylalkyl or heteroarylalkyl, wherein aryl or heteroaryl are unsubstituted or substituted by up to three substituents selected from lower alkyl, halo-lower alkyl, lower alkoxy, halogen, amino, cyano and nitro; hydroxy, lower alkoxy, halo-lower alkoxy, lower alkoxy-lower alkoxy, cycloalkyloxy, cycloalkyl-lower alkoxy, aryloxy, aryl-lower alkoxy, heteroaryloxy, heteroaryl-lower alkoxy, alkenyloxy, alkinyloxy, alkylmercapto, alkylsulfinyl, halo-lower alkylsulfinyl, alkylsulfonyl, arylsulfonyl, heteroarylsulfonyl, sulfamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, optionally substituted phenyl, optionally substituted phenyl-lower alkyl, optionally substituted heteroaryl, optionally substituted heteroaryl-lower alkyl, alkylcarbonyl, alkoxycarbonyl or carbamoyl, and wherein alkyl or lower alkyl in each case may be substituted by halogen, lower alkoxy, aryl, heteroaryl or optionally substituted amino, or wherein the two substituents on nitrogen form together with the nitrogen heterocyclyl; lower alkylcarbonyl, halo-lower alkylcarbonyl, optionally substituted phenylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; cyano, halogen, and nitro.

In particular, the substituents on heteroaryl may be independently selected from lower alkyl, halo-lower alkyl, cycloalkyl-lower alkyl, lower alkoxy-lower alkyl, lower alkoxy-lower alkoxy-lower alkyl, optionally substituted alkenyl, optionally substituted alkinyl, cycloalkyl, aryl, heteroaryl, hydroxy, lower alkoxy, cycloalkyloxy, alkenyloxy, alkinyloxy, alkyl-mercapto, alkylsulfinyl, halo-lower alkylsulfinyl, alkylsulfonyl, arylsulfonyl; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, alkylcarbonyl, alkoxycarbonyl or carbamoyl, and wherein alkyl or lower alkyl in each case may be substituted by lower alkoxy or optionally substituted amino, or wherein the two substituents on nitrogen form together with the nitrogen heterocyclyl; lower alkylcarbonyl, halo-lower alkylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, cycloalkyl-lower alkyl, hydroxy-lower alkyl, lower alkoxy-lower alkyl or cycloalkyl, or wherein the two substituents on nitrogen form together with the nitrogen heterocyclyl; cyano, halogen, and nitro.

Preferred substituents in heteroaryl are lower alkyl, halo-lower alkyl, lower alkoxy-lower alkyl, hydroxy, lower alkoxy, halo-lower alkoxy, lower alkoxy-lower alkoxy, methylenedioxy, halo, carboxy, cyano, nitro, or optionally substituted phenyl.

Alkenyl contains one or more, e.g. two or three, double bonds, and is preferably lower alkenyl, such as 1- or 2-butenyl, 1-propenyl, allyl or vinyl.

Alkinyl is preferably lower alkinyl, such as propargyl or acetylenyl.

In optionally substituted alkenyl or alkinyl, substituents are preferably lower alkyl, lower alkoxy, halo, optionally substituted aryl or optionally substituted heteroaryl, and are connected with a saturated or unsaturated carbon atom of alkenyl or alkinyl.

Heterocyclyl designates preferably a saturated, partially saturated or unsaturated, mono- or bicyclic ring containing 4-10 atoms comprising one, two or three heteroatoms selected from nitrogen, oxygen and sulfur, which may, unless otherwise specified, be carbon or nitrogen linked, wherein a ring nitrogen atom may optionally be substituted by a group selected from lower alkyl, amino-lower alkyl, aryl, aryl-lower alkyl and acyl, and a ring carbon atom may be substituted by lower alkyl, amino-lower alkyl, aryl, aryl-lower alkyl, heteroaryl, lower alkoxy, hydroxy or oxo. Examples of heterocyclyl are pyrrolidinyl, oxazolidinyl, thiazolidinyl, piperidinyl, morpholinyl, piperazinyl, dioxolanyl, tetrahydro-furanyl and tetrahydropyranyl.

Acyl designates, for example, alkylcarbonyl, cycloalkylcarbonyl, arylcarbonyl, aryl-lower alkylcarbonyl, or heteroarylcarbonyl. Lower acyl is preferably lower alkylcarbonyl, in particular propionyl or acetyl.

Hydroxyalkyl is especially hydroxy-lower alkyl, preferably hydroxymethyl, 2-hydroxyethyl or 2-hydroxy-2-propyl.

Cyanoalkyl designates preferably cyanomethyl and cyanoethyl.

Haloalkyl is preferably fluoroalkyl, especially trifluoromethyl, 3,3,3-trifluoroethyl or pentafluoroethyl.

Halogen is fluorine, chlorine, bromine, or iodine.

Lower alkoxy is especially methoxy, ethoxy, iso-propyloxy, or tert-butyloxy.

Arylalkyl includes aryl and alkyl as defined hereinbefore, and is e.g. benzyl, chlorobenzyl, methoxybenzyl, 1-phenethyl or 2-phenethyl.

Heteroarylalkyl includes heteroaryl and alkyl as defined hereinbefore, and is e.g. 2-, 3- or 4-pyridylmethyl, 1- or 2-pyrrolylmethyl, 2- or 3-thiophenylmethyl, 1-pyrazolylmethyl, or 1-imidazolylmethyl, or such compounds wherein heteroaryl is substituted, e.g. by chloro or methyl.

In substituted amino, the substituents are preferably those mentioned as substituents hereinbefore. In particular, substituted amino is alkylamino, dialkylamino, optionally substituted arylamino, optionally substituted arylalkylamino, lower alkylcarbonylamino, lower alkoxycarbonylamino or optionally substituted carbamoylamino.

Salts of compounds of the formula (I) are in particular pharmaceutically acceptable salts.

Such pharmaceutically acceptable salts are formed, for example, in compounds of formula (I) containing an acid function, e.g. carboxylic acid function, with organic or inorganic cations. Suitable inorganic cations are, for example, alkali cations, such as lithium, sodium or potassium cations, or earth alkali cations, such as magnesium, calcium, strontium and barium cations, or metallic cations, e.g. aluminium or transition metal cations. Preferred inorganic cations are sodium, potassium, magnesium and calcium cations. Suitable organic cations are, for example, tetrasubstituted ammonium cations, for example tetramethylammonium, protonated tri-, di- and mono-substituted amines, or ammonium. Suitable cations are derived by protonation from primary, secondary or tertiary amines containing, for example, lower alkyl, hydroxy-lower alkyl or hydroxy-lower alkoxy-lower alkyl groups, e.g., 2-hydroxyethylammonium, 2-(2-hydroxyethoxy)ethyldimethyl-ammonium, diethylammonium, di(2-hydroxyethyl)ammonium, trimethylammonium, triethylammonium, 2-hydroxyethyldimethylammonium, or di(2-hydroxyethyl)methyl-ammonium, also from correspondingly substituted cyclic secondary and tertiary amines, e.g., N-methylpyrrolidinium, N-methylpiperidinium, N-methylmorpholinium, N-2-hydroxy-ethylpyrrolidinium, N-2-hydroxyethylpiperidinium, or N-2-hydroxyethylmorpholinium, and the like. Preferred organic cations are 2-hydroxyethylammonium, diethylammonium or di(2-hydroxyethyl)ammonium.

In compounds of formula (I) containing a basic nitrogen function, pharmaceutically acceptable slats are formed with organic or inorganic acids. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid. For isolation or purification purposes it is also possible to use pharmaceutically unacceptable salts, for example picrates or perchlorates.

For therapeutic use, only pharmaceutically acceptable salts or free compounds are employed (where applicable in the form of pharmaceutical preparations), and these are therefore preferred.

In view of the close relationship between the novel compounds in free form and those in the form of their salts, any reference to the free compounds hereinbefore and hereinafter is to be understood as referring also to the corresponding salts, as appropriate and expedient. The compounds of formula (I), including their salts, are also obtainable in the form of hydrates, or their crystals can include, for example, the solvent used for crystallization, i.e. be present as solvates. Any reference to the free compounds hereinbefore and hereinafter is also to be understood as referring to the corresponding hydrates and solvates.

The compound of the formula (I) may be administered in the form of a pro-drug which is broken down in the human or animal body to give a compound of the formula (I). Examples of pro-drugs include in vivo hydrolysable esters and amides of a compound of the formula (I), for example esters or amides of naturally occurring α-amino acids or di- or tripeptides formed from naturally occurring α-amino acids.

Compounds of formula (I) are prepared by methods known in the art, in particular by condensation reactions of carboxylic acids or suitably activated acid derivatives, with amines or amine derivatives. If one or more other functional groups, for example carboxy, hydroxy or amino, are or need to be protected in starting compounds or intermediates, because they should not take part in the reaction, these are such protecting groups as are usually applied in the synthesis of amides, in particular peptide compounds. Particular syntheses schemes and reaction conditions are explained in detail in the Examples.

The compounds of formula (I) have valuable pharmacological properties. In particular, compounds of the invention and pharmaceutical compositions containing them are useful as neurotrypsin inhibitors.

Skeletal muscle atrophy is accompanied by a substantial loss of muscle strength and plays a major role in the pathogenesis of frailty and functional impairment that occurs with progressive old age. Weakness of the lower extremities has been implicated in a number of functional impairments, such as difficulties in rising from a chair or getting out of bed, slow speed of gait and other movements, and difficulties to maintain balance, resulting in falls and injuries. Skeletal muscle fiber loss has a negative effect on both the absolute strength that a muscle can develop and the speed with which a muscle can develop strength.

Increasing age is associated with a progressive decrease of the metabolic rate, which in turn has substantial physiological consequences, including a reduced tolerance against heat and cold as well as an increased propensity to develop obesity. Skeletal muscles comprise approximately 40% of the fat-free body mass and play an important homeostatic role in the body's metabolism. Therefore, a reduction of the skeletal muscle mass with increasing age is a major contributor to the decreased metabolic rate. By preventing the progressive fiber loss, the inhibition of neurotrypsin acts against these metabolic and physiological consequences.

Progressive loss of skeletal muscle mass and strength with age has been recognized as a major contributor to the gradual reduction of bone density observed with increasing age. Conversely, it is well known that the forces exerted on the bones by muscular activity stimulate bone formation. Thus, forces generated by muscle contraction are an important determinant of bone quality. Preventing muscle fiber loss by inhibition of peripheral neurotrypsin activity may therefore prevent or linder the adverse effects on skeletal muscle quality and indirectly antagonize progression of osteoporosis.

Beneficial effects of neurotrypsin inhibition may also be expected for skeletal muscle atrophies that occur in numerous clinical situations in which muscle wasting is an accompanying problem, including cancer, AIDS, and sepsis.

The concept of synapse tuning by reducing the anti-synaptic activity of neurotrypsin and, thereby, enhancing pro-synaptic activities at the expense of anti-synaptic activities, offers a wide range of applications in the area of disturbed cognitive brain functions. In particular, inhibition of neurotrypsin is beneficial in diseases and subclinical situations where synapse formation and the increase in the size and the strength of existing synapses is needed.

Inhibition of neurotrypsin is useful in the treatment of schizophrenia. Excessive neurotrypsin at the synapse drives synaptic pruning and, thus, generates a synaptic phenotype that is in accordance with the synaptic phenotype found in the brain of patients with schizophrenia. This experimental observation qualifies neurotrypsin as one of the factors that drive synaptic pruning. In a situation, where excessive synaptic pruning occurs due to the convergent action of multiple pruning-promoting factors, controlled and subtle partial inhibition of neurotrypsin diminishes the drive for synaptic pruning. This allows a recovery from the “schizophrenic synaptic phenotype” and results in the alleviation of the schizophrenic symptoms. The reduction of synapse numbers in the CNS of neurotrypsin-overexpressing mice indicates that inhibition of neurotrypsin results in a lesser degree of synaptic pruning and, thus, increased synaptic number and enhanced neuronal connectivity and communication. Compounds according to the invention inhibiting the enzymatic function of neurotrypsin are, therefore, useful in reverting the synaptic alterations in schizophrenia and in re-establishing normal synaptic structure and function and, thus, stop or shorten schizophrenic episodes and protect from new episodes.

Neurotrypsin is implicated in the development of cognitive disorders and mental retardation. A neurotrypsin gene knockout in humans causes mental retardation. Neurotrypsin gene duplications are implicated in the development of autism disorders. It is also probable that neurotrypsin plays a role in the development of Alzheimer disease (AD), as agrin fragments are found in senile plaques in AD patients (Van Horssen, J. et al., Acta Neuropathol. 2001, 102:604-14). It is well known that loss of lean muscular mass (i.e. sarcopenia) is accelerated especially in the earlier stages of Alzheimer disease (AD) and is associated with brain atrophy and cognitive impairment. A potential explanation for these observations is that AD and sarcopenia share common underlying pathogenic mechanisms (Burns et al., Arch Neurol. 2010, 67(4):428-433). It is well accepted that the dysfunction of acetylcholine containing neurons contributes substantially to the cognitive decline observed in those with advanced age and Alzheimer's disease (AD). This premise has since served as the basis for the majority of treatment strategies and drug development approaches for AD to date. Neurotrypsin is present at cholinergic synapses in the brain and in neuromuscular junctions. Over-production of neurotrypsin could contribute to the development and/or progression of both AD and sarcopenia.

Inhibition of neurotrypsin also supports cognitive enhancement in mild cognitive impairment and other clinical and subclinical states with reduced cognitive functions. Mild cognitive impairment, as well as other clinical and subclinical states of impaired cognitive functions have been found to be associated with evidence for cerebral tissue atrophy in several CNS areas. The reduction of synapse numbers in the CNS of neurotrypsin-overexpressing mice indicates that inhibition of neurotrypsin results in an increased synaptic number and enhanced neuronal connectivity and communication. Compounds according to the invention inhibiting the enzymatic function of neurotrypsin are, therefore, useful in reverting the synaptic alterations in all clinical and subclinical disorders in which a reduced number of synapses or a reduced function of synapses is involved, and in re-establishing normal synaptic structure and function. By this, pharmaceutical inhibition of neurotrypsin may improve cognitive functions in different states with reduced cognitive functions of heterogenous origins.

Based on these facts, the invention further relates to neurotrypsin inhibitors of formula (I) as described above and below for use in the treatment and/or prophylaxis of diseases caused by deficiency of synapses, for example skeletal muscle atrophy, schizophrenia and cognitive disturbance. Skeletal muscle atrophy to be treated is in particular so-called sarcopenia, i.e. a skeletal muscle atrophy due to old age, skeletal muscle atrophy accompanied by osteoporosis, and skeletal muscle atrophy due to muscle wasting associated with a severe disease, such as cancer, AIDS and sepsis, or also skeletal muscle atrophy as a consequence of immobilization and/or bed rest due to a severe injury or a severe disease. Schizophrenia to be treated is a disorder in the entire field of schizophrenia and schizophrenia-like disorders, comprising chronic schizophrenia, chronic schizo-affective disorders, unspecific disorders, acute and chronic schizophrenia of various symptomatologies, as for example severe, non-remitting “Kraepelinic” schizophrenia or the DSM-III-R-prototype of the schizophrenia-like disorders, episodic schizophrenic disorders, delusionic schizophrenia-like disorders, schizophrenia-like personality disorders, as for example schizophrenia-like personality disorders with mild symptomatics, schizotypic personality disorders, the latent forms of schizophrenic or schizophrenia-like disorders, and non-organic psychotic disorders. Furthermore, neurotrypsin inhibitors as described herein may be used as cognitive enhancers, for improving brain performance and for ameliorating learning and memory functions. Cognitive deficiencies to be treated are mild cognitive impairment, e.g. in a potential early stage of Alzheimer's disease, impairment of cognitive function without dementia in elderly people, and impairment of cognitive functions in patients with Alzheimer's disease, Parkinson's disease, multiple sclerosis, stroke, and head trauma.

Likewise the invention relates to the use of neurotrypsin inhibitors of formula (I) for the manufacture of a medicament for the treatment and/or prophylaxis of diseases caused by deficiency of synapses, as defined hereinbefore.

Furthermore the invention relates to the treatment and/or prophylaxis of diseases caused by deficiency of synapses, for example skeletal muscle atrophy, schizophrenia, Alzheimer disease and cognitive disturbance, which comprises administering a compound of formula (I) or a pharmaceutically acceptable salt thereof, in a quantity effective against said disease, to a warm-blooded animal requiring such treatment. The compounds of formula (I) can be administered as such or especially in the form of pharmaceutical compositions, prophylactically or therapeutically, preferably in an amount effective against the said diseases, to a warm-blooded animal, for example a human, requiring such treatment. In the case of an individual having a bodyweight of about 70 kg the daily dose administered is from approximately 0.05 g to approximately 5 g, preferably from approximately 0.25 g to approximately 1.5 g, of a compound of the present invention.

A compound of formula (I) can be administered alone or in combination with one or more other therapeutic agents, possible combination therapy taking the form of fixed combinations, or the administration of a compound of the invention and one or more other therapeutic agents being staggered or given independently of one another, or the combined administration of fixed combinations and one or more other therapeutic agents.

The present invention relates also to pharmaceutical compositions that comprise a compound of formula (I) as active ingredient and that can be used especially in the treatment of the diseases mentioned hereinbefore. Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions comprise the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration.

The present invention relates especially to pharmaceutical compositions that comprise a compound of formula (I), a tautomer, a prodrug or a pharmaceutically acceptable salt, or a hydrate or solvate thereof, and at least one pharmaceutically acceptable carrier.

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories, or capsules. Further dosage forms are, for example, ointments, creams, pastes, foams, tinctures, lip-sticks, drops, sprays, dispersions, etc. Examples are capsules containing from about 0.05 g to about 1.0 g active ingredient.

The pharmaceutical compositions of the present invention are prepared in a manner known per se, for example by means of conventional mixing, granulating, coating, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80® (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methyl-cellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinyl-pyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropyl-methylcellulose phthalate. Dyes or pigments may be added to the tablets or tablet coatings, for example for identification purposes or to indicate different doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxy-ethylene sorbitan fatty acid ester type, may also be added.

Pharmaceutical compositions suitable for rectal administration are, for example, suppositories that consist of a combination of the active ingredient and a suppository base. Suitable suppository bases are, for example, natural or synthetic triglycerides, paraffin hydrocarbons, polyethylene glycols or higher alkanols.

For parenteral administration, aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Solutions such as are used, for example, for parenteral administration can also be employed as infusion solutions.

Preferred preservatives are, for example, antioxidants, such as ascorbic acid, or microbicides, such as sorbic acid or benzoic acid.

With the groups of preferred compounds of formula (I) mentioned hereinafter, definitions of substituents from the general definitions mentioned hereinbefore may reasonably be used, for example, to replace more general definitions with more specific definitions or especially with definitions characterized as being preferred.

Preferred are compounds of the formula (I) wherein

### A is —CONR³R⁴;

W is hydrogen, hydroxy or carboxymethoxy;
Y is carboxy, methoxycarbonyl or 2H-tetrazol-5-yl;
R¹ is C₃-C₇-cycloalkyl, optionally substituted phenyl, optionally substituted benzo-C₅- or C₆-cycloalkyl or -cycloalkenyl, optionally substituted thiophenyl or benzothiophenyl, optionally substituted indol-2-yl, optionally substituted 1H-benz[d]imidazol-2-yl, optionally substituted 1,3-thiazol-2-yl, or thiophenylmethyl;
R² is hydrogen or methyl;
R³ is alkyl, methylamino-lower alkyl, carbamimidoyl-lower alkyl; C₅- or C₆-cycloalkylmethyl, optionally substituted benzyl, optionally substituted phenylethyl, optionally substituted thiophenylmethyl, C₃-C₇-cycloalkyl, bicyclo[2.2.1]heptyl, adamantyl, optionally substituted benzo-C₅- or -C₆-cycloalkyl or -cycloalkenyl, optionally substituted phenyl, 2-oxo-pyrrolidino or -piperidino; optionally substituted pyridyl, optionally substituted thiophenyl or benzothiophenyl, 1-methyl-1H-pyrazol-3-yl, pyridazin-4-yl, isoxazol-3-yl, or optionally substituted 1,3-thiazol-2-yl;
R⁴ is hydrogen, lower alkyl, carboxymethyl, lower alkoxycarbonylmethyl, dimethyl-carbamoylmethyl, hydroxy-lower alkyl or methoxy-lower alkyl;
or R³ and R⁴ together with the nitrogen atom, to which they are bound, are optionally substituted pyrrolidino, optionally substituted piperidino, tetrahydro-quinolyl or -isoquinolyl, morpholino, or optionally substituted piperazino;
and pharmaceutically acceptable salts thereof.

Also preferred are compounds of formula (I) wherein

### A is —NR⁵COR⁶;

W is hydrogen;
Y is carboxy, methoxycarbonyl or 2H-tetrazol-5-yl;
R¹ is optionally substituted C₃-C₇-cycloalkyl, optionally substituted phenyl, optionally substituted benzo-C₅- or C₆-cycloalkyl or -cycloalkenyl, optionally substituted thiophenyl or benzothiophenyl, optionally substituted 1H-benz[d]imidazol-2-yl, optionally substituted 1,3-thiazol-2-yl;
R² is hydrogen or methyl;
R⁵ is hydrogen; and
R⁶ is optionally substituted phenyl, optionally substituted thiophenyl or benzothiophenyl, optionally substituted 1,3-thiazol-2-yl, optionally substituted alkyl- or phenyl- or benzylamino, optionally substituted pyrrolidino, optionally substituted piperidino, or morpholino; with the proviso that R⁶ cannot be 2-thiophenyl if R¹ is 2-thiophenyl; or
R⁵ and R⁶ together with the nitrogen atom and the carbonyl group, to which they are bound, are optionally substituted 2-oxopyrrolidino, optionally substituted 2-oxopiperidino, or optionally substituted 2-oxo-oxazolidin-3-yl;
and pharmaceutically acceptable salts thereof.

Also preferred are compounds of formula (I) wherein

### A is —NHR⁷, —OR⁸ or —SR⁹;

W is hydrogen;
Y is carboxy, methoxycarbonyl or 2H-tetrazol-5-yl;
R¹ is optionally substituted phenyl, optionally substituted thiophenyl or benzothiophenyl, or optionally substituted 1,3-thiazol-2-yl;
R² is hydrogen; and
R⁷ is C₃-C₇-cycloalkyl, optionally substituted phenyl, optionally substituted benzyl or phenethyl, optionally substituted benzo-C₅- or C₆-cycloalkyl or -cycloalkenyl, optionally substituted alkylsulfonyl, or optionally substituted phenylsulfonyl;
R⁸ is optionally para- or meta-substituted benzyl, optionally substituted phenethyl, optionally substituted benzo-C₅- or C₆-cycloalkyl or -cycloalkenyl, optionally substituted alkylcarbamoylmethyl, dimethylcarbamoylmethyl, optionally substituted phenylcarbamoylmethyl, pyrrolidinocarbonylmethyl, piperidinocarbonylmethyl, morpholinocarbonylmethyl, or piperazinocarbonylmethyl; and
R⁹ is optionally substituted phenyl;
or A is —OR⁸; W is hydrogen; Y is carboxy; R¹ is optionally substituted benzimidazolyl-phenyl or chloro-substituted benzothiophenyl; R² is hydrogen; and R⁸ is phenyl;
and pharmaceutically acceptable salts thereof.

Also preferred are compounds of formula (I) wherein

### A is —CH₂NR¹⁰R¹¹;

W is hydrogen;
Y is carboxy, methoxycarbonyl or 2H-tetrazol-5-yl;
R¹ is optionally substituted phenyl, optionally substituted thiophenyl, optionally substituted benzothiophenyl, or optionally substituted 1,3-thiazol-2-yl;
R² is hydrogen;
R¹⁰ is arylcarbonyl, heteroarylcarbonyl or optionally substituted alkylcarbonyl;
R¹¹ is hydrogen or methyl; or
R¹⁰ and R¹¹ with the nitrogen atom, to which they are bound, are optionally substituted 2-oxopyrrolidino, optionally substituted 2-oxopiperidino or optionally substituted 2-oxo-oxazolidin-3-yl;
and pharmaceutically acceptable salts thereof.

Also preferred are compounds of formula (I) wherein

### A is —(CH₂)₂—R¹², —CH═CH—R¹² or —C≡C—R¹²;

W is hydrogen;
Y is carboxy, methoxycarbonyl or 2H-tetrazol-5-yl;
R¹ is optionally substituted benzimidazolyl-phenyl or chloro-substituted benzothiophenyl R² is hydrogen; and
R¹² is aryl or aryl-lower alkyl;
and pharmaceutically acceptable salts thereof.

Also preferred are compounds of formula (I) wherein

A is phenyl, halo-, methoxy- or cyanophenyl, thiophenyl, or halo- or carbamoyl-thiophenyl;
W is hydrogen;
Y is carboxy, methoxycarbonyl or 2H-tetrazol-5-yl;
R¹ is optionally substituted benzimidazolyl-phenyl or chloro-substituted benzothiophenyl; and
R² is hydrogen;
and pharmaceutically acceptable salts thereof.

Also preferred are compounds of formula (I) wherein

A is optionally substituted 1,2,3-triazol-4-yl;
W is hydrogen;
Y is carboxy, methoxycarbonyl or 2H-tetrazol-5-yl;
R¹ is optionally substituted benzimidazolyl-phenyl, optionally substituted thiophenyl, or optionally substituted benzothiophenyl; and
R² is hydrogen;
and pharmaceutically acceptable salts thereof.

More preferred are compounds of formula (I) wherein

### A is —CONR³R⁴;

W is hydrogen, hydroxy or carboxymethoxy;
Y is carboxy, methoxycarbonyl or 2H-tetrazol-5-yl;
R¹ is C₃-C₇-cycloalkyl;
optionally substituted phenyl with one to three substituents, wherein the substituents are selected from the group consisting of lower alkyl, hydroxy, lower alkoxy, halo, cyano, halobenzyl, thiophen-2-yl, and 1H-benzimidazol-2-yl optionally substituted at nitrogen by methyl or carboxymethyl and at the benzo residue by carboxy, chloro or dichloro;
2-indanyl or 2-indenyl, optionally substituted by chloro and/or phenyl;
2- or 3-thiophenyl, optionally substituted by lower alkyl, propen-1-yl, vinyl, halo, cyano, phenyl, halophenyl, methoxyphenyl, ethylenedioxyphenyl, or 4-(4-methylpiperazin-1-ylmethyl);
benzo[b]thiophen-2- or -3-yl, optionally substituted by halo, dihalo or ethylenedioxy; 1H-indol-2-yl, optionally substituted by chloro and/or phenyl;
1H-benz[d]imidazol-2-yl, optionally substituted by halo, dihalo, carboxy or methoxycarbonyl;
1,3-thiazol-2-yl, optionally substituted by lower alkyl, halo, or acetoxy, or with an annullated cyclopenta, benzo, or halobenzo ring;
or 2-thiophenylmethyl;
R² is hydrogen or methyl;
R³ is alkyl, in particular lower alkyl or n-heptyl;
methylamino-lower alkyl, carbamimidoyl-lower alkyl; cyclohexylmethyl, optionally halogenated or carbamimidoylated benzyl, optionally halogenated phenylethyl; thiophenylmethyl, optionally substituted by halo or chlorophenyl, benzo[b]thiophenylmethyl;
C₃-C₇-cycloalkyl, bicyclo[2.2.1]heptyl, adamantyl, indanyl, tetrahydronaphthalenyl;
optionally substituted phenyl with one to three substituents, wherein the substituents are selected from the group consisting of halo, cyano, lower alkyl, hydroxy-lower alkyl, phenyl-hydroxy-lower alkyl, optionally halogenated benzyl, methylamino-lower alkyl, dimethyl-amino-lower alkyl, carbamidoyl-lower alkyl, hydroxy, lower alkoxy, hydroxy-lower alkoxy, phenoxy, benzyloxy, pyridoxy, phenyl, carboxy, phenylcarbonyl, carbamimidoyl, methyl-sulfonyl, N,N-dimethylsulfamoyl, N-carbamimidoylsulfamoyl, and 5-oxo-2,5-dihydro-1,2,4-oxadiazol-3-yl;
2-oxo-pyrrolidino; optionally halogenated pyridyl;
2- or 3-thiophenyl, optionally substituted by chloro, cyano or vinyl;
1-methyl-1H-pyrazol-3-yl, pyridazin-4-yl, isoxazol-3-yl, 1,3-thiazol-2-yl, optionally halogenated 1,3-thiazol-2-yl or benzo[d]-1,3-thiazol-2-yl;
R⁴ is hydrogen, lower alkyl, carboxymethyl, ethoxycarbonylmethyl, dimethylcarbamoylmethyl, hydroxy-lower alkyl or methoxy-lower alkyl;
or R³ and R⁴ together with the nitrogen atom, to which they are bound, are pyrrolidino, optionally substituted by keto, phenyl, chlorophenyl or phenyoxy; piperidino, optionally substituted by phenoxy, optionally substituted phenyl wherein the substituents on phenyl are fluoro, chloro, hydroxy, methoxy, trifluoromethyl or methyl, hydroxy, optionally substituted benzyl wherein the substituents on benzyl are fluoro, chloro or methoxy; tetrahydro-quinolyl or -isoquinolyl; morpholino; or piperazino, optionally substituted by keto, 4-benzyl or 4-tert-butyl;
and A and Y are in position 2 and 3, 2 and 4, 2 and 5, 2 and 6, 3 and 2, 3 and 4, 3 and 5, 3 and 6, 4 and 2, and 4 and 3 of the phenyl ring, respectively;
and pharmaceutically acceptable salts thereof.

Also more preferred are compounds of formula (I) wherein

### A is —NR⁵COR⁶;

W is hydrogen;
Y is carboxy, methoxycarbonyl or 2H-tetrazol-5-yl;
R¹ is optionally substituted phenyl, optionally substituted thiophenyl, or optionally substituted 1,3-thiazol-2-yl;
R² is hydrogen;
R⁵ is hydrogen; and
R⁶ is optionally substituted phenyl, optionally substituted thiophenyl, optionally substituted 1,3-thiazol-2-yl, optionally substituted phenylamino, optionally substituted pyrrolidino or optionally substituted piperidino; with the proviso that R⁶ cannot be 2-thiophenyl if R¹ is 2-thiophenyl; or
R⁵ and R⁶ together with the nitrogen atom and the carbonyl group, to which they are bound, are optionally substituted 2-oxopyrrolidino, optionally substituted 2-oxopiperidino, or optionally substituted 2-oxo-oxazolidin-3-yl;
and A and Y are in position 2 and 3, 2 and 4, 2 and 5, 2 and 6, 3 and 2, 3 and 4, 3 and 5, 3 and 6, 4 and 2, and 4 and 3 of the phenyl ring, respectively;
and pharmaceutically acceptable salts thereof.

Also more preferred are compounds of formula (I) wherein

### A is —NHR⁷, —OR⁸ or —SR⁹;

W is hydrogen;
Y is carboxy, methoxycarbonyl or 2H-tetrazol-5-yl;
R¹ is optionally substituted phenyl, optionally substituted thiophenyl or benzothiophenyl, or optionally substituted 1,3-thiazol-2-yl;
R² is hydrogen;
R⁷ is optionally substituted benzyl or phenethyl, optionally substituted phenyl, indanyl, 1,2,3,4-tetrahydronaphthalenyl, or optionally substituted phenylsulfonyl;
R⁸ is optionally para- or meta-substituted benzyl or optionally substituted phenylcarbamoylmethyl; and
R⁹ is optionally substituted phenyl;
or A is —OR⁸; W is hydrogen; Y is carboxy; R¹ is chloro-substituted benzothiophenyl; R² is hydrogen; and R⁸ is phenyl;
and A and Y are in position 2 and 4, 2 and 5, 3 and 4, 3 and 5, 3 and 6, 4 and 2, and 4 and 3 of the phenyl ring, respectively;
and pharmaceutically acceptable salts thereof.

Also more preferred are compounds of formula (I) wherein

### A is —CH₂NR¹⁰R¹¹;

W is hydrogen;
Y is carboxy, methoxycarbonyl or 2H-tetrazol-5-yl;
R¹ is optionally substituted phenyl, optionally substituted thiophenyl or benzothiophenyl, or optionally substituted 1,3-thiazol-2-yl;
R² is hydrogen;
R¹⁰ is arylcarbonyl or optionally substituted alkylcarbonyl;
R¹¹ is hydrogen; or
R¹⁰ and R¹¹ with the nitrogen atom, to which they are bound, are optionally substituted 2-oxopyrrolidino, optionally substituted 2-oxopiperidino or optionally substituted 2-oxo-oxazolidin-3-yl,
and A and Y are in position 2 and 4, 2 and 5, 3 and 4, 3 and 5, 3 and 6, 4 and 2, and 4 and 3 of the phenyl ring, respectively;
and pharmaceutically acceptable salts thereof.

Also more preferred are compounds of formula (I) wherein

### A is —(CH₂)₂—R¹², —CH═CH—R¹² or —C≡C—R¹²;

W is hydrogen;
Y is carboxy, methoxycarbonyl or 2H-tetrazol-5-yl;
R¹ is optionally substituted benzimidazolyl-phenyl or chloro-substituted benzothiophenyl;
R² is hydrogen; and
R¹² is optionally substituted phenyl or optionally substituted benzyl;
and A and Y are in position 2 and 4, 2 and 5, 3 and 4, 3 and 5, 3 and 6, 4 and 2, and 4 and 3 of the phenyl ring, respectively;
and pharmaceutically acceptable salts thereof.

Also more preferred are compounds of formula (I) wherein

A is phenyl, o-chloro, p-chloro-, p-methoxy- or p-cyanophenyl, 2- or 3-thiophenyl, or 5-chloro- or 5-carbamoyl-2-thiophenyl;
W is hydrogen;
Y is carboxy, methoxycarbonyl or 2H-tetrazol-5-yl;
R¹ is optionally substituted benzimidazolyl-phenyl or chloro-substituted benzothiophenyl; and
R² is hydrogen;
and A and Y are in position 2 and 4, 2 and 5, 3 and 4, 3 and 5, 3 and 6, 4 and 2, and 4 and 3 of the phenyl ring, respectively;
and pharmaceutically acceptable salts thereof.

Also more preferred are compounds of formula (I) wherein

A is 1,2,3-triazol-4-yl, 1-benzyl-, 1-p-chlorobenzyl-, 1-(2,6-difluorobenzyl)-, 1-p-chlorophenyl-, 1-p-chloro-m-trifluoromethyl-phenyl- or 1-(5-chloro-2-thiophenyl)-1,2,3-triazol-4-yl;
W is hydrogen;
Y is carboxy, methoxycarbonyl or 2H-tetrazol-5-yl;
R¹ is optionally substituted benzimidazolyl-phenyl, optionally substituted thiophenyl, or optionally substituted benzothiophenyl; and
R² is hydrogen;
and A and Y are in position 2 and 4, 2 and 5, 3 and 4, 3 and 5, 3 and 6, 4 and 2, and 4
and 3 of the phenyl ring, respectively;
and pharmaceutically acceptable salts thereof.

Particularly preferred are compounds of formula (I) wherein

### A is —CONR³R⁴;

W is hydrogen, hydroxy or carboxymethoxy;
Y is carboxy, methoxycarbonyl or 2H-tetrazol-5-yl;
R¹ is cyclopropyl, cyclopentyl;
optionally substituted phenyl, in particular phenyl, o-methyl-, o-hydroxy-, o-methoxy-, o-fluoro-, or o-chloro-phenyl, m-chloro- or m-cyano-phenyl, p-chloro-, p-cyano-, p-(p-chlorobenzyl)-phenyl, 4-(1H-benzimidazol-2-yl)phenyl or 4-(1H-benzimidazol-2-yl)-3-hydroxy-phenyl, wherein benzimidazolyl is optionally substituted at nitrogen by methyl or carboxymethyl and at the benzo residue by carboxy, chloro or dichloro, for example as 5-chloro-, 5-carboxy- or 5,6-dichloro-1H-benzimidazol-2-yl, 2,6-difluorophenyl, 3-chloro-4-cyanophenyl, 2-hydroxy-4-(2-thiophenyl)phenyl or 3,5-dibromo-2-hydroxyphenyl;
2-indanyl; optionally substituted 1H-2-indenyl, in particular 1H-2-indenyl or 3-chloro-1H-2-indenyl;
optionally substituted 2-thiophenyl, in particular 2-thiophenyl, 3-bromo- or 3-chloro-thiophen-2-yl; 4-methyl-, 4-chloro-, 4-(4-methylpiperazin-1-ylmethyl)- or 3-chloro-4-(4-methylpiperazin-1-ylmethyl)-thiophen-2-yl; 5-methyl-, 5-chloro-, 5-cyano-, 5-phenyl-, 5-p-fluorophenyl-, 5-p-chlorophenyl-, 5-p-methoxyphenyl- or 5-(3,4-ethylenedioxyphenyl)-thiophen-2-yl; 3-chloro-4,5-divinyl-thiophen-2-yl, or 3-chloro-5-(propen-1-yl)-4-vinyl-thiophen-2-yl;
optionally substituted benzo[b]thiophen-2-yl, in particular benzo[b]thiophen-2-yl, 3- or 6-chloro-, 3,6-dichloro- or 5,6-ethylenedioxy-3-chloro-benzo[b]thiophen-2-yl; 6-chloro-benzo[b]thiophen-3-yl;
optionally substituted indol-2-yl, in particular 1H-indol-2-yl, 3-phenyl-1H-indol-2-yl, or 5-chloro-3-phenyl-1H-indol-2-yl;
optionally substituted 1H-benz[d]imidazol-2-yl, in particular 1H-benz[d]imidazol-2-yl, 5- or 6-chloro, carboxy or methoxycarbonyl-1H-benz[d]imidazol-2-yl or 5,6-dichloro-1H-benz[d]imidazol-2-yl;
optionally substituted 1,3-thiazol-2-yl, in particular 1,3-thiazol-2-yl, 5-chloro-, 5-acetoxy- or 4-methyl-1,3-thiazol-2-yl, 4H-cylopenta-1,3-thiazol-2-yl, benzo[d]thiazol-2-yl or 5-fluorobenzo[d]thiazol-2-yl;
or 2-thiophenylmethyl;
R² is hydrogen or methyl;
R³ is alkyl, in particular methyl, iso-butyl, iso-pentyl and n-heptyl;
4-methylaminobutyl, 4-carbamimidoylbutyl, 3-carbamimidoylpropyl, 2-carbamimidoylethyl;
cyclohexylmethyl, benzyl, p-chlorobenzyl, 3- or 4-carbamimidoylbenzyl, 2,6-difluorobenzyl, 1- or 2-phenylethyl, 1- or 2-p-chlorophenylethyl;
5-chloro-thiophen-2-ylmethyl, 5-p-chlorophenyl-thiophen-2-ylmethyl, benzo[b]thiophen-2-ylmethyl;
cyclopentyl, cyclohexyl, cycloheptyl, bicyclo[2.2.1]hept-2-yl, adamantyl, indan-2-yl, tetralin-1-yl;
optionally substituted phenyl, in particular 2-, 3- or 4-chlorophenyl, 2- or 4-fluorophenyl, 2-, 3- or 4-bromophenyl, 2-fluoro-4-chlorophenyl, 3- or 4-cyanophenyl, 3-hydroxymethyl-phenyl, 3-hydroxymethyl-4-chloro-phenyl, 4-benzylphenyl, 4-p-chlorobenzylphenyl, 4-α-hydroxybenzyl)phenyl, 4-lower alkylphenyl, such as 4-methyl-, 4-iso-propyl, 4-n-butyl-, 4-iso-butyl- or 4-tert-butyl-phenyl, 4-dimethylaminomethyl-phenyl, 4-methylamino-lower alkyl-phenyl, such as 4-methylamino-pentyl-, -butyl-, -propyl- or -ethyl-phenyl, 4-carbamimidoyl-lower alkyl-phenyl, such as 4-carbamimidoyl-butyl-, -propyl- or -ethyl-phenyl, 2-hydroxyphenyl, 4-methoxyphenyl, 4-n-propoxyphenyl, 4-tert-butoxyphenyl, 4-(2-hydroxyethoxy)phenyl, 4-biphenylyl, 3- or 4-phenoxyphenyl, 4-benzyloxyphenyl, 4-p-chlorobenzyloxy-phenyl, 4-(3-pyridoxy)phenyl, 4-carboxyphenyl, 4-phenylcarbonyl-phenyl, 3- or 4-carbamidoyl-phenyl, 4-methylsulfonyl-phenyl, 4-(N,N-dimethylsulfamoyl)phenyl, 4-(N-carbamimidoylsulfamoyl)phenyl, or 4-(5-oxo-2,5-dihydro-1,2,4-oxadiazol-3-yl)phenyl; 2-oxo-pyrrolidino; 2-, 3- or 4-pyridyl, 5-chloro-2-pyridyl;
optionally substituted thiophen-2-yl, in particular 3- or 5-chloro-thiophen-2-yl, 3-cyano-thiophen-2-yl, or 4,5-divinyl-thiophen-2-yl; 1-methyl-1H-pyrazol-3-yl, pyridazin-4-yl, isoxazol-3-yl;
1,3-thiazol-2-yl, 5-chloro-1,3-thiazol-2-yl, 4-methyl-1,3-thiazol-2-yl, 5-acetyl-4-methyl-1,3-thiazol-2-yl, benzo[d]-1,3-thiazol-2-yl, 6-chloro- or 6-fluoro-benzo[d]-1,3-thiazol-2-yl; cyclopenta[d]-1,3-diazol-2-yl, or 1,3,4-thiadiazol-2-yl; and
R⁴ is hydrogen, lower alkyl, in particular methyl or iso-butyl, carboxymethyl ethoxycarbonylmethyl, dimethylcarbamoylmethyl, hydroxy-lower alkyl, in particular 2-hydroxyethyl, 3-hydroxypropyl, 2-hydroxy-2-propyl or 2,3-dihydroxypropyl, or methoxy-lower alkyl, in particular 2-methoxyethyl or 3-methoxypropyl;
or R³ and R⁴ together with the nitrogen atom, to which they are bound, are optionally substituted pyrrolidino, in particular 2-keto-, 3-phenyl, 3-p-chlorophenyl- or 3-phenoxy-pyrrolidino; optionally substituted piperidino, in particular 3- or 4-phenoxy-piperidino, 4-phenyl-, 4-p-fluorophenyl-, 4-p-chlorophenyl-, 4-p-hydroxyphenyl-, 4-p-methoxyphenyl- or 4-(2,6-dimethylphenyl)-piperidino, 4-benzyl-, 4-p-fluorobenzyl-, 4-p-chlorobenzyl- or 4-p-hydroxybenzyl-piperidino, 4-hydroxy-4-phenyl-, 4-hydroxy-4-p-chlorophenyl-, 4-hydroxy-4-benzyl- or 4-hydroxy-4-(4-chloro-3-trifluoromethylphenyl)piperidino; 1,2,3,4-tetrahydro-quinolyl, 1,2,3,4-tetrahydroisoquinolyl, morpholino, or optionally substituted piperazino, in particular 3-keto-, 4-benzyl- or 4-tert-butyl-piperazino;
and pharmaceutically acceptable salts thereof.

Also particularly preferred are compounds of formula (I) wherein

### A is —NR⁵COR⁶;

W is hydrogen;
Y is carboxy, methoxycarbonyl or 2H-tetrazol-5-yl;
R¹ is optionally substituted phenyl, in particular phenyl or p-chlorophenyl; optionally substituted thiophenyl, in particular 2-thiophenyl, 3-chloro-, 5-chloro-, 5-phenyl- or 5-p-chlorophenyl-thiophen-2-yl;
optionally substituted benzothiophenyl, in particular benzo[b]thiophen-2-yl or 3-chloro-benzo[b]thiophen-2-yl; or
optionally substituted 1,3-thiazol-2-yl, in particular 1,3-thiazol-2-yl or 5-chloro-1,3-thiazol-2-yl
R² is hydrogen;
R⁵ is hydrogen; and
R⁶ is optionally substituted phenyl, in particular 4-chlorophenyl, optionally substituted thiophenyl, in particular 2-thiophenyl or 3- or 5-chloro-2-thiophenyl, optionally substituted benzothiophenyl, in particular benzo[b]thiophen-2-yl or 3-chlorobenzo[b]thiophen-2-yl, or
1,3-thiazol-2-yl; p-chlorophenylamino or 4-phenylpiperidino; with the proviso that R⁶ cannot be 2-thiophenyl if R¹ is 2-thiophenyl; or
R⁵ and R⁶ together with the nitrogen atom and the carbonyl group, to which they are bound, are 4-chlorophenyl-2-oxopyrrolidino, 4-chlorophenyl-2-oxopiperidino, or 5-benzyl- or 5-(4-chlorophenyl)-2-oxo-oxazolidin-3-yl;
and pharmaceutically acceptable salts thereof.

Also particularly preferred are compounds of formula (I) wherein

### A is —NHR⁷;

W is hydrogen;
Y is carboxy;
R¹ is 3-chloro-benzo[b]thiophen-2-yl;
R² is hydrogen; and
R⁷ is p-chlorobenzyl, p-chlorophenyl, indan-2-yl, 1,2,3,4-tetrahydronaphthalen-1-yl, 1-(4-chlorophenyl)ethyl, or p-chlorophenylsulfonyl;
and pharmaceutically acceptable salts thereof.

Also particularly preferred are compounds of formula (I) wherein

### A is —OR⁸;

W is hydrogen;
Y is carboxy or methoxycarbonyl;
R¹ is 4-(1H-benzimidazol-2-yl)phenyl, wherein benzimidazolyl is optionally substituted at the benzo residue by dichloro, for example as 5,6-dichloro-1H-benzimidazol-2-yl, or optionally substituted benzo[b]thiophen-2-yl, in particular benzo[b]thiophen-2-yl or 3-chloro-benzo[b]thiophen-2-yl;
R² is hydrogen; and
R⁸ is p-chlorobenzyl or p-chlorophenylcarbamoylmethyl, preferably p-chlorophenylcarbamoylmethyl;
or A is —OR⁸; W is hydrogen; Y is carboxy; R¹ is 3-chloro-benzo[b]thiophen-2-yl; R² is hydrogen; and R⁸ is phenyl;
and pharmaceutically acceptable salts thereof.

Also particularly preferred are compounds of formula (I) wherein

### A is —SR⁹;

W is hydrogen;
Y is carboxy;
R¹ is 3-chloro-benzo[b]thiophen-2-yl;
R² is hydrogen; and
R⁹ is p-chlorophenyl;
and pharmaceutically acceptable salts thereof.

Also particularly preferred are compounds of formula (I) wherein

### A is —CH₂NR¹⁰R¹¹;

W is hydrogen;
Y is carboxy;
R¹ is 3-chloro-benzo[b]thiophen-2-yl;
R² is hydrogen;
R¹⁰ is benzoyl; and
R¹¹ is hydrogen; or
R¹⁰ and R¹¹ with the nitrogen atom, to which they are bound, are 2-oxopyrrolidino or 5-(4-chlorophenyl)-2-oxo-oxazolidin-3-yl;
and pharmaceutically acceptable salts thereof.

Also particularly preferred are compounds of formula (I) wherein

### A is —(CH₂)₂—R¹², —CH═CH—R¹² or —C≡C—R¹²;

W is hydrogen;
Y is carboxy;
R¹ is 3-chloro-benzo[b]thiophen-2-yl;
R² is hydrogen; and
R¹² is phenyl, p-chlorophenyl or benzyl;
and pharmaceutically acceptable salts thereof.

Also particularly preferred are compounds of formula (I) wherein

A is phenyl, o-chloro, p-chloro-, p-methoxy- or p-cyanophenyl, 2- or 3-thiophenyl, 5-chloro- or 5-carbamoyl-2-thiophenyl, 1,2,3-triazol-4-yl, 1-benzyl-, 1-p-chlorobenzyl-, 1-(2,6-difluorobenzyl)-, 1-p-chlorophenyl-, 1-p-chloro-m-trifluoromethyl-phenyl- or 1-(5-chloro-2-thiophenyl)-1,2,3-triazol-4-yl;
W is hydrogen;
Y is carboxy, methoxycarbonyl or 2H-tetrazol-5-yl;
R¹ is 4-(1H-benzimidazol-2-yl)phenyl, wherein benzimidazolyl is optionally substituted at the benzo residue by dichloro, for example as 5,6-dichloro-1H-benzimidazol-2-yl, or chloro-substituted benzo[b]thiophen-2-yl, in particular 3-chloro-benzo[b]thiophen-2-yl; and
R² is hydrogen;
and pharmaceutically acceptable salts thereof.

Most preferred are the compounds of the Examples, in particular the compounds of Examples 1, 5, 16, 18, 23, 24, 26, 27, 33, 35, 36, 37, 38, 40, 42, 43, 44, 45, 46, 47, 48, 49, 51, 59, 62, 65, 69, 73, 75, 84, 85, 87, 90, 91, 99, 101, 103, 107, 108, 110, 112, 113, 114, 115, 116, 117, 118, 119, 120, 121, 122, 123, 124, 125, 126, 129, 130, 131, 132, 133, 134, 135, 137, 138, 139, 140, 141, 142, 143, 148, 151, 156, 157, 158, 159, 160, 161, 162, 163, 164, 165, 188, 194, 227, 228, 229, 249, 250, 255, 257, 258, 261, 262, 263, 266, 267, 273, 274, 275, 276, 277, 279, 280, 283, 287, 288, 290, 291, 293, 301, 302, 303, 304, 305, 306, and 311; more particularly the compounds of Examples 5, 23, 24, 26, 27, 33, 35, 36, 37, 38, 40, 42, 43, 44, 45, 46, 47, 48, 51, 69, 73, 84, 85, 87, 90, 103, 107, 108, 110, 112, 113, 114, 115, 116, 117, 118, 119, 120, 121, 123, 124, 125, 126, 129, 131, 133, 134, 135, 137, 140, 141, 142, 143, 148, 151, 156, 157, 158, 160, 161, 162, 163, 164, 229, 249, 250, 255, 257, 263, 266, 267, 275, 277, 280, 283, 287, 290, 291, 302, 304, 305, 306, and 311.

### EXAMPLES

### Abbreviations

DCM=dichloromethane
DIEA=diisopropylethylamine
DMF=dimethylformamide
EDCl=1-ethyl-3-(3-dimethylaminopropyl)carbodiimide hydrochloride
eq.=equivalent
Et₂O=diethyl ether
EtOH=ethanol
h=hours
HOBt=1-hydroxy-1H-benzotriazole
NMR=nuclear magnetic resonance
MeOH=methanol
min=minutes
MP=melting point
MS=mass spectrum
RT=room temperature
THF=tetrahydrofuran
TFA=trifluoroacetic acid
TMS=tetramethylsilane

¹H NMR spectra were recorded on Bruker Avance II 300M Hz or Bruker AVANCE 400 MHz instruments. The ¹H chemical shifts are reported in ppm relative to TMS as an internal standard. Mass spectra were run on a MDS SCIEX-API-2000, UPLC-MS: Waters Acquity 2996 PDA/Micromass ZQ, or HPLC-MS: Waters Acquity 2996 PDA/Micromass ZQ instrument.

### Preparation 1: 4-(5,6-Dichloro-1H-benzo[d]imidazol-2-yl)benzoic acid

In a 1 L Erlenmeyer flask, 4-formylbenzoic acid (10 g, 66.6 mmol) was dissolved in 400 ml of EtOH, then a solution of NaHSO₃ (6.93 g, 66.6 mmol) in 45 ml of water was added and the mixture was stirred while cooling in an ice bath for about 1 h. The white precipitate was collected and washed with a little amount of EtOH. After drying, 15.8 g of sodium (4-carboxyphenyl)-hydroxy-methanesulfonate (62.3 mmol, 94% yield) were obtained.

In a 250 ml round-bottomed flask equipped with a condenser, a mixture of sodium (4-carboxyphenyl)-hydroxy-methanesulfonate (6.77 g, 26.6 mmol) and 4,5-dichlorobenzene-1,2-diamine (4.71 g, 26.6 mmol) in 60 ml of DMF was stirred at 170° C. overnight. The mixture was cooled to RT, poured into ice and acidified with 2 N HCl. The black precipitate was collected by filtration. The solid was stirred in 400 ml of methanol and the insoluble tarry portion was discarded by filtration through a Celite pad. The solvent was evaporated under vacuum and the resulting dark brown solid was washed with DCM to obtain 7.0 g of pure 4-(5,6-dichloro-1H-benzo[d]imidazol-2-yl)benzoic acid (22.79 mmol, 86% yield) as a pale brown solid.

### Preparation 2: Methyl 4-carboxy-3-nitrobenzoate

A solution of 2-nitroterephthalic acid (10 g) in methanol (150 ml) was treated with 5 ml conc. sulfuric acid and refluxed for 4 h. After cooling, the reaction mixture was poured into 200 ml of saturated cold sodium bicarbonate solution and washed with ethyl acetate. The aqueous layer was adjusted to pH 5-6 and extracted with ethyl acetate, and washed with water and brine. The organic layer was dried and evaporated under vacuum to afford 5.1 g (48%) of the title compound.

### Preparation 3: t-Butyl 5-methoxycarbonyl-2-nitrobenzoate

A mixture of methyl 4-carboxy-3-nitrobenzoate (5 g) in dioxane, a catalytic amount of sulfuric acid and condensed 2-methylpropene at −15° C. in a sealed tube was stirred at room temperature for 48 h. The reaction mixture was poured into cold 10% NaOH solution and extracted with ethyl acetate, and washed with water and brine. Concentration of the dried organic layer afforded 4.6 g (74%) of the title compound.

### Preparation 4: 4-Nitro-1,3-benzenedicarboxylic acid

To a stirred mixture of 4-nitro-m-xylene and water, KMnO₄ (5 eq.) was added portionwise during 30 minutes. The reaction was heated at 90° C. for 4-5 h, cooled and filtered through a Celite pad. The pH of the filtrate was adjusted to pH 2 using conc. H₂SO₄. The precipitate was collected by suction filtration and dried, to yield 45% of the title compound.

### Preparation 5: Methyl 3-carboxy-4-nitrobenzoate

A solution of 4-nitro-1,3-benzenedicarboxylic acid (10 g) in methanol (150 ml) was treated with 5 ml conc. sulfuric acid and refluxed for 4 h. After cooling, the solvent was removed under vacuum and the residue was diluted with 200 ml of cold saturated solution of sodium bicarbonate and extracted with ethyl acetate. The aqueous layer was adjusted the pH 5-6 and extracted with ethyl acetate, washed with water and brine, dried over sodium sulfate and concentrated under vacuum to afford 45% of the title compound.

### Preparation 6: Methyl 4-bromo-2-[(3-chloro-benzo[b]thiophene-2-carboxamido)benzoate

A mixture of methyl 2-amino-4-bromo-benzoate (2.92 g, 12.7 mmol), 3-chlorobenzo[b]-thiophene-2-carbonyl chloride (3.26 g, 14.1 mmol), pyridine (1.14 ml, 14.1 mmol) and dry THF (80 ml) was stirred at room temperature overnight. The solvent was reduced to half the original volume and the precipitate was collected and washed with acetone. The title compound was obtained as a white solid (3.73 g, 69%). ¹H NMR (300 MHz, DMSO-d₆) δ 11.70 (s, 1H), 8.77 (d, 1H), 8.15-8.25 (m, 1H), 7.96-8.04 (m, 1H), 7.97 (d, 1H), 7.59-7.74 (m, 2H), 7.52 (dd, 1H), 3.91 (s, 3H) ppm. MS: m/z=424 [MH]⁺

### Preparation 7: Methyl 4-amino-2-(3-chloro-benzo[b]thiophene-2-carboxamido)benzoate

A mixture of 2-amino-4-nitro-benzoic acid methyl ester (0.79 g, 4.0 mmol), 3-chloro-benzo[b]thiophene-2-carbonyl chloride (0.98 g, 4.23 mmol), pyridine (0.341 ml, 4.23 mmol) and dry THF (30 ml) was stirred at room temperature overnight. The precipitate was recovered and washed with plenty of ethyl ether, then dried under vacuum at 40° C. over night. A portion of this compound (1 g, 2.56 mmol) was added in one portion to a suspension of PtO₂.x H₂O (0.30 g) in methanol (200 ml). The mixture was hydrogenated at 30 psi overnight. The mixture was filtered through a Celite pad, and the solvent was evaporated under vacuum to obtain the title compound as a white solid (0.90 g, 98%).

MS: m/z=361 [MH]⁺

### Example 1

### 2-(3-Chlorobenzo[b]thiophene-2-carboxamido)-4-(4-chlorophenylcarbamoyl)-benzoic acid

### Method 1

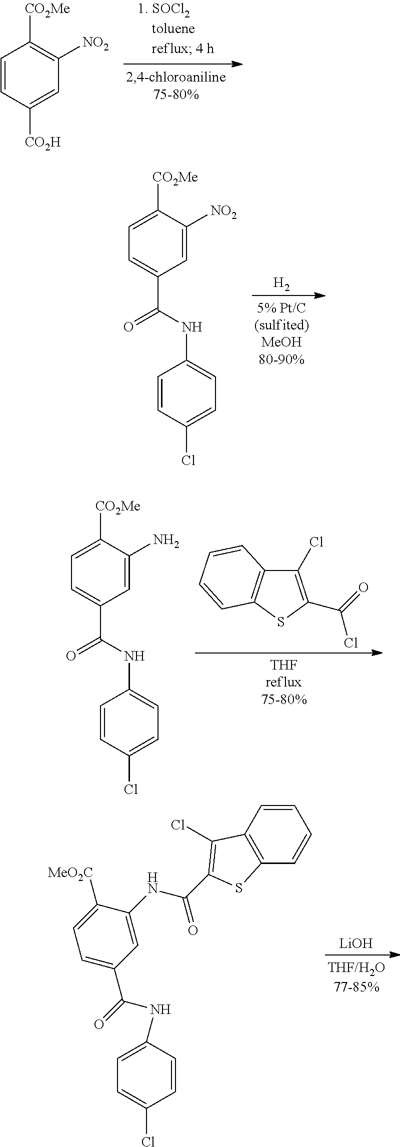

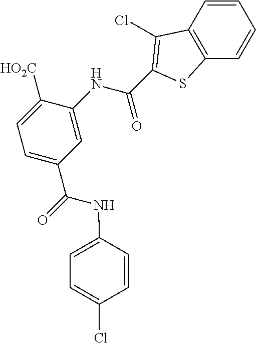 [SMILES: O=C(Nc1ccc(Cl)cc1)c1ccc(C(=O)O)c(NC(=O)c2sc3ccccc3c2Cl)c1]

To a solution of 2-nitroterephthalic acid 1-methyl ester (1 eq.) in toluene was added thionyl chloride (2.5 eq.) and a catalytic amount of pyridine, and the mixture was refluxed for 4 h. The reaction mixture was concentrated to half of its original volume. Some more volume of toluene was added to the reaction mixture, and the pH of the solution was made neutral. A toluene solution of p-chloroaniline (1.6 eq.) was added and the solution stirred for 12-14 h at room temperature. The reaction mixture was concentrated and dissolved in ethyl acetate. The ethyl acetate solution was washed with dil. HCl and washed with water twice. The dried ethyl acetate solution was concentrated and chromatographed over silica gel. Eluting with 12-15% ethyl acetate-hexane afforded 75-80% of the desired product. This was dissolved in MeOH and hydrogenated for 18-20 h in the presence of 10% platinum sulfide on carbon. After filtration of the catalyst through a Celite pad, the filtrate was concentrated under vacuum to afford a residue which was dissolved in THF. 1.1 eq. of 3-chloro-benzo[b]thiophene-2-carbonyl chloride were added. The reaction mixture was refluxed for 10-14 h and concentrated under vacuum. The crude reaction mass was washed with ether two times and dissolved in THF-water (3:1). LiOH (1.1 eq.) was added and the solution was stirred overnight at room temperature, concentrated and the residue was dissolved in the minimum volume of water. The pH was adjusted to pH 6-7 and the aqueous solution was extracted with ethyl acetate. The organic layer was concentrated under vacuum to afford the title compound (50% overall) mp>200° C.

¹H-NMR (300 MHz, DMSO-d₆, 300° K), δ ppm: 10.40 (br.s, 1H), 8.95-9.09 (m, 1H), 8.05-8.19 (m, 2H), 7.91-8.01 (m, 1H), 7.81 (m, 2H), 7.53-7.68 (m, 3H), 7.38 (d, 2H);

MS: 483 [M-H]⁻

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 2 | 2-(3-Bromothiophene-2- | >250 | 12.20 (br.s, 2H); 10.60 (s, 1H, NH) | 476.9 |
| | carboxamido)-4-(4-chloro- | | 8.98 (d, J 1.52 Hz, 1 Ar—H); 8.16- | [M − H]− |
| | phenylcarbamoyl)benzoic | | 7.30 (m, 8 Ar H) | |
| | acid | | | |
| 3 | 2-(3-Chlorobenzo[b]- | >250 | 14.10 (br. s, 1H, CO₂H); 12.17 (s, | 387 |
| | thiophene-2-carboxamido)-4- | | 1H, NH); 8.99 (d, J 1.44 Hz, 1 Ar—H); | [M − H]− |
| | (methylcarbamoyl)benzoic | | 8.66 (d, 1H, NH); 8.20-7.61 (m, 6 | |
| | acid | | Ar—H); 2.81-2.80 (d, 3H) | |
| 4 | 2-(3-Chlorobenzo[b]- | >250 | 16.01 (s, 1H, CO₂H); 8.87(s, 1 Ar—H); | 455 |
| | thiophene-2-carboxamido)-4- | | 8.21-7.44 (m, 7 Ar—H + NH); 3.75 | [M − H]− |
| | (cyclohexylcarbamoyl)- | | (m, 1H); 1.81 (m, 2H); 1.74 (m, 2H); | |
| | benzoic acid | | 1.60 (m, 2H); 1.31 (m, 4H) | |
| 5 | 2-(3-Chlorobenzo[b]- | >250 | 12.75 (br.s, 1H, CO₂H); 10.44 (s, 1H, | 501 |
| | thiophene-2-carboxamido)-4- | | NH); 9.11(d, J 1 Hz, 1 Ar—H); 8.19- | [M − H]− |
| | (4-chloro-2-fluoro-phenyl- | | 7.33 (m, 9 Ar—H) | |
| | carbamoyl)benzoic acid | | | |
| 6 | 4-(4-Chlorophenyl- | >250 | 13.9 (br.s, 1H, CO₂H); 10.53 (s, 1H, | 413 |
| | carbamoyl)-2-(4-methyl- | | NH); 9.01(s, 1 Ar—H); 8.14-7.40 (m, 8 | [M − H]− |
| | thiophene-2-carboxamido)- | | Ar—H); 2.32 (s, 3H) | |
| | benzoic acid | | | |
| 7 | 4-(4-Chlorophenyl- | >250 | 12.5 (br.s, 1H, CO₂H); 10.64 (s, 1H, | 413 |
| | carbamoyl)-2-(5-methyl- | | NH); 9.02 (d, J 1.56 Hz, 1 Ar—H); | [M − H]− |
| | thiophene-2-carboxamido)- | | 8.14-6.98 (m, 8 Ar—H ); 2.50 (s, 3H) | |
| | benzoic acid | | | |
| 8 | 4-(4-Chlorophenyl- | >250 | 12.01 (s, 1H, NH); 10.60 (s, 1H, NH); | 433 |
| | carbamoyl)-2-(5-chloro- | | 8.90 (d, J 1.52 Hz, 1 Ar—H); 8.14-7.21 | [M − H]− |
| | thiophene-2-carboxamido)- | | (m, 8 Ar—H) | |
| | benzoic acid | | | |
| 9 | 4-(4-Chlorophenyl- | >250 | 10.39 (br.s, 1H); 9.02 (s, 1H); 8.12 | 475 |
| | carbamoyl)-2-(5-phenyl- | | (d, 1H); 7.69-7.91 (m, 5H); 7.65 (d, | [M − H]− |
| | thiophene-2-carboxamido)- | | 1H); 7.33-7.57 (m, 6H) | |
| | benzoic acid | | | |
| 10 | 4-(4-Chlorophenyl- | >250 | 11.28 (s, 1H, NH); 10.57 (s, 1H, NH); | 433 |
| | carbamoyl)-2-(indane-2- | | 8.91(d, J 1.44 Hz, 1 Ar—H); 8.17-6.95 | [M − H]− |
| | carboxamido)benzoic acid | | (m, 10H, Ar—CH); 3.52-3.48 (m, 2H); | |
| | | | 3.26-3.16 (m, 2H) | |
| 11 | 4-(4-Chlorophenyl- | >250 | 12.5 (br.s, 1H, CO₂H); 10.48 (s, 1H, | 385 |
| | carbamoyl)-2-(cyclopentane- | | NH); 8.95 (d, J 1.6 Hz, 1 Ar—H); 8.08- | [M − H]− |
| | carboxamido)benzoic acid | | 7.39 (m, 6 Ar—H); 2.80-2.76 (m, 1H); | |
| | | | 1.94-1.91 (m, 2H); 1.90-1.88 (m, 2H) | |
| | | | 1.86-1.80 (m, 2H); 1.78-1.55 (m, 2H) | |
| 12 | 2-(3-Chlorobenzo[b]- | >250 | 12.18 (s, 1H, NH); 11.45 (s, 1H, NH); | 452 |
| | thiophene-2-carboxamido)-4- | | 9.29-7.62 (m, 11 Ar—H) | [MH]⁺ |
| | (pyridin-3-ylcarbamoyl)- | | | |
| | benzoic acid | | | |
| 13 | 2-(3-Chlorobenzo[b]- | >250 | 14.7 (br.s, 1H, CO₂H); 12.23 (s, 1H, | 452 |
| | thiophene-2-carboxamido)-4- | | NH); 11.71 (s, 1H, NH); 9.16-7.06 | [MH]⁺ |
| | (pyridin-4-ylcarbamoyl)- | | (m, 11 Ar—H) | |
| | benzoic acid | | | |
| 14 | 3-(4-Carboxyphenyl- | >250 | [400 MHz] 13.0 (m, 2H), 11.55 (br.s, | 442.9 |
| | carbamoyl)-4-(5-chloro- | | 1H), 11.0 (br.s, 1H), 8.43 (d, 1H), | [M − H]− |
| | thiophene-2-carboxamido)- | | 8.31 (d, 1H), 8.15 (dd, 1H), 7.95 (d, | |
| | benzoic acid | | 2H), 7.85 (d, 2H), 7.68 (d, 1H), 7.31 | |
| | | | (d, 1H) | |
| 15 | 4-(4-Carboxyphenyl- | >250 | [400 MHz] 14.0 (br.s, 1H), 12.80 | 493.1 |
| | carbamoyl)-2-(3-chloro- | | (br.s, 1H), 12.36 (br.s, 1H), 10.79 (s, | [M − H]− |
| | benzo[b]thiophene-2- | | 1H), 9.10 (d, 1H), 8.19 (m, 2H), 7.96 | |
| | carboxamido)benzoic acid | | (m, 5H), 7.80 (m, 1H), 7.65 (m, 2H) | |
| 16 | 2-(3-Chlorobenzo[b]- | >250 | 10.92 (s, 1H), 8.99-9.15 (m, 1H), | 484 |
| | thiophene-2-carboxamido)-4- | | 8.44 (d, 1H), 8.25 (d, 1H), 8.05-8.19 | [M − H]− |
| | (5-chloropyridin-2-yl- | | (m, 2H), 7.90-8.01 (m, 2H), 7.55- | |
| | carbamoyl)benzoic acid | | 7.70 (m, 3H) | |
| 17 | Methyl 2-(3-chlorobenzo[b]- | >250 | 11.81 (s, 1H, NH); 11.62 (s, 1H, NH); | 467 |
| | thiophene-2-carboxamido)-4- | | 9.09-7.66 (m, 10 Ar—H); 3.92 (s, 3H) | [MH]⁺ |
| | (pyrimidin-4-ylcarbamoyl)- | | | |
| | benzoate | | | |
| 18 | 4-(4-Chlorophenyl- | >200 | 14.09 (br.s, 1H), 12.22 (br.s, 1H), | 519.00 |
| | carbamoyl)-2-(3,6-dichloro- | | 10.60 (s, 1H), 9.07 (d, 1H), 8.38 (d, | [MH]⁺ |
| | benzo[b]thiophene-2- | | 1H), 8.18 (d, 1H), 7.98 (d, 1H), 7.74- | |
| | carboxamido)benzoic acid | | 7.90 (m, 3H), 7.67 (dd, 1H), 7.44 (m, | |
| | | | 2H) | |
| 19 | 4-(4-Chlorophenyl- | >200 | 11.90 (s, 1H), 10.57 (s, 1H), 9.04 (d, | 435.07 |
| | carbamoyl)-2-(3-chloro- | | 1H), 8.16 (d, 1H), 8.02 (d, 1H), 7.82 | [MH]⁺ |
| | thiophene-2-carboxamido)- | | (m, 2H), 7.75 (dd, 1H), 7.43 (m, 2H), | |
| | benzoic acid | | 7.28 (d, 1H) | |
| 20 | 4-(4-Chlorophenyl- | >200 | 14.03 (br.s, 1H), 12.73 (br.s, 1H), | 402.15 |
| | carbamoyl)-2-(thiazole-2- | | 10.59 (s, 1H) 9.20 (d, 1H), 8.20 (d, | [MH]⁺ |
| | carboxamido)benzoic acid | | 1H), 8.19 (d, 1H), 8.15 (d, 1H), 7.82 | |
| | | | (m, 2H), 7.76 (dd, 1H), 7.44 (m, 2H) | |
| 21 | 2-(5-Chlorobenzo[b]- | >200 | [MeOD, 328° K] 8.37 (d, 1H), 7.32- | 485.03 |
| | thiophene-3-carboxamido)-4- | | 7.49 (m, 1H), 7.29 (d, 1H), 7.05 (d, | [MH]⁺ |
| | (4-chlorophenylcarbamoyl)- | | 1H), 6.95-7.11 (m, 5H), 6.69 (d, 1H), | |
| | benzoic acid | | 5.44 (dd, 1H), 5.38-5.50 (m, 1H), | |
| | | | 4.88 (s, 2H), 3.59-3.77 (m, 1H), | |
| | | | 3.39-3.59 (m, 3H), 3.21-3.29 (m) | |
| 22 | 2-(Benzothiazole-2- | >200 | 14.15 (br.s, 1H), 12.99 (s, 1H), 10.62 | 452.18 |
| | carboxamido)-4-(4-chloro- | | (s, 1H), 9.23 (d, 1H), 8.27-8.36 (m, | [MH]⁺ |
| | phenylcarbamoyl)benzoic | | 1H), 8.22 (d, 1H), 8.16-8.26 (m, 1H), | |
| | acid | | 7.84 (m, 2H), 7.80 (dd, 1H), 7.54- | |
| | | | 7.75 (m, 2H), 7.45 (m, 2H) | |
| 23 | 2-(6-Chlorobenzo[b]- | >200 | 14.13 (s, 1H), 12.29 (br.s, 1H), 10.59 | 485.1 |
| | thiophene-2-carboxamido-4- | | (s, 1H), 9.00 (d, 1H), 8.29 (d, 1H), | [MH]⁺ |
| | (4-chlorophenylcarbamoyl)- | | 8.16 (s, 1H), 8.17 (d, 1H), 8.10 (d, | |
| | benzoic acid | | 1H), 7.83 (m, 2H), 7.76 (dd, 1H), | |
| | | | 7.54 (dd, 1H), 7.44 (m, 2H) | |
| 24 | 4-(4-Benzylpiperidine-1- | >250 | [400 MHz] 13.0 (m, 2H), 11.55 (br.s, | 531.3 |
| | carbonyl)-2-(3-chloro- | | 1H), 11.0 (br.s, 1H), 8.43 (d, 1H), | [M − H]− |
| | benzo[b]thiophene-2- | | 8.31 (d, 1H), 8.15 (dd, 1H), 7.95 (d, | |
| | carboxamido)benzoic acid | | 2H), 7.85 (d, 2H), 7.68 (d, 1H), 7.31 | |
| | | | (d, 1H) | |
| 25 | 4-(4-Chlorophenyl- | >250 | [400 MHz] 14.0 (br.s, 1H), 12.80 | 475.1 |
| | carbamoyl)-3-(5-phenyl- | | (br.s, 1H), 12.36 (br.s, 1H), 10.79 (s, | [M − H]− |
| | thiophene-2-carboxamido)- | | 1H), 9.10 (d, 1H), 8.19 (m, 2H), 7.96 | |
| | benzoic acid | | (m, 5H), 7.80 (m, 1H), 7.65 (m, 2H) | |
| 26 | 2-(3-Chlorobenzo[b]- | >200 | 14.18 (br.s, 1H), 12.43 (br.s, 1H), | 484.71 |
| | thiophene-2-carboxamido)-5- | | 10.53 (s, 1H), 8.75 (d, 1H), 8.68 (d, | [MH]⁺ |
| | (4-chlorophenylcarbamoyl)- | | 1H), 8.28 (dd, 1H), 8.10-8.24 (m, | |
| | benzoic acid | | 1H), 7.92-8.08 (m, 1H), 7.83 (m, 2H), | |
| | | | 7.57-.74 (m, 2H), 7.43 (m, 2H) | |
| 27 | 4-(5-Chlorothiazol-2-yl- | >200 | [400 MHz] 13.12 (br.s, 1H), 12.79 | 502.0 |
| | carbamoyl)-2-(5-(4- | | (br.s, 1H), 9.13 (s, 1H), 8.15 (m, 1H), | [MH]⁺ |
| | fluorophenyl)thiophene-2- | | 7.84-7.77 (m, 4H), 7.66 (m, 2H), | |
| | carboxamido)benzoic acid | | 7.32 (m, 2H). | |
| 28 | 2-(3-Chlorobenzo[b]- | >200 | [400 MHz] 14.10 (br.s, 1H), 12.31 (s, | 535.1 |
| | thiophene-2-carboxamido)-4- | | 1H), 8.64 (d, 1H), 8.00-8.30 (m, 2H), | [MH]⁺ |
| | (4-phenoxypiperidine-1- | | 7.90 (m, 1H), 7.60 (m, 2H), 7.30 (m, | |
| | carbonyl)benzoic acid | | 3H), 6.80-7.00 (m, 3H), 4.67 (m, 1H), | |
| | | | 4.00 (m, 1H), 3.35 (m, 2H), 3.14 (m, | |
| | | | 1H), 2.00( m, 2H), 1.61 (m, 2H) | |
| 29 | 2-(3-Chlorobenzo[b]- | >200 | [400 MHz] 14.10 (br.s, 1H), 12.31 (s, | 535.2 |
| | thiophene-2-carboxamido)-4- | | 1H), 8.64 (d, 1H), 8.00-8.30 (m, 2H), | [MH]⁺ |
| | (3-phenoxypiperidine-1- | | 7.70 (m, 2H), 6.70-7.40 (m, 7H), | |
| | carbonyl)benzoic acid | | 4.50-4.75( m, 2H), 3.40-4.20 (m, | |
| | | | 3H), 1.50-2.05 (m, 4H) | |
| 30 | 4-(4-Chlorophenyl- | >200 | [400 MHz] 14.10 (br.s, 1H), 12.20 (s, | 409.2 |
| | carbamoyl)-2-(2-hydroxy- | | 1H), 11.50 (s, 1H), 10.60 (s, 1H), | [M − H]− |
| | benzamido)benzoic acid | | 9.10 (m, 1H), 8.15 (m, 1H), 7.80- | |
| | | | 8.00 (m, 3H), 7.60 (m, 1H), 7.45- | |
| | | | 7.55 (m, 3H), 7.00 (m, 2H) | |
| 31 | 5-(4-tert-Butylpiperazine-1- | 195 | 12.32 (br.s, 1H), 9.37 (br.s, 1H), 8.69 | 499.96 |
| | carbonyl)-2-(3-chloro- | | (d, 1H), 8.11-8.26 (m, 2H), 7.94-8.03 | [MH]⁺ |
| | benzo[b]thiophene-2- | | (m, 1H), 7.83 (dd, 1H), 7.59-7.75 (m, | |
| | carboxamido)benzoic acid | | 2H), 3.72-4.63 (m, 2H), 3.52-3.70 | |
| | | | (m, 2H), 2.98-3.23 (m, 4H), 1.35 (s, | |
| | | | 9H) | |
| 32 | 2-(3-Chlorobenzo[b]- | >200 | 12.18 (br.s, 1H), 9.05 (d, 1H), 8.98 | 504.91 |
| | thiophene-2-carboxamido)-4- | | (d, 1H), 8.18 (ddd, 1H), 8.11 (d, 1H), | [MH]⁺ |
| | (1,2,3,4-tetrahydro- | | 7.94-8.04 (m, 1H) 7.73 (dd, 1H), | |
| | naphthalen-1-ylcarbamoyl)- | | 7.54-7.70 (m, 2H), 7.05-7.31 (m, | |
| | benzoic acid | | 4H), 5.06-5.39 (m, 1H), 2.75-2.87 | |
| | | | (m, 2H), 1.60-2.11 (m, 4H) | |
| 33 | 2-(3-Chlorobenzo[b]- | >200 | 12.22 (br.s, 1H), 8.89 (d, 1H), 8.12- | 508.95 |
| | thiophene-2-carboxamido)-4- | | 8.23 (m, 1H), 8.07 (d, 1H), 7.93-8.03 | [MH]⁺ |
| | (adamantylcarbamoyl)- | | (m, 1H), 7.82 (s, 1H), 7.61-7.72 (m, | |
| | benzoic acid | | 2H), 7.58 (dd, 1H), 2.01-2.19 (m, | |
| | | | 9H), 1.56-1.80 (m, 6H) | |
| 34 | 4-((1R,2R,4S)-Bicyclo[2.2.1]- | >200 | 12.18 (br.s, 1H), 8.94 (d, 1H), 8.30 | 468.94 |
| | heptan-2-ylcarbamoyl)-2-(3- | | (d, 1H), 8.14-8.23 (m, 1H), 8.09 (d, | [MH]⁺ |
| | chlorobenzo[b]thiophene-2- | | 1H), 7.95-8.05 (m, 1H), 7.57-7.75 | |
| | carboxamido)benzoic acid | | (m, 3H), 3.68-3.85 (m, 1H), 2.16- | |
| | | | 2.34 (m, 2H), 1.37-1.73 (m, 5H), | |
| | | | 0.97-1.34 (m, 3H) | |
| 35 | 2-(3-Chlorobenzo[b]- | >200 | [353° K] 12.13 (br.s, 1H), 8.68 (d, | 586.8 |
| | thiophene-2-carboxamido)-5- | | 1H), 8.07-8.18 (m, 2H), 7.92-8.04 | [MH]+ |
| | (4-(2,6-dichlorophenyl)- | | (m, 1H), 7.72 (dd, 1H), 7.59-7.69 (m, | |
| | piperidine-1-carbonyl)benzoic | | 2H), 7.40-7.46 (m, 2H), 7.26 (dd, | |
| | acid | | 1H), 4.08-4.34 (m, 2H), 3.84 (tt, 1H), | |
| | | | 3.10 (td, 3H), 2.39-2.48 (m, 1H), | |
| | | | 1.54-1.72 (m, 2H) | |
| 36 | 2-(4-(1H-Benzo[d]imidazol-2- | >200 | 12.46 (s, 1H), 10.49 (s, 1H) 8.86 (d, | 510.96 |
| | yl)benzamido)-5-(4-chloro- | | 1H), 8.70 (d, 1H), 8.40 (m, 2H), 8.29 | [MH]⁺ |
| | phenylcarbamoyl)benzoic | | (dd, 1H), 8.16 (m, 2H), 7.83 (m, 2H), | |
| | acid | | 7.66 (dd, 2H), 7.43 (m, 2H), 7.27 | |
| | | | (dd, 2H) | |
| 37 | 2-(4-(5-Chloro-1H-benzo[d]- | >200 | 12.43 (s, 1H), 10.49 (s, 1H), 8.85 (d, | 544.98 |
| | imidazol-2-yl)benzamido)-5- | | 1), 8.70 (d, 1H), 8.39 (m, 2H), 8.29 | [MH]⁺ |
| | (4-chlorophenylcarbamoyl)- | | (dd, 1H), 8.17 (m, 2H), 7.83 (m, 2H), | |
| | benzoic acid | | 7.72 (d, 1H), 7.67 (d, 1H), 7.43 (m, | |
| | | | 2H), 7.29 (dd, 1H) | |
| 38 | 2-(4-(5,6-Dichloro-1H- | >200 | [353° K] 12.13 (s, 1H), 8.76 (d, 1H), | 631.01 |
| | benzo[d]imidazol-2-yl)- | | 8.30-8.40 (m, 2H), 8.07-8.21 (m, | [MH]⁺ |
| | benzamido)-5-(4-(4- | | 3H), 7.86 (s, 2H), 7.75 (dd, 1H), | |
| | fluorophenyl)piperidine-1- | | 7.24-7.41 (m, 2H), 7.01-7.20 (m, | |
| | carbonyl)benzoic acid | | 2H), 3.89-4.38 (m, 2H), 3.00-3.19 | |
| | | | (m, 2H), 2.82-2.96 (m, 1H), 1.78- | |
| | | | 1.94 (m, 2H), 1.54-1.78 (m, 2H) | |
| 39 | 2-(4-(5-Carboxy-1H-benzo[d]- | >200 | 12.43 (s, 1H), 10.51 (s, 1H), 8.85 (d, | 555 |
| | imidazol-2-yl)benzamido)-4- | | 1H), 8.70 (d, 1H), 8.45 (m, 2H), 8.15- | [MH]⁺ |
| | (4-chlorophenylcarbamoyl)- | | 8.35 (m, 4H), 7.93 (dd, 1H), 7.83 (m, | |
| | benzoic acid | | 2H), 7.76 (d, 1H), 7.43 (m, 2H) | |
| 40 | 4-(4-Chlorophenyl- | >200 | [353° K] 11.98 (s, 1H), 10.35 (s, 1H), | n.a. |
| | carbamoyl)-2-(4-(5,6-di- | | 9.14 (d, 1H) 8.37 (m, 2H), 8.09-8.23 | |
| | chloro-1H-benzo[d]imidazol- | | (m, 3H), 7.87 (s, 2H), 7.82 (m, 2H), | |
| | 2-yl)benzamido)benzoic acid | | 7.76 (dd, 1H), 7.42 (m, 2H) | |
| 41 | 5-(4-Chlorophenyl- | >200 | 12.43 (s, 1H), 10.49 (s, 1H), 8.85 (d, | 568.87 |
| | carbamoyl)-2-(4-(5- | | 1H), 8.70 (d, 1H), 8.42 (m, 2H) 8.23- | [MH]⁺ |
| | (methoxycarbonyl)-1H- | | 8.33 (m, 2H), 8.18 (m, 2H,) 7.91 (dd, | |
| | benzo[d]imidazol-2-yl)- | | 1H), 7.83 (m, 2H), 7.75 (d, 1H), 7.43 | |
| | benzamido)benzoic acid | | (m, 2H), 3.90 (s, 3H) | |
| 42 | 2-(4-(5,6-Dichloro-1H- | >200 | 13.42 (br.s, 1H), 12.65 (br.s, 1H), | 597.01 |
| | benzo[d]imidazol-2-yl)-3- | | 12.27 (br.s, 1H), 8.75 (d, 1H), 8.31 | [MH]⁺ |
| | hydroxybenzamido)-5- | | (d, 1H) 7.98 (d, 1H), 8.00 (br.s, 2H), | |
| | (diisobutylcarbamoyl)benzoic | | 7.67 (dd, 1H), 7.64 (d, 1H), 7.60 (dd, | |
| | acid | | 1H), 2.90-3.21 (m, 4H), 1.66-2.22 | |
| | | | (m, 2H), 0.68-1.03 (m, 12H) | |
| 43 | 2-(4-(5,6-Dichloro-1H- | >200 | 12.29 (s, 1H), 8.76 (d, 1H), 8.40 (m, | 552.91 |
| | benzo[d]imidazol-2-yl)- | | 2H), 8.15 (m, 2H), 8.05 (d, 1H), 7.93 | [MH]⁺ |
| | benzamido)-5-(N-isopentyl-N- | | (s, 2H), 7.73 (dd, 1H), 3.11-3.68 (m, | |
| | methylcarbamoyl)benzoic | | 2H), 2.96 (s, 3H), 1.27-1.69 (m, 3H), | |
| | acid | | 0.15-1.14 (m, 6H) | |
| 44 | 5-(N-4-Butylphenyl-N-methyl- | >200 | 12.22 (s, 1H), 8.51 (d, 1H), 8.36 (m, | 614.98 |
| | carbamoyl)-2-(4-(5,6- | | 2H), 8.09 (m, 2H), 8.00 (d, 1H), 7.91 | [MH]⁺ |
| | dichloro-1H-benzo[d]- | | (s, 2H), 7.50 (dd, 1H), 7.11 (s, 4H), | |
| | imidazol-2-yl)benzamido)- | | 3.38 (s, 3H), 2.53-2.57 (m, 2H), | |
| | benzoic acid | | 1.35-1.63 (m, 2H), 1.23 (m, 2H), | |
| | | | 0.85 (t, 3H) | |
| 45 | 5-(N-4-Chlorophenyl-N- | >200 | 12.21 (s, 1H), 8.54 (d, 1H), 8.35 (m, | 634.91 |
| | isobutylcarbamoyl)-2-(4-(5,6- | | 2H), 8.10 (m, 2H), 7.98 (d, 1H), 7.91 | [M − H]− |
| | dichloro-1H-benzo[d]- | | (s, 2H), 7.52 (dd, 1H), 7.32-7.42 (m, | |
| | imidazol-2-yl)benzamido)- | | 2H), 7.20-7.32 (m, 2H), 3.75 (d, 2H), | |
| | benzoic acid | | 1.68-1.94 (m, 1H), 0.91 (d, 6H) | |
| 46 | 2-(4-(5,6-Dichloro-1-methyl- | >200 | [353° K] 12.20 (br.s, 1H), 8.75 (d, | 593.13 |
| | 1H-benzo[d]imidazol-2- | | 1H), 8.13-8.20 (m, 2H), 8.06-8.12 | [M − H]− |
| | yl)benzamido)-5-(diisobutyl- | | (m, 2H), 8.02 (d, 1H), 8.00 (s, 1H), | |
| | carbamoyl)benzoic acid | | 7.95 (s, 1H), 7.65 (dd, 1H), 3.95 (s, | |
| | | | 3H), 3.25 (d, 4H), 1.87-2.08 (m, 2H), | |
| | | | 0.86 (d, 12H) | |
| 47 | 5-((5-Chlorothiophen-2-yl- | >200 | 12.39 (s, 1H), 9.33 (t, 1H), 8.79 (d, | 596.31 |
| | methyl)carbamoyl)-2-(4-(5,6- | | 1H) 8.62 (d, 1H), 8.33-8.48 (m, 2H), | [M − H]− |
| | dichloro-1H-benzo[d]- | | 8.10-8.24 (m, 3H), 7.92 (s, 2H), 6.96 | |
| | imidazol-2-yl)benzamido)- | | (d, 1H), 6.92 (d, 1H), 4.56 (d, 2H) | |
| | benzoic acid | | | |
| 48 | 2-(4-(5,6-Dichloro-1H- | >200 | 13.48 (br.s, 1H), 12.59 (br.s, 1H), | 562.9 |
| | benzo[d]imidazol-2-yl)- | | 10.27 (s, 1H), 8.85 (d, 1H), 8.72 (d, | [MH]⁺ |
| | benzamido)-5-(2-fluoro- | | 1H) 8.34-8.44 (m, 2H), 8.28 (dd, | |
| | phenylcarbamoyl)benzoic | | 1H), 8.14-8.21 (m, 2H), 7.79-8.03 | |
| | acid | | (m, 2H), 7.56-.67 (m, 1H), 7.11-7.39 | |
| | | | (m, 3H) | |
| 49 | 5-(N-Cyclohexylmethyl-N- | >200 | [353° K] 12.16 (br.s, 1H), 8.73 (d, | 578.99 |
| | methylcarbamoyl)-2-(4-(5,6- | | 1H), 8.30-8.43 (m, 2H), 8.10-8.24 | [MH]⁺ |
| | dichloro-1H-benzo[d]- | | (m, 2H), 8.05 (d, 1H), 7.86 (s, 2H), | |
| | imidazol-2-yl)benzamido)- | | 7.68 (dd, 1H), 3.29 (d, 2H), 2.98 (s, | |
| | benzoic acid | | 3H), 1.47-1.82 (m, 6H), 1.03-1.37 | |
| | | | (m, 3H), 0.74-1.03 (m, 2H) | |
| 50 | 5-(4-Chlorophenyl- | >200 | 12.55 (br.s, 1H), 10.50 (s, 1H), 8.87 | 523.04 |
| | carbamoyl)-2-(4-(1-methyl- | | (d, 1H), 8.71 (d, 1H), 8.29 (dd, 1H), | [M − H]− |
| | 1H-benzo[d]imidazol-2-yl)- | | 8.16-8.24 (m, 2H), 8.09-8.16 (m, | |
| | benzamido)benzoic acid | | 2H), 7.80-7.90 (m, 2H), 7.74 (d, 1H), | |
| | | | 7.68 (d, 1H), 7.40-7.50 (m, 2H), 7.36 | |
| | | | (td, 1H), 7.30 (ddd, 1H), 3.97 (s, 3H) | |
| 51 | 5-(4-Chloro-2-fluorophenyl- | >200 | 13.45 (br.s, 1H), 12.56 (br.s, 1H), | 596.82 |
| | carbamoyl)-2-(4-(5,6- | | 10.34 (s, 1H), 8.85 (d, 1H), 8.71 (d, | [MH]⁺ |
| | dichloro-1H-benzo[d]- | | 1H) 8.33-8.45 (m, 2H), 8.27 (dd, | |
| | imidazol-2-yl)benzamido)- | | 1H), 8.07-8.23 (m, 2H), 7.91 (br.s, | |
| | benzoic acid | | 2H), 7.66 (t, 1H), 7.54 (dd, 1H), 7.33 | |
| | | | (d, 1H) | |

### Example 52

### 2-(3-Chlorobenzo[b]thiophene-2-carboxamido)-4-(thiazol-2-ylcarbamoyl)-benzoic acid

### Method 2

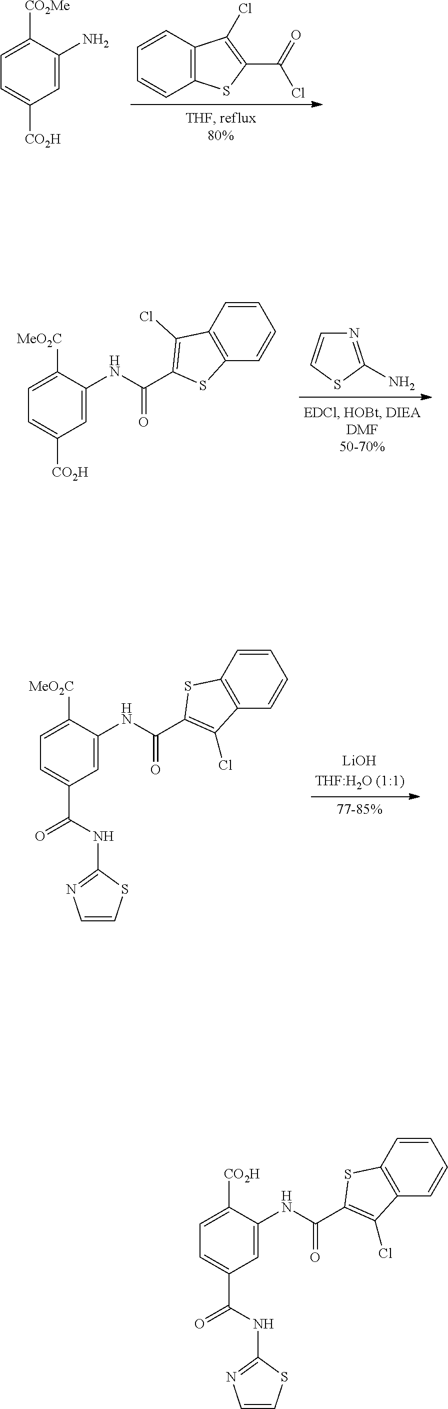

2-Aminoterephthalic acid 1-methyl ester was dissolved in THF, and 1.1 eq. of 3-chloro-benzo[b]thiophene-2-carbonyl chloride was added. The reaction mixture was refluxed for 14 h, then cooled at room temperature and concentrated under vacuum. The crude residue was triturated with diethyl ether, dissolved in DMF, and 2-aminothiazole (1.2 eq.), EDCl (1.5 eq.), HOBt (0.5 eq.) and DIEA (2.5 eq.) were added at 5-10° C. The reaction mixture was stirred at room temperature for 16 h, then diluted with water. The precipitated solid was collected by suction filtration and dried, to provide 70% methyl 2-(3-chloro-benzo[b]thiophene-2-carboxamido)-4-(thiazol-2-ylcarbamoyl)benzoate. This product was dissolved in THF-water (3:1) and treated with LiOH (1.1 eq.). The solution was stirred overnight at room temperature, concentrated under vacuum, and the residue dissolved in the minimum volume of water, adjusting the pH to 6-7. The aqueous solution was extracted with ethyl acetate, and the organic layer was collected, dried and concentrated under vacuum to afford the title compound, mp>200° C., in 80% yield.

¹H-NMR (300 MHz, DMSO-d₆, 300° K), δ ppm: 13.00 (br.s, 1H, CO₂H); 12.14 (s, 1H, NH); 9.23 (d, J=0.8 Hz, 1 Ar—H); 8.19-7.31 (m, 8 Ar—H); MS: m/z=458 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 53 | 3-(3-Chlorobenzo[b]- | >250 | [500 MHz] 12.03 (br.s, 1H), 10.95 (s, | 483.3 |
| | thiophene-2-carboxamido)-5- | | 1H), 10.66 (s, 1H), 8.57 (m, 1H), | [M − H]− |
| | (4-chlorophenylcarbamoyl)- | | 8.51 (m, 1H), 8.33 (m, 1H), 8.19 (m, | |
| | benzoic acid | | 1H), 7.98 (m, 1H), 7.83 (m, 2H), | |
| | | | 7.65 (m, 2H), 7.43 (m, 2H) | |
| 54 | 2-(3-Chlorobenzo[b]- | >250 | [400 MHz] 12.9 (br.s, 2H), 10.8 (s, | |
| | thiophene-2-carboxamido)-4- | | 1H), 9.10 (s, 1H), 8.20 (m, 2H), 7.99 | 533 |
| | (4-(5-oxo-2,5-dihydro-1,2,4- | | (m, 3H), 7.76 (m, 2H), 7.68 (m, 1H), | [M − H]− |
| | oxadiazol-3-yl)phenyl- | | 7.64 (m, 2H). | |
| | carbamoyl)benzoic acid | | | |
| 55 | 2-(3-Chlorobenzo[b]- | >200 | 12.15 (br.s, 1H), 8.95 (d, 1H), 8.49 | 443.09 |
| | thiophene-2-carboxamido)-4- | | (d, 1H), 8.14-8.22 (m, 1H), 8.10 (d, | [MH]⁺ |
| | (cyclopentylcarbamoyl)- | | 1H), 7.93-8.03 (m, 1H), 7.53-7.74 | |
| | benzoic acid | | (m, 3H), 4.13-4.37 (m, 1H), 1.83- | |
| | | | 2.00 (m, 2H), 1.65-1.83 (m, 2H), | |
| | | | 1.42-1.65 (m, 4H) | |
| 56 | 2-(3-Chlorobenzo[b]- | >200 | 12.08 (s, 1H), 8.94 (d, 1H), 8.45 (d, | 471.11 |
| | thiophene-2-carboxamido)-4- | | 1H), 8.14-8.21 (m, 1H), 8.10 (d, 1H), | [MH]⁺ |
| | (cycloheptylcarbamoyl)- | | 7.93-8.03 (m, 1H), 7.57-7.71 (m, | |
| | benzoic acid | | 3H), 3.84-4.11 (m, 1H), 1.77-1.99 | |
| | | | (m, 2H), 1.32-1.77 (m, 10H) | |
| 57 | 2-(3-Chlorobenzo[b]- | >200 | 12.24 (s, 1H), 8.63 (d, 1H), 8.15- | 445.04 |
| | thiophene-2-carboxamido)-4- | | 8.21 (m, 1H), 8.12 (d, 1H), 7.91-8.04 | [MH]⁺ |
| | (morpholine-4-carbonyl)- | | (m, 1H), 7.53-7.76 (m, 2H), 7.29 (dd, | |
| | benzoic acid | | 1H), 3.52-3.79 (m, 4H), 3.08-3.43 | |
| | | | (m, 4H) | |
| 58 | 2-(Benzo[b]thiophene-3- | >200 | 13.95 (s, 1H), 11.99 (s, 1H), 10.60 | 451.08 |
| | carboxamido)-4-(4-chloro- | | (s, 1H), 9.10 (d, 1H), 8.57 (s, 1H), | [MH]⁺ |
| | carbamoyl)benzoic acid | | 8.48-8.55 (m, 1H), 8.17 (d, 1H), | |
| | | | 8.10-8.15 (m, 1H), 7.84 (m, 2H), | |
| | | | 7.74 (dd, 1H), 7.47-7.57 (m, 2H), | |
| | | | 7.45 (m, 2H) | |
| 59 | 2-(3-Chlorobenzo[b]- | >200 | 14.03 (br.s, 1H), 13.22 (br.s, 1H), | 492.06 |
| | thiophene-2-carboxamido)-4- | | 12.15 (s, 1H), 9.22 (d, 1H), 8.11- | [MH]⁺ |
| | ((5-chlorothiazol-2-yl)- | | 8.22 (m, 1H), 8.17 (d, 1H), 7.96-8.04 | |
| | carbamoyl)benzoic acid | | (m, 1H), 7.93 (dd, 1H), 7.54-7.78 (m, | |
| | | | 3H) | |
| 60 | 2-(3-Chlorobenzo[b]- | >200 | 14.25 (br.s, 1H), 13.44 (br.s, 1H), | 459.06 |
| | thiophene-2-carboxamido)-4- | | 12.17 (s, 1H), 9.27 (d, 1H), 9.26 (s, | [MH]⁺ |
| | (([1,3,4]thiadiazol-2-yl)- | | 1H), 8.20 (d, 1H), 8.11-8.20 (m, 1H), | |
| | carbamoyl)benzoic acid | | 7.97-8.05 (m, 1H), 7.97 (dd, 1H), | |
| | | | 7.58-7.79 (m, 2H) | |
| 61 | 2-(5-Phenylthiophene-2- | >250 | 12.9 (br.s, 1H, CO₂H); 12.42 (br.s, | 448 |
| | carboxamido)-4-(thiazol-2-yl- | | 1H, NH); 9.15 (s, 1 Ar—H); 8.15-7.31 | [M − H]− |
| | carbamoyl)benzoic acid | | (m, 11 Ar—H) | |
| 62 | 4-(5-Chlorothiazol-2-yl- | >250 | [400 MHz] 13.15 (br.s, 1H), 12.47 | 482 |
| | carbamoyl)-2-(5-phenyl- | | (br.s, 1H), 9.14 (s, 1H), 8.16 (m, | [M − H]− |
| | thiophene-2-carboxamido)- | | 1H), 7.86 (m, 1H), 7.80 (m, 3H), | |
| | benzoic acid | | 7.77 (m, 1H), 7.64 (m, 1H), 7.48 (m, | |
| | | | 2H), 7.39 (m, 1H). | |
| 63 | 2-(3-Chlorobenzo[b]- | >250 | [400 MHz] 12.80 (br.s, 1H), 10.40 (s, | 553.5 |
| | thiophene-2-carboxamido)-4- | | 1H), 9.07 (s, 1H), 8.16 (m, 2H), 7.74 | [M − H]− |
| | (4-phenethylphenyl- | | (m, 1H), 7.66 (m, 1H), 7.64 (m, 4H), | |
| | carbamoyl)benzoic acid | | 7.15-7.29 (m, 7H), 2.87 (m, 4H). | |
| 64 | 2-(3-Chlorobenzo[b]- | >200 | 14.14 (br.s, 1H), 12.21 (br.s, 1H), | 543.13 |
| | thiophene-2-carboxamido)-4- | | 10.50 (s, 1H), 9.09 (d, 1H), 8.08- | [MH]⁺ |
| | (4-phenoxyphenyl- | | 8.30 (m, 2H), 7.90-8.08 (m, 1H), | |
| | carbamoyl)benzoic acid | | 7.73-7.90 (m, 3H), 7.54-7.73 (m, | |
| | | | 2H), 7.27-7.48 (m, 2H), 6.90-7.21 | |
| | | | (m, 5H) | |
| 65 | 2-(3-Chlorobenzo[b]- | >200 | 14.16 (br.s, 1H), 12.23 (br.s, 1H), | 543.13 |
| | thiophene-2-carboxamido)-4- | | 10.53 (s, 1H), 9.06 (d, 1H), 8.08- | [MH]⁺ |
| | (3-phenoxyphenyl- | | 8.28 (m, 2H), 7.87-8.07 (m, 1H), | |
| | carbamoyl)benzoic acid | | 7.76 (dd, 1H), 7.56-7.71 (m, 3H), | |
| | | | 7.48-7.56 (m, 1H), 7.29-7.48 (m, | |
| | | | 3H), 7.12-7.23 (m, 1H), 6.99-7.12 | |
| | | | (m, 2H), 6.67-6.89 (m, 1H) | |
| 66 | 4-(4-(N-carbamimidoyl- | >200 | 12.12 (br.s, 1H), 10.51 (s, 1H), 9.09 | 572.09 |
| | sulfamoyl)phenylcarbamoyl)- | | (d 1H) 8.07-8.28 (m, 2H), 7.94-8.05 | [MH]⁺ |
| | 2-(3-chlorobenzo[b]- | | (m, 1H), 7.89 (d, 2H), 7.71-7.84 (m, | |
| | thiophene-2-carboxamido)- | | 3H), 7.56-7.70 (m, 2H), 6.57 (s, 4H) | |
| | benzoic acid | | | |
| 67 | 4-(4-Benzylphenyl- | >200 | 14.10 (br.s, 1H), 12.21 (br.s, 1H), | 541.17 |
| | carbamoyl)-2-(3-chloro- | | 10.42 (s, 1H), 9.08 (s, 1H), 8.07- | [MH]⁺ |
| | benzo[b]thiophene-2- | | 8.28 (m, 2H), 7.92-8.06 (m, 1H), | |
| | carboxamido)benzoic acid | | 7.77 (dd, 1H), 7.56-7.73 (m, 4H), | |
| | | | 7.07-7.40 (m, 7H), 3.93 (s, 2H) | |
| 68 | 4-(3-Benzylphenyl- | >200 | 14.10 (br.s, 1H), 12.17 (s, 1H), | 541.18 |
| | carbamoyl)-2-(3-chloro- | | 10.42 (s, 1H), 9.08 (d, 1H), 8.09- | [MH]⁺ |
| | benzo[b]thiophene-2- | | 8.32 (m, 2H), 7.87-8.09 (m, 1H), | |
| | carboxamido)benzoic acid | | 7.77 (dd, 1H), 7.57-7.71 (m, 4H), | |
| | | | 7.11-7.40 (m, 6H), 7.02 (d, 1H), 3.95 | |
| | | | (s, 2H) | |
| 69 | 4-(4-tert-Butylphenyl- | >200 | 14.13 (br.s, 1H), 12.22 (br.s, 1H), | 507.17 |
| | carbamoyl)-2-(3-chloro- | | 10.40 (s, 1H), 9.09 (d, 1H), 8.18 (m, | [MH]⁺ |
| | benzo[b]thiophene-2- | | 2H), 7.92-8.06 (m, 1H), 7.79 (dd, | |
| | carboxamido)benzoic acid | | 1H), 7.60-7.74 (m, 4H), 7.39 (m, | |
| | | | 2H), 1.29 (s, 9H) | |
| 70 | 4-(4-Chlorophenyl- | >200 | 14.09 (br.s, 1H), 12.16 (br.s, 1H), | 477.1 |
| | carbamoyl)-2-(4-phenyl- | | 10.59 (s, 1H), 8.99 (d, 1H), 8.26 (d, | [MH]⁺ |
| | thiophene-2-carboxamido)- | | 1H), 8.20 (d, 1H), 8.16 (d, 1H), 7.83 | |
| | benzoic acid | | (m, 2H), 7.68-7.79 (m, 3H), 7.27- | |
| | | | 7.54 (m, 5H) | |
| 71 | (Z)-4-(4-Benzoylphenyl- | >200 | [400 MHz] 14.20 (br.s, 1H), 12.25 | 555.2 |
| | carbamoyl)-2-(3-chloro-5- | | (br.s, 1H), 10.86 (s, 1H), 9.13 (s, | [MH]⁺ |
| | (prop-1-enyl)-4-vinyl- | | 1H), 8.20 (m, 2H), 8.00 (m, 3H), | |
| | thiophene-2-carboxamido)- | | 7.90 (m, 3H), 7.70 (m, 2H), 7.60 (m, | |
| | benzoic acid | | 3H), 7.56 (m, 2H). | |
| 72 | 2-(3-Chlorobenzo[b]- | >200 | [400 MHz] 14.20 (br.s, 1H), 12.20 | 555.4 |
| | thiophene-2-carboxamido)-4- | | (br.s, 1H), 10.45 (s, 1H), 9.07 (s, | [M − H]− |
| | (4-(α-hydroxybenzyl)phenyl- | | 1H), 8.20 (m, 2H), 8.10 (m, 1H), | |
| | carbamoyl)benzoic acid | | 7.70-8.00 (m, 5H), 7.10-7.50 (m, | |
| | | | 7H), 5.86 (s, 1H), 5.70 (s, 1H) | |
| 73 | 4-(3-Chloro-4-phenoxy- | >200 | [400 MHz] 14.20 (br.s, 1H), 12.21 | 575.1 |
| | phenylcarbamoyl)-2-(3- | | (br.s, 1H), 10.69 (s, 1H), 9.11 (s, | [M − H]− |
| | chlorobenzo[b]thiophene-2- | | 1H), 8.15-8.30 (m, 3H), 8.00 (m, | |
| | carboxamido)benzoic acid | | 1H), 7.70 (m, 2H), 7.60 (m, 2H), | |
| | | | 7.35 (m, 2H), 7.00-7.25 (m, 2H), | |
| | | | 6.90 (m, 2H) | |
| 74 | 4-(N-Carboxymethyl-N-(4- | >200 | [400 MHz] 14.10 (br.s, 1H), 12.96 | 543.4 |
| | chlorophenyl)carbamoyl)-2- | | (br.s, 1H), 12.30 (s, 1H), 8.60 (s, | [MH]⁺ |
| | (3-chlorobenzo[b]thiophene- | | 1H), 8.18 (d, 1H), 7.70-8.00 (m, 2H), | |
| | 2-carboxamido)benzoic acid | | 7.60 (m, 2H), 7.10-7.50 (m, 4H), | |
| | | | 7.00 (s, 1H), 4.52 (s, 2H) | |
| 75 | 4-(3-tert-Butylphenyl- | >200 | 14.14 (br.s, 1H), 12.22 (br.s, 1H), | 507.17 |
| | carbamoyl)-2-(3-chloro- | | 10.40 (s, 1H), 9.09 (d, 1H), 8.18 (d, | [MH]⁺ |
| | benzo[b]thiophene-2- | | 1H) 8.11-8.28 (m, 1H), 7.92-8.07 | |
| | carboxamido)benzoic acid | | (m, 1H), 7.74-7.87 (m, 2H), 7.56- | |
| | | | 7.74 (m, 3H), 7.30 (t, 1H), 7.17 (ddd, | |
| | | | 1H), 1.31 (s, 9H) | |
| 76 | 2-(3-Chlorobenzo[b]- | >200 | 14.13 (br.s, 1H), 12.15 (s, 1H), | 441.72 |
| | thiophene-2-carboxamido)-4- | | 11.69 (s, 1H), 9.16 (d, 1H), 8.88 (d, | [MH]⁺ |
| | (isoxazol-3-ylcarbamoyl)- | | 1H), 8.09-8.29 (m, 1H), 8.17 (d, 1H), | |
| | benzoic acid | | 7.99 (dd, 1H), 7.86 (dd, 1H), 7.54- | |
| | | | 7.73 (m, 2H), 7.07 (d, 1H) | |
| 77 | 2-(3-Chlorobenzo[b]- | >200 | 14.09 (br.s, 1H), 12.17 (br.s, 1H), | 454.74 |
| | thiophene-2-carboxamido)-4- | | 11.04 (s, 1H), 9.12 (d, 1H), 8.18 (dd, | [MH]⁺ |
| | (1-methyl-1H-pyrazol-3-yl- | | 1H), 8.12 (d, 1H), 7.93-8.05 (m, 1H), | |
| | carbamoyl)benzoic acid | | 7.82 (dd, 1H), 7.46-7.73 (m, 3H), | |
| | | | 6.62 (d, 1H), 3.80 (s, 3H) | |
| 78 | 4-(4-tert-Butoxyphenyl- | >200 | 14.14 (br.s, 1H), 12.35 (br.s, 1H), | 523.06 |
| | carbamoyl)-2-(3-chloro- | | 10.39 (s, 1H), 9.08 (d, 1H), 8.11- | [MH]⁺ |
| | benzo[b]thiophene-2- | | 8.26 (m, 2H), 7.92-8.06 (m, 1H), | |
| | carboxamido)benzoic acid | | 7.76 (dd, 1H), 7.59-7.73 (m, 4H), | |
| | | | 6.98 (m, 2H), 1.30 (s, 9H) | |
| 79 | 2-(3-Chlorobenzo[b]- | >200 | 14.15 (br.s, 1H), 12.26 (br.s, 1H), | 480.98 |
| | thiophene-2-carboxamido)-4- | | 10.33 (s, 1H), 9.07 (d, 1H), 8.10- | [MH]⁺ |
| | (4-methoxyphenyl- | | 8.24 (m, 2H), 7.99 (dd, 1H), 7.77 | |
| | carbamoyl)benzoic acid | | (dd, 1H), 7.57-7.73 (m, 4H), 6.95 (m, | |
| | | | 2H), 3.76 (s, 3H) | |
| 80 | 2-(3-Chlorobenzo[b]- | >200 | 14.03 (s, 1H), 12.25 (br.s, 1H), | 534.04 |
| | thiophene-2-carboxamido)-4- | | 10.53 (s, 1H), 9.09 (d, 1H), 8.11- | [MH]⁺ |
| | (4-(2-oxopyrrolidin-1-yl)- | | 8.23 (m, 2H), 7.94-8.04 (m, 1H), | |
| | phenylcarbamoyl)benzoic | | 7.75-7.87 (m, 3H), 7.59-7.71 (m, | |
| | acid | | 4H), 3.84 (t, 2H), 2.45-2.47 (m, 2H), | |
| | | | 1.98-2.18 (m, 2H) | |
| 81 | 2-(3-Chlorobenzo[b]- | >200 | 12.02 (br.s, 1H), 10.06 (s, 1H), 9.06 | |
| | thiophene-2-carboxamido)-4- | | (d, 1H), 8.16 (d, 1H), 8.08-8.23 (m, | 511.01 |
| | (4-(2-hydroxyethoxy)- | | 1H), 7.87-8.03 (m, 1H), 7.77 (dd, | [MH]⁺ |
| | phenylcarbamoyl)benzoic | | 1H), 7.45-7.73 (m, 4H), 6.96 (m, | |
| | acid | | 2H), 4.04 (t, 2H), 3.75 (t, 2H) | |
| 82 | 2-(3-Chlorobenzo[b]- | >200 | 14.14 (br.s, 1H), 12.22 (br.s, 1H), | 466.90 |
| | thiophene-2-carboxamido)-4- | | 9.78 (s, 1H), 9.64 (s, 1H), 9.13 (d, | [MH]⁺ |
| | (2-hydroxyphenyl- | | 1H), 8.18 (d, 1H), 8.06-8.28 (m, 1H), | |
| | carbamoyl)benzoic acid | | 7.91-8.08 (m, 1H), 7.81 (dd, 1H), | |
| | | | 7.73 (dd, 1H), 7.56-7.71 (m, 2H), | |
| | | | 6.99-7.12 (m, 1H), 6.94 (dd, 1H), | |
| | | | 6.86 (td, 1H) | |
| 83 | 4-(4-Benzylpiperazine-1- | >200 | 12.22 (s, 1H), 10.86 (br.s, 1H), 8.67 | 533.97 |
| | carbonyl)-2-(3-chloro- | | (d, 1H), 8.14 (d, 1H), 8.06-8.30 (m, | [MH]⁺ |
| | benzo[b]thiophene-2- | | 1H), 7.84-8.07 (m, 1H), 7.40-7.79 | |
| | carboxamido)benzoic acid | | (m, 7H), 7.32 (dd, 1H), 4.32 (br.s, | |
| | | | 2H), 2.73-4.04 (m, 8H) | |
| 84 | 2-(3-Chlorobenzo[b]- | >200 | 14.09 (br.s, 1H) 12.25 (br.s, 1H) | 543.99 |
| | thiophene-2-carboxamido)-4- | | 10.54 (s, 1H) 9.10 (d, 1H) 8.29-8.44 | [MH]⁺ |
| | (4-(pyridin-3-yloxy)phenyl- | | (m, 2H) 8.12-8.25 (m, 2H) 7.93-8.07 | |
| | carbamoyl)benzoic acid | | (m, 1H) 7.73-7.92 (m, 3H) 7.57-7.73 | |
| | | | (m, 2H) 7.33-7.50 (m, 2H) 7.13 (m, | |
| | | | 2H) | |
| 85 | 4-(3-Chloro-4-(pyridin-3- | >200 | 14.20 (br.s, 1H), 12.37 (br.s, 1H), | 577.94 |
| | yloxy)phenylcarbamoyl)-2-(3- | | 10.69 (s, 1H), 9.11 (d, 1H), 8.28- | [MH]⁺ |
| | chlorobenzo[b]thiophene-2- | | 8.43 (m, 2H), 8.08-8.27 (m, 3H), | |
| | carboxamido)benzoic acid | | 7.91-8.06 (m, 1H), 7.72-7.87 (m, | |
| | | | 2H), 7.58-7.72 (m, 2H), 7.42 (dd, | |
| | | | 1H), 7.34 (ddd, 1H), 7.29 (d, 1H) | |
| 86 | 2-(3-Chlorobenzo[b]- | >200 | 14.05 (br.s, 1H), 12.19 (br.s, 1H), | 470.94 |
| | thiophene-2-carboxamido)-4- | | 9.32 (t, 1H), 9.03 (d, 1H), 8.13 (d, | [MH]⁺ |
| | (thiophen-2-ylmethyl- | | 1H), 8.05-8.34 (m, 2H), 7.84-8.05 | |
| | carbamoyl)benzoic acid | | (m, 1H), 7.51-7.82 (m, 3H), 7.40 (dd, | |
| | | | 1H), 7.05 (d, 1H), 6.98 (dd, 1H), | |
| | | | 4.66 (d, 2H) | |
| 87 | 2-(3-Chlorobenzo[b]- | >200 | 13.27 (br.s, 1H), 12.34 (br.s, 1H), | 541.56 |
| | thiophene-2-carboxamido)-4- | | 9.28 (s, 1H), 8.09-8.37 (m, 4H), | [MH]⁺ |
| | (6-chlorobenzo[d]thiazol-2-yl- | | 7.88-8.09 (m, 2H), 7.72-7.86 (m, 1H) | |
| | carbamoyl)benzoic acid | | 7.57-7.72 (m, 2H), 7.36-7.57 (m, 1H) | |
| 88 | 4-(Benzo[b]thiophen-2-yl- | >200 | 14.07 (br.s, 1H), 12.35 (br.s, 1H), | 520.98 |
| | methylcarbamoyl)-2-(3- | | 9.42 (t, 1H), 9.06 (d, 1H), 8.07-8.25 | [MH]⁺ |
| | chlorobenzo[b]thiophene-2- | | (m, 2H), 7.85-8.03 (m, 2H), 7.54- | |
| | carboxamido)benzoic acid | | 7.85 (m, 4H), 7.15-7.47 (m, 3H), | |
| | | | 4.76 (d, 2H) | |
| 89 | 2-(3-Chlorobenzo[b]- | >200 | 14.07 (br.s, 1H), 12.38 (br.s, 1H), | 528.98 |
| | thiophene-2-carboxamido)-4- | | 10.87 (s, 1H), 9.12 (d, 1H), 8.14- | [MH]⁺ |
| | (4-(methylsulfonyl)phenyl- | | 8.27 (m, 2H), 8.00 (m, 5H), 7.75- | |
| | carbamoyl)benzoic acid | | 7.86 (m, 1H), 7.56-7.72 (m, 2H), | |
| | | | 3.20 (s, 3H) | |
| 90 | 2-(3-Chlorobenzo[b]- | >200 | 14.09 (s, 1H), 12.25 (br.s, 1H), 9.35 | 504.78 |
| | thiophene-2-carboxamido)-4- | | (t, 1H), 9.03 (s, 1H), 8.07-8.27 (m, | [MH]⁺ |
| | ((5-chlorothiophen-2-yl- | | 2H), 7.92-8.05 (m, 1H), 7.54-7.79 | |
| | methyl)carbamoyl)benzoic | | (m, 3H), 6.96 (d, 1H), 6.92 (d, 1H), | |
| | acid | | 4.56 (d, 2H) | |
| 91 | 2-(3-Chlorobenzo[b]- | >200 | 14.06 (br.s, 1H), 12.17 (br.s, 1H), | 580.58 |
| | thiophene-2-carboxamido)-4- | | 9.38 (t, 1H), 9.06 (s, 1H), 8.07-8.29 | [MH]⁺ |
| | ((5-(4-chlorophenyl)thiophen- | | (m, 2H), 7.86-8.06 (m, 1H), 7.53- | |
| | 2-ylmethyl)carbamoyl)- | | 7.80 (m, 5H), 7.26-7.53 (m, 3H), | |
| | benzoic acid | | 7.06 (d, 1H), 4.66 (d, 2H) | |
| 92 | 2-(3-Chlorobenzo[b]- | >200 | 13.97 (br.s, 1H) 12.10 (br.s, 1H) | 556.97 |
| | thiophene-2-carboxamido)-4- | | 8.64 (d, 1H) 8.11-8.21 (m, 1H) 7.93- | [MH]⁺ |
| | (N-4-chlorophenyl-N-(3- | | 8.02 (m, 1H) 7.87 (d, 1H) 7.58-7.71 | |
| | methoxypropyl)carbamoyl)- | | (m, 2H) 7.36 (m, 2H) 7.26 (m, 2H) | |
| | benzoic acid | | 7.05 (d, 1H) 3.83-3.96 (m, 2H) 3.33- | |
| | | | 3.41 (m, 2H) | |
| 93 | 2-(8-Chloro-2,3-dihydro- | >200 | 14.11 (br.s, 1H), 12.13 (br.s, 1H), | 542.9 |
| | thieno[2′,3′:4,5]benzo[1,2-b]- | | 10.57 (s, 1H), 9.07 (s, 1H), 8.17 (d, | [MH]⁺ |
| | [1,4]dioxine-7-carboxamido)- | | 1H), 7.59-7.91 (m, 4H), 7.28-7.52 | |
| | 4-(4-chlorophenylcarbamoyl)- | | (m, 3H), 4.36 (s, 4H) | |
| | benzoic acid | | | |
| 94 | 4-(4-Chlorophenyl- | >200 | 12.24 (br.s, 1H), 10.57 (s, 1H), 9.01 | 534.9 |
| | carbamoyl)-2-(5-(2,3-dihydro- | | (s, 1H), 8.15 (d, 1H), 7.83 (m, 2H) | [MH]⁺ |
| | benzo[b][1,4]dioxin-6-yl)- | | 7.63-7.78 (m, 2H), 7.54 (d, 1H), 7.43 | |
| | thiophene-2-carboxamido)- | | (m, 2H), 7.13-7.33 (m, 2H), 6.94 (d, | |
| | benzoic acid | | 1H), 4.29 (s, 4H) | |
| 95 | 2-(3-Chlorobenzo[b]- | >200 | 13.98 (br.s, 1H), 12.13 (br.s, 1H), | 543 |
| | thiophene-2-carboxamido)-4- | | 8.63 (s, 1H), 8.09-8.24 (m, 1H), | [MH]⁺ |
| | (N-4-chlorophenyl-N-(3- | | 7.92-8.05 (m, 1H), 7.87 (d, 1H), | |
| | hydroxypropyl)carbamoyl)- | | 7.55-7.73 (m, 2H), 7.36 (m, 2H), | |
| | benzoic acid | | 7.26 (m, 2H), 7.05 (d, 1H), 4.45 | |
| | | | (br.s, 1H), 3.79-4.03 (m, 2H), 3.45 (t, | |
| | | | 2H), 1.56-1.79 (m, 2H) | |
| 96 | 2-(3-Chlorobenzo[b]thio- | >200 | [400 MHz] 12.23 (s, 1H), 9.10 (s, | 507.2 |
| | phene-2-carboxamido)-4-(4- | | 1H), 10.40 (s, 1H), 8.20 (m, 2H), | [M − H]− |
| | (2-hydroxyprop-2-yl)phenyl- | | 8.00 (d, 1H), 7.60-7.90 (m, 5H), 7.50 | |
| | carbamoyl)benzoic acid | | (m, 2H), 4.97 (s, 1H), 1.22 (s, 6H) | |
| 97 | 2-(3-Chlorobenzo[b]- | >200 | [400 MHz] 14.00 (br.s, 1H), 12.23 (s, | 491 |
| | thiophene-2-carboxamido)-4- | | 1H), 12.20 (s, 1H), 8.70 (m, 1H), | [MH]⁺ |
| | (1,2,3,4-tetrahydroquinoline- | | 8.20 (m, 1H), 8.00 (m, 2H), 7.70 (m, | |
| | 1-carbonyl)benzoic acid | | 2H), 6.70-7.20 (m, 4H), 3.80 (t, 2H) | |
| | | | 2.80 (t, 2H), 2.00 (m, 2H) | |
| 98 | 2-(3-Chlorobenzo[b]- | 165 | [400 MHz] 14.10 (br.s, 1H), 12.20 (s, | 535.2 |
| | thiophene-2-carboxamido)-4- | | 1H), 8.80 (s, 1H), 8.20-8.30 (m, 2H), | [MH]⁺ |
| | (4-hydroxy-4-phenyl- | | 8.00 (d, 1H), 7.20-7.80 (m, 8H), 5.20 | |
| | piperidine-1-carbonyl)benzoic | | (s, 1H), 4.40 (m, 1H), 3.00-3.60 (m, | |
| | acid | | 3H), 1.90-2.00 (m, 2H), 1.50-1.80 | |
| | | | (m, 2H) | |
| 99 | 2-(3-Chlorobenzo[b]- | 178 | [400 MHz] 14.10 (br.s, 1H), 12.20 (s, | 569.1 |
| | thiophene-2-carboxamido)-4- | | 1H), 8.80 (s, 1H), 8.20-8.30 (m, 2H), | [MH]⁺ |
| | (4-(4-chlorophenyl)-4- | | 8.00 (d, 1H), 7.20-7.80 (m, 7H), 5.20 | |
| | hydroxypiperidine-1- | | (s, 1H), 4.40 (m, 1H), 3.00-3.60 (m, | |
| | carbonyl)benzoic acid | | 3H), 1.90-2.00 (m, 2H), 1.50-1.80 | |
| | | | (m, 2H) | |
| 100 | 4-(4-Benzyl-4-hydroxy- | >200 | [400 MHz] 14.10 (br.s, 1H), 12.20 (s, | 549.1 |
| | piperidine-1-carbonyl)-2-(3- | | 1H), 8.60 (s, 1H), 8.20 (m, 1H), 8.15 | [MH]⁺ |
| | chlorobenzo[b]thiophene-2- | | (m, 1H), 8.00 (m, 1H), 7.60 (m, 2H), | |
| | carboxamido)benzoic acid | | 7.20-7.30 (m, 6H), 4.70 (s, 1H), 4.20 | |
| | | | (m, 1H), 3.00-3.60 (m, 3H), 2.72 (s, | |
| | | | 2H), 1.90-2.00 (m, 2H), 1.50-1.80 | |
| | | | (m, 2H) | |
| 101 | 2-(3-Chlorobenzo[b]- | 175 | [400 MHz] 13.90 (br.s, 1H), 12.60 (s, | 521.1 |
| | thiophene-2-carboxamido)-4- | | 1H), 8.75 (m, 1H), 8.10-8.20 (m, | [MH]⁺ |
| | (3-phenoxypyrrolidine-1- | | 2H), 8.00 (m, 1H), 7.60 (m, 2H), | |
| | carbonyl)benzoic acid | | 7.20-7.50 (m, 3H), 6.80-7.00 (m, | |
| | | | 3H), 5.10 (m, 1H), 3.30-4.00 (m, | |
| | | | 4H), 2.00-2.40 (m, 2H) | |
| 102 | 2-(3-Chlorobenzo[b]- | >200 | [400 MHz] 10.40 (s, 1H), 9.10 (s, | 507.2 |
| | thiophene-2-carboxamido)-4- | | 1H), 8.10-8.20 (m, 2H), 8.00 (m, | [M − H]− |
| | (3-(2-hydroxyprop-2-yl)- | | 1H), 7.85 (m, 1H), 7.60-7.80 (m, | |
| | phenylcarbamoyl)benzoic | | 4H), 7.30 (m, 1H), 7.20 (m, 1H), | |
| | acid | | 5.05 (s, 1H), 1.45 (s, 6H) | |
| 103 | 2-(3-Chlorobenzo[b]- | 150 | [400 MHz] 12.50 (br.s, 1H), 8.75 (d, | 505 |
| | thiophene-2-carboxamido)-4- | | 1H), 8.16 (m, 1H), 8.13-8.08 (m, 1H), | [MH]⁺ |
| | (3-phenylpyrrolidine-1- | | 7.98 (m, 1H), 7.66-7.64 (m, 2H), | |
| | carbonyl)benzoic acid | | 7.49 (m, 1H), 7.40-7.35 (m, 3H), | |
| | | | 7.29-7.26 (m, 2H), 7.23 (m, 1H), | |
| | | | 4.00 (m, 1H), 3.75 (m, 1H), 3.64- | |
| | | | 3.57 (m, 1H), 3.49-3.40 (m, 2H), | |
| 104 | 2-(3-Chlorobenzo[b]- | 164 | [400 MHz] 14.0 (br.s, 1H), 12.16 (s, | 567.9 |
| | thiophene-2-carboxamido)-4- | | 1H), 8.67 (br.s, 1H), 8.17 (m, 1H), | [M − H]− |
| | (N-4-chlorophenyl-N-(di- | | 7.97 (m, 1H) 7.88 (m, 1H), 7.67- | |
| | methylcarbamoylmethyl)- | | 7.61 (m, 2H), 7.32 (m, 2H), 7.24 (m, | |
| | carbamoyl)benzoic acid | | 2H), 7.01 (m, 1H), 4.70 (s, 2H), 3.00 | |
| | | | (s, 3H), 2.87 (s, 3H). | |
| 105 | 2-(3-Chlorobenzo[b]- | 155 | [400 MHz] 14.0 (br.s, 1H), 12.48 | 519.2 |
| | thiophene-2-carboxamido)-4- | | (br.s, 1H), 8.64 (s, 1H), 8.18-8.11 | [MH]⁺ |
| | (4-phenylpiperidine-1- | | (m, 2H), 7.98 (m, 1H), 7.66 (m, 2H), | |
| | carbonyl)benzoic acid | | 7.30 (m, 5H), 7.21 (m, 1H), 4.65 (m, | |
| | | | 1H), 3.68 (m, 1H), 3.65 (m, 1H), | |
| | | | 2.93-2.81 (m, 2H), 1.89 (m, 1H), | |
| | | | 1.75 (m, 1H), 1.64 (m, 2H). | |
| 106 | 2-(3-Chlorobenzo[b]- | 160 | [400 MHz] 12.0 (br.s, 1H), 8.64 (m, | 558.9 |
| | thiophene-2-carboxamido)-4- | | 1H), 8.18-8.16 (dd, 1H), 7.98-7.96 | [MH]⁺ |
| | (N-4-chlorophenyl-N-(2,3- | | (dd, 1H), 7.86 (m, 1H), 7.67-7.61 (m, | |
| | dihydroxypropyl)carbamoyl)- | | 2H), 7.33 (m, 4H), 7.02 (m, 1H), | |
| | benzoic acid | | 3.94 (m, 1H), 3.73-3.69 (m, 2H), | |
| | | | 3.36 (m, 2H). | |
| 107 | 2-(6-Chlorobenzo[b]- | >200 | 12.60 (br.s, 1H), 9.31 (t, 1H), 8.65 | 494 |
| | thiophene-2-carboxamido)-5- | | (d, 1H), 8.60 (d, 1H), 8.29 (d, 1H), | [M − H]− |
| | ((5-chlorothiophen-2-yl- | | 8.14-8.20 (m, 1H), 8.13 (s, 1H), 8.10 | |
| | methyl)carbamoyl)benzoic | | (d, 1H), 7.54 (dd, 1H), 6.96 (d, 1H), | |
| | acid | | 6.91 (d, 1H), 4.56 (d, 2H) | |
| 108 | 2-(3-Chlorobenzo[b]- | >200 | 8.52-8.80 (m, 1H), 8.25 (br.s, 1H), | 538.87 |
| | thiophene-2-carboxamido)-5- | | 8.09-8.20 (m, 1H), 7.94-8.07 (m, | [MH]⁺ |
| | (3-(4-chlorophenyl)- | | 1H), 7.83 (d, 1H), 7.58-7.74 (m, 2H), | |
| | pyrrolidine-1-carbonyl)- | | 7.23-7.47 (m, 4H), 3.36-4.11 (m, | |
| | benzoic acid | | 5H), 1.82-2.37 (m, 2H) | |
| 109 | 2-(3-Chlorobenzo[b]- | >200 | 12.32 (s, 1H), 8.71 (d, 1H), 8.15- | 490.89 |
| | thiophene-2-carboxamido)-5- | | 8.24 (m, 1H), 8.13 (d, 1H), 7.93-8.06 | [MH]⁺ |
| | (1,2,3,4-tetrahydro- | | (m, 1H), 7.81 (dd, 1H), 7.57-7.74 (m, | |
| | isoquinoline-2-carbonyl)- | | 2H), 7.20 (s, 4H), 4.52-4.98 (m, 2H), | |
| | benzoic acid | | 3.53-4.02 (m, 2H), 2.90 (t, 2H) | |
| 110 | 5-(1-(4-Chlorophenyl)ethyl- | >200 | 12.50 (br.s, 1H), 8.95 (d, 1H), 8.64 | 538.89 |
| | carbamoyl)-2-(5-(4-chloro- | | (d, 1H), 8.60 (d, 1H), 8.15 (dd, 1H), | [MH]⁺ |
| | phenyl)thiophene-2- | | 7.79-7.85 (m, 2H), 7.78 (d, 1H), 7.72 | |
| | carboxamido)benzoic acid | | (d, 1H), 7.50-7.60 (m, 2H), 7.33-7.50 | |
| | | | (m, 4H), 5.17 (dq, 1H), 1.49 (d, 3H) | |
| 111 | 2-(5-(4-Chlorophenyl)- | >200 | 12.31 (s, 1H), 8.80 (d, 1H), 8.63 (d, | 516.9 |
| | thiophene-2-carboxamido)-5- | | 1H), 8.59 (d, 1H), 8.15 (dd, 1H), | [MH]⁺ |
| | (indan-2-ylcarbamoyl)benzoic | | 7.79-7.84 (m 2H), 7.77 (d, 1H), 7.72 | |
| | acid | | (d, 1H), 7.49-7.59 (m, 2H), 7.20-7.30 | |
| | | | (m, 2H), 7.08-7.20 (m, 2H), 4.73 (sxt | |
| | | | 1H), 3.26 (dd, 2H), 2.99 (dd, 2H) | |
| 112 | 2-(5-(4-Chlorophenyl)- | >200 | 8.74 (d, 2H), 8.61 (d, 1H), 8.56 (d, | 530.94 |
| | thiophene-2-carboxamido)-5- | | 1H), 7.89-8.02 (m, 1H), 7.75-7.85 | [MH]⁺ |
| | (1,2,3,4-tetrahydro- | | (m 3H), 7.69 (d, 1H), 7.45-7.58 (m, | |
| | naphthalen-1-ylcarbamoyl)- | | 2H), 7.04-7.26 (m, 4H), 5.01-5.43 | |
| | benzoic acid | | (m, 1H), 2.64-2.88 (m, 2H), 1.60-.12 | |
| | | | (m, 4H) | |
| 113 | 2-(3-Chlorobenzo[b]- | >200 | [353° K] 8.58 (d, 1H), 8.15 (d, 1H), | 537.01 |
| | thiophene-2-carboxamido)-5- | | 8.01-8.13 (m, 1H), 7.85-8.01 (m, | [MH]⁺ |
| | (4-(4-fluorophenyl)piperidine- | | 1H), 7.52-7.69 (m, 2H), 7.46 (dd, | |
| | 1-carbonyl)benzoic acid | | 1H), 7.21-7.40 (m, 2H), 7.00-7.19 | |
| | | | (m, 2H), 4.08-4.39 (m, 2H), 3.04- | |
| | | | 3.18 (m, 2H), 2.81-2.92 (m, 1H), | |
| | | | 1.75-1.99 (m, 2H), 1.62 (m, 2H) | |
| 114 | 2-(3-Chlorobenzo[b]- | >200 | [353° K] 8.56 (d, 1H), 8.09 (d, 1H), | 551.02 |
| | thiophene-2-carboxamido)-5- | | 8.02-8.13 (m, 1H), 7.82-8.00 (m, | [MH]⁺ |
| | (4-(4-fluorobenzyl)piperidine- | | 1H), 7.53-7.70 (m, 2H), 7.38 (dd, | |
| | 1-carbonyl)benzoic acid | | 1H), 7.15-7.30 (m, 2H), 6.94-7.15 | |
| | | | (m, 2H), 3.92-4.25 (m, 2H), 2.80- | |
| | | | 2.94 (m, 2H), 2.58 (d, 2H), 1.74-1.95 | |
| | | | (m, 1H), 1.54-1.72 (m, 2H), 1.05- | |
| | | | 1.27 (m, 2H) | |
| 115 | 2-(3-Chlorobenzo[b]- | >200 | [353° K] 12.12 (br.s, 1H), 8.66 (d, | 552.95 |
| | thiophene-2-carboxamido)-5- | | 1H), 8.07-8.21 (m, 2H), 7.92-8.05 | [MH]⁺ |
| | (4-(4-chlorophenyl)piperidine- | | (m, 1H), 7.73 (dd, 1H), 7.56-7.69 (m, | |
| | 1-carbonyl)benzoic acid | | 2H), 7.19-7.43 (m, 4H), 3.91-4.92 | |
| | | | (m, 2H), 3.00-3.25 (m, 2H), 2.79- | |
| | | | 3.00 (m, 1H), 1.78-1.98 (m, 2H), | |
| | | | 1.51-1.77 (m, 2H) | |
| 116 | 2-(3-Chlorobenzo[b]- | >200 | [353° K] 12.08 (br.s, 1H), 8.66 (d, | 549.04 |
| | thiophene-2-carboxamido)-5- | | 1H), 8.11 (d, 1H), 8.07-8.17 (m, 1H), | [MH]⁺ |
| | (4-(4-methoxyphenyl)- | | 7.91-8.05 (m, 1H), 7.73 (dd, 1H), | |
| | piperidine-1-carbonyl)benzoic | | 7.57-7.69 (m, 2 H), 7.20 (m, 2H), | |
| | acid | | 6.88 (m, 2H), 4.14-4.26 (m, 2H), | |
| | | | 3.76 (s, 3H), 2.95-3.20 (m, 2H), | |
| | | | 2.74-2.90 (m, 1H), 1.74-1.94 (m, | |
| | | | 2H), 1.49-1.74 (m, 2H) | |
| 117 | 2-(3-Chlorobenzo[b]- | >200 | [353° K] 12.26 (br.s. 1H), 8.64 (d, | 563.05 |
| | thiophene-2-carboxamido)-5- | | 1H), 8.08-8.15 (m, 1H), 8.05 (d, 1H), | [MH]⁺ |
| | (4-(4-methoxybenzyl)- | | 7.90-8.02 (m, 1H), 7.47-7.76 (m, | |
| | piperidine-1-carbonyl)benzoic | | 3H), 7.10 (m, 2H), 6.85 (m, 2H), | |
| | acid | | 3.93-4.15 (m, 2H), 3.75 (s, 3H), | |
| | | | 2.86-3.04 (m, 2H), 2.53-2.55 (m, | |
| | | | 2H), 1.74-1.96 (m, 1H), 1.52-.74 (m, | |
| | | | 2H), 1.03-1.27 (m, 2H) | |
| 118 | 2-(3-Chlorobenzo[b]- | >200 | [353° K] 8.56 (d, 1H), 8.03-8.12 (m, | 566.74 |
| | thiophene-2-carboxamido)-5- | | 2H), 7.88-8.01 (m, 1H), 7.53-7.67 | [MH]⁺ |
| | (4-(4-chlorobenzyl)piperidine- | | (m, 2H), 7.39 (dd, 1H), 7.27-7.35 (m, | |
| | 1-carbonyl)benzoic acid | | 2H), 7.17-7.26 (m, 2H), 3.86-4.20 | |
| | | | (m, 2H), 2.83-2.97 (m, 2H), 2.59 (d, | |
| | | | 2H), 1.74-1.94 (m, 1H), 1.50-1.73 | |
| | | | (m, 2H), 1.10-1.27 (m, 2H) | |
| 119 | 5-(4-Chlorophenyl- | >200 | 12.43 (s, 1H), 10.49 (s, 1H), 8.84 (d, | 578.95 |
| | carbamoyl)-2-(4-(5,6-di- | | 1H) 8.69 (d, 1H), 8.39 (m, 2H), 8.29 | [MH]⁺ |
| | chloro-1H-benzo[d]imidazol- | | (dd, 1H), 8.16 (m, 2H), 7.92 (s, 2H), | |
| | 2-yl)benzamido)benzoic acid | | 7.83 (m, 2H), 7.43 (m, 2H) | |
| 120 | 5-(4-Chlorophenyl- | >200 | 13.76 (s, 1H), 12.60 (br.s, 1H), | n.d. |
| | carbamoyl)-2-(2-hydroxy-4- | | 11.65 (br.s, 1H), 10.46 (s, 1H), 8.84 | |
| | (thiophen-2-yl)benzamido)- | | (d, 1H), 8.64 (d, 1H), 8.22 (dd, 1H), | |
| | benzoic acid | | 7.96 (d, 1H), 7.73-7.89 (m, 2H), 7.66 | |
| | | | (dd, 1H), 7.61 (dd, 1H), 7.37-7.47 | |
| | | | (m, 2H), 7.33 (dd, 1H), 7.29 (d, 1H), | |
| | | | 7.20 (dd, 1H) | |
| 121 | 2-(3-Chlorobenzo[b]- | >200 | 12.29 (s, 1H), 8.70 (d, 1H), 8.14- | 546.9 |
| | thiophene-2-carboxamido)-5- | | 8.23 (m, 1H), 8.11 (d, 1H), 7.92-8.04 | [MH]⁺ |
| | (4-(2,6-dimethylphenyl)- | | (m, 1H), 7.77 (dd, 1H), 7.57-7.70 (m, | |
| | piperidine-1-carbonyl)benzoic | | 2H), 6.97 (s, 3H), 4.61 (br.s, 2H), | |
| | acid | | 3.84 (br.s, 1H), 3.24-3.39 (m, 2H), | |
| | | | 2.39 (s, 6H), 1.91-2.21 (m, 2H), | |
| | | | 1.50-1.71 (m, 2H) | |
| 122 | 2-(3-Chlorobenzo[b]- | >200 | 14.08 (br. s., 1 H) 12.30 (br. s., 1 H) | 534.83 |
| | thiophene-2-carboxamido)-5- | | 9.13 (s, 1H), 8.68 (d, 1H), 8.13-8.24 | [MH]⁺ |
| | (4-(4-hydroxyphenyl)- | | (m, 1H), 8.10 (d, 1H), 7.92-8.05 (m, | |
| | piperidine-1-carbonyl)benzoic | | 1H), 7.76 (dd, 1H), 7.57-7.72 (m, | |
| | acid | | 2H), 7.07 (m, 2H), 6.69 (m, 2H), | |
| | | | 4.56 (br.s, 1H), 3.76 (br.s, 1H), 3.00 | |
| | | | (br.s, 2H), 2.59-2.78 (m, 1H), 1.68- | |
| | | | 1.91 (m, 2H), 1.42-1.68 (m, 2H) | |
| 123 | 2-(4-(5,6-Dichloro-1H- | >200 | 12.37 (s, 1H), 8.77 (d, 1H), 8.71 (t, | 572.85 |
| | benzo[d]imidazol-2-yl)- | | 1H), 8.58 (d, 1H), 8.39 (m, 2H), | [MH]⁺ |
| | benzamido)-5-(phenethyl- | | 8.08-8.19 (m, 3H), 7.92 (s, 2H), | |
| | carbamoyl)benzoic acid | | 7.15-7.39 (m, 5H), 3.45-3.56 (m, | |
| | | | 2H), 2.87 (t, 2H) | |
| 124 | 2-(4-(5,6-Dichloro-1H- | >200 | 12.29 (s, 1H), 8.76 (d, 1H), 8.39 (m, | 536.87 |
| | benzo[d]imidazol-2-yl)- | | 2H), 8.15 (m, 2H), 8.06 (d, 1H), 7.92 | [MH]⁺ |
| | benzamido)-5-(piperidine-1- | | (s, 2H), 7.72 (dd, 1H), 3.47 (br.s, | |
| | carbonyl)benzoic acid | | 4H), 1.42-1.72 (m, 6H) | |
| 125 | 5-(Cyclohexylcarbamoyl)-2- | >200 | [+TFA] 12.38 (s, 1H), 8.76 (d, 1H), | 550.9 |
| | (4-(5,6-dichloro-1H-benzo[d]- | | 8.58 (d, 1H), 8.26-8.48 (m, 3H), | [MH]⁺ |
| | imidazol-2-yl)benzamido)- | | 8.08-8.25 (m, 3H), 7.94 (s, 2H), 3.78 | |
| | benzoic acid | | (br.s, 1H), 1.53-1.97 (m, 4H), 1.08- | |
| | | | 1.43 (m, 6H) | |
| 126 | 5-(Cyclohexylmethyl- | >200 | [+TFA] 12.38 (s, 1H), 8.76 (d, 1H), | 564.88 |
| | carbamoyl)-2-(4-(5,6-di- | | 8.51-8.63 (m, 2H), 8.33-8.44 (m, | [MH]⁺ |
| | chloro-1H-benzo[d]imidazol- | | 2H), 8.10-8.21 (m, 3H), 7.94 (s, 2H), | |
| | 2-yl)benzamido)benzoic acid | | 3.13 (t, 2H), 1.46-1.81 (m, 5H), 1.10- | |
| | | | 1.30 (m, 4H), 0.86-1.04 (m, 2H) | |
| 127 | 2-(4-(5,6-Dichloro-1H- | >200 | 12.37 (s, 1H), 8.77 (d, 1H), 8.51- | 482.81 |
| | benzo[d]imidazol-2-yl)- | | 8.62 (m, 2H), 8.33-8.44 (m, 2H), | [MH]⁺ |
| | benzamido)-5-(methyl- | | 8.08-8.20 (m, 3H), 7.92 (s, 2H), 2.81 | |
| | carbamoyl)benzoic acid | | (d, 3H) | |
| 128 | 2-(4-(5,6-Dichloro-1H- | >200 | 12.30 (s, 1H), 8.75 (d, 1H), 8.40 (m, | 496.83 |
| | benzo[d]imidazol-2-yl)- | | 2H) 8.15 (m, 2H), 8.09 (d, 1H), 7.93 | [MH]⁺ |
| | benzamide)-5-(dimethyl- | | (s, 2H), 7.76 (dd, 1H), 2.99 (s, 6H) | |
| | carbamoyl)benzoic acid | | | |
| 129 | 5-(4-Butylphenylcarbamoyl)- | >200 | 12.42 (s, 1H), 10.30 (s, 1H), 8.83 (d, | 600.87 |
| | 2-(4-(5,6-dichloro-1H- | | 1H), 8.69 (d, 1H), 8.40 (m, 2H), 8.28 | [MH]⁺ |
| | benzo[d]imidazol-2-yl)- | | (dd, 1H) 8.17 (m, 2H), 7.92 (s, 2H), | |
| | benzamido)benzoic acid | | 7.68 (m, 2H), 7.18 (m, 2H), 2.53- | |
| | | | 2.61 (m, 2H), 1.48-1.64 (m, 2H), | |
| | | | 1.26-1.39 (m, 2H), 0.91 (t, 3H) | |
| 130 | 2-(4-(5,6-Dichloro-1H- | >200 | 12.37 (s, 1H), 8.77 (d, 1H) 8.52-8.67 | 524.89 |
| | benzo[d]imidazol-2-yl)- | | (m, 2H), 8.32-8.46 (m, 2H), 8.07- | [MH]⁺ |
| | benzamido)-5-(isobutyl- | | 8.20 (m, 3H), 7.92 (s, 2H), 3.11 (t, | |
| | carbamoyl)benzoic acid | | 2H), 1.78-1.95 (m, 1H), 0.91 (d, 6H) | |
| 131 | 2-(4-(5,6-Dichloro-1H- | >200 | [353° K] 12.50 (br.s, 1H), 8.74 (d, | 580.9 |
| | benzo[d]imidazol-2-yl)- | | 1H), 8.26-8.44 (m, 2H), 8.11-8.23 | [MH]⁺ |
| | benzamido)-5-(diisobutyl- | | (m, 2H), 8.03 (d, 1H), 7.86 (br.s, | |
| | carbamoyl)benzoic acid | | 2H), 7.61 (dd, 1H), 3.24 (d, 4H), | |
| | | | 1.78-2.21 (m, 2H), 0.86 (d, 12H) | |
| 132 | 5-(Benzylcarbamoyl)-2-(4- | >200 | 12.40 (s, 1H), 9.19 (t, 1H), 8.79 (d, | 558.84 |
| | (5,6-dichloro-1H-benzo[d]- | | 1H), 8.65 (d, 1H), 8.38 (m, 2H), 8.21 | [MH]⁺ |
| | imidazol-2-yl)benzamido)- | | (dd, 1H), 8.15 (m, 2H), 7.92 (s, 2H), | |
| | benzoic acid | | 7.31-7.39 (m, 4H), 7.19-7.30 (m, | |
| | | | 1H), 4.51 (d, 2H) | |
| 133 | 5-(1-(4-Chlorophenyl)ethyl- | >200 | [353° K] 12.24 (br.s, 1H), 8.75 (d, | 606.83 |
| | carbamoyl)-2-(4-(5,6- | | 1H), 8.67 (d, 1H), 8.60 (d, 1H), 8.36 | [MH]⁺ |
| | dichloro-1H-benzo[d]- | | (m, 2H), 8.09-8.23 (m, 3H), 7.86 (s, | |
| | imidazol-2-yl)benzamido)- | | 2H), 7.45 (m, 2H), 7.37 (m, 2H), | |
| | benzoic acid | | 5.12-5.30 (m, 1H), 1.54 (d, 3H) | |
| 134 | 2-(5-Chloro-3-phenyl-1H- | >200 | 13.73 (br.s, 1H), 12.27 (s, 1H), | 544.08 |
| | indole-2-carboxamido)-5-(4- | | 11.45 (s, 1H), 10.42 (s, 1H), 8.72 (d, | [MH]⁺ |
| | chlorophenylcarbamoyl)- | | 1H), 8.51 (d, 1H), 8.21 (dd, 1H), | |
| | benzoic acid | | 7.72-7.91 (m, 2H), 7.51-7.62 (m, | |
| | | | 4H), 7.28-7.51 (m, 6H) | |
| 135 | 5-(4-Chlorophenyl- | >200 | 12.47 (s, 1H), 10.51 (s, 1H), 8.86 (d, | 592.98 |
| | carbamoyl)-2-(4-(5,6- | | 1H), 8.70 (d, 1H), 8.30 (dd, 1H), | [MH]⁺ |
| | dichloro-1-methyl-1H- | | 8.17 (m, 2H), 8.13 (s, 1H), 8.12 (m, | |
| | benzo[d]imidazol-2-yl)- | | 2H), 8.02 (s, 1H), 7.71-7.91 (m, 2H), | |
| | benzamido)benzoic acid | | 7.23-7.51 (m, 2H), 3.97 (s, 3H) | |
| 136 | 5-(5-Chloropyridin-2-yl- | >200 | 10.86 (s, 1H), 8.77 (d, 1H), 8.71 (d, | 579.83 |
| | carbamoyl)-2-(4-(5,6- | | 1H), 8.43 (dd, 1H), 8.32-8.40 (m, | [MH]⁺ |
| | dichloro-1H-benzo[d]- | | 2H), 8.19-8.28 (m, 3H), 8.07 (dd, | |
| | imidazol-2-yl)benzamido)- | | 1H), 7.95 (dd, 1H), 7.92 (br.s, 2H) | |
| | benzoic acid | | | |
| 137 | 2-(4-(5,6-Dichloro-1H- | >200 | 12.37 (s, 1H), 8.76 (d, 1H), 8.52- | 565.07 |
| | benzo[d]imidazol-2-yl)- | | 8.62 (m, 2H), 8.40 (m, 2H), 8.09- | [M − H]− |
| | benzamido)-5-(heptyl- | | 8.21 (m, 3H), 7.92 (s, 2H), 3.21-3.35 | |
| | carbamoyl)benzoic acid | | (m, 2H), 1.54 (t, 2H), 1.15-1.42 (m, | |
| | | | 8H), 0.87 (t, 3H) | |
| 138 | 2-(4-(5,6-Dichloro-1H- | >200 | 12.43 (s, 1H), 10.37 (s, 1H), 8.85 (d, | 543.02 |
| | benzo[d]imidazol-2-yl)- | | 1H), 8.70 (d, 1H), 8.39 (m, 2H), 8.29 | [M − H]− |
| | benzamido)-5-(phenyl- | | (dd, 1H), 8.17 (m, 2H), 7.92 (s, 2H), | |
| | carbamoyl)benzoic acid | | 7.73-7.83 (m, 2H), 7.29-7.42 (m, | |
| | | | 2H), 7.06-7.19 (m, 1H) | |
| 139 | 2-(5-Chloro-3-phenyl-1H- | >200 | 13.69 (br.s, 1H), 12.27 (s, 1H), | 527.94 |
| | indole-2-carboxamido)-5-(2- | | 11.49 (s, 1H), 10.20 (s, 1H), 8.72 (d, | [MH]⁺ |
| | fluorophenylcarbamoyl)- | | 1H), 8.55 (d, 1H), 8.23 (dd, 1H), | |
| | benzoic acid | | 7.50-7.66 (m, 5H), 7.42-7.50 (m, | |
| | | | 2H), 7.14-7.41 (m, 5H) | |
| 140 | 5-(4-Chloro-2-fluorophenyl- | >200 | 13.72 (br.s, 1H), 12.26 (s, 1H), | 561.99 |
| | carbamoyl)-2-(5-chloro-3- | | 11.54 (br.s, 1H), 10.28 (s, 1H), 8.71 | [MH]⁺ |
| | phenyl-1H-indole-2- | | (d, 1H), 8.54 (d, 1H), 8.21 (dd, 1H), | |
| | carboxamido)benzoic acid | | 7.64 (t, 1H), 7.50-7.59 (m, 5H), 7.42- | |
| | | | 7.49 (m, 2H), 7.27-7.40 (m, 3H) | |

### Example 141

### 2-(4-(5,6-Dichloro-1H-benzo[d]imidazol-2-yl)benzamido)-5-(4-(4-fluorobenzyl)piperidine-1-carbonyl)benzoic acid

### Method 3

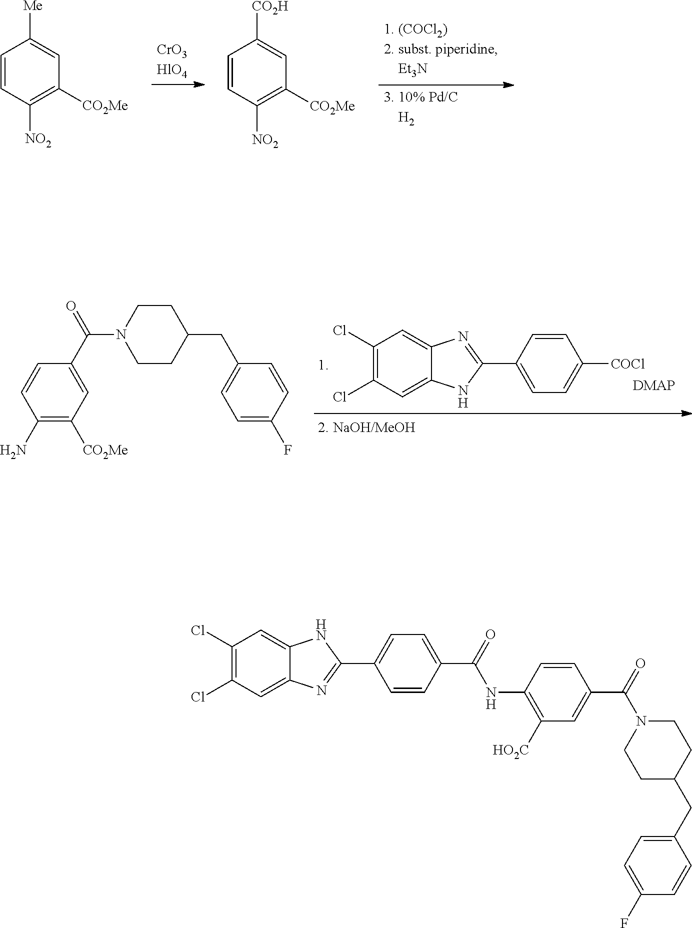

In a 1 L round-bottomed flask, to a mix of periodic acid (41.9 g, 184 mmol) and methyl 5-methyl-2-nitrobenzoate (8.97 g, 46.0 mmol) in 500 ml of acetonitrile, CrO₃ (0.919 g, 9.19 mmol) was added portionwise and the mix was stirred overnight at RT. About 200 ml of isopropanol were cautiously added to destroy CrO₃ and the mix was stirred for 2 h at RT; the inorganic solids were eliminated by filtration, then the solvent was evaporated under vacuum. The residue was partitioned between diluted HCl and AcOEt; the organic phase was further washed with brine, dried on Na₂SO₄ and evaporated; the resulting solid was washed with DCM to afford 7.68 g of crude 3-methoxycarbonyl-4-nitrobenzoic acid (74.2% yield) as a white solid.

To a suspension of this compound (0.6 g, 2.66 mmol) in dry DCM (50 ml), oxalyl dichloride (0.338 ml, 4.00 mmol) was added in one portion at room temperature; 3 drops of DMF were also added and the mixture was heated at 50° C. for 2 h. Volatile fractions were removed under vacuum to obtain methyl 5-chlorocarbonyl-2-nitrobenzoate as a pale yellow solid, which was resuspended in dry THF (40 ml); TEA (0.439 ml, 3.15 mmol) was added at room temperature followed by 4-(4-fluorobenzyl)piperidine (0.609 g, 3.15 mmol). The reaction was stirred overnight at the same temperature. Volatile fractions were evaporated under vacuum and the crude compound was portioned between ethyl acetate and a 2M aqueous solution of HCl. The organic phase was separated, dried (NaSO₄), filtered and evaporated under vacuum to afford a crude that was purified by flash chromatography (silica, hexane:EtOAc 4:6) to afford methyl 5-(4-(4-fluorobenzyl)-piperidine-1-carbonyl)-2-nitrobenzoate (0.756 g, 71.9% yield) as a white foam. A mixture of this compound (756 mg, 1.9 mmol) and 10% Pd/C (53 mg) in 50 ml of 1:1 methanol/ethyl acetate mixture was hydrogenated at 30 psi in a Parr apparatus overnight. The mixture was filtered through a Celite pad and evaporated to obtain methyl 2-amino-5-(4-(4-fluorobenzyl)piperidine-1-carbonyl)benzoate (660 mg, 94% yield) as a white foam.

A mixture of 4-(5,6-dichloro-1H-benzo[d]imidazol-2-yl)benzoic acid (199 mg, 0.648 mmol) and SOCl₂ (2 ml, 27.4 mmol) was stirred at 70° C. for a total of 5 h. SOCl₂ was evaporated completely, the residue was resuspended in 10 ml of dry THF, methyl 2-amino-5-(4-(4-fluorobenzyl)piperidine-1-carbonyl)benzoate (200 mg, 0.540 mmol) and DMAP (79 mg, 0.648 mmol) were added, and the mix was refluxed overnight. The mix was diluted with methanol and the precipitate was collected, then washed with aqueous K₂CO₃, aqueous HCl, water, and finally with ethyl ether. After drying 272 mg (76%) of methyl 2-(4-(5,6-dichloro-1H-benzo[d]imidazol-2-yl)benzamido)-5-(4-(4-fluorobenzyl)piperidine-1-carbonyl)-benzoate were obtained. This compound was dissolved in 20 ml of THF and 10 ml of MeOH, 6 N NaOH (274 μl, 1.65 mmol) was added and the solution was heated to 70° C. for 3 h. The mixture was filtered through a Celite pad, then poured into acidic water; the precipitate was collected, washed with plenty of water and with MeOH. After drying, 138 mg (52%) of the title compound were obtained as an off-white solid, mp.>200.

¹H-NMR (300 MHz, DMSO-d₆, 353° K), δ ppm: 12.14 (br.s, 1H), 8.73 (d, 1H), 8.36 (m, 2H), 8.14 (m, 2H), 8.07 (d, 1H), 7.86 (s, 2H), 7.67 (dd, 1H), 7.22 (dd, 2H), 6.97-7.15 (m, 2H), 3.87-4.21 (m, 2H), 2.82-3.10 (m, 2H), 2.59 (d, 2H), 1.85 (br.s, 1H), 1.66 (m, 2H), 1.09-1.34 (m, 2H); MS, m/z=645.03 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 142 | 2-(3-Chloro-6-methoxy- | >200 | 10.54 (br.s, 1H), 9.06 (s, 1H), 8.16 | 514.58 |
| | benzo[b]thiophene-2-carbox)- | | (d, 1H), 7.78-7.90 (m, 3H), 7.60-7.78 | [MH]⁺ |
| | 4-(4-chlorophenylcarbamoyl)- | | (m, 2H), 7.43 (m, 2H), 7.23 (dd, 1H), | |
| | benzoic acid | | 3.89 (s, 3H) | |
| 143 | 4-(4-Chlorophenyl- | >200 | 12.27 (s, 1H), 10.57 (s, 1H), 8.76 (d, | 566.78 |
| | carbamoyl)-2-(3,5-dibromo-2- | | 1H) 7.98-8.24 (m, 7.69-7.93 | [MH]⁺ |
| | hydroxybenzamido)benzoic | | (m, 3H), 7.27-7.54 (m, 2H) | |
| | acid | | | |
| 144 | 4-(4-Chlorophenyl- | >200 | [+TFA] 12.11 (s, 1H), 10.59 (s, 1H), | 477.98 |
| | carbamoyl)-2-(5-(pyrid-2-yl)- | | 9.05 (d, 1H), 8.61 (d, 1H), 8.17 (d, | [MH]⁺ |
| | thiophene-2-carboxamido)- | | 1H), 7.99-8.10 (m, 1H), 7.78-7.98 | |
| | benzoic acid | | (m, 5H), 7.73 (dd, 1H), 7.44 (m, 2H), | |
| | | | 7.39 (ddd, 1H) | |
| 145 | 4-(4-Chlorophenyl- | >200 | 12.38 (br.s, 1H), 10.56 (s, 1H), 9.03 | 507 |
| | carbamoyl)-2-(5-(4-methoxy- | | (s, 1H), 8.16 (d, 1H), 7.83 (m, 2H), | [MH]⁺ |
| | phenyl)thiophene-2- | | 7.76 (d, 1H), 7.66-7.74 (m, 3H), 7.55 | |
| | carboxamido)benzoic acid | | (d, 1H), 7.43 (m, 2H), 7.04 (m, 2H), | |
| | | | 3.82 (s, 3H) | |

### Example 146

### 2-(3-Chlorobenzo[b]thiophene-2-carboxamido)-4-(N-4-chlorophenyl-N-(2-hydroxyethyl)carbamoyl)benzoic acid

### Method 4

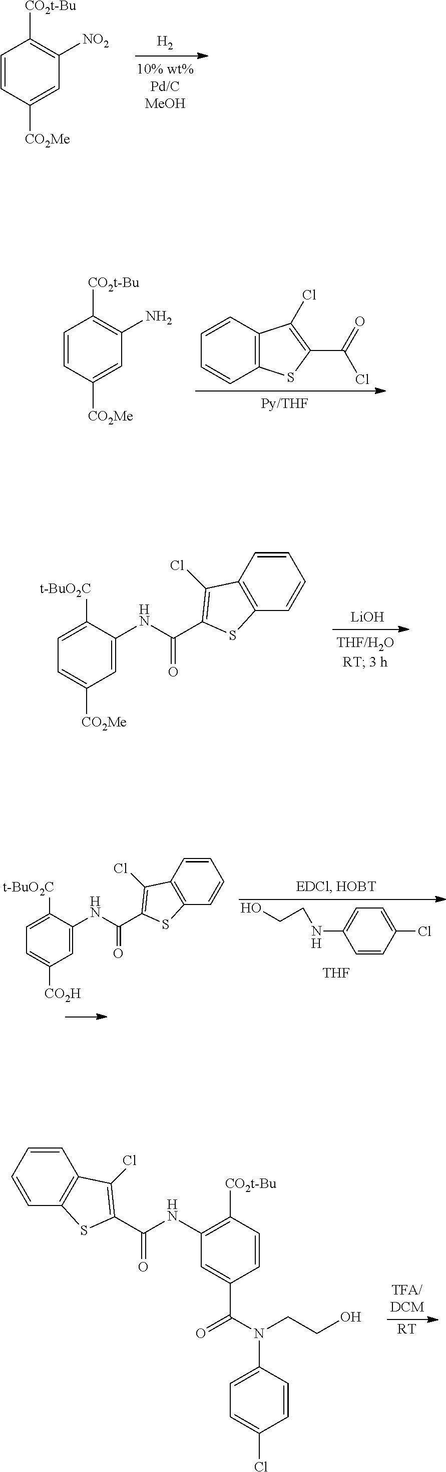

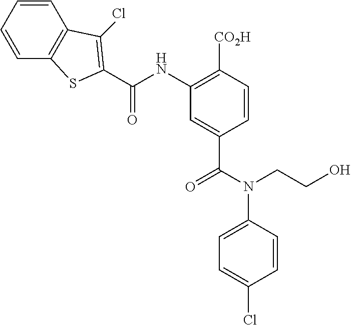 [SMILES: O=C(O)c1ccc(C(=O)N(CCO)c2ccc(Cl)cc2)cc1NC(=O)c1sc2ccccc2c1Cl]

A solution of t-butyl 4-methoxycarbonyl-2-nitrobenzoate in MeOH was hydrogenated for 18-20 h in the presence of 10% palladium on carbon. After filtration of the catalyst through a Celite pad, the filtrate was concentrated under vacuum to afford t-butyl 2-amino-4-methoxycarbonyl benzoate (75%). This compound was dissolved in THF and 1.1 eq. of 3-chlorobenzo[b]thiophenecarbonyl chloride were added. The reaction mixture was refluxed for 10-14 h and concentrated under vacuum. The crude residue was dissolved in THF-water (3:1) and LiOH (1.1 eq.) was added at a time. The solution was stirred overnight at room temperature, concentrated under vacuum and the residue was dissolved in the minimum volume of water, adjusting the pH to 6-7. The aqueous solution was extracted with ethyl acetate, and the organic layer was evaporated under vacuum. The residue was dissolved in DMF and 4-chloro-N-(2-hydroxyethyl)aniline (1.2 eq.), EDCl (1.5 eq.), HOBt (0.5 eq.) and DIEA (2.5 eq.) were added at 5-10° C. The reaction mixture was stirred at room temperature for 16 h, then was diluted with water. The precipitated solid was collected by suction filtration and dried. This compound was dissolved in dichloromethane, TFA was added and the reaction mixture was stirred for 1 h at room temperature. The solvent was evaporated under vacuum and the residue was triturated with diethyl ether to afford the title compound (21% overall), mp 200° C.

¹H-NMR (300 MHz, DMSO-d₆, 300° K), δ ppm: 14.25 (br.s, 1H, CO₂H); 12.20 (br.s, 1H, NH); 9.17 (d, J 1.2 Hz, 1 Ar—H). 8.19-6.65 (m, 10 Ar—H); 6.15 (br.s, 1H, OH); 4.45-4.42 (t, 2H); 3.46-3.43 (t, 2H); MS, m/z=528.9 [M-H]⁻

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 147 | 2-(3-Chloro-benzo[b]- | >250 | 15.12 (br.s, 1H, CO₂H); 8.95 (d, J | 411 |
| | thiophene-2-carboxamido)-4- | | 9.2 Hz, 1 Ar—H); 8.58 (s, 1H, NH); | [M − H]− |
| | (2-(4-chlorophenyl)ethyl- | | 8.15-7.28 (m, 10 Ar—H); 3.48-3.47 | |
| | carbamoyl)benzoic acid | | (m, 2H); 2.87-2.83 (m, 2H) | |
| 148 | 2-(3-Chloro-benzo[b]- | >250 | 12.19 (s, 1H, NH); 9.31-9.28 (t, 1H, | 497 |
| | thiophene-2-carboxamido)-4- | | NH); 9.04 (d, J 1.44 Hz, 1 Ar—H) | [M − H]− |
| | (4-chlorobenzylcarbamoyl)- | | 8.19-7.35 (m, 10 Ar—H); 4.49-4.47 (d, | |
| | benzoic acid | | 2H) | |
| 149 | 2-(3-Chloro-benzo[b]- | >250 | 14.2 (br.s, 1H, CO₂H); 12.16 (s, 1H, | 452 |
| | thiophene-2-carboxamido)-4- | | NH); 11.07 (s, 1H, NH); 9.14 (d, J | [MH]⁺ |
| | (pyridin-2-ylcarbamoyl)- | | 1.2 Hz, 1 Ar H); 8.42-7.19 (m, 10 | |
| | benzoic acid | | Ar—H) | |
| 150 | 2-(3-Chloro-benzo[b]- | >250 | 14.25 (br.s, 1H, CO₂H); 12.25 (br.s, | 451 |
| | thiophene-2-carboxamido)-4- | | 1H, NH); 11.54 (s, 1H, NH); 9.15 (d, | [M − H]− |
| | (pyrimidin-4-ylcarbamoyl)- | | J 1.44 Hz, 1 Ar—H). 8.98 (s, 1 Ar—H); | |
| | benzoic acid | | 8.76 (d, J 5.6Hz, 1 Ar—H); 8.24-7.64 | |
| | | | (m, 6 Ar—H) | |
| 151 | 4-(4-(4-Chloro-3-(trifluoro- | 200 | [400 MHz] 14.10 (br.s, 1H), 12.20 (s, | 637 |
| | methyl)phenyl)-4-hydroxy- | | 1H), 8.80 (s, 1H), 7.20-8.30 (m, 9H), | [MH]⁺ |
| | piperidine-1-carbonyl)-2-(3- | | 5.60 (s, 1H), 4.40 (m, 1H), 3.00-3.60 | |
| | chlorobenzo[b]thiophene-2- | | (m, 3H), 1.90-2.00 (m, 2H), 1.50- | |
| | carboxamido)benzoic acid | | 1.80 (m, 2H) | |
| 152 | 4-(4-Chloro-3-(hydroxy- | >200 | [400 MHz] 12.19 (s, 1H), 10.63 (s, | 512.9 |
| | methyl)phenylcarbamoyl)-2- | | 1H), 9.10 (s, 1H), 8.18 (m, 2H), 8.00 | [M − H]− |
| | (3-chlorobenzo[b]thiophene- | | (m, 2H) 7.81-7.78 (m, 2H), 7.66- | |
| | 2-carboxamido)benzoic acid | | 7.64 (m, 2H), 7.49 (m, 1H), 5.5 (br.s, | |
| | | | 1H), 4.57 (s, 2H). | |
| 153 | 2-(3-Chlorobenzo[b]- | >200 | [400 MHz] 14.0 (br.s, 1H), 12.23 (s, | 458 |
| | thiophene-2-carboxamido)-4- | | 1H), 8.86 (br.s, 1H), 8.18-8.12 (m, | [MH]⁺ |
| | (3-oxopiperazine-1-carbonyl)- | | 3H), 7.98 (m, 1H), 7.68-7.62 (m, 2H), | |
| | benzoic acid | | 7.32 (m, 1H), 4.12 (m, 1H), 3.92 (m, | |
| | | | 1H), 3.81 (m, 1H), 3.40-3.35 (m, 1H), | |
| | | | 3.25 (m, 2H). | |

### Example 154

### 2-(3-Chlorobenzo[b]thiophene-2-carboxamido)-4-(N-(4-chlorophenyl)-N-methylcarbamoyl)benzoic acid

### Method 5

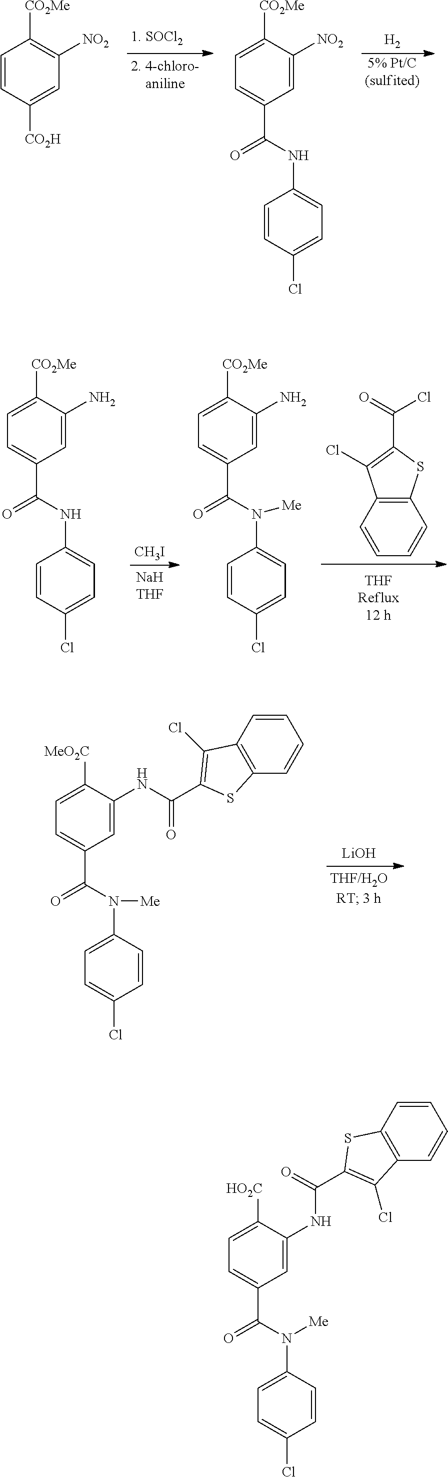

A solution of 2-nitro-terephthalic acid 1-methyl ester (1 eq.) in toluene was treated with thionyl chloride (2.5 eq.) and a catalytic amount of pyridine, and refluxed for 4 h. The reaction mixture was concentrated under vacuum to half volume, diluted again with toluene and concentrated again to half volume. This operation was repeated until all the acidity was removed. Then this solution was added dropwise to a stirred a solution of 4-chloroaniline (1.6 eq.) in toluene and stirring was continued for 14 h at room temperature. The reaction mixture was concentrated under vacuum and the residue was dissolved in ethyl acetate. The obtained solution was washed with dil. HCl and water, dried (MgSO₄) and evaporated under vacuum. The residue was chromatographed over silica gel, eluting with 12-15% ethyl acetate-hexane. The collected fractions were evaporated to dryness, dissolved in methanol and hydrogenated for 18-20 h in the presence of 10% platinum sulfide on carbon. After filtration of the catalyst through a Celite pad, the filtrate was concentrated under vacuum to afford the desired aniline.

This compound was dissolved in THF, and sodium hydride (1.1 eq.) was added portion wise at 0-5° C. Stirring was continued for 1 h, then methyl iodide (1.2 eq.) was added. Stirring was continued for 12 h at room temperature, the reaction mixture was diluted with cold water and extracted with ethyl acetate. The organic layer was dried and evaporated to dryness under vacuum. The residue was chromatographed over silica gel, eluting with 15% ethyl acetate-hexane. The collected fractions were evaporated under vacuum and the residue was dissolved in THF and treated with 1.1 eq. of 3-chloro-benzo[b]thiophene-carbonyl chloride. The reaction mixture was refluxed for 14 h, cooled and concentrated under vacuum. The residue was triturated with diethyl ether to afford the methyl 2-(3-chlorobenzo[b]thiophene-2-carboxamido)-4-(N-(4-chlorophenyl)-N-methylcarbamoyl)-benzoic acid.

This compound was dissolved in THF-water (3:1), treated with LiOH (1.1 eq.) and stirred over night at room temperature. The organic solvent was removed under vacuum, the pH was adjusted to 6-7 and the solution was extracted with ethyl acetate. The organic layer was dried (MgSO₄) and concentrated under vacuum to afford the title compound (15% overall), mp>200° C.

¹H-NMR (300 MHz, DMSO-d₆, 300° K), δ ppm: 14.08 (br.s, 1H, CO₂H); 12.31 (br.s, 1H, NH); 8.64 (d, J 1.2 Hz, 1 Ar—H), 8.18-7.03 (m, 10 Ar—H); 3.33 (s, 3H); MS, m/z=527[M-H]⁻

### Example 155

### 2-[N-(3-Chlorobenzo[b]thiophene-2-carbonyl)-N-methylamino]-4-(4-chloro-phenylcarbamoyl)benzoic acid

### Method 6

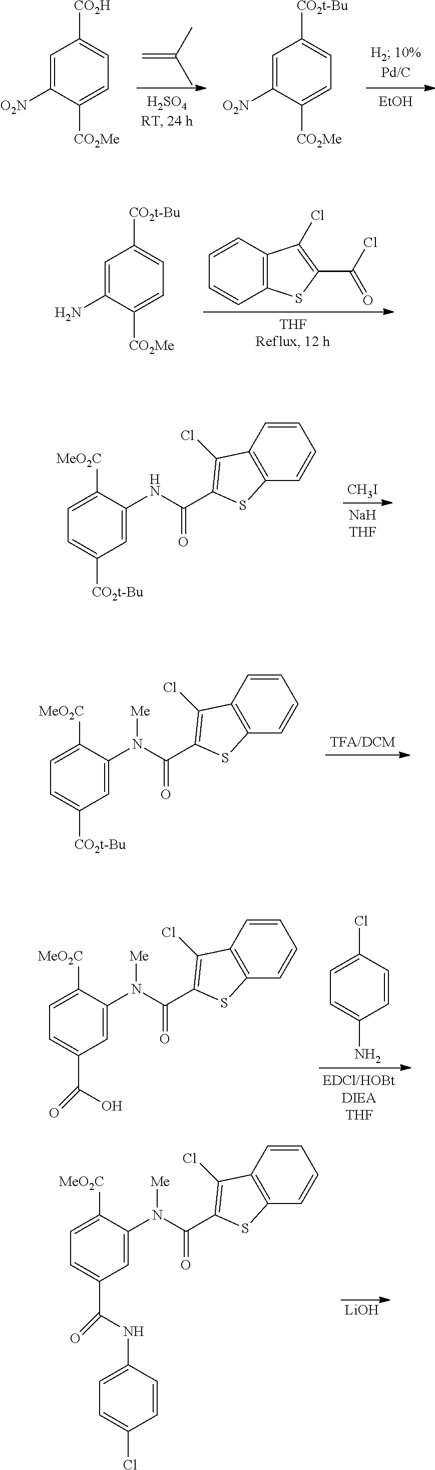

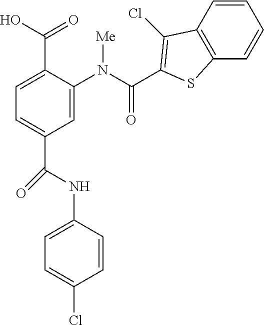 [SMILES: CN(C(=O)c1sc2ccccc2c1Cl)c1cc(C(=O)Nc2ccc(Cl)cc2)ccc1C(=O)O]

A solution of methyl 2-nitroterephthalate in dioxane was treated with a catalytic amount of sulfuric acid and condensed 2-methylpropene at −15° C. in a sealed tube. The reaction mixture was stirred at room temperature for 48 h, poured into 10% NaOH and extracted with ethyl acetate. The organic phase was washed with water, brine and dried over MgSO₄. The solvent was evaporated under vacuum to afford a residue that was dissolved in MeOH and hydrogenated for 20 h in the presence of 10% palladium on carbon. After filtration of the catalyst through a Celite pad, the filtrate was concentrated under vacuum to afford the corresponding aniline in 62% overall yield.

The aniline was dissolved in THF and 1.1 eq. of 3-chloro-benzo[b]thiophenecarbonyl chloride were added. The reaction mixture was refluxed for 14 h, cooled and concentrated under vacuum. The residue was triturated with diethyl ether, dried and dissolved in THF. Sodium hydride (1.1 eq.) was added portion wise at 0-5° C. and stirring was continued for 1 h. Methyl iodide (1.2 eq.) was added dropwise and stirring continued for 12 h at room temperature. The reaction mixture was diluted with cold water and extracted with ethyl acetate. The organic layer was evaporated to dryness and the residue was chromatographed over silica gel, eluting with 15% ethyl acetate-hexane. The collected fractions were evaporated to dryness, to afford the methylated amide in 33% overall yield. This compound was dissolved in dichloromethane, treated with TFA and stirred for 1 h. The reaction mixture was evaporated under vacuum and the residue was dissolved in DMF and 4-chlorophenylamine (1.2 eq.), EDCl (1.5 eq.), HOBt (0.5 eq.) and DIEA (2.5 eq.) were added at 5-10° C., and the reaction mixture was stirred at room temperature for 16 h. The reaction mixture was diluted with water and the precipitated solid was collected by suction filtration, dried and dissolved in THF-water (3:1). LiOH (1.1 eq.) was added and the solution was stirred at room temperature overnight. The organic solvent was evaporated under vacuum and the pH of the resulting mixture was adjusted to 6-7. The aqueous solution was extracted with ethyl acetate. The organic phase was dried over MgSO₄ and concentrated under vacuum to afford the title compound (58% overall) mp>250° C.

¹H-NMR (300 MHz, DMSO-d₆, 300° K), δ ppm: 13.55 (br.s, 1H, CO₂H); 10.50 (s, 1H, NH); 8.18-7.16 (m, 11 Ar—H); 3.41 (s, 3H); MS, m/z=497[M-H]−

Analogously, the compound of the following example was prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 156 | 2-(3-Chlorobenzo[b]- | >200 | [400 MHz] 14.10 (br.s, 1H), 12.13 (s, | 571.3 |
| | thiophene-2-carboxamido)-4- | | 1H), 8.68 (d, 1H), 7.80-8.20 (m, 3H), | [MH]⁺ |
| | (N-(4-chlorophenyl)-N- | | 7.60 (m, 2H), 7.10-7.50 (m, 4H), | |
| | (ethoxycarbonylmethyl)- | | 7.00 (d, 1H), 4.61 (s, 2H), 4.16 (q, | |
| | carbamoyl)benzoic acid | | 2H), 1.22 (t, 3H) | |

### Example 157

### 5-(4-Benzylpiperidine-1-carbonyl)-2-(3-chlorobenzo[b]thiophene-2-carboxamido)benzoic acid

### Method 7

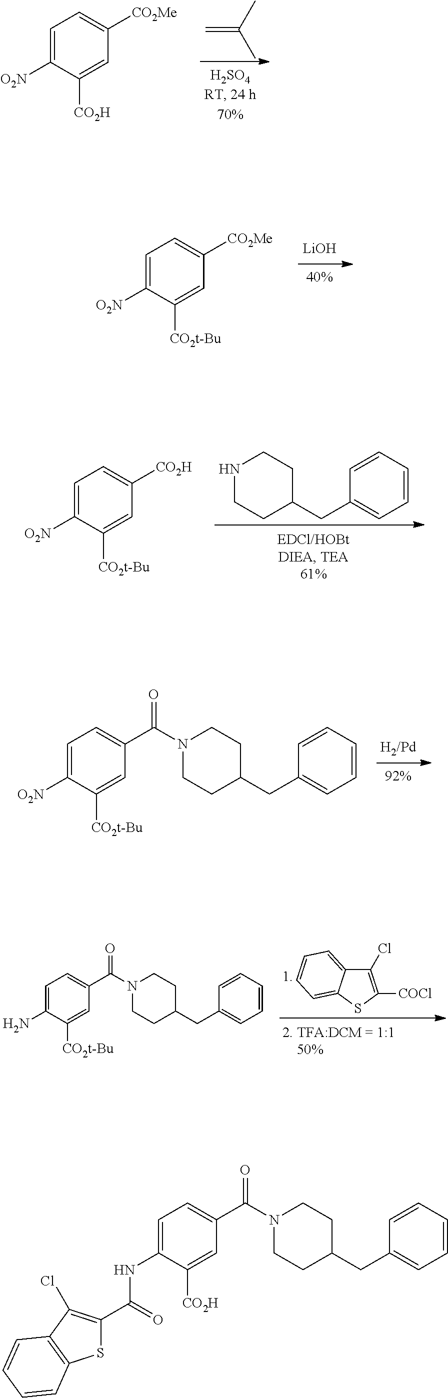

To a solution of methyl 3-carboxy-4-nitrobenzoate in dioxane was added a catalytic amount of sulfuric acid and condensed 2-methylpropene at −15° C. in a sealed tube. The reaction mixture was stirred at room temperature for 48 h. After pouring into 10% NaOH solution and extraction with ethyl acetate, the organic layer was washed with water and brine. Concentration of dried ethyl acetate solution afforded 70% of the corresponding t-butyl ester. This was dissolved in THF-water (3:1) and LiOH (1.1 eq.) was added. The solution was stirred over night at room temperature and concentrated under vacuum. The residue was dissolved in the minimum volume of water and pH was adjusted to 6-7. The aqueous solution was extracted with ethyl acetate, and the organic layer was dried and concentrated under vacuum. The residue was treated in DMF with 4-benzylpiperidine (1.2 eq.), EDCl (1.5 eq.), HOBt (0.5 eq.) and DIEA (2.5 eq.) at 5-10° C., and the reaction mixture was stirred at room temperature for 16 h. The reaction mixture was diluted with water and the precipitated solid was filtered, dried, dissolved in MeOH and hydrogenated for 20 h in presence of 10% palladium on carbon. After filtration of the catalyst through a Celite pad, the filtrate was concentrated under vacuum to afford the aniline derivative. This product was dissolved in THF and 1.1 equivalent of 3-chlorobenzo[b]thiophene-carbonyl chloride was added. The reaction mixture was refluxed for 14 h, cooled to room temperature and evaporated under vacuum. The residue was triturated with diethyl ether to afford t-butyl 5-(4-benzylpiperidine-1-carbonyl)-2-(3-chlorobenzo[b]thiophene-2-carboxamido)benzoate in 11% overall yield.

The t-butyl ester was dissolved in dichloromethane, TFA was added and the reaction was stirred at room temperature for 1 h. Concentration of the reaction mixture and trituration with diethyl ether afforded the desired carboxylic acid, mp>200° C. in 81% yield.

¹H-NMR (400 MHz, DMSO-d₆, 300° K), δ ppm: 14.0 (br.s, 1H), 12.50 (br.s, 1H), 8.65 (s, 1H), 8.18 (m, 1H), 8.02-7.98 (m, 2H), 7.70-7.65 (m, 3H), 7.27 (m, 2H), 7.19 (m, 3H), 4.42 (m, 1H), 3.63 (m, 1H), 3.17-3.02 (m, 2H), 2.73-2.67 (m, 1H), 1.79 (m, 1H), 1.58 (m, 2H), 1.23 (m 1H); MS, m/z=533.1 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 158 | 5-(4-Benzylpiperidine-1- | >200 | [400 MHz] 13.44 (br.s, 1H), 11.68 | 533.4 |
| | carbonyl)-2-(6-chloro- | | (br.s, 1H), 8.59 (d, 1H), 8.27 (m, | [MH]⁺ |
| | benzo[b]thiophene-2- | | 1H), 8.11 (m, 1H), 8.08-8.04 (m, | |
| | carboxamido)benzoic acid | | 2H), 7.67 (d, 1H), 7.50 (d, 1H), 7.29- | |
| | | | 7.26 (m, 2H), 7.20-7.15 (m, 3H), | |
| | | | 4.43 (br s, 1H), 3.8-2.60 (m, 4H), | |
| | | | 2.55 (m, 2H), 1.80-1.55 (m, 2H), | |
| | | | 1.17-1.08 (m, 2H) | |
| 159 | 2-(3-Chlorobenzo[b]- | >200 | [400 MHz] 12.35 (s, 1H), 8.86 (d, | 491.3 |
| | thiophene-2-carboxamido)-5- | | 1H), 8.68 (d, 1H), 8.59 (d, 1H), 8.18 | [MH]⁺ |
| | (indan-2-ylcarbamoyl)benzoic | | (m, 2H), 8.00 (d, 1H), 7.65 (m, 2H), | |
| | acid | | 7.25 (m, 2H), 7.15 (m, 2H), 4.72 (m, | |
| | | | 1H), 3.26 (dd, 1H), 2.98 (dd, 1H) | |

### Example 160

### 2-(6-Chlorobenzo[b]thiophene-2-carboxamido)-5-(4-phenoxypiperidine-1-carbonyl)benzoic acid

### Method 8

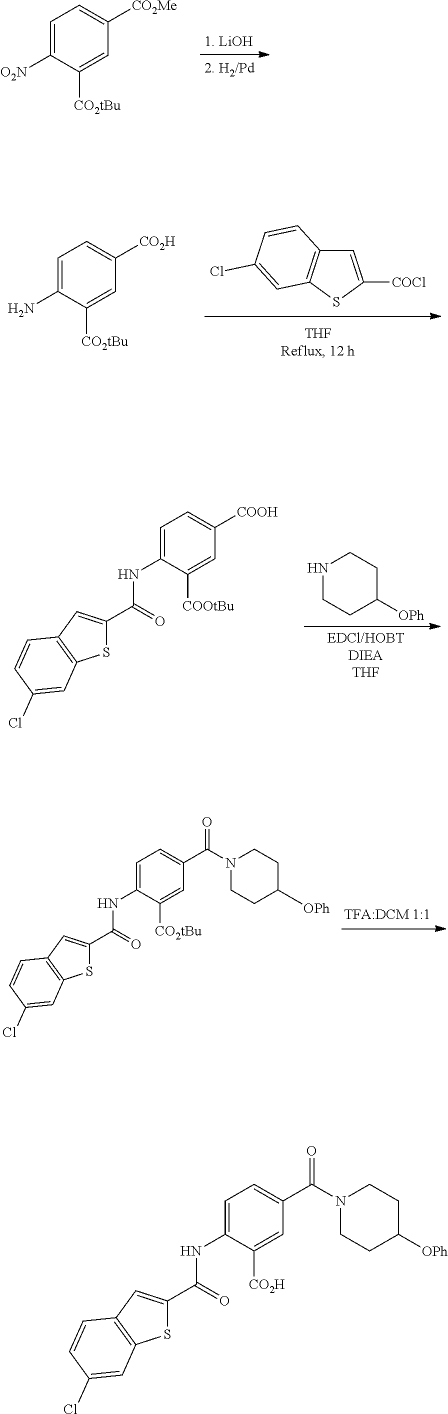

A solution of t-butyl 5-methoxycarbonyl-2-nitrobenzoate (1 eq.) in THF-water (3:1) was treated with LiOH (1.1 eq.) and stirred over night at room temperature. The organic solvent was evaporated under vacuum, a small amount of water was added to obtain solution and the pH was adjusted to 6-7. The aqueous mixture was extracted with ethyl acetate, the organic layer was dried and concentrated under vacuum to afford a residue which was dissolved in MeOH and hydrogenated for 20 h in the presence of 10% palladium on carbon. After filtration of the catalyst through a Celite pad, the filtrate was concentrated under vacuum to afford t-butyl 2-amino-5-carboxybenzoate in 80% yield. This compound was dissolved in THF, 6-chlorobenzo[b]thiophenecarbonyl chloride (1.1 eq.) was added and the reaction mixture was refluxed for 14 h, cooled and concentrated under vacuum. The obtained residue was dissolved in DMF, 4-phenoxypiperidine (1.2 eq.), EDCl (1.5 eq.), HOBt (0.5 eq.) and DIEA (2.5 eq.) were added at 5-10° C., and stirring was continued at room temperature for 16 h. The reaction mixture was diluted with water and the precipitated solid was collected by suction filtration and dried.

The compound was dissolved in dichloromethane, treated with TFA and stirred for 1 h at room temperature. Concentration of reaction mixture under vacuum and trituration with diethyl ether afforded the title compound, mp>200° C., in 27% overall yield.

¹H-NMR (400 MHz, DMSO-d₆, 300° K), δ ppm: 12.70 (br.s, 1H), 8.61 (m, 1H), 8.28 (m, 1H), 8.12-8.08 (m, 3H), 7.73 (m, 1H), 7.53 (m, 1H), 7.55-7.51 (m, 2H), 6.99 (m, 2H), 6.92 (m, 1H), 4.67 (m, 1H), 4.20-3.30 (m, 4H), 1.98 (m, 2H), 1.56 (m, 2H).

MS, m/z=535.3 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 161 | 5-(4-Benzylpiperidine-1- | >200 | [400 MHz] 12.80 (br.s, 1H), 8.60 (d, | 559.2 |
| | carbonyl)-2-(5-(4-chloro- | | 1H), 8.03 (m, 1H), 7.81-7.75 (m, | [MH]⁺ |
| | phenyl)thiophene-2- | | 3H), 7.70 (d, 1H), 7.60 (m, 1H), 7.53 | |
| | carboxamido)benzoic acid | | (m, 2H), 7.29-7.26 (m, 2H), 7.19- | |
| | | | 7.17 (m, 3H), 4.41 (m, 1H), 3.63 (m, | |
| | | | 1H), 3.01-2.66 (m, 2H), 2.55 (d, 2H), | |
| | | | 1.79-1.58 (m, 3H), 2.57 (m, 2H). | |
| 162 | 2-(5-(4-Chlorophenyl)- | >200 | [400 MHz] 14.10 (br.s, 1H), 12.24 (s, | 561.2 |
| | thiophene-2-carboxamido)-5- | | 1H), 8.62 (d, 1H), 8.08 (s, 1H), 7.80- | [MH]⁺ |
| | (4-phenoxypiperidine-1- | | 7.60 (m, 5H), 7.55-7.60 (m, 2H), | |
| | carbonyl)benzoic acid | | 7.35-7.20 (m, 2H), 7.00-6.88 (m, | |
| | | | 3H), 4.66 (m, 1H), 3.98-3.30 (m, | |
| | | | 4H), 1.98 (m, 2H), 1.65 (m, 2H). | |
| 163 | 2-(6-Chlorobenzo[b]- | >200 | [400 MHz] 12.70 (br.s, 1H), 8.62 (d, | 519.3 |
| | thiophene-2-carboxamido)-5- | | 1H), 8.28 (m, 1H), 8.15-8.07 (m, | [MH]⁺ |
| | (4-phenylpiperidine-1- | | 3H), 7.75 (m, 1H), 7.53 (m, 1H), | |
| | carbonyl)benzoic acid | | 7.33-7.28 (m, 4H), 7.20 (m, 1H), | |
| | | | 4.62 (m, 1H), 3.80-2.79 (m, 4H), | |
| | | | 1.65 (m, 2H), 1.57 (m, 2H). | |
| 164 | 2-(6-Chlorobenzo[b]- | >200 | [400 MHz] 13.50 (br.s, 1H), 8.59 (d, | 567.2 |
| | thiophene-2-carboxamido)-5- | | 1H), 8.28 (m, 1H), 8.15-8.00 (m, | [MH]⁺ |
| | (4-(4-chlorobenzyl)piperidine- | | 4H), 7.63 (m, 1H), 7.53 (m, 1H), | |
| | 1-carbonyl)benzoic acid | | 7.33 (m, 2H), 7.21 (m, 2H), 4.44 (m, | |
| | | | 1H), 3.65-2.50 (m, 4H), 1.85-1.55 | |
| | | | (m, 4H), 1.23-1.14 (m, 2H). | |
| 165 | 2-(6-Chlorobenzo[b]- | >200 | [400 MHz] 14.10 (br.s, 1H), 12.50 (s, | 501.2 |
| | thiophene-2-carboxamido)-5- | | 1H), 9.04 (m, 1H), 8.65-8.55 (m, | [MH]⁺ |
| | (2,6-difluorobenzyl- | | 2H), 8.29 (m, 1H), 8.16-8.04 (m, | |
| | carbamoyl)benzoic acid | | 3H), 7.53 (m, 1H), 7.40 (m, 1H), | |
| | | | 7.15-7.05 (m, 2H), 4.54 (s, 2H). | |

### Example 166

### 3-(Benzo[b]thiophene-2-carboxamido)-4-(4-chlorophenylcarbamoyl)-benzoic acid

### Method 9

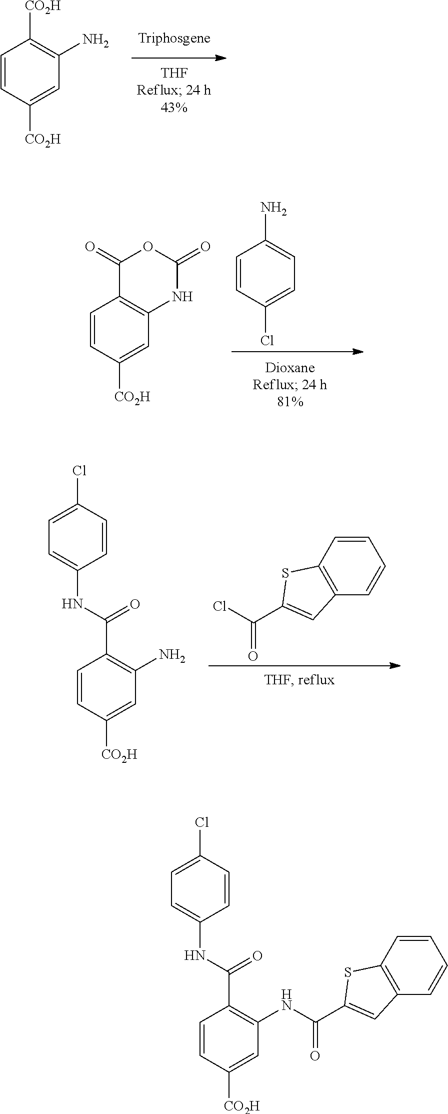

A solution of 2-aminoterepthalic acid (1 eq.) in dry THF was treated with triphosgene (0.33 eq.) and refluxed for 24 h. After cooling to room temperature the solvent was removed under vacuum and the residue was crystallized from THF-hexane to afford the cyclic anhydride as a white solid (43%).

The anhydride (0.4 eq.) was dissolved in the minimum volume of DMF, 4-chloroaniline (0.44 eq.) and a catalytic amount of DMAP were added and the reaction mixture was heated at 70-80° C. for 10 h. After cooling, the reaction mixture was diluted with ice-cold water and stirring was continued for half an hour. The precipitate was collected by suction filtration, washed with ether and dried.

This compound was dissolved in dry THF, benzo[b]thiophenecarbonyl chloride (1.1 eq.) was added and the reaction mixture was refluxed for 12 h. The solvent was evaporated under vacuum and the residue was triturated with diethyl ether to afford the title compound, mp>280° C., in 45% overall yield.

¹H-NMR (300 MHz, DMSO-d₆, 300° K), δ ppm: 13.40 (br.s, 1H, CO₂H); 11.50 (s, 1H, NH); 10.78 (s, 1H, NH); 8.77 (s, 1 Ar—H); 8.15-7.42 (m, 11 Ar—H); MS, m/z=449 [M-H]⁻

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 167 | 3-(3-Chlorobenzo[b]- | >250 | 13.42 (br.s, 1H, CO₂H); 11.44 (s, | 451 |
| | thiophene-2-carboxamido)-4- | | 1H, NH); 10.76 (s, 1H, NH); 8.94 (s, | [MH]⁺ |
| | (phenylcarbamoyl)-benzoic | | 1 Ar—H); 8.17-7.15 (m, 11 Ar—H). | |
| | acid | | | |
| 168 | 3-(3-Chlorobenzo[b]- | >250 | 13.42 (br.s, 1H, CO₂H); 11.44 (s, | 469 |
| | thiophene-2-carboxamido)-4- | | 1H, NH); 10.80 (s, 1H, NH); 8.93 (s, | [MH]⁺ |
| | (4-fluorophenylcarbamoyl)- | | 1 Ar—H); 8.17-7.20 (m, 10 Ar—H). | |
| | benzoic acid | | | |
| 169 | 3-(3-Chlorobenzo[b]- | >250 | 13.40 (br.s, 1H, CO₂H); 11.50 (s, | 465 |
| | thiophene-2-carboxamido)-4- | | 1H, NH); 10.68 (s, 1H, NH); 8.95 (s, | [MH]⁺ |
| | (p-tolylcarbamoyl)-benzoic | | 1 Ar—H); 8.17-7.17 (m, 10 Ar—H); | |
| | acid | | 2.28 (s, 3H) | |
| 170 | 3-(3-Chlorobenzo[b]- | >280 | 13.41 (br.s, 1H, CO₂H); 11.58 (s, 1H, | 479 |
| | thiophene-2-carboxamido)-4- | | NH); 10.65 (s, 1H, NH); 8.97 (s, 1 | [M − H]− |
| | (4-methoxyphenyl- | | Ar—H); 8.17-6.94 (m, 10 Ar—H); 3.74 | |
| | carbamoyl)-benzoic acid | | (s, 3H) | |
| 171 | 4-(4-Chlorophenyl- | 277 | 13.39 (br.s, 1H, CO₂H); 11.05 (s, | 435 |
| | carbamoyl)-3-(3-chloro- | | 1H, NH); 10.83 (s, 1H, NH); 8.85 (s, | [MH]⁺ |
| | thiophene-2-carboxamido)- | | 1 Ar—H); 8.00-7.23 (m, 8 Ar—H). | |
| | benzoic acid | | | |
| 172 | 4-(4-Chlorophenyl- | 274 | 13.37 (br.s, 1H, CO₂H); 11.36 (s, | 403 |
| | carbamoyl)-3-(thiophene-2- | | 1H, NH); 10.75 (s, 1H, NH); 8.78 (s, | [MH]⁺ |
| | carboxamido)benzoic acid | | 1 Ar—H); 7.97-7.23 (m, 9 Ar—H). | |
| 173 | 3-Benzamido-4-(4-chloro- | 280 | 13.38 (br.s, 1H, CO₂H); 11.38 (s, | 256 |
| | phenylcarbamoyl)benzoic | | 1H, NH); 10.77 (s, 1H, NH); 8.90 (s, | [MH]⁺ |
| | acid | | 1 Ar—H); 7.98-7.42 (m, 11 Ar—H). | |
| 174 | 4-(4-Chlorophenyl- | >250 | 13.37 (br.s, 1H, CO₂H); 11.01 (s, | 413 |
| | carbamoyl)-3-(3-methyl- | | 1H, NH); 10.78 (s, 1H, NH); 8.88 (s, | [M − H]− |
| | thiophene-2-carboxamido)- | | 1 Ar—H); 7.95-7.04 (m, 8 Ar—H); 2.51 | |
| | benzoic acid | | (s, 3H) | |
| 175 | 4-(4-Chlorophenyl- | >250 | 13.36 (br.s, 1H, CO₂H); 10.81 (s, | 407 |
| | carbamoyl)-3-(2-methyl- | | 1H, NH); 10.70 (s, 1H, NH); 8.69 (s, | [M − H]− |
| | benzamido)benzoic acid | | 1 Ar—H); 7.88-7.29 (m, 10 Ar—H); | |
| | | | 2.35 (s, 3H) | |
| 176 | 4-(4-Chlorophenyl- | >250 | 13.34 (br.s, 1H, CO₂H); 11.52 (s, | 423 |
| | carbamoyl)-3-(2-methoxy- | | 1H, NH); 10.84 (s, 1H, NH); 9.16 (s, | [M − H]− |
| | benzamido)benzoic acid | | 1 Ar—H); 8.05-7.10 (m, 10 Ar—H); | |
| | | | 3.97 (s, 3H) | |
| 177 | 4-(4-Chlorophenyl- | >250 | 13.43 (br.s, 1H, CO₂H); 11.05 (s, | 429 |
| | carbamoyl)-3-(2,6-difluoro- | | 1H, NH); 10.68 (s, 1H, NH); 8.58 (s, | [M − H]− |
| | benzamido)benzoic acid | | 1 Ar—H); 7.88-7.21 (m, 9 Ar—H); 2.35 | |
| | | | (s, 3H) | |
| 178 | 3-(3-Chlorobenzamido)-4-(4- | >250 | 13.39 (br.s, 1H, CO₂H); 11.24 (s, | 427 |
| | chlorophenylcarbamoyl)- | | 1H, NH); 10.74 (s, 1H, NH); 8.71 (d, | [M − H]− |
| | benzoic acid | | J 4 Hz, 1 Ar—H); 7.93-7.41 (m, 10 Ar—H) | |
| 179 | 3-(4-Chlorobenzamido)-4-(4- | >250 | 13.39 (br.s, 1H, CO₂H); 11.33 (s, | 427 |
| | chlorophenylcarbamoyl)- | | 1H, NH); 10.76 (s, 1H, NH); 8.81 (d, | [M − H]− |
| | benzoic acid | | J 8 Hz, 1 Ar—H); 7.96-7.41 (m, 10 Ar—H) | |
| 180 | 3-(2-Chlorobenzamido)-4-(4- | >250 | 13.40 (br.s, 1H, CO₂H); 10.90 (s, | 427 |
| | chlorophenylcarbamoyl)- | | 1H, NH); 10.70 (s, 1H, NH); 8.64 (s, | [M − H]− |
| | benzoic acid | | 1 Ar—H); 7.86-7.39 (m, 10 Ar—H) | |
| 181 | 3-(3-Chlorobenzo[b]- | >250 | 13.45 (br.s, 1H, CO₂H); 11.19 (s, | 474 |
| | thiophene-2-carboxamido)-4- | | 1H, NH); 11.09 (s, 1H, NH); 8.2 (d, J | [M − H]− |
| | (4-cyanophenylcarbamoyl)- | | 0.8 Hz, 1 Ar—H); 7.98-7.60 (m, 11 Ar—H) | |
| | benzoic acid | | | |
| 182 | 3-(Benzo[b]thiophene-2- | >250 | 13.4 (br.s, 1H, CO₂H); 11.34 (s, 1H, | 440 |
| | carboxamido)-4-(3-cyano- | | NH); 10.94 (s, 1H, NH); 8.69 (s, 1 | [M − H]− |
| | phenylcarbamoyl)benzoic | | Ar—H); 7.18-7.47 (m, 11 Ar—H) | |
| | acid | | | |
| 183 | 3-(3-Chlorobenzo[b]- | >250 | 13.46 (br.s, 1H, CO₂H); 11.38 (s, | 433 |
| | thiophene-2-carboxamido)-4- | | 1H, NH); 10.62 (s, 1H, NH); 8.94 (s, | [M − H]− |
| | (2-chlorophenylcarbamoyl)- | | 1 Ar—H); 8.08-7.23 (m, 8 Ar—H) | |
| | benzoic acid | | | |
| 184 | 4-(N-(4-chlorophenyl)-N- | >250 | 13.28 (br.s, 1H, CO₂H); 10.11 (s, | 447 |
| | methylcarbamoyl)-3-(3- | | 1H, NH); 8.46 (s, 1 Ar—H); 8.03 (d, 1 | [M − H]− |
| | chlorobenzo[b]thiophene-2- | | Ar—H); 7.48-7.14 (m, 7 Ar—H); 3.37 (s, | |
| | carboxamidobenzoic acid | | 3H) | |
| 185 | 4-(4-Chlorophenyl- | >250 | 13.39 (br.s, 1H, CO₂H); 11.16 (s, | 411 |
| | carbamoyl)-3-(2-fluoro- | | 1H, NH); 10.77 (s, 1H, NH); 8.91 (s, | [M − H]− |
| | benzamido)benzoic acid | | 1 Ar—H); 7.99-7.35 (m, 10 Ar—H) | |
| 186 | 4-(4-Chlorophenyl- | >250 | 13.43 (br.s, 1H, CO₂H); 12.26 (s, | 394 |
| | carbamoyl)-3-(pyridine-2- | | 1H, NH); 10.86 (s, 1H, NH); 9.26 (d, | [M − H]− |
| | carboxamido)benzoic acid | | J 1.6 Hz, 1 Ar—H); 8.74-8.73 (m, 10 | |
| | | | Ar—H); 8.20-7.45 (m, 9 Ar—H) | |
| 187 | 4-((3Z,5E)-5-Chloro-2- | >250 | 13.36 (br.s, 1H, CO₂H); 11.74 (s, | 448 |
| | methylene-hepta-3,5-dienyl- | | 1H, NH); 9.58-9.55 (t, 1H, NH); 9.00 | [MH]⁺ |
| | carbamoyl)-3-(3-chloro- | | (d, J 1.44 Hz, 1 Ar—H); 8.01-7.23 (m, | |
| | thiophene-2-carboxamido)- | | 8 Ar—H); 4.48-4.47 (d, 2H). | |
| | bezoic acid | | | |
| 188 | 3-(3-Chlorobenzo[b]- | >250 | 13.44 (br.s, 1H, CO₂H); 11.35 (s, | 483 |
| | thiophene-2-carboxamido)-4- | | 1H, NH); 10.86 (s, 1H, NH); 8.90 (d, | [M − H]− |
| | (4-chlorophenylcarbamoyl)- | | J 1.28 Hz, 1 Ar—H); 8.17-7.43 (m, 10 | |
| | benzoic acid | | Ar—H) | |
| 189 | 4-(4-Chlorophenyl- | >250 | 13.28 (br.s, 1H, CO₂H); 10.64 (s, | 413 |
| | carbamoyl)-3-(thiophen-2-yl- | | 1H, NH); 10.39 (s, 1H, NH); 8.62 (s, | [M − H]− |
| | acetamido)benzoic acid | | 1 Ar—H); 7.81-6.96 (m, 8 Ar—H); 3.94 | |
| | | | (s, 2H) | |
| 190 | 4-(3-Chlorophenyl- | >250 | 13.5 (br.s, 1H, CO₂H); 10.99 (s, 1H, | 433 |
| | carbamoyl)-3-(3-chloro- | | NH); 10.85 (s, 1H, NH); 8.82 (d, J | [M − H]− |
| | thiophene-2-carboxamido)- | | 1.28 Hz, 1 Ar—H); 7.99-7.20 (m, 8 Ar—H) | |
| | benzoic acid | | | |
| 191 | 4-(4-Chlorophenyl- | >250 | 13.25 (br.s, 1H, CO₂H); 11.20 (s, | 418 |
| | carbamoyl)-3-(3-cyano- | | 1H, NH); 10.80 (s, 1H, NH); 8.65- | [M − H]− |
| | benzamido)benzoic acid | | 8.63 (d, 1 Ar—H); 8.31-7.40 (m, 10 | |
| | | | Ar—H) | |
| 192 | 3-(3-Chloro-4-cyano- | >250 | 13.45 (br.s, 1H, CO₂H); 11.51 (s, | 460 |
| | benzamido)-4-(4-isopropyl- | | 1H, NH); 10.60 (s, 1H, NH); 8.70 (d, | [M − H]− |
| | phenylcarbamoyl)-benzoic | | J 1.08 Hz, 1 Ar—H); 8.20-7.20 (m, 8 | |
| | acid | | Ar—H); 2.89-2.82 (m, 1H); 1.19-1.13 | |
| | | | (m, 6H) | |
| 193 | 3-(3-Chlorothiophene-2- | >250 | [400 MHz] 13.5 (br.s, 1H), 11.06 (s, | 441.9 |
| | carboxamido)-4-(4-(methyl- | | 1H), 10.83 (s, 1H), 8.84 (d, 1H), 8.8- | [M − H]− |
| | aminomethyl)phenyl- | | 8.7 (m, 2H), 8.0-7.93 (m, 2H), 7.86- | |
| | carbamoyl)benzoic acid | | 7.76 (m, 3H), 7.47-7.75 (m, 2H), | |
| | | | 7.24 (d, 1H), 4.10 (s, 2H), 2.5 (d, 3H) | |
| 194 | 4-(4-tert-Butoxyphenyl- | >240 | 13.38 (br.s, 1H, CO₂H); 11.49 (s, | 521 |
| | carbamoyl)-3-(3-chloro- | | 1H, NH); 10.67 (s, 1H, NH); 8.95 (s, | [M − H]− |
| | thiophene-2-carboxamido)- | | 1 Ar—H); 8.16-6.97 (m, 10 Ar—H); | |
| | benzoic acid | | 1.10 (s, 9H) | |
| 195 | 4-(4-Benzyloxyphenyl- | >240 | 13.38 (br.s, 1H, CO₂H); 11.26 (s, | 505 |
| | carbamoyl)-3-(3-chloro- | | 1H, NH); 10.63 (s, 1H, NH); 8.91 (s, | [M − H]− |
| | thiophene-2-carboxamido)- | | 1 Ar—H); 7.99-7.02 (m, 13 Ar—H); | |
| | benzoic acid | | 5.09 (s, 2H) | |
| 196 | 4-(4-Benzylphenyl- | >240 | 11.21 (br.s, 1H), 10.45 (br.s, 1H), | 491.3 |
| | carbamoyl)-3-(3-chloro- | | 8.72 (s, 1H), 7.93 (d, 1H), 7.52-7.82 | [MH]⁺ |
| | thiophene-2-carboxamido)- | | (m, 4H), 7.06-7.35 (m, 8H), 3.91 (s, | |
| | benzoic acid | | 2H) | |
| 197 | 4-(Biphenyl-4-ylcarbamoyl)-3- | >200 | 13.38 (br.s, 1H, CO₂H); 11.14 (s, 1H, | 475 |
| | (3-chlorothiophene-2- | | NH); 10.80 (s, 1H, NH); 8.89 (s, 1 | [M − H]− |
| | carboxamido)benzoic acid | | Ar—H); 7.98-7.24 (m, 13 Ar—H) | |
| 198 | 4-(5-Chlorothiazol-2-yl- | >250 | [400 MHz] 13.36-13.09 (br.s, 2H), | 482.2 |
| | carbamoyl)-3-(5-phenyl- | | 10.97 (br.s, 1H), 8.54 (br.s, 1H), | [M − H]− |
| | thiophene-2-carboxamido)- | | 7.94 (m, 2H), 7.76 (m, 1H), 7.67 (m, | |
| | benzoic acid | | 2H), 7.62 (m, 1H), 7.49 (m, 1H), | |
| | | | 7.45 (m, 2H), 7.38 (m, 1H). | |
| 199 | 4-(4-(4-Chlorobenzyl)phenyl- | >200 | [400 MHz] 13.36 (br.s, 1H), 11.15 | 523.1 |
| | carbamoyl)-3-(3-chloro- | | (br.s, 1H), 10.67 (s, 1H), 8.88 (s, | [M − H]− |
| | thiophene-2-carboxamido)- | | 1H), 7.97 (m, 1H), 7.93 (m, 1H), | |
| | benzoic acid | | 7.82 (m, 1H), 7.63 (m, 2H), 7.33 (m, | |
| | | | 2H), 7.25-7.21 (m, 5H), 3.91 (s, 2H). | |
| 200 | 3-(4-(4-Chlorobenzyl)phenyl- | >200 | [400 MHz] 13.2 (br.s, 1H), 11.49 | 522.9 |
| | carbamoyl)-4-(3-chloro- | | (br.s, 1H), 10.76 (br.s, 1H), 8.48 (m, | [MH]⁺ |
| | thiophene-2-carboxamido)- | | 1H), 8.40 (m, 1H), 8.13 (m, 1H), | |
| | benzoic acid | | 7.99 (m, 1H), 7.63 (m, 2H), 7.34 (m, | |
| | | | 2H), 7.26-7.21 (m, 5H), 3.92 (s, 2H). | |

### Example 201

### 4-(4-Chlorophenylcarbamoyl)-3-(1H-indole-2-carboxamido)benzoic acid

### Method 10

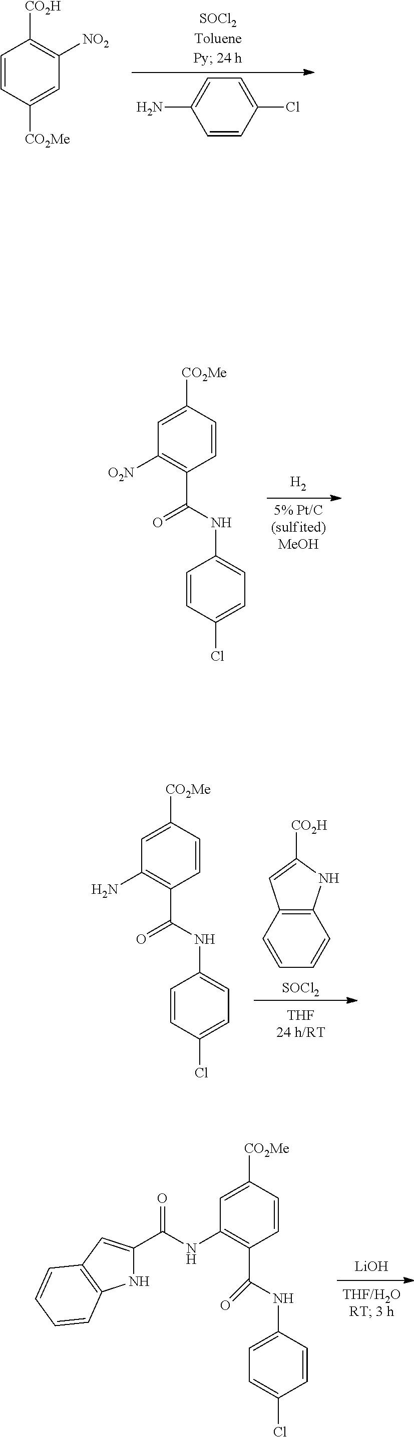

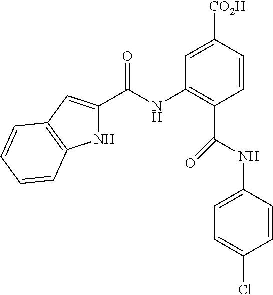 [SMILES: O=C(O)c1ccc(C(=O)Nc2ccc(Cl)cc2)c(NC(=O)c2cc3ccccc3[nH]2)c1]

To a solution of methyl 4-carboxy-3-nitrobenzoate (1 eq.) in toluene thionyl chloride (2.5 eq.) and catalytic amount of pyridine were added, and the reaction mixture was refluxed for 4 h, then cooled to room temperature. The reaction mixture was concentrated under vacuum to half volume, diluted again with toluene and concentrated again to half volume. This operation was repeated until all the acidity was removed. Then this solution was added dropwise to a stirred a solution of 4-chloroaniline (1.6 eq.) in toluene and stirring was continued for 14 h at room temperature. The reaction mixture was concentrated under vacuum and the residue was dissolved in ethyl acetate. The obtained solution was washed with dil. HCl and water, dried (MgSO₄) and evaporated under vacuum. The residue was chromatographed over silica gel, eluting with 12% ethyl acetate-hexane. The collected fractions were evaporated to dryness, dissolved in methanol and hydrogenated for 18-20 h in the presence of 10% platinum sulfide on carbon. After filtration of the catalyst through a Celite pad, the filtrate was concentrated under vacuum to afford the desired aniline. This crude compound was dissolved in THF and treated with 1.1 eq of indol-2-ylcarbonyl chloride. The reaction mixture was stirred at room temperature for 14 h, cooled and concentrated under vacuum. The residue was triturated with diethyl ether to afford methyl 4-(4-chlorophenylcarbamoyl)-3-(1H-indole-2-carboxamido)-benzoate. This compound was dissolved in THF-water (3:1), treated with LiOH (1.1 eq.) and stirred overnight at room temperature. The organic solvent was removed under vacuum, the pH was adjusted to 6-7 and the solution was extracted with ethyl acetate. The organic layer was dried (MgSO₄) and concentrated under vacuum to afford the title compound (9.7% overall), mp>240° C.

¹H-NMR (300 MHz, DMSO-d₆, 300° K), 6 ppm: 11.87 (s, 1H, NH); 11.46 (s, 1H, NH); 10.79 (s, 1H, NH); 8.90 (s, 1 Ar—H); 7.99-7.08 (m, 12 Ar—H); MS, m/z=432 [M-H]⁻

Analogously the compound of the following example was prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 202 | 3-(3-Chloro-1H-indene-2- | >240 | 13.37 (br.s, 1H, CO₂H); 11.57 (s, | 506 |
| | carboxamido)-4-(4-propoxy- | | 1H, NH); 10.62 (s, 1H, NH); 8.90 (s, | [MH]− |
| | phenylcarbamoyl)benzoic | | 1 Ar—H); 8.15-6.93 (m, 10 Ar—H); | |
| | acid | | 3.90-3.92 (m, 2H); 1.70-1.72 (m, 2H); | |
| | | | 0.98-0.96 (m, 3H) | |

### Example 203

### 2-(Benzo[b]thiophene-2-carboxamido)-4-(4-carbamimidoylphenyl-carbamoyl)benzoic acid

### Method 11

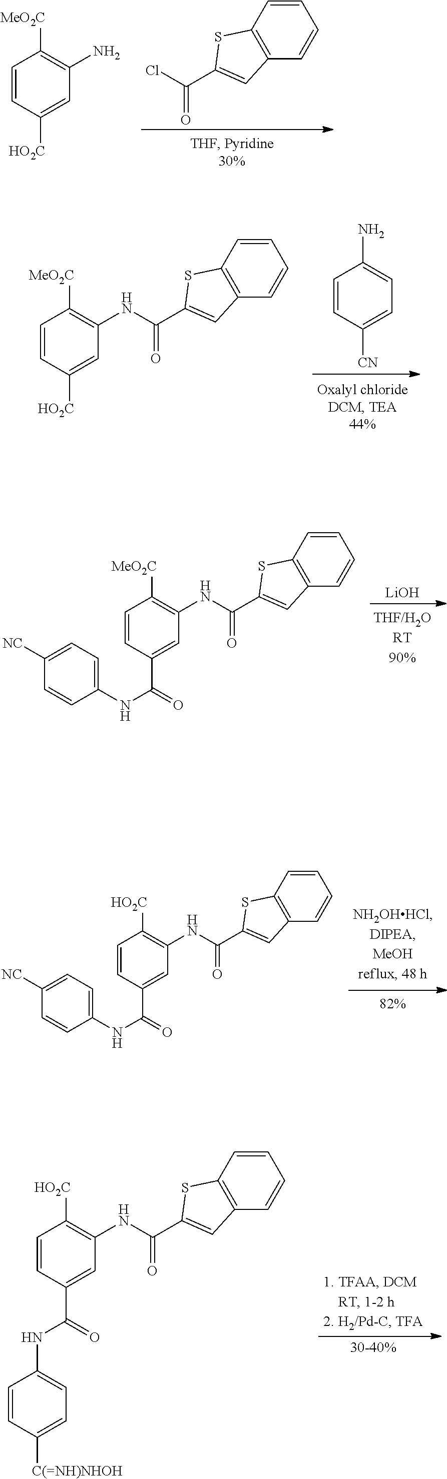

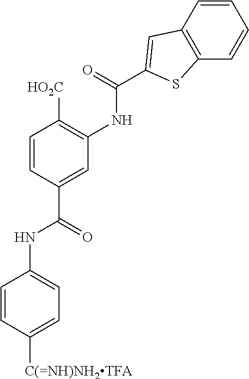

2-Amino-terephthalic acid 1-methyl ester (1 eq.) was dissolved in THF, and 1.1 eq. benzo[b]thiophenecarbonyl chloride was added. The reaction mixture was refluxed for 14 h, cooled to room temperature, and the solvent was evaporated under vacuum. The crude residue was triturated with diethyl ether and dissolved in dichloromethane. Freshly distilled oxalyl chloride (1.2 eq.) was added and the reaction mixture stirred at room temperature for 2 h. Then 4-cyanoaniline (4 eq.) and triethylamine (TEA, 2.5 eq.) were added, and stirring was continued at room temperature for 16 h. The reaction mixture was washed successively with water, diluted sulfuric acid and water again. The organic phase was evaporated under vacuum to dryness and the residue was triturated with diisopropyl ether-hexane.

The obtained product was dissolved in THF-water (3:1) and treated with LiOH (1.1 eq.). The solution was stirred overnight at room temperature, the organic solvent was removed under vacuum and the residue was dissolved in the minimum volume of water. The pH of the solution was adjusted to 6-7 and the solution was extracted with ethyl acetate. The organic layer was collected, dried and evaporated under vacuum.

The residue was dissolved in methanol and treated with hydroxylamine hydrochloride (1.5 eq.) and diisopropylethyl amine (DIPEA, 1.5 eq.), and the reaction mixture was refluxed for 48 h. After cooling, the solvent was removed under vacuum and the residue was triturated with diisopropyl ether-hexane. This hydroxyamidine compound was dissolved in dichloromethane (DCM), trifluoroacetic acid anhydride (3 eq.) was added and the reaction mixture was stirred at room temperature for 2 h. The solvent was removed under vacuum, the residue was taken up in trifluoroacetic acid and hydrogenated in the presence of palladium on charcoal catalyst. After removal of the solvent under vacuum, the residue was crystallized from isopropanol-diisopropyl ether to afford title compound as the trifluoroacetate salt, mp>250° C., in 4.6% overall yield.

¹H-NMR (400 MHz, TFA, 300° K), δ ppm: 8.80 (s, 1H), 8.45-8.36 (m, 2H), 8.21-8.11 (m, 2H), 8.02-7.89 (m, 2H), 7.83-7.74 (m, 3H), 7.62-7.5 (m, 2H); MS, m/z=459.1 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 204 | 5-(5-Amino-5-iminopentyl- | >250 | [500 MHz] 15.98 (br.s, 1H), 8.96 (m, | 473.1 |
| | carbamoyl)-2-(3-chloro- | | 2H), 8.86 (m, 2H), 8.54 (m, 3H), 8.15 | [MH]⁺ |
| | benzo[b]thiophene-2- | | (br.s, 1H), 7.96 (m, 1H), 7.84 (d, | |
| | carboxamido)benzoic acid | | 1H), 7.63 (m, 2H), 3.28 (t, 2H), 2.38 | |
| | | | (m, 2H), 1.64 (m, 2H), 1.55 (m, 2H) | |
| 205 | 5-(4-Amino-4-iminobutyl- | >250 | [500 MHz] 16.22 (br.s, 1H), 8.94 (m, | 459.1 |
| | carbamoyl)-2-(3-chloro- | | 2H), 8.70 (m, 2H), 8.59 (m, 3H), | [MH]⁺ |
| | benzo[b]thiophene-2- | | 8.15 (m, 1H), 7.96 (m, 1H), 7.85 (d, | |
| | carboxamido)benzoic acid | | 1H), 7.63 (m, 2H), 3.45 (t, 2H), 2.41 | |
| | | | (m, 2H), 1.87 (m, 2H). | |
| 206 | 5-(3-Amino-3-iminopropyl- | >250 | [500 MHz, CDCl₃] 15.82 (br.s, 1H), | 444.9 |
| | carbamoyl)-2-(3-chloro- | | 9.06-8.65 (m, 4H), 8.58 (m, 2H), | [MH]⁺ |
| | benzo[b]thiophene-2- | | 8.15 (d, 1H), 7.96-7.90 (m, 2H), 7.62 | |
| | carboxamido)benzoic acid | | (m, 2H), 7.01 (br.s, 1H), 3.63 (t, 2H), | |
| | | | 2.67 (t, 2H) | |

### Example 207

### 4-(3-Carbamimidoylphenylcarbamoyl)-3-(3-chlorobenzo[b]thiophene-2-carboxamido)benzoic acid

### Method 12

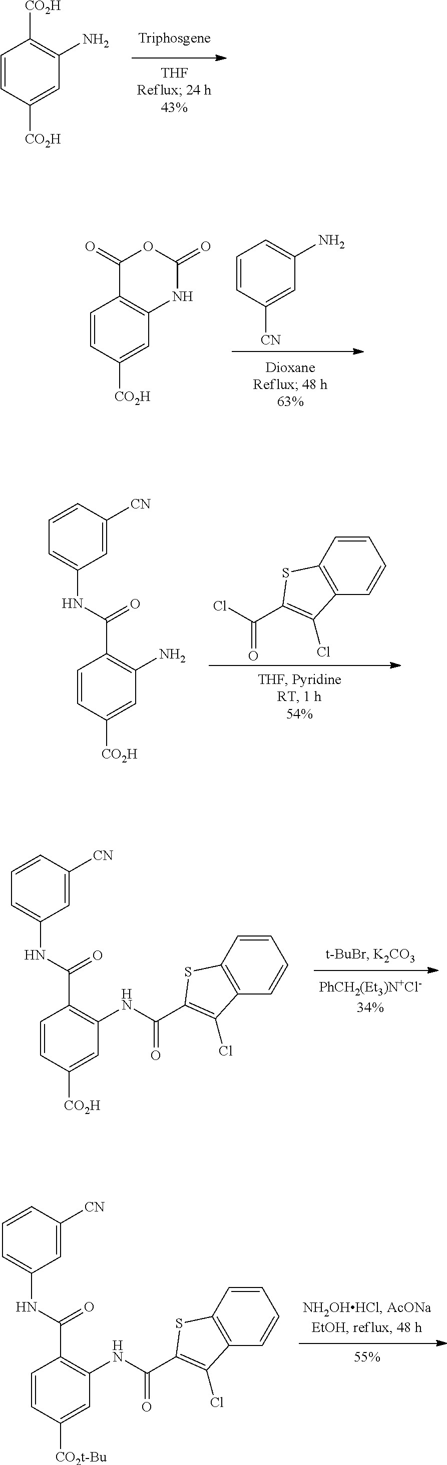

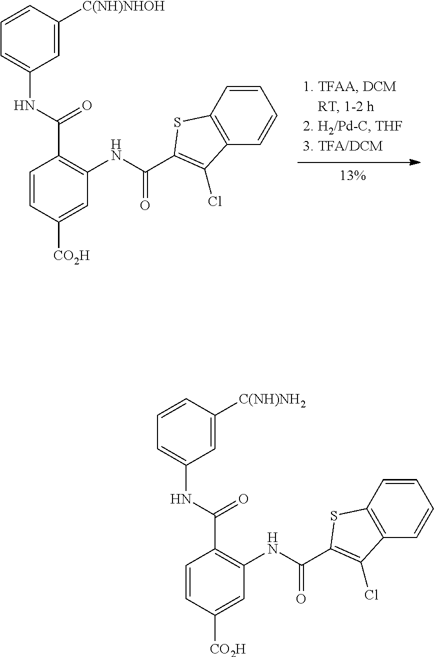

A solution of 2-aminoterepthalic acid (1 eq.) in dry THF was treated with triphosgene (0.33 eq.) and refluxed for 24 h. After cooling to room temperature the solvent was removed under vacuum and the residue was crystallized from THF-hexane to afford the cyclic anhydride as a white solid, which was dissolved in the minimum volume of dioxane. 3-Cyanoaniline (4 eq.) and a catalytic amount of DMAP were added. The reaction mixture was refluxed for 48 h, cooled and concentrated under vacuum. The residue was crystallized from ethyl acetate-hexane to afford the 3-cyanoanilide derivative. This compound was dissolved in a 9:1 mixture of dry THF and pyridine, 3-chloro-benzo[b]thiophene acid chloride (1.1 eq.) was added and the resulting mixture was stirred at RT for 1 h. The solvent was removed under vacuum and the residue was poured into ice-cold diluted sulfuric acid and extracted with ethyl acetate. The organic layer was dried, evaporated to dryness under vacuum and the crude residue was triturated with diisopropyl ether/hexane to afford 4-(3-cyanophenylcarbamoyl)-3-(3-chlorobenzo[b]thiophene-2-carboxamido)benzoic acid.

A solution of this compound in dimethylacetamide was treated with t-butyl bromide (1.1 eq.), anhydrous potassium carbonate (1.1 eq.) and benzyltriethylammonium chloride (0.2 eq.), then stirred at 55° C. for 21 h. After cooling, the reaction mixture was poured into water, the precipitate was collected and crystallized with diisopropyl ether. The obtained product was dissolved in methanol and treated with hydroxylamine hydrochloride (1.5 eq.) and DIPEA (1.5 eq.) and refluxed for 48 h. After cooling, the solvent was removed under vacuum and the residue was triturated with diisopropyl ether-hexane to afford a residue which was dissolved in dichloromethane (DCM). Trifluoroacetic acid anhydride (3 eq.) was added and the reaction mixture was stirred at room temperature for 2 h. The solvent was removed under vacuum, the residue was taken up in tetrahydrofuran and hydrogenated in the presence of palladium on charcoal catalyst. The solvent was removed under vacuum and the residue was dissolved in a 1:1 mixture of trifluoroacetic acid and dichloromethane. After stirring for 6 h at room temperature, the solvent was evaporated and the residue was crystallized from diisopropyl ether to afford the title compound (1.2% overall) as the trifluoroacetate salt, mp>250° C.

¹H-NMR (400 MHz, DMSO-d₆, 300° K), δ ppm: 8.76 (br.s, 1H), 8.07 (br.s, 2H), 7.93-7.87 (m, 4H), 7.62-7.58 (m, 3H), 7.51-7.49 (m, 1H); MS: m/z=493.1 [MH]⁺

Analogously the compound of the following example was prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 208 | 4-(4-Carbamimidoylbenzyl- | >250 | [400 MHz] 13.5 (br.s, 1H), 11.76 (s, | 457.1 |
| | carbamoyl)-3-(3-chloro- | | 1H), 9.64 (br.s, 1H), 9.37 (m, 2H), | [MH]⁺ |
| | thiophene-2-carboxamido)- | | 9. 21 (m, 2H), 8.98 (s, 1H), 7.99 (d, | |
| | benzoic acid | | 1H), 7.93-7.91 (m, 1H), 7.79-7.76 | |
| | | | (m, 3H), 7.58-7.56 (m, 2H), 7.24 (d, | |
| | | | 1H), 4.58 (d, 2H) | |

### Example 209

### 2-(3-Chlorobenzo[b]thiophene-2-carboxamido)-5-(4-(methylamino)butyl-carbamoyl)benzoic acid

### Method 13

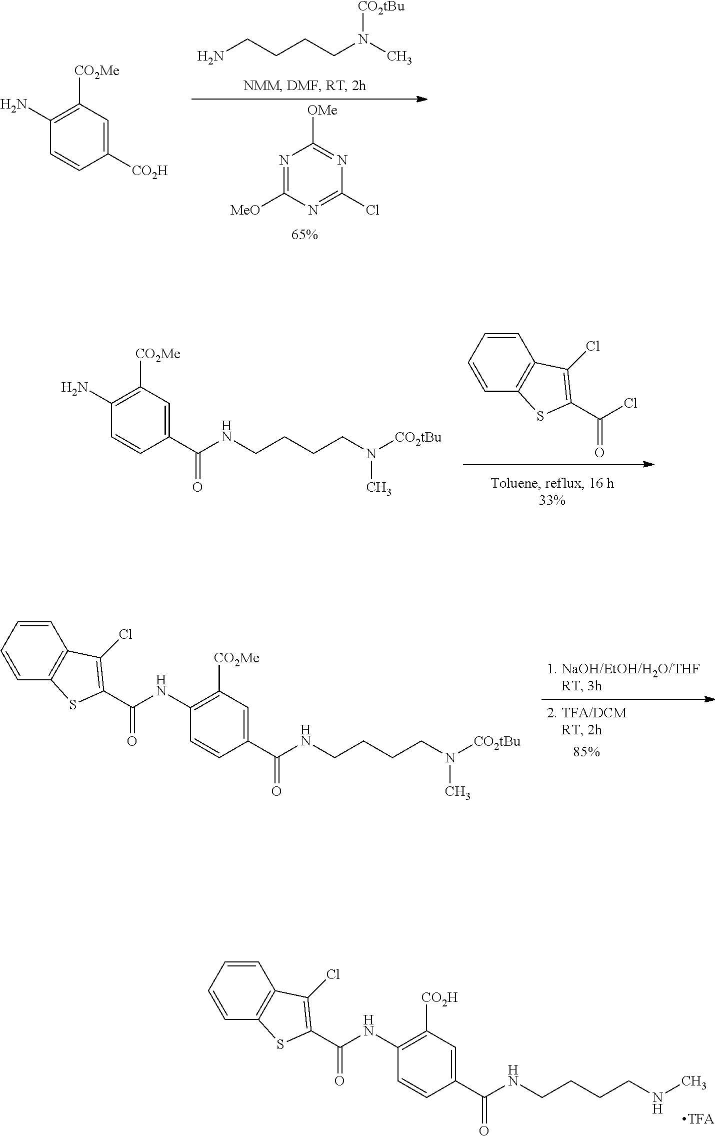

A solution of methyl 2-amino-5-carboxybenzoate (0.4 mmol), 5-(N-methyl-N-t-butoxy-carbonyl)aminobutylamine (0.4 mmol) and 2-chloro-4,6-dimethoxy-1,3,5-triazine (0.4 mmol) in N-methylmorpholine/dimethylformamide was stirred at room temperature for 2 h. The reaction was poured into water and extracted with ethyl acetate. The organic layer was dried and evaporated under vacuum. The crude residue was chromatographed over a short column of Florisil and the collected fractions evaporated and dissolved in THF. To this solution, 3-chloro-benzo[b]thiophene carbonyl chloride (0.4 mmol) was added. The reaction mixture was refluxed for 10-14 h and concentrated under vacuum. The residue was triturated with diisopropyl ether and dissolved in 20 ml of THF and 10 ml of EtOH. 6 N NaOH (1.65 mmol) was added and the solution was stirred at room temperature for 3 h. The mixture was filtered through a Celite pad, then poured into acidic water; the precipitate was collected, washed with water and MeOH, and dried. The obtained carboxylic acid was dissolved in a 1:1 mixture of trifluoroacetic acid and dichloromethane and stirred for 2 h at room temperature. The solvent was evaporated and the residue was crystallized to afford the title compound as the trifluoroacetate salt, mp>250° C., in 16% overall yield.

¹H-NMR (500 MHz, DMSO-d₆, 300° K), δ ppm: 12.34 (br.s, 1H), 8.71-8.69 (m, 2H), 8.58 (m, 1H), 8.30 (m, 2H), 8.20-8.16 (m, 2H), 8.01 (m, 1H), 7.68-7.65 (m, 2H), 3.52 (m, 2H), 2.93 (m, 2H), 2.56 (s, 3H), 1.60 (m, 4H); MS, m/z=459.9 [MH]⁺

### Example 210

### 3-(3-Chlorobenzo[b]thiophene-2-carboxamido)-4-(3-chlorophenyl-carbamoyl)benzoic acid

### Method 14

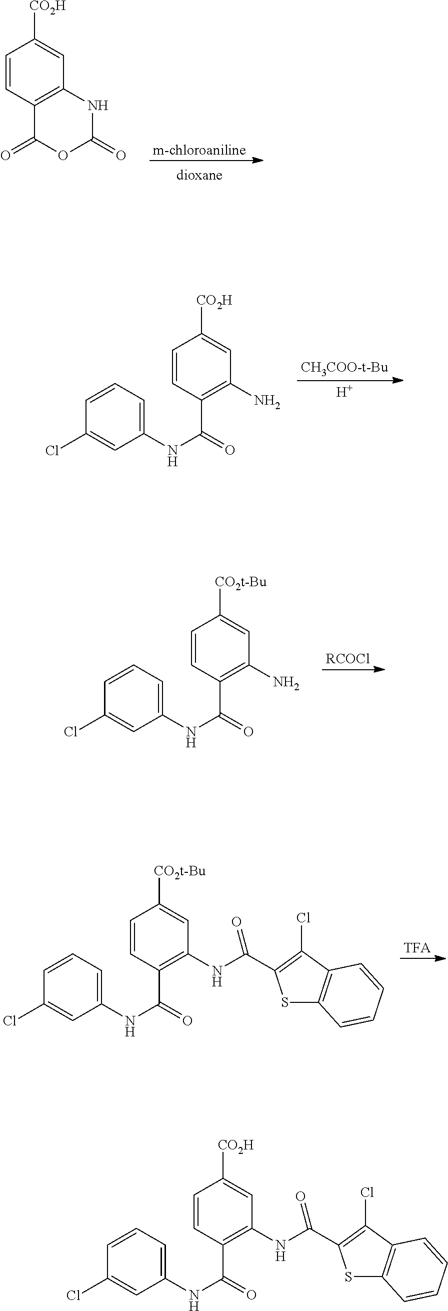

A mixture of 4-carboxyisatoic anhydride (600 mg, 2.90 mmol), 3-chloroaniline (370 mg, 2.90 mmol) and dioxane (5 mL) was refluxed overnight, then cooled to room temperature. The precipitate was collected and washed with diethyl ether to give 3-amino-N-(3-chloro-phenylcarbamoyl)benzoic acid as a yellow solid (670 mg, 80%). A mixture of this compound (293 mg, 1.01 mmol), perchloric acid (1.4 mL) and tert-butyl acetate (45 mL) was stirred overnight at room temperature. The mixture was diluted with water and cautiously basified without exceeding pH 8, then extracted three times with ethyl acetate; the combined organic phases were washed with water and brine, then dried over sodium sulfate and evaporated to give the corresponding tert-butyl ester as a white solid (71 mg, 20%). A mixture of this tert-butyl ester (95 mg, 0.274 mmol), 3-chlorobenzo[b]thiophene-2-carbonyl chloride (64 mg, 0.274 mmol), pyridine (0.024 mL, 0.30 mmol) and THF (5 mL) was stirred at room temperature for 1 hour, then evaporated. The residue was treated with ethyl acetate and the precipitate was collected by filtration to give 3-(3-chlorobenzo[b]-thiophene-2-carboxamido)-4-(3-chlorophenylcarbamoyl)benzoic acid tert-butyl ester (60 mg, 40%). A mixture of this tert-butyl ester (142 mg, 0.262 mmol), trifluoroacetic acid (1 mL) and DCM (15 mL) was stirred for 40 h at room temperature. The mixture was concentrated to a small volume under vacuum and the precipitate was collected and washed with pentane. The title compound was obtained pure as a white solid (80 mg, 58%), mp>200° C.

¹H-NMR (300 MHz, DMSO-d₆, 300° K), δ ppm: 13.37 (br.s, 1H), 11.28 (s, 1H), 10.84 (s, 1H), 8.89 (d, 1H), 8.11-8.26 (m, 1H), 7.93-8.03 (m, 2H), 7.80-7.93 (m, 2H), 7.53-7.76 (m, 3H), 7.41 (t, 1H), 7.21 (ddd, 1H); MS: m/z=485 (MH)⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 211 | 3-[(3-Chloro-benzo[b]- | >200 | 13.37 (br.s, 1H), 11.68 (s, 1H), | 485.07 |
| | thiophene-2-carboxamide]-4- | | 10.61 (s, 1H), 9.03 (d, 1H), 8.13- | [MH]⁺ |
| | (2-chloro-phenylcarbamoyl)- | | 8.21 (m, 1H), 8.10 (d, 1H), 7.92-8.00 | |
| | benzoic acid | | (m, 1H), 7.89 (dd, 1H), 7.53-7.69 (m, | |
| | | | 4H), 7.29-7.47 (m, 2H) | |

### Example 212

### 4-(3-Chlorobenzo[b]thiophene-2-carboxamido]-3-(4-chlorophenyl-carbamoyl)benzoic acid

### Method 15

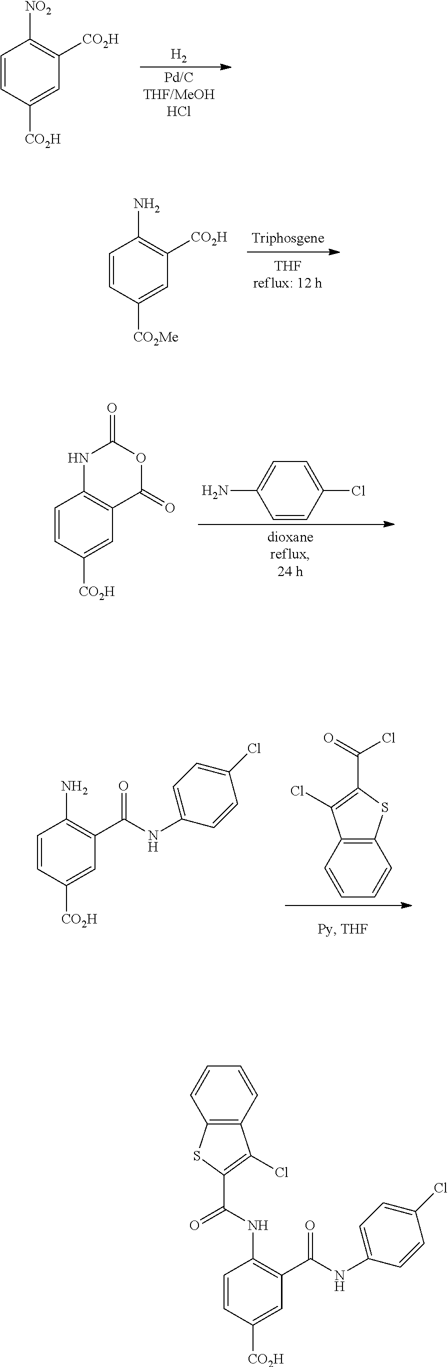

A solution of 4-nitro-1,3-benzenedicarboxylic acid (1 eq.) in THF-MeOH (3:1) was hydrogenated for 20 h in the presence of 10% palladium on carbon. After filtration of the catalyst through a Celite pad, the filtrate was concentrated under vacuum and the residue was dissolved in ahydrous THF. To this solution, triphosgene (0.33 eq.) was added and the reaction mixture was refluxed for 24 h. After cooling to room temperature, the solvent was removed under vacuum and the residue was triturated with THF-hexane to afford the corresponding cyclic anhydride as a white solid. This compound was dissolved in a minimum volume of DMF and 4-chloroaniline (1.1 eq.) was added together with a catalytic amount of DMAP. The reaction mixture was heated at 70-80° C. for 10 h, cooled to room temperature and diluted with ice-cold water. After stirring at room temperature for 0.5 h, the precipitate was collected by suction filtration and crystallized from diethyl ether. The obtained compound was dissolved in anhydrous THF, 3-chlorothiophene-2-carbonyl chloride (1 eq.) was added and the reaction mixture was refluxed for 14 h. The solvent was concentrated under vacuum and the residue was crystallized form ethyl acetate-hexane to afford the title compound, mp>250° C., in 18.9% overall yield.

¹H-NMR (300 MHz, DMSO-d₆, 300° K), δ ppm: 13.35 (br.s, 1H, CO₂H); 11.67 (br.s, 1H, NH); 10.95 (br.s, 1H, NH); 8.51-7.43 (m, 11 Ar—H); MS: m/z=483 [M-H]⁻

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 213 | 3-(4-Chlorophenyl- | >250 | 13.2 (br.s, 1H, CO₂H); 11.80 (s, 1H, | 399 |
| | carbamoyl)-4-(thiophene-2- | | NH); 10.90 (s, 1H, NH); 8.47-7.25 | [M − H]− |
| | carboxamido)benzoic acid | | (m, 12 Ar—H) | |
| 214 | 4-(Benzo[b]thiophene-2- | >250 | 13.25 (br.s, 1H, CO₂H); 11.92 (s, | 499 |
| | carboxamido)-3-(4-chloro- | | 1H, NH); 10.91 (s, 1H, NH); 8.49- | [M − H]− |
| | phenylcarbamoyl)benzoic | | 7.44 (m, 12 Ar—H) | |
| | acid | | | |
| 215 | 3-(4-Chlorophenyl- | >250 | 13.25 (br.s, 1H, CO₂H); 11.39 (br.s, | 433 |
| | carbamoyl)-4-(3-chloro- | | 1H, NH); 10.91 (br.s, 1H, NH); 8.45- | [M − H]− |
| | thiophene-2-carboxamido)- | | 7.24 (m, 9 Ar—H) | |
| | benzoic acid | | | |
| 216 | 3-(4-Chlorophenyl- | >200 | 13.18 (br.s, 1H), 11.67 (br.s, 1H), | 516.95 |
| | carbamoyl)-4-(3,6-dichloro- | | 10.92 (br.s, 1H), 8.51 (d, 1H), 8.45 | [M − H]− |
| | benzo[b]thiophene-2- | | (d, 1H), 8.36 (d, 1H), 8.17 (dd, 1H), | |
| | carboxamido)benzoic acid | | 7.96 (d, 1H), 7.77 (m, 2H), 7.65 (dd, | |
| | | | 1H), 7.44 (m, 2H) | |

### Example 217

### 3-(4-Cyanophenylcarbamoyl)-4-(thiophene-2-carboxamido)benzoic acid

### Method 16

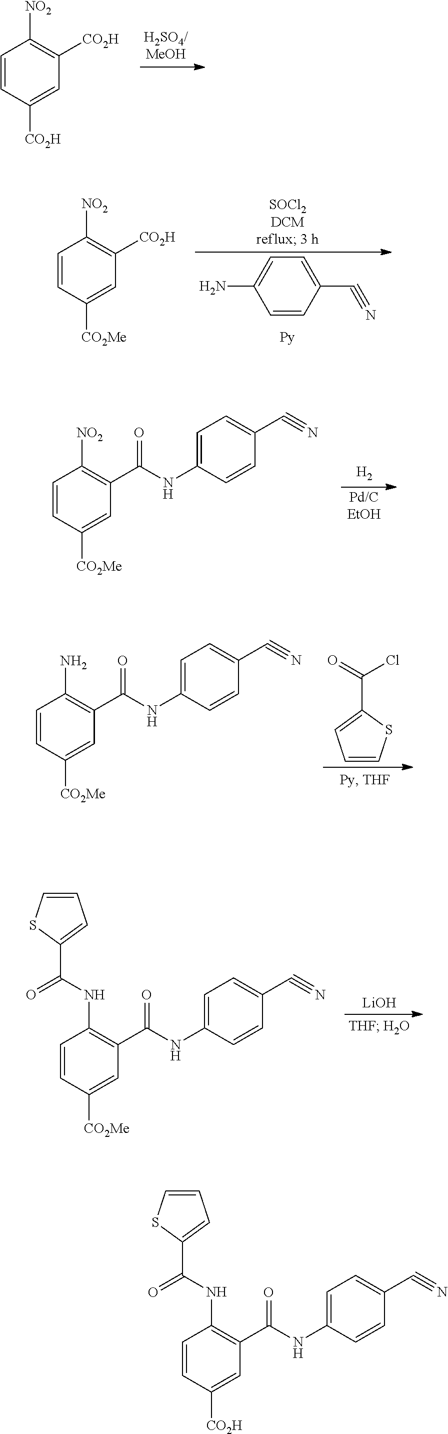

A solution of 4-nitro-1,3-benzenedicarboxylic acid (1 eq.), thionyl chloride (2.5 eq.) and a catalytic amount of DMF in dichloromethane was refluxed for 4 h. After cooling, the solvent was evaporated under vacuum, taken up in little dichloromethane and evaporated again, until all acidity had been removed. The residue was dissolved in dichloromethane, and 4-cyanoaniline (1.2 eq.) in dichloromethane was added and the solution was stirred at room temperature for 14 h. The reaction mixture was concentrated under vacuum, the residue was dissolved in ethyl acetate and washed with dil. HCl and water (twice). The organic layer was collected, dried with MgSO₄ and evaporated under vacuum. The residue was dissolved in MeOH and hydrogenated for 18-20 h in the presence of 10% palladium on carbon. After filtration of the catalyst through a Celite pad, the filtrate was concentrated under vacuum. The residue was dissolved in dry THF, treated with thiophene-2-carbonyl chloride (1 eq.) and refluxed for 14 h. The solvent was concentrated under vacuum and the residue was crystallized form ethyl acetate-hexane to afford the methyl ester of 3-(4-cyanophenylcarbamoyl)-4-(thiophene-2-carboxamido)benzoic acid, which was dissolved in THF-water (3:1) and treated with a solution of LiOH (1.1 eq.) in water. Stirring was continued overnight at room temperature, then the organic solvent was evaporated under vacuum and the residue was dissolved in the minimum of water, adjusting the pH to 6-7. The precipitated solid was collected by suction filtration and dried to yield the title compound, mp>250° C., in 12.6% overall yield.

¹H-NMR (300 MHz, DMSO-d₆, 300° K), δ ppm: 13.2 (br.s, 1H, CO₂H); 11.50 (br.s, 1H, NH); 11.10 (br.s, 1H, NH); 8.43-7.25 (m, 10H, Ar—H); MS: m/z=390 [N−H]⁻

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 218 | 3-(4-Chlorophenyl- | >250 | 13.21 (br.s, 1H, CO₂H); 11.72 (s, | 433 |
| | carbamoyl)-4-(5-chloro- | | 1H, NH); 10.87 (s, 1H, NH); 8.44- | [M − H]− |
| | thiophene-2-carboxamido)- | | 7.30 (m, 9 Ar—H) | |
| | benzoic acid | | | |
| 219 | 3-(4-Chlorophenyl- | >250 | 13.24 (br.s, 1H, CO₂H); 11.81 (s, | 424 |
| | carbamoyl)-4-(5-cyano- | | 1H, NH); 10.86 (s, 1H, NH); 8.43- | [M − H]− |
| | thiophene-2-carboxamido)- | | 7.42 (m, 9 Ar—H) | |
| | benzoic acid | | | |

### Example 220

### 4-(5-Amino-5-iminopentylcarbamoyl)-2-(3-chlorobenzo[b]thiophene-2-carboxamido)benzoic acid

### Method 17

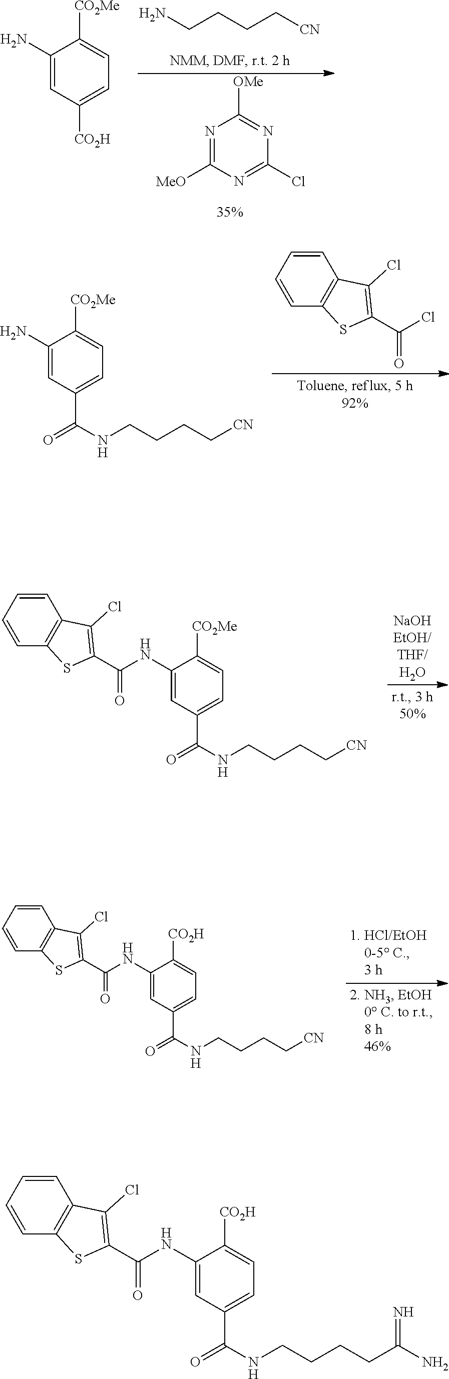

A solution of methyl 2-amino-5-carboxybenzoate (0.4 mmol), 5-cyanopentylamine (0.4 mmol) and 2-chloro-4,6-dimethoxy-1,3,5-triazine (0.4 mmol) in N-methylmorpholine/dimethylformamide was stirred at room temperature for 2 h. The reaction mixture was poured into water and extracted with ethyl acetate. The organic layer was dried and evaporated under vacuum. The crude residue was chromatographed over a short column of Florisil and the collected fractions evaporated and dissolved in THF. To this solution, 3-chlorobenzo[b]thiophene carbonyl chloride (0.4 mmol) was added. The reaction mixture was refluxed for 5 h and concentrated under vacuum. The residue was triturated with di-isopropyl ether and dissolved in 20 ml THF and 10 ml EtOH, 6 N NaOH (1.65 mmol) was added and the solution was stirred at room temperature for 3 h. The mixture was filtered through a Celite pad, then poured into acidic water; the precipitate was collected, washed with water and MeOH, and dried. The obtained carboxylic acid was dissolved in anhydrous ethanol saturated with HCl and stirred at 0-5° C. for 3 h under vacuum. The residue was dissolved in anhydrous ethanol, cooled at 0° C. and saturated with ammonia. The resulting solution was stirred for 8 h letting the temperature to rise until room temperature. The solvent was evaporated and the residue was crystallized to afford the title compound, mp>250° C., in about 10% overall yield.

¹H-NMR (300 MHz, DMSO-d₆, 300° K), δ ppm: 15.81 (br.s, 1H), 8.94 (m, 1H), 8.52 (m, 1H), 8.15 (m, 1H), 8.06 (m, 1H), 7.96 (m, 1H), 7.64 (m, 2H), 7.48 (m, 1H), 3.29 (m, 2H), 2.42 (m, 2H), 1.68 (m, 2H), 1.57 (m, 2H); MS: m/z=473.1 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 221 | 4-(3-Amino-3-iminopropyl- | >250 | 16.10 (br.s, 1H), 8.97 (m, 1H), 8.52 | 445.1 |
| | carbamoyl)-2-(3-chloro- | | (m, 1H) 8.15 (m, 1H), 8.07 (m, 1H), | [MH]⁺ |
| | benzo[b]thiophene-2- | | 7.96 (m, 1H), 7.64 (m, 2H), 7.48 (m, | |
| | carboxamido)benzoic acid | | 1H), 3.62 (m, 2H), 2.64 (m, 2H) | |
| 222 | 2-(3-Chlorobenzo[b]- | >250 | [500 MHz] 12.15 (s, 1H), 9.06 (m, | 474.2 |
| | thiophene-2-carboxamido)-4- | | 1H), 8.70 (m, 1H), 8.32 (m, 2H), | [MH]⁺ |
| | (5-(methylamino)pentyl- | | 8.15 (m, 2H), 7.99 (m, 1H), 7.66 (m, | |
| | carbamoyl)benzoic acid | | 3H), 3.30 (m, 2H), 2.89 (m, 2H), | |
| | | | 2.56 (s, 3H), 1.59 (m, 4H), 1.36 (t, | |
| | | | 2H) | |
| 223 | 2-(3-Chlorobenzo[b]- | >250 | [500 MHz] 12.16 (s, 1H), 9.03 (m, | 446.1 |
| | thiophene-2-carboxamide)-4- | | 1H), 8.84 (m, 1H), 8.35 (m, 2H), | [MH]⁺ |
| | (3-(methylamino)propyl- | | 8.17 (m, 2H), 8.01 (m, 1H), 7.66 (m, | |
| | carbamoyl)benzoic acid | | 3H), 3.37 (m, 2H), 2.96 (m, 2H), | |
| | | | 2.60 (s, 3H), 1.86 (t, 2H) | |

### Example 224

### 3-Chlorothiophene-2-carboxylic acid[2-(4-chlorophenylcarbamoyl)-5-(2H-tetrazol-5-yl)-phenyl]amide

### Method 18

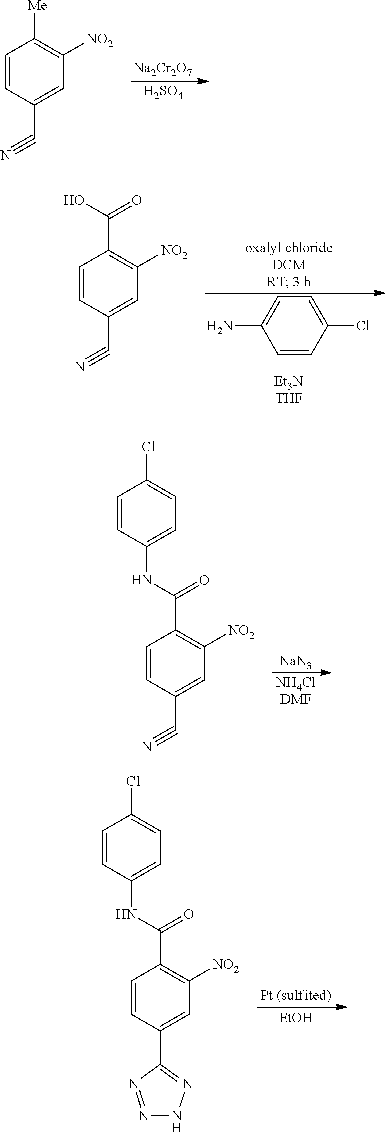

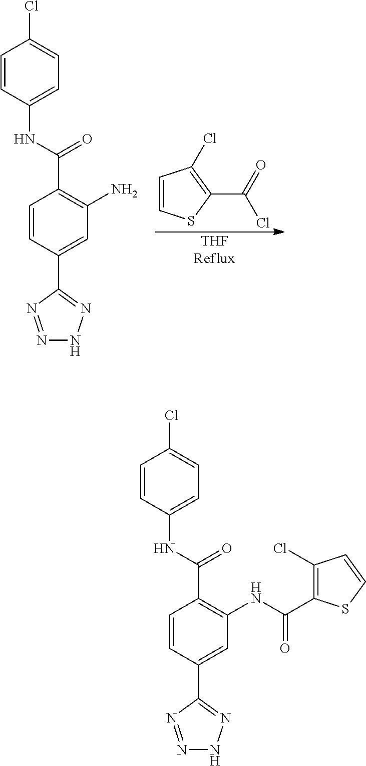

To a solution of 4-cyano-2-nitrotoluene (1 eq.) in 70% sulfuric acid at 0-10° C. was added sodium dichromate (1.5 eq.) portionwise and the reaction mixture was stirred at room temperature for 5 h. The reaction mixture was poured into crushed ice and extracted with ethyl acetate. The organic layer was collected, dried over MgSO₄ and evaporated under vacuum. The residue was dissolved in dichloromethane, oxalyl chloride (4 eq.) and a catalytic amount of DMF were added and the reaction mixture was stirred at room temperature for 4 h. The solvent was removed under vacuum, the residue was diluted with little dichloromethane and evaporated again until all acidity was removed. The residue was dissolved in tetrahydrofuran, treated with 4-chloroaniline (1.2 eq.) and triethylamine (1.2 eq.) and the reaction mixture was stirred at room temperature for 12 h. Cold water was added and the mixture was extracted with ethyl acetate. The organic layer was collected, washed successively with diluted HCl, water and brine, dried over MgSO₄ and evaporated under vacuum. The residue was chromatographed over silica gel, eluting with 15% ethyl acetate-hexane; the collected fractions were evaporated under vacuum to afforded a residue which was dissolved in DMF; sodium azide (4 eq.) and ammonium chloride (4 eq.) were added and the reaction mixture was stirred for 24 h at 100° C. After cooling, the mixture was diluted with ice-cold water and the precipitated solid was collected by suction filtration, washed with water and dried to provide the desired tetrazole derivative.

The latter was dissolved in MeOH and hydrogenated for 20 h in the presence of 10% platinum sulfide on carbon. After filtration of the catalyst through a Celite pad, the filtrate was concentrated under vacuum to afford a residue which was dissolved in anhydrous THF. To this solution, 3-chlorothiophene-2-carbonyl chloride (1 eq.) was added and the reaction was refluxed for 14 h. The solvent was concentrated under vacuum and the residue was crystallized form ethyl acetate-hexane to afford the title compound, mp>250, in 3.3% overall yield.

¹H-NMR (300 MHz, DMSO-d₆, 300° K), δ ppm: 11.23 (s, 1H, NH); 10.84 (s, 1H, NH); 9.04 (d, J 1.08 Hz, 1 Ar—H); 8.08-7.25 (m, 8 Ar—H); MS: m/z=457 [M-H]−

### Example 225

### N,N′-(4-(2H-tetrazol-5-yl)-1,2-phenylene)bis(3-chlorothiophene-2-carboxamide)

### Method 19

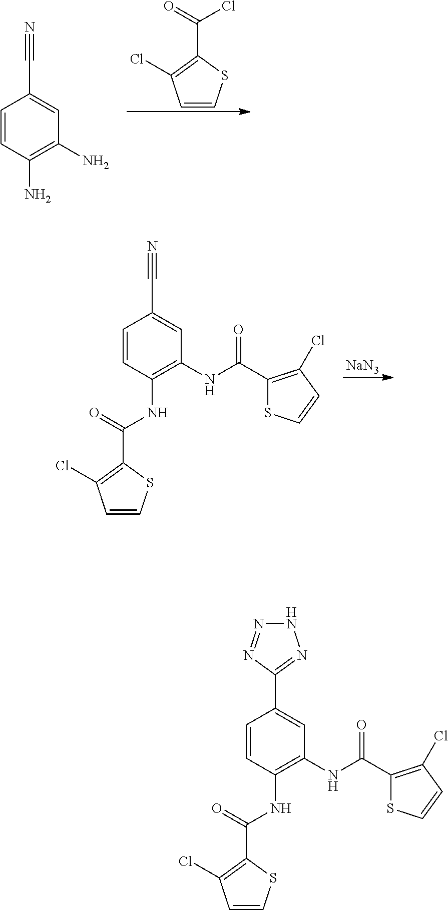 [SMILES: N#Cc1ccc(N)c(N)c1 ; O=C(Cl)c1sccc1Cl ; N#Cc1ccc(NC(=O)c2sccc2Cl)c(NC(=O)c2sccc2Cl)c1 ; O=C(Nc1ccc(-c2nn[nH]n2)cc1NC(=O)c1sccc1Cl)c1sccc1Cl ; "NaN3"]

A mixture of 3,4-diaminobenzonitrile (437 mg, 3.28 mmol) and 3-chlorothiophene-2-carbonyl chloride (1.49 g, 8.21 mmol) in THF (40 ml) was stirred at room temperature for 24 h. The precipitate was collected to obtain N,N′-(4-cyano-1,2-phenylene)bis(3-chlorothiophene-2-carboxamide) (1.23 g, 89%). A mixture of this compound (500 mg, 1.18 mmol), sodium azide (307 mg, 4.72 mmol) and ammonium chloride (252 mg, 4.72 mmol) was stirred at 100° C. for 18 h. The mixture was poured into ice and the precipitate was collected and washed with water, methanol and ethyl ether to obtain 280 mg (51%) of the title compound as a white solid mp>200° C.

¹H-NMR (300 MHz, DMSO-d₆, 353° K), δ ppm: 9.50-9.75 (m, 2H), 8.41 (d, 1H), 7.95-8.09 (m, 2H), 7.85-7.95 (m, 2H), 7.09-7.27 (m, 2H); M S: m/z=465 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 226 | 3-Chloro-N-(5-(4-chloro- | >200 | [400 MHz] 13.14 (br.s, 1H), 12.11 | 516.2 |
| | phenylcarbamoyl)-2-(2H- | | (br s, 1H), 9.15 (s, 1H), 8.23 (m, | [MH]⁺ |
| | tetrazol-5-yl)phenyl)- | | 2H), 8.06 (m, 1H), 8.00 (m, 1H), | |
| | thiophene-2-carboxamide | | 7.65 (m, 3H). | |
| 227 | 3-Chloro-N-(5-(5-chloro-1,3- | >200 | [400 MHz] 13.11(br.s, 1H), 12.29 | 508.3 |
| | thiazol-2-ylcarbamoyl)-2-(2H- | | (br.s, 1H), 9.17 (s, 1H), 8.26 (m, | [MH]⁺ |
| | tetrazol-5-yl)phenyl)benzo[b]- | | 1H), 8.24-8.01 (m, 2H), 7.80-7.75 | |
| | thiophene-2-carboxamide | | (m, 3H), 7.65 (m, 1H), 7.50-7.43 (m, | |
| | | | 2H), 7.41 (m, 1H). | |
| 228 | N-(5-(5-Chloro-1,3-thiazol-2- | >200 | [353° K] 9.50-9.75 (m, 2H), 8.41 (d, | 464.88 |
| | ylcarbamoyl)-2-(2H-tetrazol- | | 1H), 7.95-8.09 (m, 2H), 7.85-7.95 | [MH]⁺ |
| | 5-yl)phenyl)-5-phenyl- | | (m, 2H), 7.09-7.27 (m, 2H) | |
| | thiophene-2-carboxamide | | | |

### Example 229

### 3-Chloro-benzo[b]thiophene-2-carboxylic acid [5-(4-chloro-phenyl-carbamoyl)-2-(2H-tetrazol-5-yl)phenyl]amide

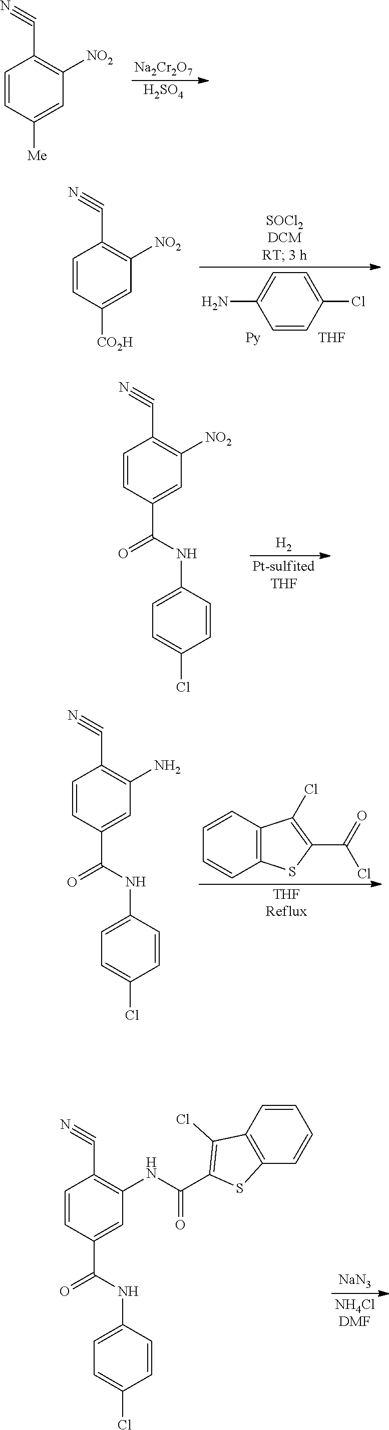

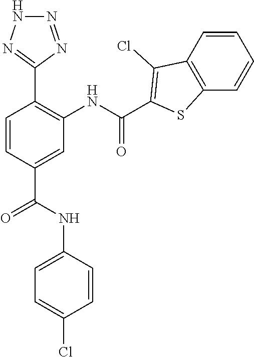 [SMILES: O=C(Nc1ccc(Cl)cc1)c1ccc(-c2nn[nH]n2)c(NC(=O)c2sc3ccccc3c2Cl)c1]

To a solution of 4-cyano-2-nitrotoluene (1 eq.) in 70% sulfuric acid at 0-10° C. sodium dichromate (1.5 eq.) was added portionwise under stirring, and stirring was continued at room temperature for 5 h. The reaction mixture was poured into crushed ice and extracted with ethyl acetate. The organic layer was washed with water and brine, dried over Na₂SO₄ and evaporated to dryness. The residue was dissolved in dichloromethane, treated with oxalyl chloride (4 eq.) and a catalytic amount of DMF. After stirring at room temperature for 4 h, the solvent was evaporated under vacuum, the residue re-dissolved in dichloro-methane and evaporated again to dryness. The residue was dissolved in dichloro-methane, treated with triethylamine (1.2 eq.) and 4-chloroaniline (1.2 eq.) and stirred at room temperature for 12 h. After evaporation of solvent under vacuum, the residue was partitioned between water and ethyl acetate. The organic layer was washed with diluted HCl and water, dried over Na₂SO₄ and evaporated under vacuum. The residue was chromatographed over silica gel (15% ethyl acetate-hexane). The collected fractions were evaporated under vacuum to afford 0.38 eq. (38%) of 4′-chloro-4-cyano-3-nitrobenzoylaniline.

This product was dissolved in MeOH and hydrogenated for 20 h in the presence of 10% platinum sulfide on carbon. After filtration of the catalyst through a Celite pad, the filtrate was concentrated under vacuum and the residue was dissolved in dry THF. 3-Chloro-thiophene-2-carbonyl chloride was added and the reaction mixture was refluxed for 14 h. The solvent was concentrated under vacuum and the residue was crystallized form ethyl acetate-hexane. The compound obtained (0.25 eq.) was dissolved in DMF and treated with sodium azide (1 eq.) and ammonium chloride (1 eq.) and the reaction mixture was stirred for 24 h at 100° C. After cooling, the reaction mixture was diluted with ice cold water. The precipitated solid was collected by suction filtration, washed with water and dried to afford the title compound, mp>250° C., in 12% overall yield.

¹H-NMR (300 MHz, DMSO-d₆, 300° K), δ ppm: 11.68 (s, 1H, NH); 10.63 (s, 1H, NH); 8.95 (s, 1 Ar—H); 8.21-7.44 (m, 10 Ar—H); MS: m/z=507 [M-H]⁻

### Example 230

### 4-(3-Chloro-benzo[b]thiophene-2-carboxamido)-3-[(E)-2-(4-chloro-phenyl)vinyl]benzoic acid

### Method 21

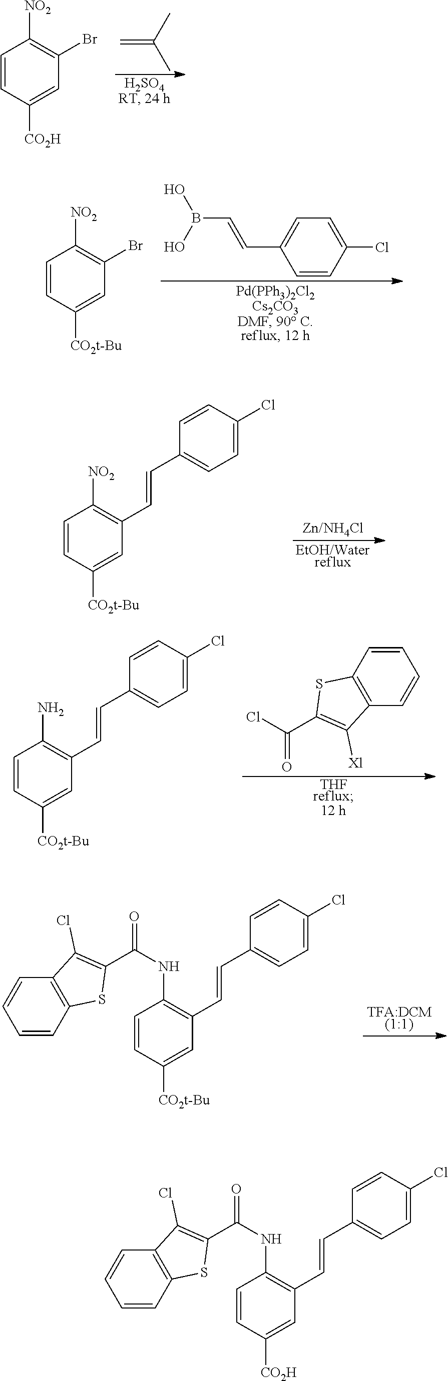

To a solution of 3-bromo-4-nitrobenzoic acid (1 eq.) in dioxane was added a catalytic amount of sulfuric acid and condensed 2-methyl propene −15° C. in a sealed tube. The reaction mixture was stirred at room temperature for 48 h, then poured into 10% NaOH solution and extracted with ethyl acetate. The organic layer was washed with water and brine, dried over MgSO₄ and evaporated under vacuum. The residue was dissolved in DMF and the resulting solution was degassed for 20 minutes; cesium carbonate (1.5 eq.), 4-chlorophenylvinyl boronic acid (1 eq.) and bis-triphenylphosphine palladium(II)chloride were added and the reaction mixture was stirred for 14 h at 90° C. After cooling, the reaction mixture was filtered through a Celite pad and the filtrate was diluted with cold water. The mixture was extracted with ethyl acetate, the organic layer was washed with water and brine, dried over MgSO₄ and concentrated under vacuum. The residue was chromatographed over silica gel (2% ethyl acetate-hexane) and the collected fractions were evaporated under vacuum. ̂ The residue was dissolved in THF-ethanol (2:1) and zinc dust (7 eq.) and ammonium chloride were added to the solution. After heating at reflux for 3 h, the solution was cooled to room temperature, filtered through a Celite pad and concentrated under vacuum. The residue was partitioned between water and ethyl acetate, the organic layer was dried over MgSO₄ and concentrated to dryness under vacuum. The residue was dissolved in anhydrous THF, 3-chlorobenzo[b]thiophene-2-carbonyl chloride was added and the reaction mixture was refluxed for 14 h. After cooling, the solvent was concentrated under vacuum and the residue was crystallized form ethyl acetate-hexane to provide the t-butyl ester of 4-[(3-chloro-benzo[b]thiophene-2-carbonyl)-amino]-3-[(E)-2-(4-chloro-phenyl)vinyl]benzoic in 23% overall yield. This compound was dissolved in a 1:1 mixture of dichloromethane and TFA, and stirring was continued at room temperature for 1 h. The solvent was evaporated under vacuum and the residue was crystallized from diethyl ether, to afford 80% of the title compound, mp>250° C.

¹H-NMR (300 MHz, DMSO-d₆, 300° K), δ ppm: 13.10 (br.s, 1H, CO₂H); 10.46 (s, 1H, NH); 8.35-7.33 (m, 13H); MS: m/z=466 [M-H]⁻

### Example 231

### 2-(3-Chlorobenzo[b]thiophene-2-carboxamido)-3-(4-chlorophenyl-carbamoyl)benzoic acid

### Method 22

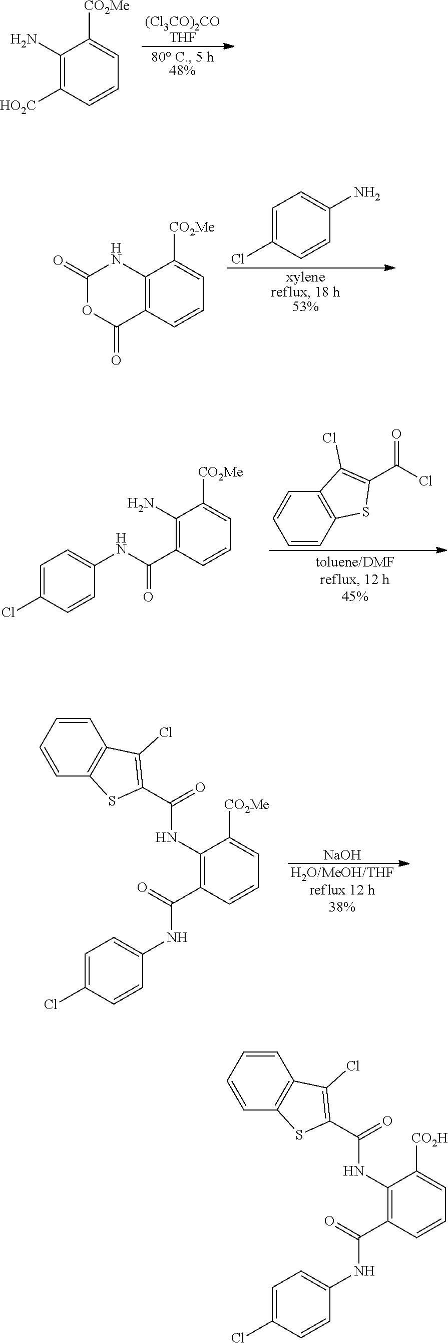

A solution of methyl 2-amino-3-carboxybenzoate in dry THF was added triphosgene (0.33 eq.) and refluxed for 5 h. After cooling to room temperature the solvent was removed under vacuum and the residue was triturated with THF-hexane to afford the corresponding cyclic anhydride as a white solid. The anhydride was dissolved in xylene and 4-chloro-aniline (1.1 eq.) was added together with a catalytic amount of DMAP. The reaction mixture was refluxed for 18 h. After cooling, ice-cold water was added and the mixture was stirred the solution for half an hour. The precipitated solid was filtered, washed with ether and dried. This was dissolved in dry THF, treated with 3-chlorobenzo[b]thiophene-2-carbonyl chloride (1.1 eq.) and refluxed for 10-12 h. The solvent was concentrated under vacuum and the residue was dissolved in 20 ml of THF and 10 ml of EtOH. 6 N NaOH (1.65 mmol) was added and the solution was stirred at room temperature for 3 h. The mixture was filtered through a Celite pad, then poured into acidic water; the precipitate was collected, washed with water and MeOH, and dried to afford the title compound, mp>250° C., in 7.3% overall yield.

¹H-NMR (500 MHz, DMSO-d₆, 300° K), δ ppm: 13.55 (br.s, 1H), 11.30 (br.s, 1H), 10.60 (s, 1H), 8.13 (m, 1H), 8.12 (m, 1H), 8.08 (m, 1H), 7.97 (m, 1H), 7.91 (m, 2H), 7.72 (m, 2H), 7.49 (m, 1H), 7.35 (m, 2H); MS: m/z=507.1 [M+Na]⁺

### Example 232

### 3-(3-Chlorobenzo[b]thiophene-2-carboxamido)-4-cyclohexylcarbamoyl)-benzoic acid

### Method 23

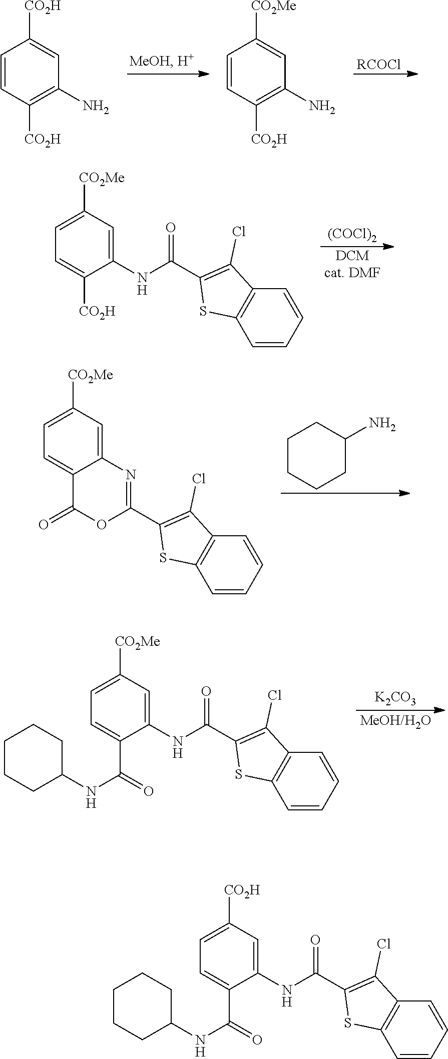

A mixture of 2-amino-terephthalic acid (10.0 g, 55.1 mmol), 96% sulfuric acid (10 mL) and MeOH (100 mL) was stirred at 60° for 7 h, then concentrated to half its volume, poured into ice and cautiously brought to pH 8 with about 20 g of solid sodium carbonate. The solution was washed with DCM (3×40 mL), then cautiously acidified to pH 2 with conc. HCl. The precipitate was collected by filtration, washed repeatedly with water and dried under vacuum at 40° C. overnight. 2-Amino-terephthalic acid 4-methyl ester was obtained as a yellow solid (8.56 g, 80%). To a suspension of this ester (2.0 g, 10.3 mmol) in DCM (65 mL), triethylamine (1.78 mL, 12.8 mmol) was added; after dissolution, 3-chloro-benzo[b]thiophene-2-carbonyl chloride (1.97 g, 8.55 mmol) was added and the mixture was stirred over 3 days at room temperature. The mixture was evaporated under vacuum and the residue was crystallized from about 50 mL of ethyl acetate, then washed with fresh ethyl acetate. 2-(3-Chloro-benzo[b]thiophene-2-carboxamido)terephthalic acid 4-methyl ester was obtained as a white solid (2.91 g, 73%). A mixture of this compound (200 mg, 0.514 mmol), oxalyl chloride (0.052 mL, 0.617 mmol), DCM (5 mL) and 2 drops of DMF was refluxed for 1 h, then evaporated to dryness. The residue was mixed with cyclohexylamine (102 mg, 1.02 mmol) in DMF (5 mL) and the resulting solution was stirred for 1.5 h at room temperature. The solution was diluted with DCM (5 mL) and washed with diluted HCl, dried over sodium sulfate, filtered and evaporated under vacuum. The white solid residue was mixed with potassium carbonate (426 mg, 3.1 mmol), water (2 mL) and methanol (6 mL) and stirred at 90° C. for 5 h. The clear solution thus formed was acidified with conc. HCl and stirring was continued for 1 h at room temperature, then the white precipitate was collected by suction filtration and washed repeatedly with water to obtain the title compound (93 mg, 40% overall) as a white solid, mp>200° C.

¹H-NMR (300 MHz, DMSO-d₆, 300° K), δ ppm: 13.31 (br.s, 1H), 12.09 (s, 1H), 9.04 (d, 1H), 8.77 (d, 1H), 8.11-8.28 (m, 1H), 7.93-8.06 (m, 1H), 7.88 (d, 1H), 7.78 (dd, 1H), 7.53-7.73 (m, 2H), 3.73-3.86 (m, 1H), 1.50-2.05 (m, 5H), 0.91-1.49 (m, 5H); MS: m/z=457 [MH]⁺

### Example 233

### 3-(3-Chlorobenzo[b]thiophene-2-carboxamido)-4-(3-cyanophenyl-carbamoyl)benzoic acid

### Method 24

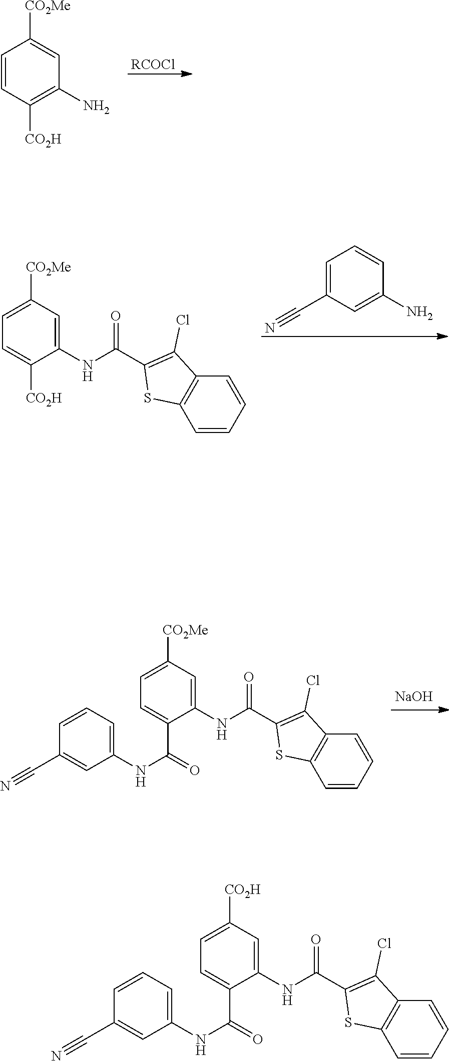

To a suspension of 2-amino-terephthalic acid 4-methyl ester (2.0 g, 10.3 mmol) in DCM (65 mL), triethylamine (1.78 mL, 12.8 mmol) was added; after dissolution, 3-chloro-benzo[b]thiophene-2-carbonyl chloride (1.97 g, 8.55 mmol) was added and the mixture was stirred over 3 days at room temperature. The mixture was evaporated under vacuum and the residue was crystallized from about 50 mL of ethyl acetate, then washed with fresh ethyl acetate. 2-(3-Chloro-benzo[b]thiophene-2-carboxamido)terephthalic acid 4-methyl ester was obtained as a white solid (2.91 g, 73%). A mixture of this compound (600 mg, 1.54 mmol), 3-aminobenzonitrile (182 mg, 1.54 mmol), phosphorous trichloride (0.148 mL, 1.69 mmol) and THF (5 mL) was submitted to microwave heating at 110° C. for 6 h. The solvent was removed under vacuum, the residue treated with DCM and the white precipitate thus formed was collected and further washed with DCM. 3-(3-Chloro-benzo[b]thiophene-2-carboxamido)-4-(3-cyanophenylcarbamoyl)benzoic acid methyl ester was obtained as a pure compound (190 mg, 25%). To a suspension of this compound (103 mg, 0.21 mmol) in THF (10 mL), 6 N sodium hydroxide (0.5 mL, 3.0 mmol) was added and the mixture was stirred at room temperature for 6 h. The reaction mixture was poured into ice containing 0.3 mL of conc. HCl, then the organic solvent was partially removed under vacuum. The white precipitate thus formed was filtered, washed repeatedly with water and dried under vacuum at 40° C. overnight to obtain the title compound (71 mg, 72%) as a white solid mp>200° C.

¹H-NMR (300 MHz, DMSO-d₆, 300° K), δ ppm: 13.39 (br.s, 1H), 11.26 (s, 1H), 10.99 (s, 1H), 8.87 (d, 1H), 8.11-8.22 (m, 2H), 7.99 (d, 1H), 7.93-8.09 (m, 2H), 7.89 (dd, 1H), 7.46-7.73 (m, 4H); MS: m/z=476.08 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 234 | 4-(4-Cyanophenyl- | >200 | 12.24 (br.s, 1H), 10.87 (s, 1H), 9.09 | 510.02 |
| | carbamoyl)-2-(3,6-dichloro- | | (d, 1H) 8.38 (d, 1H), 8.20 (d, 1H), | [MH]⁺ |
| | benzo[b]thiophene-2- | | 7.93-8.08 (m, 3H), 7.85 (m, 2H), | |
| | carboxamido)benzoic acid | | 7.81 (dd, 1H), 7.67 (dd, 1H) | |

### Example 235

### 3-(3-Chlorobenzo[b]thiophene-2-carboxamido-5-(4-chlorophenyl-carbamoyl)benzoic acid

### Method 25

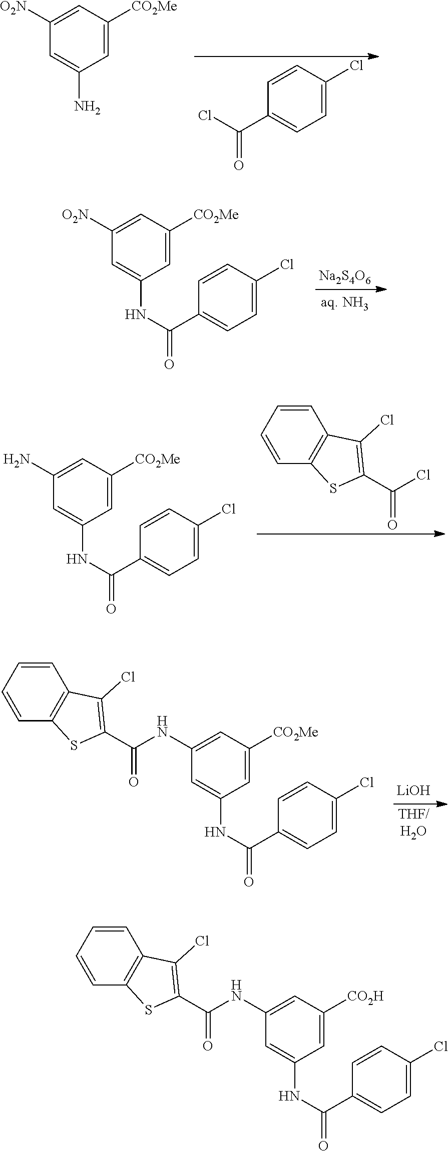

A mixture of 3-amino-5-nitro benzoic acid methyl ester (1 g, 5.09 mmol) and 4-chloro benzoyl chloride (0.981 mg, 5.6 mmol) in 1,2-dichloroethane (25 ml) was refluxed at 90° C. for 1.5 h. Reaction mixture was concentrated and the residue was recrystallised from methanol to yield 3-(4-chlorophenylcarbamoyl)-5-nitrobenzoic acid methyl ester (1.2 g, 70%) as a yellow solid. To a solution of this compound (0.6 g, 1.79 mmol) in dioxane (15 ml) and aqueous ammonia (4 ml) was added sodium dithionate (1.87 g, 10.8 mmol) in small portions at 80° C. the reaction mixture was refluxed at 80° C. overnight. The hot solution was filtered and from the filtrate, dioxane was removed under reduced pressure. The residue was diluted with dichloromethane and water, and insolubles were filtered. The organic layer of the filtrate was separated, washed with water and brine, dried over sodium sulphate and concentrated to yield 3-amino-5-(4-chlorophenylcarbamoyl)benzoic acid methyl ester (160 mg, 29%). A mixture of this ester (160 mg, 0.52 mmol) and 3-chlorobenzo[b]thiophene-2-carbonyl chloride (133 mg, 0.57 mmol) in 1,2-dichloroethane (15 ml) was refluxed at 85° C. for 1 h. The reaction mixture was concentrated and the residue was crystallized from methanol to yield 3-(3-chlorobenzo[b]thiophene-2-carboxamido)-5-(4-chlorophenylcarbamoyl)benzoic acid methyl ester (230 mg, 88%). To a solution of this ester (230 mg, 0.46 mmol) in tetrahydrofuran (5 ml) was added lithium hydroxide hydrate (39 mg, 0.92 mmol) in water (2 ml). The reaction mixture was stirred at 45° C. for 16 h, and tetrahydrofuran was removed under reduced pressure. The residue was acidified with 1.5 N hydrochloric acid, the solid was filtered, washed with water and dried to yield the title compound (105 mg, 45%) as a colourless powder, m.p. 316-317° C.

¹H-NMR (300 MHz, DMSO-d₆), δ ppm: 7.6-7.7 (m, 4H), 7.9-8.3 (m, 6H), 8.56 (s, 1H), 10.58, (s, 1H), 10.82 (s, 1H), 13.1 (s, 1H); MS: m/z=482.5 [M−1]⁻

### Example 236

### 4-(3-Chlorobenzo[b]thiophene-2-carboxamido)-3-(5-methylthiazol-2-yl-carbamoyl)benzoic acid

### Method 26

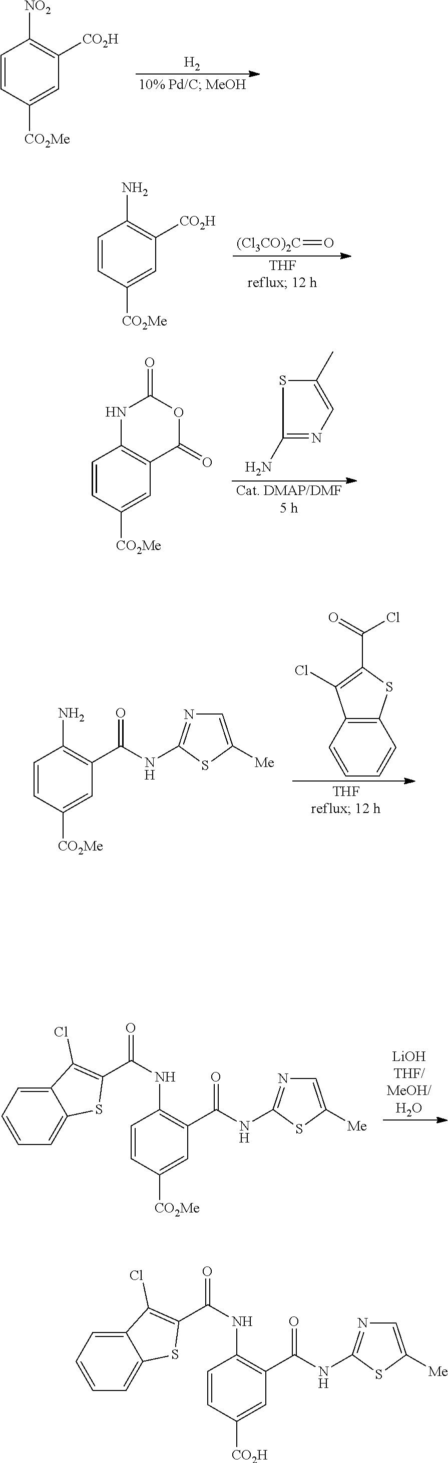

A solution of methyl 3-carboxy-4-nitrobenzoate (1 eq.) in MeOH was hydrogenated for 20 h in the presence of 10% palladium on carbon. After filtration of the catalyst through a Celite pad, the filtrate was concentrated under vacuum. The residue was dissolved in anhydrous THF, treated with triphosgene (0.33 eq.) and refluxed for 24 h. After cooling to room temperature, the solvent was removed under vacuum and the residue was crystallized with THF-hexane, which gave the anhydride as a white solid. This compound was dissolved in the minimum volume of DMF, and 2-amino-5-methylthiazole (1.1 eq.) and a catalytic amount of DMAP were added. The reaction mixture was heated at 70-80° C. for 10 h. After cooling, the reaction mixture was diluted with ice-cold water and stirring was continued at room temperature for 0.5 h. The precipitate was collected by suction filtration, washed with diethyl ether and dried. This compound was dissolved in anhydrous THF, 3-chlorobenzo[b]thiophene-2-carbonyl chloride was added and the reaction mixture was refluxed for 12 h. The solvent was evaporated under vacuum and the residue was crystallized form ethyl acetate-hexane. The obtained compound was dissolved in THF-water (3:1), and LiOH (1.1 eq.) was added. The solution was stirred overnight at room temperature, the organic solvent was evaporated under vacuum, and the aqueous residue dissolved was diluted with a small amount of water, adjusting the pH to 6-7. The precipitated solid was collected by filtration and dried to afford the title compound, mp>200° C., in about 21% overall yield.

¹H-NMR (300 MHz, DMSO-d₆), δ ppm: 8.66-7.39 (m, 8 Ar—H), 2.47 (s, 3H); MS: m/z=472.2 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 237 | 3-(4-Chlorophenyl- | >250 | 11.80 (s, 1H, NH), 10.87 (s, 1H, | 438 |
| | carbamoyl)-4-(5-cyano- | | NH), 8.43-7.42 (m, 9 Ar—H) | [M − H]− |
| | thiophene-2-carboxamido)- | | | |
| | benzoic acid methyl ester | | | |
| 238 | 4-(3-Chloro-benzo[b]- | >200 | 8.89-6.89 (m, 8 Ar—H), 2.47 (s, 3H) | 471.9 |
| | thiophene-2-carboxamido)-3- | | | [MH]⁺ |
| | (4-methyl-1,3-thiazol-2-yl- | | | |
| | carbamoyl)benzoic acid | | | |
| 239 | 3-(5-Acetyl-4-methyl-1,3- | >200 | 8.74-7.52 (m, 7 Ar—H), 2.85 (s, 3H), | 513.9 |
| | thiazol-2-ylcarbamoyl)-4-(3- | | 2.74 (s, 3H) | [MH]⁺ |
| | chlorobenzo[b]thiophene-2- | | | |
| | carboxamido)benzoic acid | | | |
| 240 | 3-(Benzo[d]-1,3-thiazol-2-yl- | >200 | 8.76-7.06 (m, 9 Ar—H) | 458 |
| | carbamoyl)-4-(3-chloro- | | | [MH]⁺ |
| | thiophene-2-carboxamido)- | | | |
| | benzoic acid | | | |
| 241 | 4-(3-Chloro-4,5-divinyl- | >200 | 8.76-7.53 (m, 8 Ar—H) | 491.8 |
| | thiophene-2-carboxamido)-3- | | | [MH]⁺ |
| | (5-chloro-1,3-thiazol-2-yl- | | | |
| | carbamoyl)benzoic acid | | | |
| 242 | 4-(3-Chlorothiophene-2- | >250 | 8.87-7.18 (m, 8 Ar—H) | 475.8 |
| | carboxamido)-3-(6-fluoro- | | | [MH]⁺ |
| | benzothiazol-2-ylcarbamoyl)- | | | |
| | benzoic acid | | | |
| 243 | 4-(3-Chlorothiophene-2- | >250 | 8.69-7.09 (m, 5 Ar—H), 3.01-3.03 (s, | 448 |
| | carboxamido)-3-(5,6-dihydro- | | 4H), 2.67-2.70 (s, 2H) | [MH]⁺ |
| | 4H-cyclopenta-1,3-thiazol-2- | | | |
| | ylcarbamoyl)benzoic acid | | | |

### Example 244

### 4′-Chloro-3-(3-chlorobenzo[b]thiophene-2-carboxamido)biphenyl-4-carboxylic acid

### Method 27

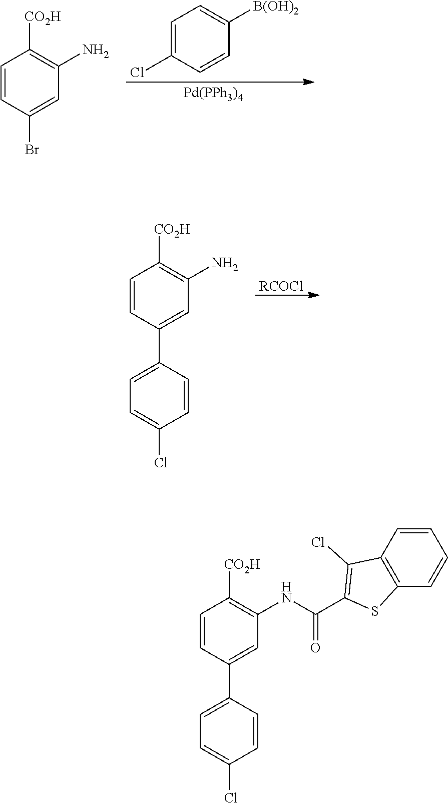

To a solution of 4-chloro-phenylboronic acid (0.6 g, 3.8 mmol) in degassed dioxane (50 mL), Pd(PPh₃)₄ (0.26 g, 3.8 mmol), a 2 M solution of aqueous potassium carbonate (5.6 mL, 11.5 mmol) and 2-amino-4-bromo-benzoic acid (1.0 g, 4.6 mmol) were added at room temperature, and the mixture was heated to 80° C. for 3 h. The reaction mixture was cooled at room temperature and the solvent was removed under vacuum. The crude product thus obtained was portioned between water (20 mL) and ethyl acetate (30 mL). The organic layer was separated and the aqueous layer was further extracted with ethyl acetate (3×15 mL). The combined organics were dried over anhydrous sodium sulfate, filtered and evaporated under vacuum. 3-Amino-4′-chloro-biphenyl-4-carboxylic acid was obtained as a brownish solid (0.1 g, 12% yield). A solution of this compound (0.08 g, 0.32 mmol) in dry THF (5 mL), was cooled at 0° C. 3-Chlorobenzo[b]thiophene-2-carbonyl chloride (0.06 g, 0.27 mmol) was added portionwise. The reaction was allowed to reach room temperature and stirring was continued overnight. The mixture was evaporated and the residue was triturated in a 1:1 MeOH:Et₂O mixture (about 20 mL). The solid was filtered, washed with plenty of Et₂O and then dried under vacuum at 40° C. overnight to provide the title compound (0.047 g, 40%) as a white solid, mp>200° C.

¹H-NMR (300 MHz, DMSO-d₆) δ ppm: 13.90 (br.s, 1H), 12.27 (s, 1H), 8.96 (d, 1H), 8.14-8.24 (m, 1H), 8.15 (d, 1H), 7.93-8.05 (m, 1H), 7.78 (m, 2H), 7.62-7.71 (m, 2H), 7.60 (m, 2H), 7.58 (dd, 1H); MS: m/z=442 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 245 | 2-(3-Chlorobenzo[b]- | >200 | 13.85 (br.s, 1H), 12.30 (s, 1H), 8.99 | 414.14 |
| | thiophene-2-carboxamide)-4- | | (d, 1H), 8.13-8.23 (m, 1H), 8.09 (d, | [MH]⁺ |
| | thiophen-2-ylbenzoic acid | | 1H), 7.94-8.04 (m, 1H), 7.50-7.80 | |
| | | | (m, 5H), 7.22 (dd, 1H) | |
| 246 | 2-(3-Chlorobenzo[b]- | >200 | 13.79 (br.s, 1H), 12.21 (s, 1H), 8.65 | 448.13 |
| | thiophene-2-carboxamido)-4- | | (d, 1H), 8.11-8.22 (m, 1H), 7.99 (d, | [MH]⁺ |
| | ((E)-3-phenylpropenyl)- | | 1H), 7.87-7.99 (m, 1H), 7.56-7.73 | |
| | benzoic acid | | (m, 2H), 7.10-7.43 (m, 6H), 6.64 (dd, | |
| | | | 1H), 6.56 (d, 1H), 3.59 (d, 2H) | |
| 247 | 2-(3-Chlorobenzo[b]- | >200 | 13.71 (br.s, 1H), 12.20 (s, 1H), 8.54 | 470.00 |
| | thiophene-2-carboxamide)-4- | | (d, 1H), 8.11-8.23 (m, 1H), 7.89-8.03 | [MH]⁺ |
| | [2-(4-chlorophenyl)ethyl]- | | (m, 2H), 7.59-7.70 (m, 2H), 7.22- | |
| | benzoic acid | | 7.40 (m, 4H), 7.12 (dd, 1H), 2.86- | |
| | | | 3.07 (m, 4H) | |
| 248 | 2-(3-Chlorobenzo[b]- | >200 | 14.04 (br.s, 1H), 12.24 (s, 1H), 8.79 | 465.93 |
| | thiophene-2-carboxamide)-4- | | (d, 1H), 8.13-8.22 (m, 1H), 8.09 (d, | [MH]⁺ |
| | ((4-chlorophenyl)ethynyl)- | | 1H), 7.93-8.03 (m, 1H), 7.60-7.72 | |
| | benzoic acid | | (m, 4H), 7.53 (m, 2H), 7.44 (dd, 1H) | |
| 249 | 3-(3-Chlorobenzo[b]- | >200 | 13.82 (br.s, 1H), 12.32 (br.s, 1H), | 437.98 |
| | thiophene-2-carboxamido)-4′- | | 8.94 (d, 1H), 8.07-8.28 (m, 2H), | [MH]⁺ |
| | methoxybiphenyl-4-carboxylic | | 7.94-8.05 (m, 1H), 7.59-7.80 (m, | |
| | acid | | 4H), 7.54 (dd, 1H), 7.11 (m, 2H), | |
| | | | 3.84 (s, 3H) | |
| 250 | 6′-Chloro-3-(3-chloro- | >200 | 13.95 (br.s, 1H), 12.32 (br.s, 1H), | 442.02 |
| | benzo[b]thiophene-2- | | 8.73 (d, 1H), 8.10-8.24 (m, 2H), | [MH]⁺ |
| | carboxamido)biphenyl-4- | | 7.90-8.05 (m, 1H), 7.57-7.72 (m, | |
| | carboxylic acid | | 3H), 7.43-7.55 (m, 3H), 7.35 (dd, 1H) | |
| 251 | 2-(3-Chlorobenzo[b]- | >200 | 13.74 (br.s, 1H), 12.25 (s, 1H), 8.96 | 413.91 |
| | thiophene-2-carboxamide)-4- | | (d, 1H), 8.13-8.22 (m, 1H), 8.09 (d, | [MH]⁺ |
| | (thiophen-3-yl)benzoic acid | | 1H), 8.04 (dd, 1H), 7.94-8.02 (m, | |
| | | | 1H), 7.72 (dd, 1H), 7.55-7.70 (m, 4H) | |
| 252 | 4-(5-Phenylthiophen-2-yl)-2- | >200 | 12.39 (br.s, 1H), 11.02 (br.s, 1H), | 489.86 |
| | (3-chlorobenzo[b]thiophene- | | 8.22 (d, 1H), 8.02 (d, 1H), 7.92 (m, | [MH]⁺ |
| | 2-carboxamido)benzoic acid | | 2H), 7.80 (m, 2H), 7.37 (q, 1H), 2.43 | |
| | | | (d, 3H) | |
| 253 | 4-(5-Carbamoylthiophen-2- | >200 | 13.93 (br.s, 1H), 12.30 (br.s, 1H), | 456.93 |
| | yl)-2-(3-chlorobenzo[b]- | | 9.02 (s, 1H), 7.91-8.23 (m, 4H), 7.80 | [MH]⁺ |
| | thiophene-2-carboxamido)- | | (d, 1H), 7.42-7.75 (m, 5H) | |
| | benzoic acid | | | |
| 254 | 2-(3-Chlorobenzo[b]- | >200 | 13.90 (br.s, 1H), 12.30 (br.s, 1H), | 447.91 |
| | thiophene-2-carboxamido)-4- | | 8.90 (br.s, 1H), 7.92-8.24 (m, 3H), | [MH]⁺ |
| | (5-chlorothiophen-2-yl)- | | 7.43-7.76 (m, 4H), 7.11-7.37 (m, 1H) | |
| | benzoic acid | | | |
| 255 | 3-(3-Chlorobenzo[b]- | >200 | [353° K] 12.10 (br.s, 1H), 8.98 (d, | 432.96 |
| | thiophene-2-carboxamido)-4′- | | 1H), 8.19 (d, 1H), 8.04-8.17 (m, 1H), | [MH]⁺ |
| | cyanobiphenyl-4-carboxylic | | 7.83-8.05 (m, 5H), 7.50-7.77 (m, 3H) | |
| | acid | | | |

### Example 256

### 4′-Chloro-6-(3-chlorobenzo[b]thiophene-2-carboxamido)biphenyl-3-carboxylic acid

### Method 28

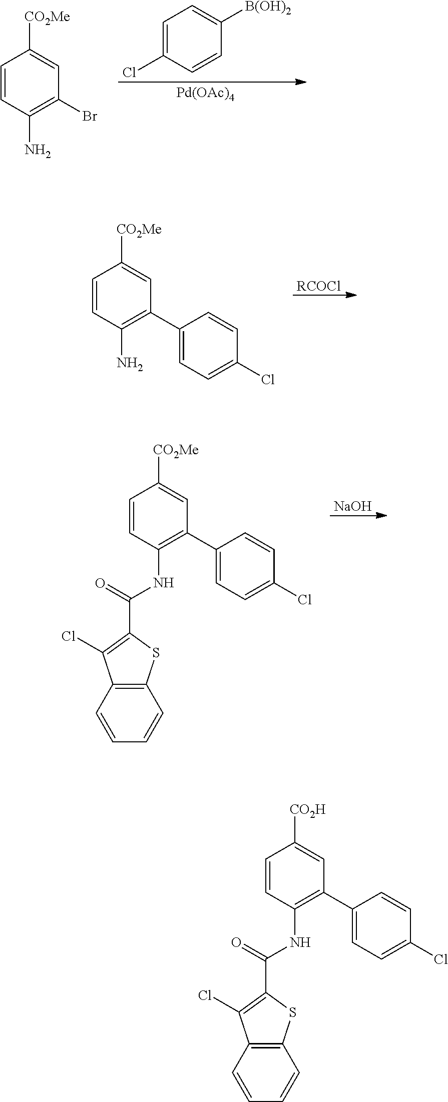

Methyl 4-amino-3-bromobenzoate (300 mg, 1.3 mmol), 4-chlorophenylboronic acid (408 mg, 2.6 mmol) and Pd(OAc)₂ (15 mg, 0.058 mmol) were mixed and grinded, then triethylamine was added (1 ml). The mixture was stirred at room temperature overnight, then another portion of 4-chlorophenylboronic acid (150 mg, 0.96 mmol) was added and the mixture was stirred overnight. The mixture was diluted with ethyl ether and washed with water and brine. The organic phase was dried and evaporated, and the crude product was purified by flash chromatography to give 6-amino-4′-chlorobiphenyl-3-carboxylic acid methyl ester (230 mg, 70%). A solution of this ester (215 mg, 0.82 mmol), 3-chloro-benzo[b]thiophene-2-carbonyl chloride (418 mg, 1.80 mmol) and pyridine (0.09 ml, 1.07 mmol) in THF (10 ml) was stirred at room temperature overnight. After reducing the mixture to half its volume, the precipitate was collected and washed with ethyl ether to give 4-chloro-6-(3-chlorobenzo[b]thiophene-2-carboxamido)biphenyl-3-carboxylic acid methyl ester (230 mg, 61%). To a solution of this compound (160 mg, 0.35 mmol) in THF (15 ml), 6 N NaOH (0.80 ml, 5.26 mmol) was added and the mixture was stirred at room temperature for 2 days. The reaction mixture was poured into ice containing 1 ml of conc. HCl and the precipitate was collected and washed with ethyl ether/acetone to obtain the title compound (70 mg, 45%) as a white solid mp>200° C.

¹H NMR (300 MHz, DMSO-d₆), δ ppm: 13.06 (br.s, 1H), 9.87 (s, 1H), 8.08-8.17 (m, 1H), 8.06 (d, 1H), 8.03 (dd, 1H), 7.84-7.94 (m, 2H), 7.57-7.67 (m, 2H), 7.47-7.59 (m, 4H); MS: m/z=442 [MH]⁺

### Example 257

### 2-(3-Chlorobenzo[b]thiophene-2-carboxamido)-4-(1-(4-chlorophenyl)-1H-1,2,3-triazol-4-yl)benzoic acid

### Method 29

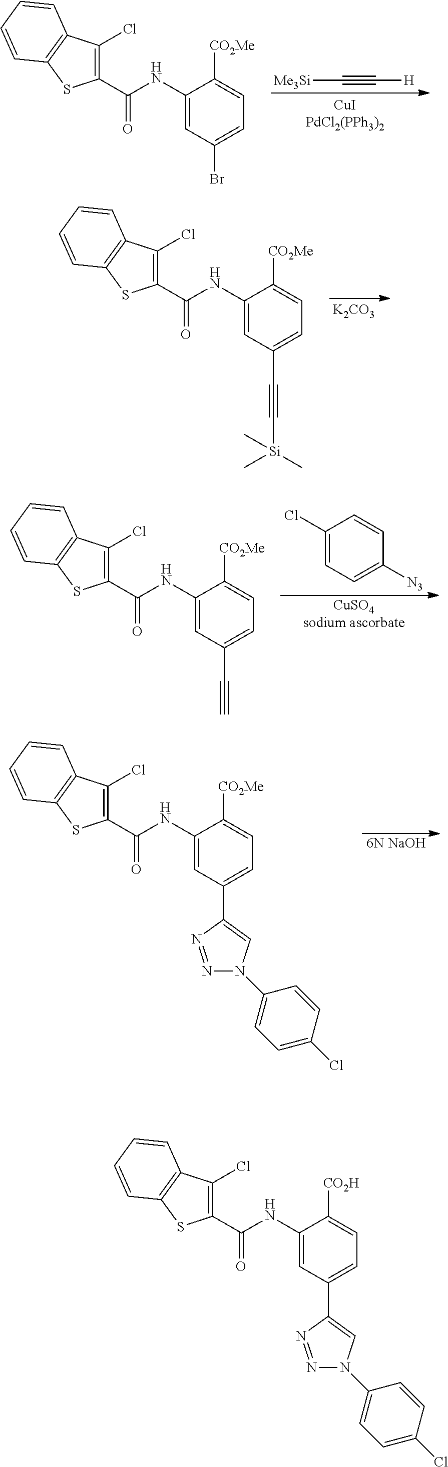

A mixture of 4-bromo-2-(3-chloro-benzo[b]thiophene-2-carboxamido)benzoic acid methyl ester (0.40 g, 0.94 mmol), CuI (18 mg, 0.094 mmol), PdCl₂(PPh₃)₂ (66 mg, 0.094 mmol) and ethynyl-trimethylsilane (0.267 ml, 1.88 mmol) in triethylamine/acetonitrile (1:1, 10 ml) was heated with microwave at 100° C. for 1 h in a sealed tube. After evaporation of the solvents, the crude mixture was purified by flash chromatography to obtain pure 2-(3-chloro-benzo[b]thiophene-2-carboxamido)-4-trimethylsilanylethynyl-benzoic acid methyl ester as a pale yellow solid (0.315 mg, 76%).

To a solution of this compound (0.631 mg, 1.43 mmol) in dichloromethane/methanol (1:1, 30 ml), solid potassium carbonate was added (0.395 g, 2.86 mmol); after stirring at room temperature for 30 min, the solid was eliminated by filtration and the solvents were evaporated. The residue was redissolved in dichloromethane and washed with water and brine, then dried and evaporated. 2-(3-Chloro-benzo[b]thiophene-2-carboxamido)-4-ethynyl-benzoic acid methyl ester was obtained as a yellow solid (0.471 g, 89%). To a suspension of this compound (100 mg, 0.271 mmol) in a mixture of water and dichloromethane (5 ml/10 ml), CuSO₄ (3 mg, 0.016 mmol) and sodium ascorbate (10 mg, 0.049 mmol) were added, followed by 4-chlorophenylazide (50 mg, 0.326 mmol). The mixture was stirred at 50° C. for 4 h, then diluted with dichloromethane; the separated organic phase was further washed with water and brine, then evaporated. The residue was purified by washing with dichloromethane/ethyl ether to obtain pure 4-(1-(4-chloro-phenyl)-1H-[1,2,3]triazol-4-yl)-2-(3-chloro-benzo[b]thiophene-2-carboxamido)-benzoic acid methyl ester as a light brown solid (58 mg, 41%).

To a solution of this (58 mg, 0.111 mmol) in THF/methanol (1:1, 8 ml), 6 N sodium hydroxide (0.185 ml, 1.11 mmol) was added and the mixture was stirred at 50° C. for 4 h. The reaction mixture was poured into ice containing 0.1 ml of conc. HCl. The precipitate thus formed was washed repeatedly with water, then ethyl ether, and finally dried under vacuum at 40° C. overnight to afford the title compound (32 mg, 57%) as a light brown solid, mp>200° C.

¹H NMR (300 MHz, DMSO-d₆) δ ppm: 13.90 (br.s, 1H), 12.51 (br.s, 1H), 9.51 (s, 1H), 9.26 (s, 1H), 8.13-8.26 (m, 2H), 7.94-8.12 (m, 3H), 7.56-7.87 (m, 5H); MS: m/z=509 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 258 | 4-(1-Benzyl-1H-1,2,3-triazol- | >200 | 13.86 (br.s, 1H), 12.29 (s, 1H), 9.17 | 489.06 |
| | 4-yl)-2-(3-chlorobenzo[b]- | | (d, 1H), 8.84 (s, 1H), 8.15-8.22 (m, | [MH]⁺ |
| | thiophene-2-carboxamido)- | | 1H), 8.13 (d, 1H), 7.89-8.05 (m, 1H), | |
| | benzoic acid | | 7.73 (dd, 1H), 7.59-7.70 (m, 2H), | |
| | | | 7.21-7.52 (m, 5H), 5.69 (s, 2H) | |
| 259 | 2-(3-Chlorobenzo[b]- | >200 | 13.85 (br.s, 1H), 12.34 (br.s, 1H), | 523 |
| | thiophene-2-carboxamido)-4- | | 9.17 (s, 1H) 8.83 (s, 1H), 8.05-8.32 | [MH]⁺ |
| | (1-(4-chlorobenzyl)-1H-1,2,3- | | (m, 2H), 7.91-8.05 (m, 1H) 7.59-7.82 | |
| | triazol-4-yl)benzoic acid | | (m, 3H), 7.45 (m, 4H), 5.69 (s, 2H) | |
| 260 | 2-(3-Chlorobenzo[b]- | >200 | [400 MHz] 13.96 (br.s, 1H), 12.32 | 525.1 |
| | thiophene-2-carboxamido)-4- | | (br.s, 1H), 9.15 (s, 1H), 8.82 (m, | [MH]⁺ |
| | (1-(2,6-difluorobenzyl)-1H- | | 1H), 8.19 (m, 1H), 8.12 (d, 1H), 7.98 | |
| | 1,2,3-triazol-4-yl)benzoic acid | | (m, 1H), 7.75 (m, 1H), 7.68-7.62 (m, | |
| | | | 2H), 7.54 (m, 1H), 7.23 (m, 2H), | |
| | | | 5.75 (s, 2H). | |
| 261 | 4-(1-(4-Chloro-3-(trifluoro- | >200 | [400 MHz] 12.48 (br.s, 1H), 9.69 (s, | 575.2 |
| | methyl)phenyl)-1H-1,2,3- | | 1H), 9.26 (m, 1H), 8.49 (d, 1H), 8.39 | [MH]⁺ |
| | triazol-4-yl)-2-(3-chloro- | | (dd, 1H), 8.21-8.18 (m, 2H), 8.05- | |
| | benzo[b]thiophene-2- | | 7.98 (m, 2H), 7.83 (m, 1H), 7.68- | |
| | carboxamido)benzoic acid | | 7.62 (m, 2H) | |
| 262 | 2-(3-Chlorobenzo[b]- | | [400 MHz] 13.40 (br.s, 1H), 9.12 (s, | 529.1 |
| | thiophene-2-carboxamido)-4- | | 1H), 8.77 (s, 1H), 8.18 (d, 1H), 8.11 | [MH]⁺ |
| | (1-((5-chlorothiophen-2-yl)- | 170 | (d, 1H), 7.98 (d, 1H), 7.67-7.62 (m, | |
| | methyl)-1H-1,2,3-triazol-4-yl)- | | 3H), 7.16 (d, 1H), 7.07 (d, 1H), 5.84 | |
| | benzoic acid | | (s, 2H). | |
| 263 | 2-(3-Chlorobenzo[b]- | >200 | 9.44 (s, 1H), 8.74 (d, 1H), 8.65 (d, | 508.85 |
| | thiophene-2-carboxamido)-5- | | 1H), 8.10-8.23 (m, 2H), 8.01-8.09 | [MH]⁺ |
| | (1-(4-chlorophenyI)-1H-1,2,3- | | (m, 2H), 7.93-8.01 (m, 1H), 7.69- | |
| | triazol-4-yl)benzoic acid | | 7.80 (m, 2H), 7.56-7.68 (m, 2H) | |

### Example 264

### 4′-Chloro-5-(3-chlorobenzo[b]thiophene-2-carboxamido)biphenyl-2-carboxylic acid

### Method 30

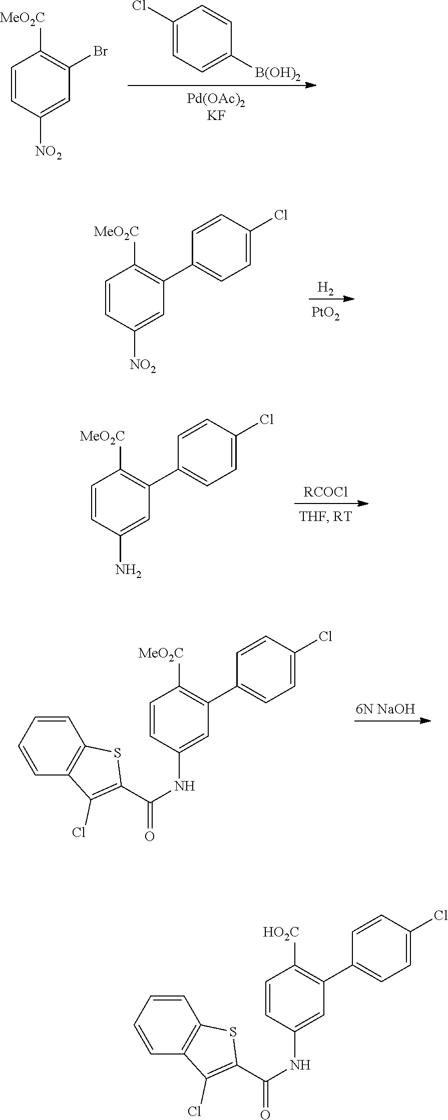

A mixture of methyl 2-bromo-4-nitro-benzoate (500 mg, 1.92 mmol), 4-chlorophenyl-boronic acid (330 mg, 2.11 mmol), palladium acetate (50 mg, 0.22 mmol) and potassium fluoride (279 mg, 4.80 mmol) in methanol (12 ml) was heated with microwave at 100° C. for 45 min in a sealed tube. The mixture was filtered through a Celite pad and the solvent was evaporated; the crude product was purified by flash chromatography to obtain 4′-chloro-5-nitro-biphenyl-2-carboxylic acid methyl ester as a pale yellow solid (432 mg, 77%). A mixture of this compound (334 mg, 1.14 mmol) and PtO₂.x H₂O (35 mg) in methanol/ethyl acetate (2:1, 30 ml) was hydrogenated at 30 psi in a Parr apparatus for 1.5 h. The mixture was then filtered through a Celite pad and evaporated to obtain 5-amino-4′-chloro-biphenyl-2-carboxylic acid methyl ester (304 mg, 100%).

A mixture of this compound (304 mg, 1.16 mmol) and 3-chlorobenzo[b]thiophene-2-carbonyl chloride (224 mg, 0.96 mmol) in dry THF (20 ml) was stirred at room temperature overnight. The precipitate was discarded by filtration and the solution was evaporated. The solid residue was treated with ethyl ether and methanol and further washed with acetonitrile to obtain 4′-chloro-5-(3-chloro-benzo[b]thiophene-2-carboxamido)-biphenyl-2-carboxylic acid methyl ester (224 mg, 51%).

To a solution of this compound (224 mg, 0.49 mmol) in THF/methanol (1:1, 30 ml), 6 N sodium hydroxide (1.22 ml, 7.37 mmol) was added and the mixture was stirred at 50° C. for 20 h. The reaction mixture was poured into ice containing 1 ml of conc. HCl. The precipitate was collected and washed repeatedly with water, then with ethyl ether/methanol and ethyl ether/acetonitrile, and finally dried under vacuum at 40° C. overnight to yield the title compound (57 mg, 26%) as a white solid, mp>200° C.

¹H NMR (300 MHz, DMSO-d₆) δ ppm: 12.66 (br.s, 1H), 10.80 (s, 1H), 8.11-8.24 (m, 1H), 7.91-8.03 (m, 1H), 7.78-7.91 (m, 2H), 7.71-7.78 (m, 1H), 7.58-7.69 (m, 2H), 7.47 (m, 2H), 7.35 (m, 2H); MS: m/z=442 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 265 | 4′-Chloro-4-(3-chloro- | >200 | 12.91 (br.s, 1H), 10.77 (s, 1H), 8.12- | 441.99 |
| | benzo[b]thiophene-2- | | 8.24 (m, 2H), 7.87-8.02 (m, 2H), | [MH]⁺ |
| | carboxamido)biphenyl-2- | | 7.58-7.69 (m, 2H), 7.47 (m, 2H), | |
| | carboxylic acid | | 7.41 (d, 1H), 7.35 (m, 2H) | |
| 266 | 3-(4-(5,6-Dichloro-1H- | >200 | 12.33 (s, 1H), 9.03 (d, 1H), 8.28- | 531.9 |
| | benzo[d]imidazol-2-yl)- | | 8.48 (m, 2H), 8.14-8.19 (m, 2H), | [MH]⁺ |
| | benzamido)-4′-methoxy- | | 8.11 (d, 1H), 7.92 (s, 2H), 7.65-7.76 | |
| | biphenyl-4-carboxylic acid | | (m, 2H), 7.50 (dd, 1H), 7.03-7.17 (m, | |
| | | | 2H), 3.84 (s, 3H) | |
| 267 | 4′-Cyano-3-(4-(5,6-dichloro- | >200 | 12.28 (s, 1H), 9.08 (d, 1H), 8.30- | 526.88 |
| | 1H-benzo[d]imidazol-2-yl)- | | 8.44 (m, 2H), 8.10-8.23 (m, 3H), | [MH]⁺ |
| | benzamido)biphenyl-4- | | 7.98-8.06 (m, 2H), 7.92-7.97 (m, | |
| | carboxylic acid | | 2H), 7.92 (s, 2H), 7.61 (dd, 1H) | |

### Example 268

### 2-(3-Chlorobenzo[b]thiophene-2-carboxamido)-4-(3-phenylpropyl)benzoic acid

### Method 31

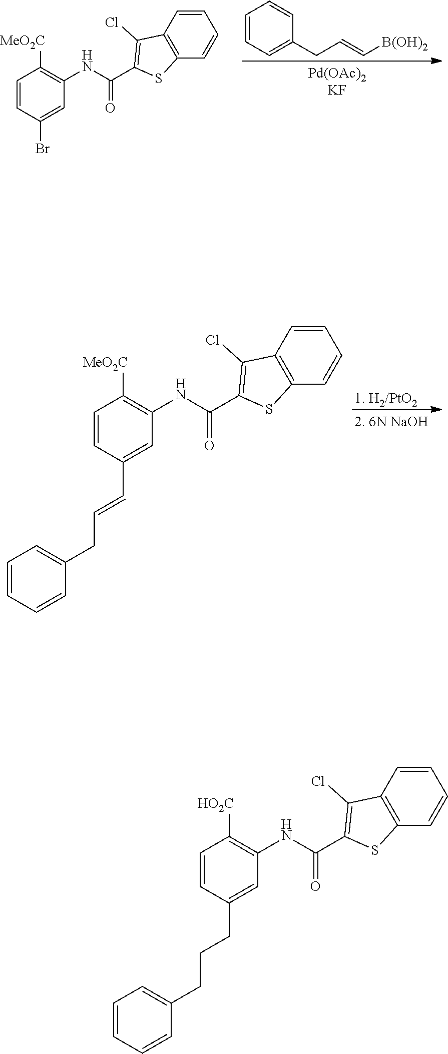

To a solution of trans-3-phenylpropen-1-yl-boronic acid (0.229 g, 1.41 mmol) in degassed MeOH (6 ml), palladium(II) acetate (16 mg, 0.071 mmol), potassium fluoride (0.103 g, 1.77 mmol) and 4-bromo-2-(3-chloro-benzo[b]thiophene-2-carboxamido)-benzoic acid methyl ester (0.3 g, 0.7 mmol) were added at room temperature and the mixture was submitted to a first reaction cycle, under microwave irradiation, for 2 h at 120° C. Fresh trans-3-phenylpropen-1-yl-boronic acid (0.229 g, 1.41 mmol), palladium(II) acetate (16 mg, 0.071 mmol) and potassium fluoride (0.103 g, 1.77 mmol) were added and the mixture was submitted to a second cycle, under the same conditions. The precipitate that formed during the reaction was filtered, dissolved in ethyl acetate and passed through a Celite pad to remove the catalyst. The solvent was evaporated under vacuum. 2-(3-Chloro-benzo[b]thiophene-2-carboxamido)-4-(E)-3-phenyl-propenyl)-benzoic acid methyl ester was obtained as a green solid (0.267 g, 82% yield).

This compound (0.140 g, 0.303 mmol) was added in one portion to a suspension of platinum oxide hydrate (0.15 g) in ethyl acetate (50 ml). The mixture was hydrogenated at 30 psi for 30 h. The mixture was filtered through a Celite pad, and the solvent was evaporated under vacuum. 2-(3-Chloro-benzo[b]thiophene-2-carboxamido)-4-(3-phenyl-propyl)-benzoic acid methyl ester was obtained as a crude compound and used as such in the following reaction.

A mixture of THF and MeOH (2:1, 30 ml) was added to the crude compound and 6 N sodium hydroxide (0.8 ml, 4.8 mmol) was added to the suspension. The reaction mixture was stirred at room temperature for 3 days. The mixture was poured into ice containing 0.8 ml of conc. HCl. The precipitate thus formed was separated, washed with plenty of water, then with a MeOH/Et₂O mixture and with hot acetonitrile, and finally dried under vacuum at 40° C. overnight to provide the title compound (0.095 g, 70%) as an off-white solid, mp>200° C.

¹H-NMR (300 MHz, DMSO-d₆) δ ppm: 13.71 (br.s, 1H), 12.23 (br.s, 1H), 8.51 (d, 1H), 8.11-8.29 (m, 1H), 7.99 (d, 1H), 7.85-8.05 (m, 1H), 7.49-7.73 (m, 2H), 7.15-7.40 (m, 5H), 7.12 (dd, 1H), 2.58-2.83 (m, 4H), 1.84-2.05 (m, 2H); MS: m/z=450 [MH]⁺

### Example 269

### 4-(4-Chlorophenylcarbamoyl)-3-(3,6-dichloro-benzo[b]thiophene-2-carboxamido)benzoic acid

### Method 32

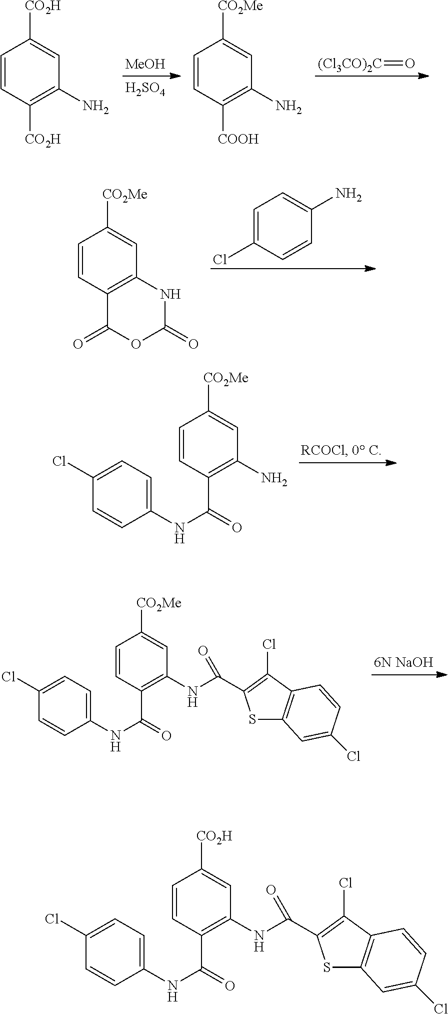

A mixture of 2-amino-terephthalic acid (10.0 g, 55.1 mmol), 96% sulfuric acid (10 ml) and methanol (100 ml) was stirred at 60° C. for 7 h, then concentrated to half its volume, poured into ice and cautiously basified to pH 8 with about 20 g of solid sodium carbonate. The solution was washed three times with DCM, which was discarded, then acidified to pH 2 with conc. HCl. The precipitate was collected by filtration, washed repeatedly with water and dried in a vacuum oven. 2-Amino-terephthalic acid 4-methyl ester (8.56 g, 80%) was obtained as a yellow solid.

To a solution of this compound (1.0 g, 5.1 mmol) in dry THF (15 ml) a solution of triphosgene (0.52 g, 1.7 mmol) in dry THF (10 mL) was added dropwise, over 10 min. The mixture was heated at 40° C. and stirred overnight, under nitrogen atmosphere. The reaction mixture was cooled at room temperature and the solvent was reduced under vacuum. Hexanes (10 ml) were added and the mixture of solvents was further concentrated under vacuum until a white precipitate formed. The precipitate was filtered and washed with plenty of hexanes, ethyl acetate and Et₂O, then dried under vacuum at 40° C. overnight to provide 4-carboxymethyl isatoic anhydride as a yellow solid (0.86 g, 76%).

A mixture this anhydride (0.5 mg, 2.26 mmol), 4-chloroaniline (0.29 mg, 2.26 mmol) and dioxane (20 ml) was refluxed overnight, and then stirred at room temperature for a further 8 days. Solvent was removed under vacuum and the resulting crude solid was washed with Et₂O, ethyl acetate and then dried under vacuum. 3-Amino-N-(4-chloro-phenyl)-terephthalamic acid methyl ester was obtained as a yellow solid (0.4 g, 58%).

A solution of this ester (0.25 g, 0.8 mmol) in dry THF (40 ml) was cooled at 0° C. 3,6-Dichlorobenzo[b]thiophene-2-carbonyl chloride (0.18 g, 0.68 mmol) was added portionwise. The mixture was warmed to room temperature and stirred at the same temperature for 3 h. The mixture was evaporated and the residue was triturated in a 1:1 MeOH:Et₂O mixture (10 mL). The solid was filtered, washed with plenty of Et₂O and then dried under vacuum at 40° C. overnight. N-(4-Chloro-phenyl)-3-(3,6-dichloro-benzo[b]-thiophene-2-carboxamido)-terephthalamic acid methyl ester was obtained as an off-white solid (0.26 g, 72%).

To a suspension of this compound (0.26 g, 0.48 mmol) in a 1:1 THF:MeOH mixture (100 ml), 6 N sodium hydroxide (1.2 ml, 7.3 mmol) was added and the reaction mixture was stirred at room temperature for 48 h. The mixture was poured into ice containing 1 ml of conc. HCl. The solution was concentrated under vacuum until a precipitate formed. The precipitate was then separated by filtration, washed with plenty of water, MeOH and Et₂O and then dried under vacuum at 40° C. overnight to afford the title compound (0.197 g, 79%) as an off-white solid, mp>200° C.

¹H-NMR (300 MHz, DMSO-d₆, 300° K) δ ppm: 13.35 (s, 1H), 11.35 (s, 1H), 10.82 (s, 1H), 8.90 (d, 1H), 8.36 (d, 1H), 7.98 (d, 1H), 7.96 (d, 1H), 7.88 (dd, 1H), 7.77 (m, 2H), 7.65 (dd, 1H), 7.44 (m, 2H); MS: m/z=519 [MH]⁺

### Example 270

### 5-(3-Chlorobenzo[b]thiophene-2-carboxamido)-2-(4-chlorophenyl-carbamoyl)benzoic acid

### Method 33

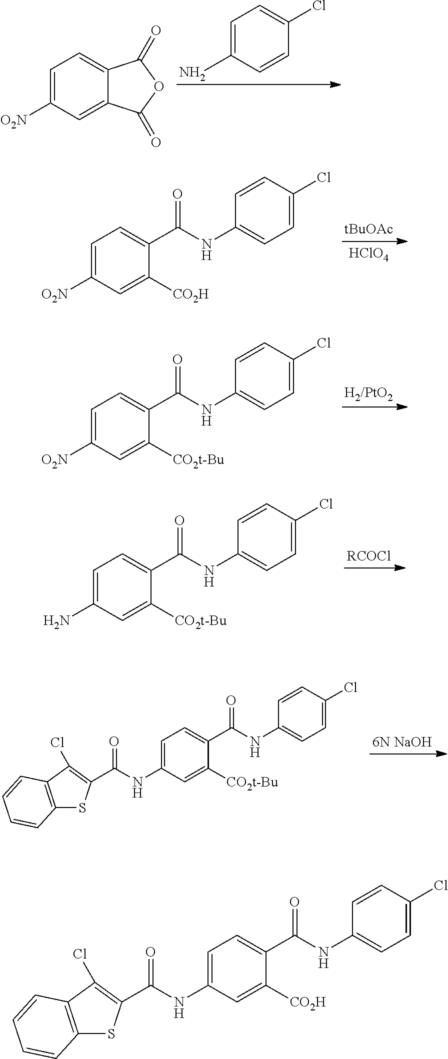

A solution of 4-nitrophthalic anhydride (4.0 g, 20.8 mmol) and 4-chloroaniline (2.64 g, 20.8 mmol) in dioxane (20 ml) was stirred at room temperature overnight. The precipitate that formed during the reaction was collected by filtration, rinsed with dioxane (about 15 ml) and dried under vacuum at 40° C. overnight. N-(4-Chlorophenyl)-5-nitro-phthalamic acid was obtained as a pure compound (2.85 g, 42.7%).

To a solution of this acid (2.85 g, 8.89 mmol) in tert-butyl-acetate (200 ml), perchloric acid (1.0 ml, 15.3 mmol) was added in one portion. The reaction mixture was stirred at room temperature for 1 h and 30 min. The mixture was washed with water (60 ml), then stirred at room temperature in the presence of aqueous sodium carbonate (50 ml) for 30 min. The organic phase was separated, washed with water (50 ml), brine (50 ml), then dried (anhydrous sodium sulfate), filtered and evaporated under vacuum. The crude compound thus obtained was purified by flash chromatography (SiO₂, hexanes:Et₂O 4:6). N-(4-Chlorophenyl)-5-nitro-phthalamic acid tert-butyl ester was obtained as a pure compound (1.14 g, 34%).

This ester (1.14 g, 3.03 mmol) was added in one portion to a suspension of platinum oxide x H₂O (138 mg) in an ethyl acetate/MeOH mixture (10:1, 55 ml). The mixture was hydrogenated at 25 psi for 5 h. The mixture was filtered through a Celite pad, and the solvent was evaporated under vacuum. The resulting crude solid was purified by flash chromatography (SiO₂, DCM/MeOH from 98:2 to 95:5). 5-Amino-N-(4-chlorophenyl)-phthalamic acid tert-butyl ester was obtained as a white solid (0.96 g, 91%).

A mixture of this ester (0.4 g, 1.15 mmol), 3-chlorobenzo[b]thiophene-2-carbonyl chloride (0.279 g, 1.21 mmol), pyridine (0.102 ml, 1.26 mmol) and dry THF (10 ml) was stirred at room temperature for 1.5 h, then evaporated under vacuum. The residue was dissolved in DCM (10 ml) and washed with a 1 M solution of potassium carbonate (5 ml) and brine (5 ml). The organic layer was separated, dried (anhydrous sodium sulfate), filtered and evaporated under vacuum. 5-(3-Chloro-benzo[b]thiophene-2-carboxamido)-N-(4-chloro-phenyl)-phthalamic acid tert-butyl ester was obtained as a white solid (0.607 g, 98%). To a suspension of this compound (0.25 g, 0.38 mmol) in THF (20 ml), 6 N sodium hydroxide (0.47 ml, 2.84 mmol) was added. After 20 min the mixture was poured into ice containing 0.25 ml of conc. HCl. The precipitate thus formed was separated, washed with plenty of water, MeOH and Et₂O and then dried under vacuum at 40° C. overnight to obtain the title compound (0.179 g, 79%) as a white solid mp>200° C.

¹H-NMR (300 MHz, DMSO-d₆) δ ppm: 13.13 (br.s, 1H), 10.84 (s, 1H), 10.48 (br.s, 1H), 8.25 (d, 1H), 8.14-8.22 (m, 1H), 7.92-8.03 (m, 2H), 7.72 (m, 2H), 7.55-7.68 (m, 3H), 7.39 (m, 2H); MS: m/z=485 [MH]⁺

Analogously the compound of the following example was prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 271 | 5-(3-Chlorobenzo[b]- | >200 | 13.12 (br.s, 1H), 10.84 (s, 1H), | 485.16 |
| | thiophene-2-carboxamido)-2- | | 10.47 (br.s, 1H), 8.26 (s, 1H), 8.11- | [MH]⁺ |
| | (4-chlorophenylcarbamoyl)- | | 8.23 (m, 1H), 7.98 (m, 2H), 7.50- | |
| | benzoic acid | | 7.80 (m, 5H), 7.39 (m, 2H) | |

### Example 272

### 2-(3-Chlorobenzo[b]thiophene-2-carboxamido)-4-(thiazole-2-carboxamido)-benzoic acid

### Method 34

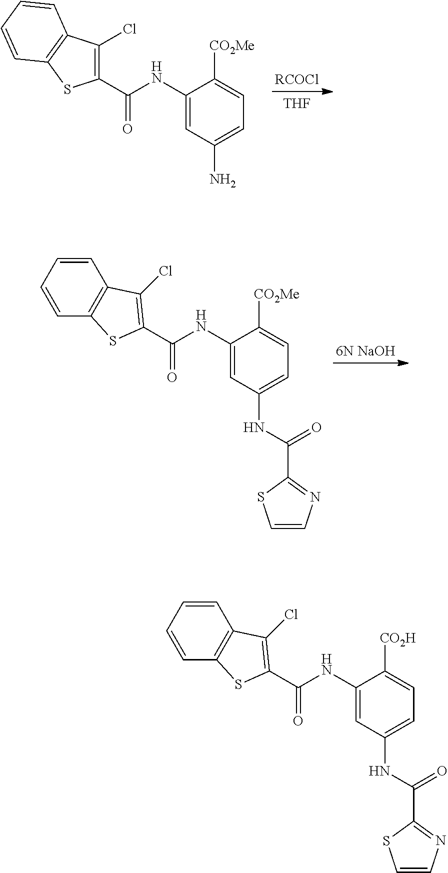

A mixture of 4-amino-2-(3-chloro-benzo[b]thiophene-2-carboxamido)-benzoic acid methyl ester (490 mg, 1.30 mmol) and 1,3-thiazole-2-carbonyl chloride (240 mg, 1.60 mmol) in THF (50 ml) was stirred at room temperature for 3 days. The precipitate was collected and washed with ethyl ether/methanol to obtain 2-(3-chloro-benzo[b]thiophene-2-carboxamido)-4-(thiazole-2-carboxamido)-benzoic acid methyl ester as a pale yellow solid (166 mg, 27%).

To a suspension of this compound (166 mg, 0.35 mmol) in THF/methanol (1:1, 30 ml), 6 N sodium hydroxide (0.88 ml, 5.2 mmol) was added and the mixture was stirred at room temperature for 24 h. The reaction mixture was poured into ice containing 0.5 ml of conc. HCl and the solvent was evaporated. The precipitate thus formed was washed repeatedly with water, then with methanol and ethyl ether, and finally dried under vacuum at 40° C. overnight to yield the title compound (69 mg, 43%) as an off-white solid, mp>200° C.

¹H NMR (300 MHz, DMSO-d₆) δ ppm: 13.67 (br.s, 1H), 12.45 (br.s, 1H), 11.13 (br.s, 1H), 9.27 (s, 1H), 8.16-8.21 (m, 2H), 8.12-8.15 (m, 1H), 8.05 (d, 1H), 7.94-8.02 (m, 1H), 7.71-7.78 (m, 1H), 7.60-7.70 (m, 2H). MS: m/z=458 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 273 | 2-(3-Chloro-benzo[b]- | >200 | 13.63 (s, 1H), 12.42 (br.s, 1H), | 485.14 |
| | thiophene-2-carboxamido)-4- | | 10.71 (s, 1H), 9.12 (d, 1H), 8.14- | [MH]⁺ |
| | (4-chloro-benzamido)- | | 8.26 (m, 1H), 7.93-8.12 (m, 4H), | |
| | benzoic acid | | 7.81 (dd, 1H), 7.52-7.73 (m, 4H) | |
| 274 | 2-(3-Chloro-benzo[b]- | >200 | 13.65 (br.s, 1H), 12.37 (br.s, 1H), | 490.94 |
| | thiophene-2-carboxamido)-4- | | 10.68 (s, 1H), 8.99-9.10 (m, 1H), | [MH]⁺ |
| | (5-chloro-thiophene-2- | | 8.12-8.28 (m, 1H), 7.91-8.12 (m, | |
| | carboxamido)-benzoic acid | | 3H), 7.77 (dd, 1H), 7.54-7.72 (m, | |
| | | | 2H), 7.30 (d, 1H) | |
| 275 | 4-(5-Chlorothiophene-2- | >200 | 13.60 (s, 1H), 12.37 (br.s, 1H), | 482.5 |
| | carboxamido)-2-(5- | | 10.66 (s, 1H) 8.98 (d, 1H), 7.94- | [MH]⁺ |
| | phenylthiophene-2- | | 8.16 (m, 2H), 7.62-7.87 (m, 5H), | |
| | carboxamido)-benzoic acid | | 7.35-7.56 (m, 3H), 7.29 (d, 1H) | |
| 276 | 2-(3-Chlorobenzo[b]- | >200 | 13.54 (br.s, 1H), 12.56 (br.s, 1H), | 499.85 |
| | thiophene-2-carboxamido)-4- | | 9.24-9.40 (m, 1H) 8.89 (s, 1H), 8.71- | [MH]⁺ |
| | (3-(4-chlorophenyl)ureido)- | | 8.79 (m, 1H), 8.10-8.24 (m, 1H), | |
| | benzoic acid | | 7.90-8.04 (m, 2H), 7.59-7.72 (m, | |
| | | | 2H), 7.44-7.56 (m, 3H), 7.34 (m, 2H) | |
| 277 | 2-(4-Chlorobenzamido)-4-(3- | >200 | 12.89 (s, 1H), 10.92 (br.s, 1H), 9.10 | 484.88 |
| | chlorobenzo[b]thiophene-2- | | (br.s, 1H), 8.12-8.23 (m, 1H), 7.88- | [MH]⁺ |
| | carboxamido)benzoic acid | | 8.12 (m, 4H), 7.49-7.80 (m, 5H) | |
| 278 | 2-(3-Chlorobenzo[b]- | >200 | 10.94 (br.s, 1H), 8.55 (d, 1H), 8.09- | 520.92 |
| | thiophene-2-carboxamido)-4- | | 8.26 (m, 1H), 7.82-8.03 (m, 4H), | [MH]⁺ |
| | (4-chlorophenylsulfon- | | 7.49-7.75 (m, 4H), 6.96 (dd, 1H) | |
| | amido)benzoic acid | | | |
| 279 | 2-(3-Chlorobenzo[b]- | >200 | [400 MHz] 13.50 (br.s, 1H), 12.40 | 534.2 |
| | thiophene-2-carboxamido)-4- | | (br.s, 1H), 9.08 (m, 1H), 8.80 (m, | [MH]⁺ |
| | (4-phenylpiperidine-1- | | 1H), 8.17 (m, 1H), 7.98-7.92 (m, | |
| | carboxamido)benzoic acid | | 2H), 7.67-7.61 (m, 2H), 7.32-7.26 | |
| | | | (m, 4H), 7.21 (m, 1H), 4.31 (m, 2H), | |
| | | | 8.91 (m, 2H), 2.72 (m, 2H), 1.83 (m, | |
| | | | 2H), 1.57 (m, 2H) | |

### Example 280

### 2-(3-Chlorobenzo[b]thiophene-2-carboxamido)-4-(4-chlorobenzylamino)-benzoic acid

### Method 35

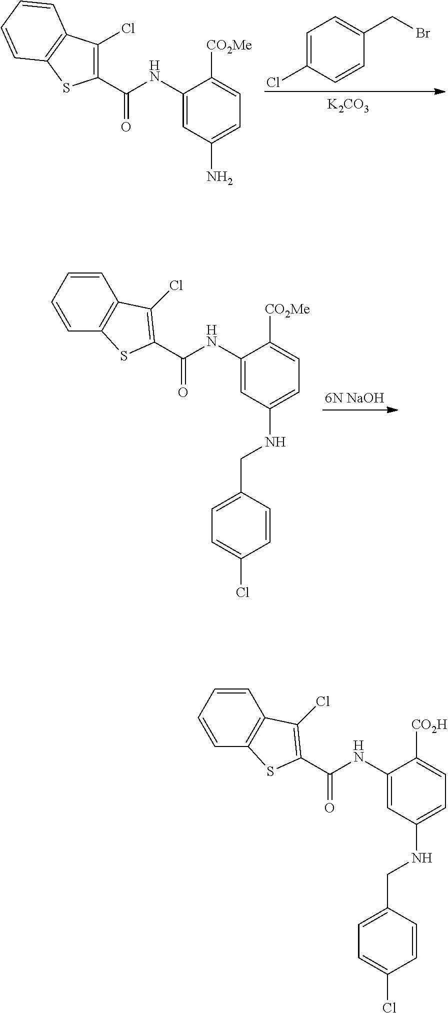

A mixture of 4-amino-2-(3-chloro-benzo[b]thiophene-2-carboxamido)-benzoic acid methyl ester (288 mg, 0.80 mmol), 4-chlorobenzylbromide (165 mg, 0.80 mmol) and potassium carbonate (132 mg, 0.96 mmol) in DMF (20 ml) was heated with microwave to 150° C. for 30 min in a sealed tube. The mixture was diluted with water and extracted twice with dichloromethane; the crude residue was purified twice by flash chromatography to obtain 2-(3-chloro-benzo[b]thiophene-2-carboxamido)-4-(4-chloro-benzylamino)-benzoic acid methyl ester as a yellow solid (85 mg, 27%).

To a solution of this compound (85 mg, 0.17 mmol) in THF/methanol (1:1, 20 ml), 6 N sodium hydroxide (0.44 ml, 2.6 mmol) was added and the mixture was stirred at 60° C. for 6 h. The reaction mixture was poured into ice containing 0.3 ml of conc. HCl and the organic solvents were removed under vacuum; the precipitate was collected and washed repeatedly with water, then with ethyl ether/methanol, and finally dried under vacuum at 40° C. overnight to afford the title compound (32 mg, 40%) as a white solid, mp>200° C.

¹H NMR (300 MHz, DMSO-d₆) δ ppm: 12.78 (br.s, 1H), 12.50 (br.s, 1H), 8.11-8.19 (m, 1H), 7.91-8.04 (m, 2H), 7.76 (d, 1H), 7.57-7.70 (m, 2H), 7.30-7.45 (m, 5H), 6.39 (dd, 1H), 4.37 (d, 2H); MS: m/z=471 [MH]⁺

### Example 281

### 2-(3-Chlorobenzo[b]thiophene-2-carboxamido)-4-(5-(4-chlorophenyl)-2-oxo-oxazolidin-3-yl)benzoic acid

### Method 36

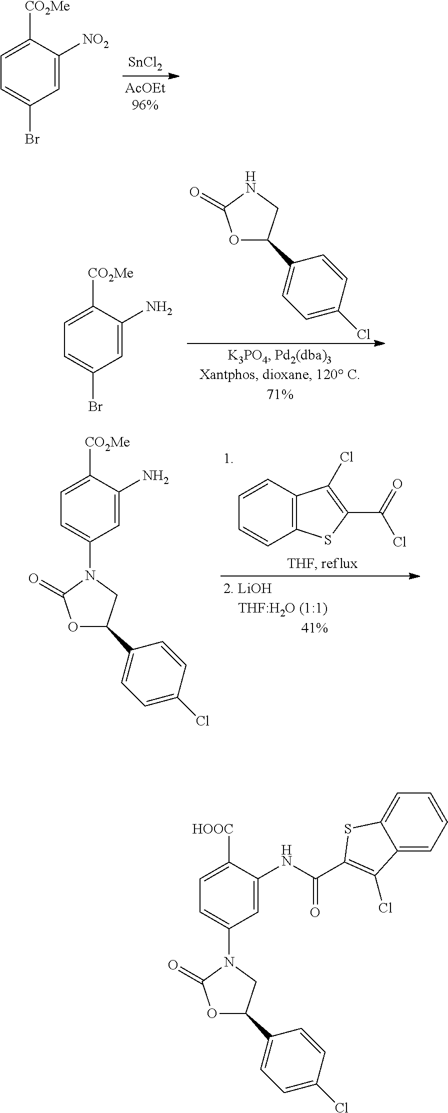

SnCl₂×2H₂O (5 g, 22 mmol) was added to a solution of methyl 4-bromo-2-nitrobenzoate (0.6 g, 2.3 mmol) in ethyl acetate (20 ml). The reaction mixture was submitted to ultrasonic irradiation for 2 h at 30° C. until the reaction was complete as indicated by TLC analysis. The solvent was removed under reduced pressure and the crude residue was washed with 2 M KOH. The aqueous layer was extracted with further portions of ethyl acetate, and the combined organic extracts were washed with brine and water, dried (MgSO₄) and concentrated under reduced pressure. The crude residue was chromatographed over silica gel (ethyl acetate/hexanes) yielding methyl 4-bromo-2-aminobenzoate (96%).

A solution of this compound (0.46 g, 2 mmol), 5-(4-chlorophenyl)oxazolidin-2-one (0.4 g, 2 mmol), Xantphos (128 mg, 0.22 mmol), Pd₂(dba)₃ (92 mg, 0.11 mmol) and K₃PO₄ (2.2 mmol) in dioxane was refluxed for 7 h. After cooling, the solvent was evaporated under vacuum and the residue partitioned between water and ethyl acetate. The aqueous layer was extracted with further portions of ethyl acetate and the combined organic extracts were washed with brine and water, dried (MgSO₄) and concentrated under reduced pressure. The crude residue was chromatographed over silica gel (ethyl acetate/hexanes) yielding methyl 2-amino-4-(5-(4-chlorophenyl)-2-oxo-oxazolidin-3-yl)benzoate (71%).

This compound (0.35 g, 1 mmol) was dissolved in THF, and 1.1 mmol of 3-chloro-benzo[b]thiophene carbonyl chloride was added. The reaction mixture was refluxed for 14 h and concentrated under vacuum. The crude reaction mass was washed with diethyl ether two times and dissolved in THF-water (3:1). LiOH (1.1 mmol) was added and the solution was stirred overnight at room temperature, concentrated under vacuum, and the residue was dissolved in the minimum volume of water. After adjusting the pH to 6-7, the aqueous solution was extracted with ethyl acetate. The organic layer was dried (MgSO₄) and concentrated under vacuum to afford the title compound (50%), mp>200° C.

¹H NMR (400 MHz, DMSO-d₆) δ ppm: 13.70 (br.s, 1H), 12.40 (br.s, 1H), 8.93 (s, 1H), 8.18 (m, 1H), 8.09 (d, 1H), 7.98 (m, 1H), 7.64 (m, 2H), 7.59 (m, 2H), 7.53 (m, 3H), 5.83 (m, 1H), 4.57 (m, 1H), 4.04 (m, 1H); MS: m/z=527 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 282 | 2-(3-Chlorobenzo[b]- | >200 | [400 MHz] 13.80 (br.s, 1H), 12.20 (s, | 523.2 |
| | thiophene-2-carboxamido)-4- | | 1H), 9.00 (s, 1H), 8.20 (m, 1H), 8.10 | [M − H]⁻ |
| | (4-(4-chlorophenyl)-2- | | (m, 1H) 8.00 (m, 1H), 7.60 (m, 3H), | |
| | oxopyrrolidin-1-yl)benzoic | | 7.40 (m, 4H), 4.30 (m, 1H), 3.90 (m, | |
| | acid | | 1H), 3.80 (m, 1H), 2.90 (m, 1H), | |
| | | | 2.70 (m, 1H) | |
| 283 | 4-(4-(4-Chlorophenyl)-2- | >200 | [400 MHz] 12.58 (br.s, 1H), 8.90 (m, | 551 |
| | oxopyrrolidin-1-yl)-2-(5-(4- | | 1H), 8.03 (d, 1H), 7.78 (m, 2H), 7.74 | [MH]⁺ |
| | chlorophenyl)thiophene-2- | | (d, 1H) 7.70 (d, 1H), 7.63 (m, 1H), | |
| | carboxamido)benzoic acid | | 7.53 (m, 2H), 7.47-7.42 (m, 4H), | |
| | | | 4.24 (m, 1H), 3.86 (m, 1H), 3.77 (m, | |
| | | | 1H), 2.92 (dd, 1H), 2.79 (dd, 1H). | |
| 284 | 4-(5-Benzyl-2-oxo-oxazolidin- | >200 | [400 MHz] 8.64 (s, 1H), 8.13 (m, 1H), | 507.1 |
| | 3-yl)-2-(3-chlorobenzo[b]- | | 7.95 (m, 2H), 7.61 (m, 2H), 7.20- | [MH]⁺ |
| | thiophene-2-carboxamido)- | | 7.38 (m, 6H), 4.97 (m, 1H), 4.15 (dd, | |
| | benzoic acid | | 1H), 3.81 (dd, 1H), 3.10 (d, 2H) | |
| 285 | 2-(3-Chlorobenzo[b]- | >200 | [400 MHz] 14.00 (br.s, 1H), 8.89 (s, | 525.2 |
| | thiophene-2-carboxamido)-4- | | 1H), 8.15 (m, 1H), 8.00 (d, 1H), 7.96 | [MH]⁺ |
| | (4-(4-chlorophenyl)-2- | | (m, 1H), 7.57-7.64 (m, 3H), 7.45 (m, | |
| | oxopyrrolidin-1-yl)benzoic | | 4H), 4.25 (m, 1H), 3.87 (m, 1H), 3.77 | |
| | acid | | (m, 1H), 2.93 (dd, 1H), 2.78 (dd, 1H) | |
| 286 | 2-(3-Chlorobenzo[b]- | >200 | 12.77 (s, 1H), 8.61 (d, 1H), 8.10- | 536.97 |
| | thiophene-2-carboxamido)-4- | | 8.28 (m, 1H), 8.05 (d, 1H), 7.92-8.02 | [MH]⁺ |
| | (5-(4-chlorophenyl)-2- | | (m, 1H), 7.57-7.75 (m, 2H), 7.35- | |
| | oxopiperidin-1-yl)benzoic | | 7.51 (m, 4H), 7.24 (dd, 1H), 3.89 (t, | |
| | acid | | 1H), 3.61-3.79 (m, 1H), 3.35-3.49 | |
| | | | (m, 1H), 2.55-2.70 (m, 2H), 2.13- | |
| | | | 2.25 (m, 1H), 1.95-2.13 (m, 1H) | |
| 287 | 2-(3-Chlorobenzo[b]- | >200 | 12.23 (br.s, 1H), 8.61 (d, 1H), 8.48 | 524.85 |
| | thiophene-2-carboxamido)-5- | | (d, 1H), 8.09-8.22 (m, 1H), 7.95-8.03 | [MH]⁺ |
| | (4-(4-chlorophenyl)-2- | | (m, 1H) 7.88 (dd, 1H), 7.59-7.70 (m, | |
| | oxopyrrolidin-1-yl)benzoic | | 2H), 7.35-7.53 (m, 4H), 4.26 (dd, | |
| | acid | | 1H), 3.90 (t, 1H), 3.77 (quin, 1H), | |
| | | | 2.93 (dd, 1H), 2.75 (dd, 1H) | |

### Example 288

### 2-(3-Chlorobenzo[b]thiophene-2-carboxamido)-4-(4-chlorophenyl-amino)benzoic acid

### Method 37

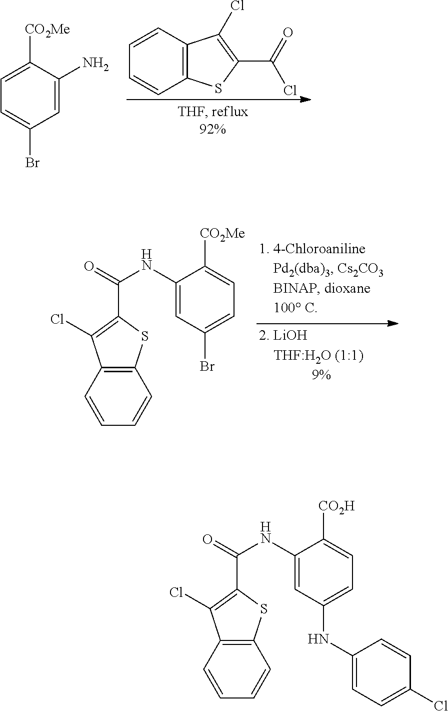

A solution of methyl 2-amino-4-bromobenzoate (231 mg, 1 mmol) and 3-chlorobenzo[b]thiophene-2-yl carbonyl chloride (230 mg, 1 mmol) in THF was refluxed for 4 h, cooled and evaporated under vacuum. The residue was triturated with diisopropyl ether, collected by suction filtration and dried.

A solution of this compound (350 mg, 0.82 mmol), 4-chloroaniline (105 mg, 0.82 mmol), Pd₂(dba)₃ (37 mg, 0.04 mmol), cesium carbonate (160 mg, 0.82 mmol) and BINAP (25 mg) in dioxane was refluxed for 7 h. After cooling, the solvent was evaporated under vacuum and the residue partitioned between water and ethyl acetate. The aqueous layer was extracted with further portions of ethyl acetate and the combined organic extracts were washed with brine and water, dried (MgSO₄) and concentrated under reduced pressure. The crude residue was chromatographed over silica gel (ethyl acetate/hexanes), the collected fractions were evaporated and the residue was dissolved in THF-water (3:1). LiOH (1.1 mmol) was added and the solution was stirred overnight at room temperature, concentrated under vacuum and the residue dissolved in the minimum volume of water. After adjusting the pH to 6-7, the aqueous solution was extracted with ethyl acetate. The organic layer was dried (MgSO₄) and concentrated under vacuum to afford the title compound (10%), mp>200° C.

¹H-NMR (400 MHz, 300° K, DMSO-d₆) δ ppm: 13.12 (br.s, 1H), 9.03 (br.s, 1H), 8.43 (s, 1H), 8.16 (m, 1H), 7.97 (m, 1H), 7.91 (m, 1H), 7.66-7.61 (m, 2H), 7.38 (dd, 2H), 7.23 (dd, 2H), 6.82 (m, 1H); MS: m/z=456.9 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 289 | 2-(3-Chlorobenzo[b]- | >200 | 8.06-8.19 (m, 1H), 7.87-7.97 (m, | 461 |
| | thiophene-2-carboxamido)-4- | | 2H), 7.76 (d, 1H), 7.51-7.67 (m, 2H), | [M − H]⁻ |
| | (indan-2-ylamino)benzoic | | 7.21-7.34 (m, 2H), 7.02-7.21 (m, | |
| | acid | | 2H), 6.30 (dd, 1H), 6.14 (d, 1H), | |
| | | | 4.08-4.37 (m, 1H), 3.33 (dd, 2H), | |
| | | | 2.86 (dd, 2H) | |
| 290 | 2-(3-Chlorobenzo[b]- | 193 | 13.02 (s, 1H), 8.05-8.20 (m, 1H), | 484.87 |
| | thiophene-2-carboxamido)-4- | | 7.86-8.03 (m, 2H), 7.70 (d, 1H), | [MH]⁺ |
| | (1-(4-chlorophenyl)ethyl- | | 7.57-7.67 (m, 2H), 7.31-7.46 (m, | |
| | amino)benzoic acid | | 4H), 7.16 (d, 1H), 6.27 (dd, 1H), | |
| | | | 4.61 (quin, 1H), 1.45 (d, 3H) | |
| 291 | 2-(3-Chlorobenzo[b]- | >200 | 12.73 (br.s, 1H), 12.55 (br.s, 1H), | 475.02 |
| | thiophene-2-carboxamido)-4- | | 8.11-8.23 (m, 1H), 8.07 (d, 1H), | [MH]⁺ |
| | (1,2,3,4-tetrahydro- | | 7.91-8.01 (m, 1H), 7.79 (d, 1H), | |
| | naphthalen-1-ylamino)- | | 7.55-7.71 (m, 2H), 7.23-7.30 (m, | |
| | benzoic acid | | 1H), 7.01-7.23 (m, 4H), 6.54 (dd, | |
| | | | 1H), 4.50-4.86 (m, 1H), 2.70-2.93 | |
| | | | (m, 2H), 1.58-2.09 (m, 4H) | |

### Example 292

### 2-(3-Chlorobenzo[b]thiophene-2-carboxamido)-5-phenoxybenzoic acid

### Method 38

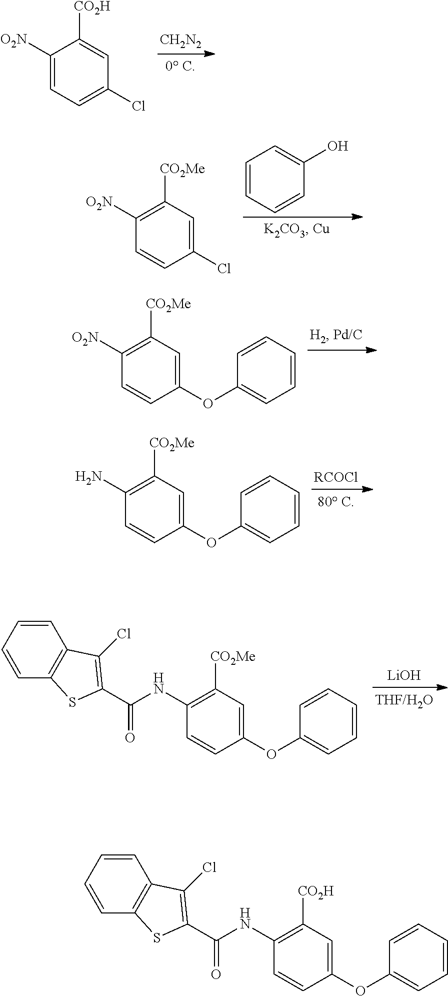

An excess of a solution of diazomethane in ether was added dropwise to a solution of 5-chloro-2-nitro benzoic acid (250 mg, 1.24 mmol) in ethyl acetate (7 ml) at 0° C. The reaction mixture was stirred at 0° C. for 1 h, and excess diazomethane was removed by bubbling nitrogen through the reaction mixture. The mixture was concentrated and the residue was purified by column chromatography over silica gel to yield methyl 5-chloro-2-nitrobenzoate quantitatively.

To a solution of this compound (450 mg, 2.09 mmol) in DMF (7.5 ml), phenol (217 mg, 2.3 mmol), K₂CO₃ (868 mg, 6.29 mmol) and copper powder (5 mg) was added. The reaction mixture was heated at 125° C. for 12 h. The mixture was diluted with water and filtered through a Celite pad. The filtrate was extracted with ethyl acetate. The organic layer was washed with water and brine, dried over Na₂SO₄, concentrated, and the residue was purified by column chromatography over silica gel to yield methyl 2-nitro-5-phenoxy-benzoate (232 mg, 70%).

To a solution of this compound (230 mg) in methanol (75 ml), 10% Pd/C (400 mg) was added. The reaction mixture was stirred under hydrogen atmosphere in a Parr shaker at 3 kg pressure for 2 h. The reaction mixture was filtered through a Celite pad, and the filtrate was concentrated to yield methyl 2-amino-5-phenoxybenzoate quantitatively. A mixture of this compound (200 mg, 0.82 mmol) and 3-chlorobenzo[b]thiophene-2-carbonyl chloride (209 mg, 0.9 mmol) in 1,2-dichloroethane (10 ml) was refluxed at 80° C. for 6 h. Reaction mixture was diluted with dichloromethane (20 ml), washed with water and brine, dried over Na₂SO₄ and concentrated. The crude product was purified by column chromatography over silica gel using 2 to 6% ethyl acetate in hexane as the eluent to yield 2-(3-chloro-benzo[b]thiophene-2-carboxamido)-5-phenoxy-benzoic acid methyl ester (200 mg, 70%) as a yellow solid.

To a solution of this compound (200 mg, 0.457 mmol) in THF (5 ml) lithium hydroxide hydrate (57 mg, 1.37 mmol) in water (2 ml) was added. The reaction mixture was stirred at 25° C. for 16 h and THF was removed under reduced pressure. The residue was acidified with 1.5 N HCl and extracted with dichloromethane. The combined organic layer was dried over anhydrous sodium sulphate, concentrated and crystallised from methanol to yield the title compound (130 mg, 80%) as a colorless powder, mp 228-229° C.

¹H-NMR (400 MHz, DMSO-d₆, 300° K), δ ppm: 7.07 (d, 2H, J=7.6 Hz), 7.18 (t, 1H, J=7.5 Hz), 7.35-7.5 (m, 3H), 7.57 (d, 1H, J=3 Hz), 7.6-7.7 (m, 2H), 7.97 (m, 1H), 8.16 (m, 1H), 8.60 (d, 1H, J=9.2 Hz), 12.1 (br, 1H). MS: m/z=421.8[M-H]⁻

Analogously the compound of the following example was prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 293 | 2-(5-(4-Chlorophenyl)- | >200 | 12.27 (br.s, 1H), 8.60 (d, 1H), 8.03 | 499.82 |
| | thiophene-2-carboxamido)-5- | | (d, 1H) 7.77-7.84 (m, 2H), 7.67-7.77 | [MH]⁺ |
| | (4-chlorophenylthio)benzoic | | (m, 3H), 7.48-7.61 (m, 2H), 7.38- | |
| | acid | | 7.49 (m, 2H), 7.23-7.36 (m, 2H) | |

### Example 294

### 3-(3-Chlorobenzo[b]thiophene-2-carboxamido)-5-(3-(methylamino)propyl-carbamoyl)benzoic acid

### Method 39

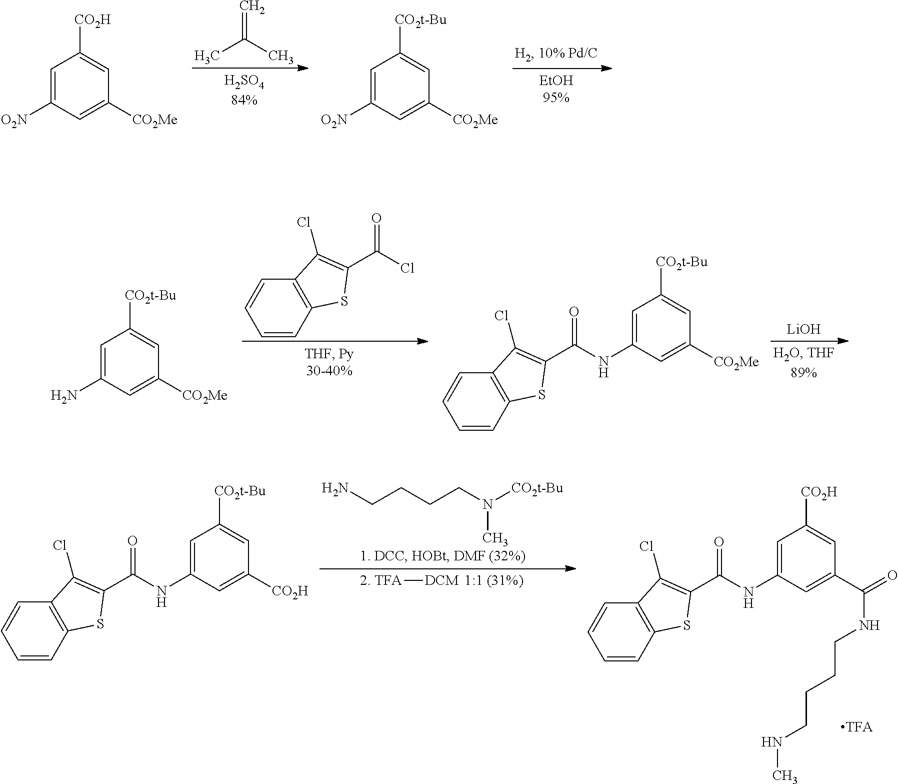

A catalytic amount of sulfuric acid and condensed 2-methyl propene was added to a solution of methyl 3-carboxy-5-nitrobenzoate in dioxane at −15° C. in a sealed tube. The reaction mixture was stirred at room temperature for 48 h, then poured into an ice cold 10% NaOH solution, extracted with ethyl acetate, and washed with water and brine. The organic layer was dried and evaporated under vacuum to afford the corresponding t-butyl ester (85% yield), which was dissolved in ethanol and hydrogenated for 18-20 h in presence of 10% palladium on carbon. After filtration of the catalyst through a Celite pad, the filtrate was concentrated under vacuum to afford the corresponding tert-butyl 3-methoxycarbonyl-5-amino-benzoate (95%).

This compound was dissolved in THF/pyridine 95:5, treated with 1.1 equivalents of 3-chloro-benzo[b]thiophene-2-carbonyl chloride, refluxed for 10 h, cooled, and concentrated under vacuum. The residue was triturated with diethyl ether and dissolved in THF-water (3:1). LiOH (1.1 eq.) was added and the solution was stirred overnight at room temperature. The solution was concentrated under vacuum and the residue dissolved in minimum volume of water, adjusting the pH to 6-7. The aqueous solution was extracted with ethyl acetate. The organic layer was dried and concentrated under vacuum to afford tert-butyl 3-carboxy-5-(3-chlorobenzo[b]thiophene-2-carboxamido)-benzoate (35% overall).

This product was dissolved in DMF and treated with 5-(N-methyl-t-butoxycarbonylamino)-pentylamine (1.2 eq.), DDC (1.5 eq.), HOBt (0.5 eq.) and DIEA (2.5 eq.) at 5-10° C., and stirring was continued at room temperature for 14-16 h. The precipitate was filtered over a Celite pad and the filtrate was concentrated under vacuum. The residue was taken up in ethyl acetate, filtered, washed with water and brine, dried, and evaporated under vacuum. The residue was triturated with diisopropyl ether, dissolved in dichloromethane and treated with TFA. Stirring was continued at room temperature for 1 h, then the solvent was removed under vacuum. The residue was crystallized from diethyl ether to afford 10% overall of the title compound, as the trifluoroacetic acid salt, mp>250° C.

¹H-NMR (400 MHz, DMSO-d₆, 300° K), δ ppm: 13.5 (br.s, 1H), 10.90 (s, 1H), 8.89-8.87 (m, 1H), 8.48-8.46 (m, 2H), 8.34-8.20 (m, 2H), 8.16-8.19 (m, 2H), 7.96-7.94 (m, 1H), 7.66-7.62 (m, 2H), 3.4-3.33 (m, 2H), 2.97-2.91 (m, 2H), 2.59 (s, 3H), 1.88-1.86 (m, 2H);

MS: m/z=446.3[MH]⁺.

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 295 | 3-(3-Amino-3-iminopropyl- | >250 | 10.68 (br.s, 2H), 9.39 (br.s, 1H), | 445.2 |
| | carbamoyl)-5-(3-chloro- | | 9.18 (br.s, 1H), 8.54-8.51 (m, 3H), | [MH]⁺ |
| | benzo[b]thiophene-2- | | 8.25 (m, 1H), 8.16 (m, 1H), 7.95 (m, | |
| | carboxamido)benzoic acid | | 2H), 3.65-3.60 (m, 2H), 2.69 (m, 2H) | |
| 296 | 3-(5-Amino-5-iminopentyl- | >250 | 1.5 (br.s, 1H), 10.87 (s, 1H), 8.75- | 471.8 |
| | carbamoyl)-5-(3-chloro- | | 8.74 (m, 1H), | [M − H]⁻ |
| | benzo[b]thiophene-2- | | | |
| | carboxamido)benzoic acid | | | |

### Example 297

### 2-(3-Chloro-4-((4-methylpiperazin-1-yl)methyl)thiophene-2-carboxamido)-4-(4-chlorophenylcarbamoyl)benzoic acid

### Method 40

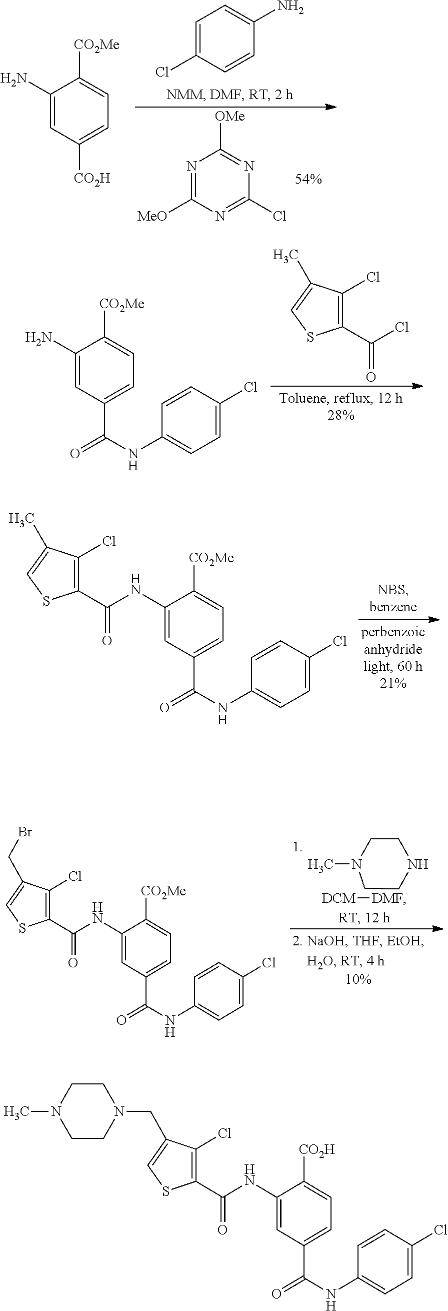

Chlorodimethoxytriazine (1.12 g, 6.36 mmol) was added to a solution of methyl 2-amino-4-carboxybenzoate (9.36 mmol) and N-methylmorpholine (0.63 g, 6.3 mmol) in DMF (20 ml) at room temperature. The mixture was stirred at room temperature for 2 h. The solvent was evaporated under vacuum and the residue was chromatographed over silica gel, eluting with ethyl acetate/hexanes. The collected fractions were evaporated under vacuum and the residue (1 g, 3.29 mmol) was mixed with 3-chloro-4-methylthiophene-2-carbonyl chloride (3.3 mmol) in toluene. The mixture was refluxed for 12 h, then cooled to room temperature and concentrated under vacuum. The crude residue was triturated with diethyl ether, dissolved in benzene, treated with perbenzoic anhydride (0.2 mmol) and N-bromosuccinimide (3.3 mmol) and stirred at room temperature for 60 h under solar lamp illumination. The insoluble matter was filtered off, the filtrate was washed with an aqueous solution of sodium bisulfite, water and brine. The organic layer was dried (Na₂SO₄) and evaporated under vacuum. The residue was chromatographed over silica gel (ethyl acetate/hexanes) and the collected fractions were evaporated under vacuum to afford methyl 2-(3-chloro-4-bromomethylthiophene-2-carboxamido)-4-(4-chlorophenyl-carbamoyl)benzoate (107 mg). This compound was dissolved in a 1:1 mixture of CH₂Cl₂ and DMF, 1-methylpiperazine (40 mg, 0.4 mmol) was added and the mixture was stirred at room temperature for 12 h. The solvent was evaporated under vacuum and the residue was partitioned between water and ethyl acetate. The organic layer was washed again with water, dried and evaporated to dryness. The residue was dissolved in a mixture of THF and ethanol (5 ml), treated with 1 N NaOH (0.5 ml) and stirred at room temperature for 4 h. After adjusting to pH=7 with diluted HCl, the solvent was evaporated under vacuum and the residue was chromatographed over silica gel to afford 15 mg (13.7%) of the title compound, mp>200° C.

¹H-NMR (500 MHz, DMSO-d₆, 300° K), δ ppm: 10.45 (s, 1H), 9.00 (s, 1H), 8.12 (d, 1H), 8.35 (m, 2H), 7.80 (m, 3H), 7.60 (d, 1H), 7.42 (d, 2H), 3.57-3.37 (m, 10H), 2.71 (s, 3H);

MS: m/z=549 (MH⁺)

Analogously the compound of the following example was prepared:

| Ex. | | mp | ¹H-NMR (500 MHz, DMSO-d₆, | MS, |
|---|---|---|---|---|
| No. | Chemical name | ° C. | 300° K), δ ppm | m/z |
| 298 | 3-(3-Chloro-4-((4-methyl- | >200 | 8.98-8.91 (m, 3H), 8.66 (m, 2H), | 547.1 |
| | piperazin-1-yl)methyl)- | | 8.10-8.67 (m, 3H), 7.89 (d, 1H), 7.73 | [MH]⁺ |
| | thiophene-2-carboxamido)-4- | | (d, 1H), 7.55 (m, 2H), 3.65, t, 2H), | |
| | (4-chlorophenylcarbamoyl)- | | 2.67 (t, 2H) | |
| | benzoic acid | | | |

### Example 299

### 4-(Benzamidomethyl)-2-(3-chlorobenzo[b]thiophene-2-carboxamido)-benzoic acid

### Method 41

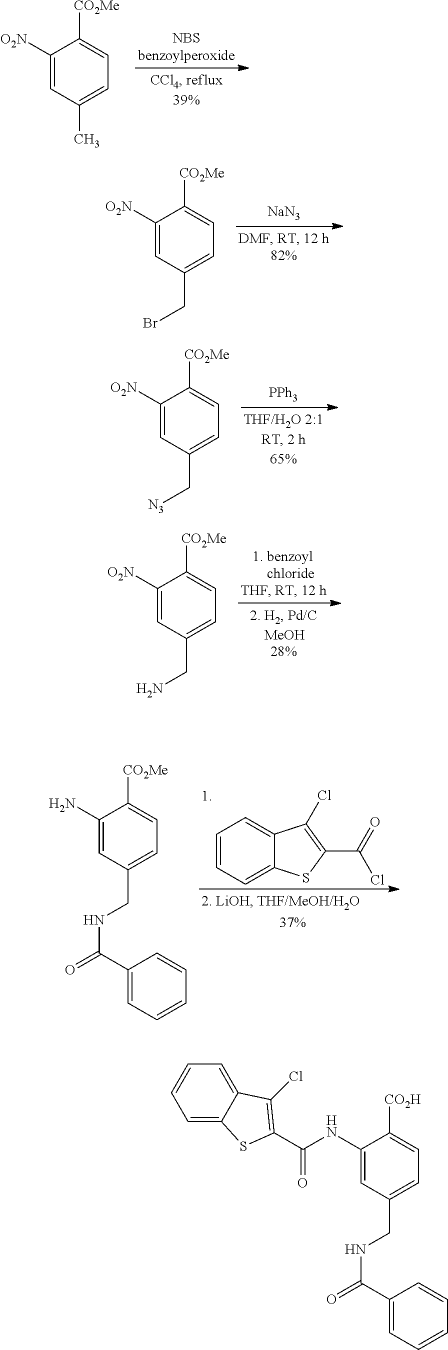

A solution of methyl 4-methyl-2-nitrobenzoate (0.97 g, 5 mmol), N-bromosuccinimide (0.89 g, 5 mmol) and a catalytic amount of benzoylperoxide in CCl₄ (25 ml) was refluxed for 8 h, then cooled to room temperature. Succinimide was filtered and washed with CCl₄, and the solvent was evaporated under reduced pressure to yield the crude benzyl bromide, which was recrystallized from a mixture of hexane-diethyl ether to afforded 0.54 g (39%) of pure methyl 4-bromomethyl-2-nitrobenzoate. This compound was dissolved in DMF at room temperature and treated with sodium azide (230 mg), and stirring was continued for 12 h. The reaction mixture was poured into water and extracted with ethyl acetate. The extracts were combined, dried (MgSO₄), and evaporated under vacuum. The residue was dissolved in a 2:1 mixture of THF and water, and triphenyl-phosphine (0.5 g, 0.2 mmol) was added portionwise. Stirring was continued for 2 h at room temperature, then the precipitate was filtered off and the organic solvent was evaporated under vacuum. The residue (0.29 g) was dissolved in THF (5 ml), TEA (0.2 mmol) and benzoyl chloride (0.2 mmol) were added, and the reaction mixture was stirred at room temperature for 12 h. Water (2 ml) was added and stirring was continued for 1 h, then the organic solvent was evaporated and the aqueous residue was extracted with ethyl acetate. The organic layer was washed with aqueous sodium bicarbonate, water and brine, dried (MgSO₄), and evaporated under vacuum. The residue was dissolved in methanol (10 ml) and hydrogenated in the presence of Pd/C catalyst. When reduction of the nitro group was complete (TLC) the catalyst was filtered off, the solvent was evaporated and the resulting methyl 2-amino-4-(benzamidomethyl)benzoate was acylated with 3-chloro-benzo[b]thiophene-2-carbonyl chloride (0.6 mmol) by refluxing in toluene for 12 h. After cooling to room temperature, the solvent was evaporated under vacuum and the residue was dissolved in THF/methanol 1:1 and treated with aqueous LiOH (0.6 mmol). The reaction mixture was stirred overnight at room temperature, concentrated and the residue was dissolved in the minimum volume of water. The pH was adjusted to 6-7 and the aqueous solution was extracted with ethyl acetate. The organic layer was dried (Na₂SO₄), concentrated under vacuum and the residue was triturated with diisopropyl ether to afford the title compound (74 mg), mp>200° C.

¹H-NMR (400 MHz, DMSO-d₆, 300° K), δ ppm: 12.31 (s, 1H), 9.20 (s, 1H), 8.65 (d, 1H), 7.80-8.30 (m, 5H), 7.40-7.70 (m, 4H), 7.15 (m, 1H), 4.55 (d, 2H); MS, m/z: 464.8 [MH]⁺

### Example 300

### 2-(3-Chlorobenzo[b]thiophene-2-carboxamido)-4-((2-oxopyrrolidin-1-yl)-methyl)benzoic acid

### Method 42

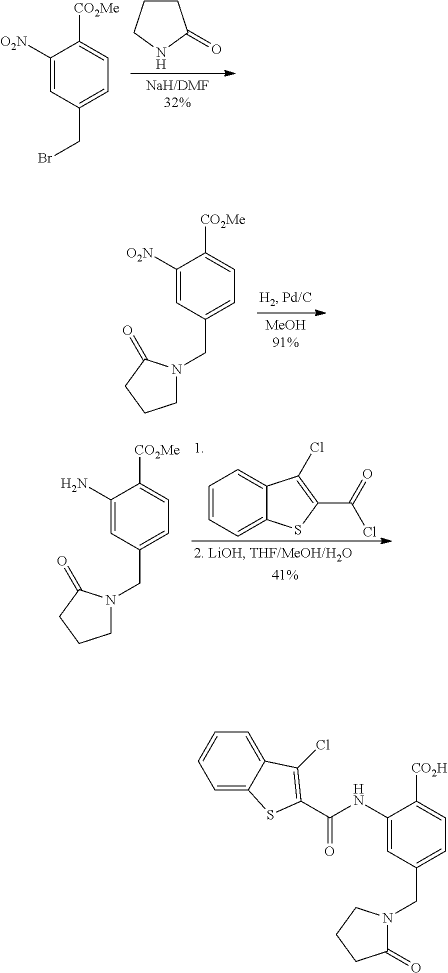

A solution of methyl 4-bromomethyl-2-nitrobenzoate (0.82 g, 3 mmol), prepared as described in Example 305, and pyrrolidinone (10 mmol) in DMF (7 ml) was treated portionwise with NaH (60% in mineral oil, 190 mg) and stirred at room temperature overnight. The reaction mixture was diluted with water (15 ml) and the pH was adjusted to 6 with diluted HCl. The mixture was extracted with ethyl acetate, the organic layer was washed again with water and brine, dried (MgSO₄) and evaporated to dryness under vacuum. The residue (0.26 g, 0.96 mmol) was dissolved in methanol (10 ml) and hydrogenated in the presence of Pd/C catalyst. When reduction of the nitro group was complete (TLC) the catalyst was filtered off, the solvent was evaporated and the resulting methyl 2-amino-4-((2-oxopyrrolidin-1-yl)methyl)benzoate (0.22 g, 0.87 mmol) was acylated with 3-chloro-benzo[b]thiophene-2-carbonyl chloride (0.23 g, 1 mmol) by refluxing in toluene for 12 h. After cooling to room temperature, the solvent was evaporated under vacuum and the residue was dissolved in THF/methanol 1:1 and treated with aqueous LiOH (1 mmol). The reaction mixture was stirred overnight at room temperature, concentrated and the residue was dissolved in the minimum volume of water. The pH was adjusted to 6-7 and the aqueous solution was extracted with ethyl acetate. The organic layer was dried (Na₂SO₄), concentrated under vacuum and the residue was triturated with diisopropyl ether to afford the title compound (153 mg), mp>200° C.

¹H-NMR (400 MHz, DMSO-d₆, 300° K), δ ppm: 14.10 (br.s, 1H), 12.34 (s, 1H), 9.50 (s, 1H), 8.20 (d, 1H), 7.90-8.10 (m, 2H), 7.60 (m, 2H), 7.10 (m, 1H), 4.46 (d, 2H), 3.20 (m, 2H), 2.32 (m, 2H), 1.98 (m, 2H); MS: m/z=429.1 [MH]⁺

Analogously the compound of the following example was prepared:

| Ex. | | mp | ¹H-NMR (400 MHz, DMSO-d₆, | MS, |
|---|---|---|---|---|
| No. | Chemical name | ° C. | 300° K), δ ppm | m/z |
| 301 | 2-(3-Chlorobenzo[b]- | >200 | 12.27 (s, 1H), 8.58 (m, 1H), 8.18 (m, | 539.2 |
| | thiophene-2-carboxamido)-4- | | 1H), 8.07 (m, 1H), 8.00 (m, 1H), | [MH]⁺ |
| | ((5-(4-chlorophenyl)-2-oxo- | | 7.68-7.62 (m, 2H), 7.48 (m, 4H), | |
| | oxazolidin-3-yl)methyl)- | | 7.18 (m, 1H), 5.67 (m, 1H), 4.51 (s, | |
| | benzoic acid | | 2H), 3.98 (m, 1H), 3.34 (m, 1H). | |

### Example 302

### 5-(Carboxymethoxy)-2-(4-(1-(carboxymethyl)-5,6-dichloro-1H-benzo[d]imidazol-2-yl)benzamido)-4-(4-chlorophenylcarbamoyl)benzoic acid

### Method 43

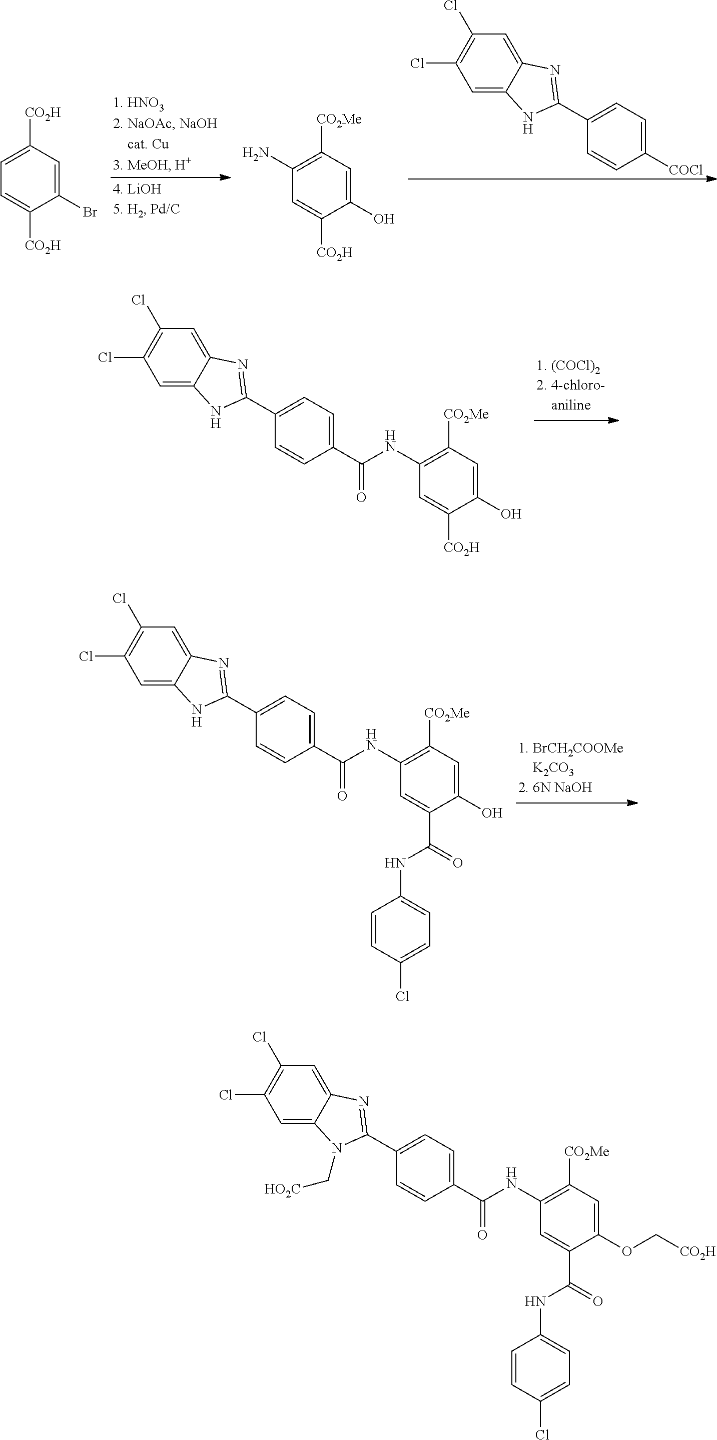 [SMILES: O=C(O)c1ccc(C(=O)O)c(Br)c1 ; COC(=O)c1cc(O)c(C(=O)O)cc1N ; O=C(Cl)c1ccc(-c2nc3cc(Cl)c(Cl)cc3[nH]2)cc1 ; COC(=O)c1cc(O)c(C(=O)O)cc1NC(=O)c1ccc(-c2nc3cc(Cl)c(Cl)cc3[nH]2)cc1 ; COC(=O)c1cc(O)c(C(=O)Nc2ccc(Cl)cc2)cc1NC(=O)c1ccc(-c2nc3cc(Cl)c(Cl)cc3[nH]2)cc1 ; COC(=O)c1cc(OCC(=O)O)c(C(=O)Nc2ccc(Cl)cc2)cc1NC(=O)c1ccc(-c2nc3cc(Cl)c(Cl)cc3n2CC(=O)O)cc1]

A suspension of 2-bromoterephthalic acid (11.5 g, 46.9 mmol) in conc. sulfuric acid (75 ml, 46.9 mmol), was cooled at 0° C. Conc. nitric acid (15 ml, 46.9 mmol) was added dropwise, the mixture was warmed to room temperature and stirred overnight. The mixture was poured into ice water and extracted with EtOAc. The organic phase was separated, dried on sodium sulfate, filtered and evaporated under vacuum to afford 2-bromo-5-nitroterephthalic acid (12.5 g, 43.1 mmol, 92% yield) as a light brown solid.

In a microwave sealed tube 2-bromo-5-nitroterephthalic acid (5.0 g, 17.24 mmol), sodium acetate (3.11 g, 37.9 mmol), sodium hydroxide (2.069 g, 51.7 mmol) and a catalytic amount of copper (0.022 g, 0.345 mmol) were suspended in 60 ml of water. The reaction was heated for 2 hr at 120° C. under microwave irradiation. The catalyst was filtered off and the basic solution was acidified with a 2 M aqueous solution of HCl. The precipitate thus formed was filtered and washed with plenty of water to afford 2-hydroxy-5-nitroterephthalic acid (3.2 g, 14.09 mmol, 82% yield) as a brown solid.

To a solution of this acid (3.2 g, 14.09 mmol) in MeOH (150 ml), cooled in an ice water bath, sulfuric acid (16 ml, 14.09 mmol) was added dropwise. The reaction was stirred at 70° C. for 90 h. Volatile fractions were removed under vacuum and the crude product portioned between water and EtOAc. The organic phase was separated, washed with a 5% aqueous solution of NaHCO₃, dried on sodium sulfate, filtered and evaporated under vacuum to afford dimethyl 2-hydroxy-5-nitroterephthalate (2.6 g, 10.19 mmol, 72.3% yield) as a light brown solid.

To a solution of this compound (2.6 g, 10.19 mmol) in a THF:MeOH:H₂O mixture (200 ml, 6:3:1 ratio), lithium hydroxide 1 M aq. solution (30.6 ml, 30.6 mmol) was added in one portion at room temperature. The reaction was refluxed overnight. Volatile solvents were removed under vacuum and the remaining aqueous solution was acidified with 2 M aqueous solution of HCl. The aqueous layer was then extracted with EtOAc. The organic layer was separated, dried over sodium sulfate, filtered and evaporated under vacuum to afford 2-hydroxy-4-(methoxycarbonyl)-5-nitrobenzoic acid (2.2 g, 9.12 mmol, 90% yield) as a brown solid.

This acid was added to a suspension of Pd/C (0.014 g, 0.012 mmol) in MeOH in one portion. The reaction was hydrogenated in a Parr apparatus at 20 psi for 4 h. The mixture was filtered through a Celite pad and the clarified solution was evaporated under vacuum to afford 5-amino-2-hydroxy-4-(methoxycarbonyl)benzoic acid (0.123 g, 100%) as a yellow oil.

A mixture of 4-(5,6-dichloro-1H-benzo[d]imidazol-2-yl)benzoic acid (0.43 g, 1.40 mmol), SOCl₂ (15 ml) and 1 drop of DMF was stirred overnight at 80° C. The mixture was evaporated and the residue was re-suspended in about 30 ml of dry THF; and 5-amino-2-hydroxy-4-(methoxycarbonyl)benzoic acid (0.296 g, 1.400 mmol) was added and the mixture was stirred for 3 days at room temperature. The precipitate was recovered by filtration and washed twice with methanol, then digested in hot n-BuOH to obtain 276 mg of 5-(4-(5,6-dichloro-1H-benzo[d]imidazol-2-yl)benzamido)-2-hydroxy-4-(methoxycarbonyl)benzoic acid (0.552 mmol, 39% yield) as a yellow solid (85% purity).

A mixture of this compound (85% pure, 276 mg, 0.552 mmol), (COCl)₂ (194 μl, 2.20 mmol) and traces of DMF in 10 ml of THF were stirred at 60° C. for 3 h. The mixture was evaporated and the residue was resuspended in 15 ml of dry THF; 4-chloroaniline (155 mg, 1.214 mmol) was added and the mixture was refluxed overnight. The precipitate was recovered by filtration, dissolved in DMF and purified by chromatography on a PE-AX cartridge, eluting with MeOH, MeOH/DMF 9:1, MeOH/DMF 2:1 and finally MeOH/AcOH 20:1. The fractions containing the pure product were combined and evaporated. The residue was diluted with a small amount of methanol and the resulting yellow precipitate was collected to obtain methyl 4-(4-chlorophenylcarbamoyl)-2-(4-(5,6-dichloro-1H-benzo[d]imidazol-2-yl)benzamido)-5-hydroxybenzoate (46 mg, 0.075 mmol, 14% yield). To a suspension of this compound (48 mg, 0.079 mmol) in dry DMF (3 ml), K₂CO₃ (21.76 mg, 0.157 mmol) and methyl bromoacetate (14.86 μl, 0.157 mmol) were added and the mixture was stirred overnight at room temperature. The mixture was diluted with water and the resulting precipitate was collected. 41.6 mg of methyl 4-(4-chlorophenyl-carbamoyl)-2-(4-(5,6-dichloro-1-(methoxycarbonylmethyl)-1H-benzo[d]imidazol-2-yl)-benzamido)-5-(methoxycarbonylmethoxy)benzoate (41.6 mg, 0.055 mmol, 70% yield) were obtained.

A mixture of this compound (41.6 mg, 0.055 mmol) and 6 N NaOH (0.046 ml, 0.276 mmol) in 10 ml of THF was stirred at 60° C. for 3.5 h. The mixture was then diluted with water, acidified and evaporated. The residue was stirred in 5 ml of water and the resulting precipitate was collected and washed with water and a small amount of methanol. The title compound (24 mg, 61%) was obtained as an off-white solid, mp>200° C.

¹H-NMR (300 MHz, DMSO-d₆, 300° K), δ ppm: 13.46 (br.s, 1H), 11.93 (br.s, 1H), 10.76 (s, 1H), 9.02 (s, 1H), 8.09-8.20 (m, 3H), 8.04 (s, 1H), 7.95 (m, 2H), 7.89 (m, 2H), 7.70 (s, 1H), 7.45 (m, 2H), 5.25 (s, 2H), 5.00 (s, 2H); MS: m/z=710.86 [MH]⁺

Analogously the compounds of the following examples were prepared:

| Ex. | | mp | ¹H-NMR (300 MHz, DMSO-d₆, | MS, |
|---|---|---|---|---|
| No. | Chemical name | ° C. | 300° K), δ ppm | m/z |
| 303 | 2-(3-Chlorobenzo[b]- | >200 | [400 MHz] 12.12 (br.s, 1H), 11.29 | 498.9 |
| | thiophene-2-carboxamido)-4- | | (br.s, 1H), 10.56 (s, 1H), 8.90 (s, | [M − H]⁻ |
| | (4-chlorophenylcarbamoyl)-5- | | 1H), 8.17-8.14 (m, 1H), 7.78 (m, | |
| | hydroxybenzoic acid | | 2H), 7.67 (m, 1H), 7.66-7.60 (m, | |
| | | | 2H), 7.43 (m, 2H). | |
| 304 | 5-(Carboxymethoxy)-2-(3- | >200 | 13.59 (br.s, 2H), 11.95 (br.s, 1H), | 558.77 |
| | chlorobenzo[b]thiophene-2- | | 10.74 (s, 1H), 9.04 (s, 1H), 8.12- | [MH]⁺ |
| | carboxamido)-4-(4-chloro- | | 8.21 (m, 1H), 7.93-8.02 (m, 1H), | |
| | phenylcarbamoyl)benzoic | | 7.88 (m, 2H), 7.71 (s, 1H), 7.58-7.70 | |
| | acid | | (m, 2H), 7.45 (m, 2H), 5.00 (s, 2H) | |
| 305 | 5-(Carboxymethoxy)-4-(4- | >200 | 11.20 (s, 1H), 10.76 (s, 1H), 8.74 | 680.92 |
| | chlorophenylcarbamoyl)-2-(4- | | (s, 1H), 8.06-8.22 (m, 5H), 8.02 | [MH]⁺ |
| | (5,6-dichloro-1-methyl-1H- | | (s, 1H), 7.89 (m, 2H), 7.65 (s, 1H), | |
| | benzo[d]imidazol-2-yl)- | | 7.45 (m, 2H), 5.02 (s, 2H), 3.96 (s, | |
| | benzamido)benzoic acid | | 3 H), 3.91 (s, 3H) | |
| | methyl ester | | | |
| 306 | 5-(Carboxymethoxy)-4-(4- | >200 | 11.91 (br. s., 1H), 10.76 (s, 1H), | 666.9 |
| | chlorophenylcarbamoyl)-2-(4- | | 9.05 (s, 1H), 8.06-8.22 (m, 6H), | [MH]⁺ |
| | (5,6-dichloro-1-methyl-1H- | | 8.01 (s, 1H), 7.84-7.94 (m, 2H), | |
| | benzo[d]imidazol-2-yl)- | | 7.71 (s, 1H), 7.36-7.53 (m, 2H), | |
| | benzamido)benzoic acid | | 5.00 (s, 1H), 3.95 (s, 3H) | |

### Example 307

### 3,4-Di(3-chlorothiophene-2-carboxamido)benzoic acid

### Method 44

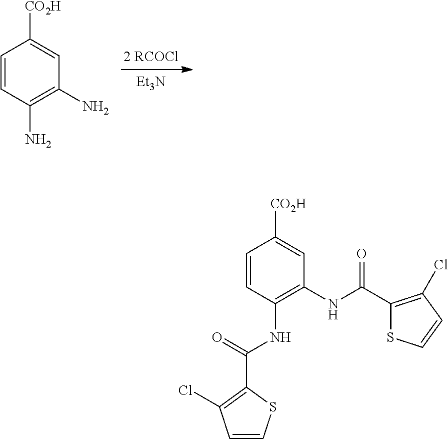 [SMILES: "Et3N" ; Nc1ccc(C(=O)O)cc1N ; O=C(O)c1ccc(NC(=O)c2sccc2Cl)c(NC(=O)c2sccc2Cl)c1]

A mixture of 3,4-diamino-benzoic acid (500 mg, 3.28 mmol), 3-chlorothiophene-2-carbonyl chloride (1.49 g, 8.21 mmol) and triethylamine (0.91 ml, 6.56 mmol) in THF (40 ml) was stirred at room temperature for 24 h. The precipitate was collected and washed repeatedly with methanol and ethyl ether to obtain the title compound (300 mg, 21%).

¹H-NMR (300 MHz, DMSO-d₆, 300° K), δ ppm: 13.04 (br.s, 1H), 9.77 (br.s, 2H), 8.22 (d, 1H), 7.93-8.06 (m, 3H), 7.89 (dd, 1H), 7.23 (d, 2H); MS: m/z=441 [MH]⁺

Analogously the compounds of the following examples were prepared:

| | | mp | ¹H-NMR (300 MHz, DMSO-d₆, | MS, |
|---|---|---|---|---|
| Ex. No. | Chemical name | ° C. | 300° K), δ ppm | m/z |
| 308 | 2,4-Di(3-chlorothiophene-2- | >200 | 13.61 (br.s, 1H), 12.11 (br.s, 1H), | 440.84 |
| | carboxamido)benzoic acid | | 10.62 (s, 1H), 8.97 (d, 1H), 8.02 (d, | [MH]⁺ |
| | | | 1H), 7.99 (d, 1H), 7.93 (d, 1H), 7.62 | |
| | | | (dd, 1H), 7.25 (d, 1H), 7.22 (d, 1H) | |
| 309 | 2,3-Di(5-chlorothiophene-2- | >200 | [400 MHz] 12.94 (br.s, 1H), 10.08 | 441 |
| | carboxamido)benzoic acid | | (br.s, 1H), 7.81-7.76 (m, 3H), 7.69 | [MH]⁺ |
| | | | (m, 1H), 7.39 (m, 1H), 7.26 (m, 2H) | |

### Example 310

### 4-(3-Chlorobenzo[b]thiophene-2-carboxamido)-3-(4-chlorobenzyloxy)-benzoic acid

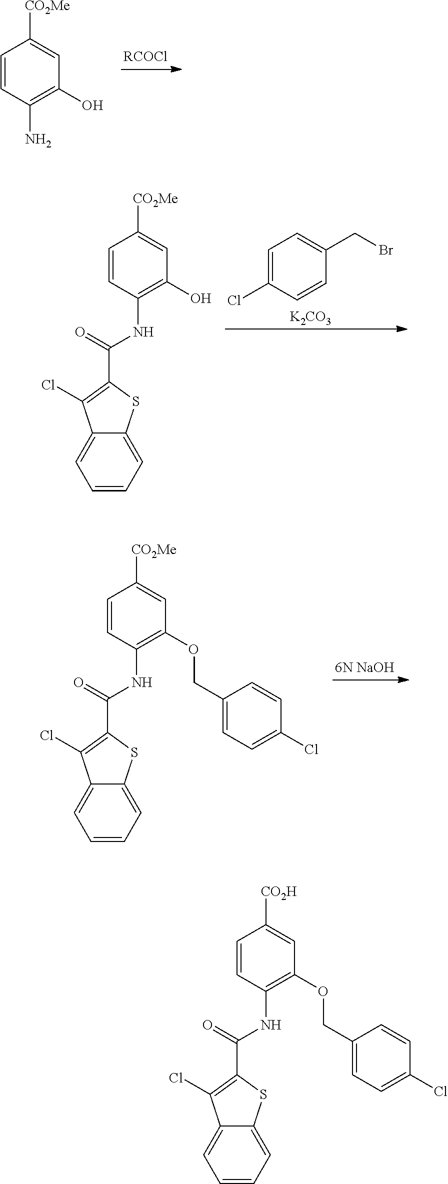

A mixture of methyl 4-amino-3-hydroxy-benzoate (400 mg, 2.40 mmol) and 3-chloro-benzo[b]thiophene-2-carbonyl chloride (553 mg, 2.40 mmol) in THF (10 ml) was stirred overnight at room temperature. The precipitate was collected and washed with ethyl ether to obtain 4-(3-chlorobenzo[b]thiophene-2-carboxamido)-3-hydroxy-benzoic acid methyl ester as a white solid (690 mg, 80%).

To a solution of this ester (300 mg, 0.831 mmol) in DMF (15 ml), potassium carbonate (138 mg, 1.00 mmol) and 4-chlorobenzylbromide (188 mg, 0.91 mmol) were added. After stirring 1 h at room temperature, the mixture was poured into water and the precipitate was collected and washed with water. After drying in a vacuum oven, 4-(3-chloro-benzo[b]thiophene-2-carboxamido)-3-(4-chlorobenzyloxy)-benzoic acid methyl ester was obtained as a white solid (396 mg, 98%).

To a solution of this ester (394 mg, 0.81 mmol) in THF/methanol (1:1, 50 ml), 6 N sodium hydroxide (1.35 ml, 8.1 mmol) was added and the mixture was stirred at 50° C. for 7 h. The reaction mixture was poured into ice containing 1 ml of conc. HCl, and the organic solvents were evaporated. The precipitate was collected and washed repeatedly with water, then with THF/methanol, and finally dried under vacuum at 40° C. overnight. The title compound (236 mg, 62%) was recovered as a white solid mp>200° C.

¹H-NMR (300 MHz, DMSO-d₆, 300° K), δ ppm: 12.93 (br.s, 1H), 9.91 (s, 1H), 8.43 (d, 1H), 8.14 (d, 1H), 7.89 (d, 1H), 7.46-7.79 (m, 8H), 5.34 (s, 2H); MS: m/z=472 [MH]⁺

### Example 311

### 5-(4-Chlorophenylcarbamoylmethoxy)-2-(4-(5,6-dichloro-1H-benzo[d]imidazol-2-yl)benzamido)benzoic acid

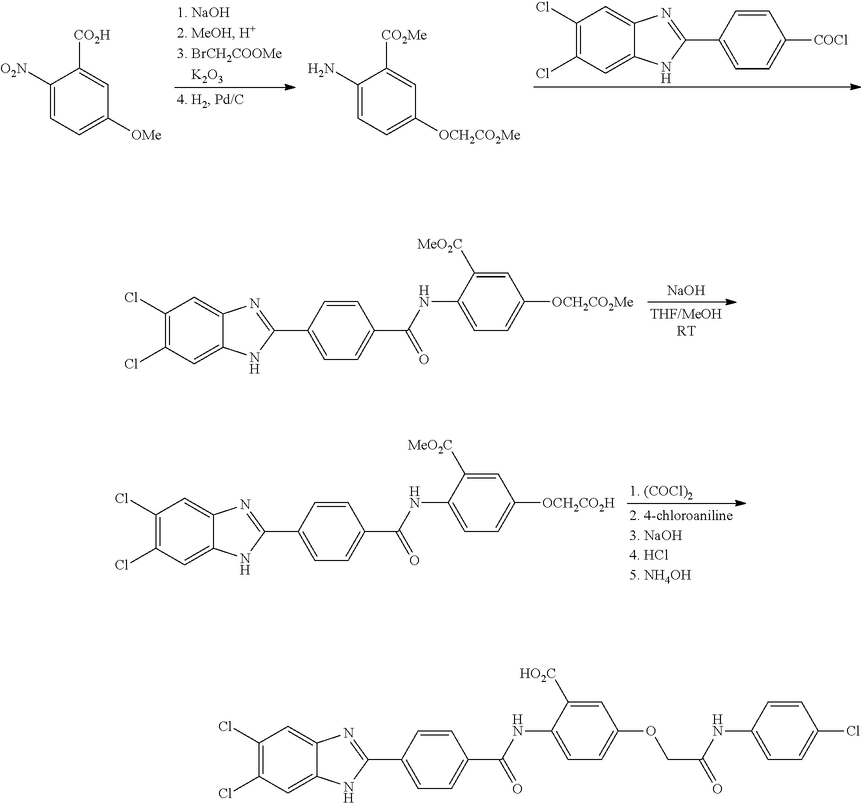

A solution of 5-methoxy-2-nitrobenzoic acid (1 g, 5.07 mmol) and sodium hydroxide (1 g, 25.00 mmol) in 5 ml of water was refluxed overnight. The mixture was acidified and evaporated; the residue was suspended in DCM/MeOH 8:2 and the unsoluble salts were discarded. After evaporation 5-hydroxy-2-nitrobenzoic acid (0.91 g, 98% yield) was obtained.

A mixture of this compound (0.91 g, 4.97 mmol) and 5 ml of conc. H₂SO4 in 20 ml of MeOH was refluxed overnight. The mixture was poured into ice and extracted with AcOEt, which was then washed with brine, dried and evaporated to obtain 0.91 g of methyl 5-hydroxy-2-nitrobenzoate (4.62 mmol, 93% yield) as oil which solidifies under vacuum. A mixture of this compound (468 mg, 2.374 mmol) and K₂CO₃ (394 mg, 2.85 mmol) in 15 ml of DMF was stirred for 30 min, then methyl 2-bromoacetate (263 μl, 2.85 mmol) was added and the resulting mixture was stirred for 3 h at room temperature. The mixture was poured into ice and extracted with AcOEt, which was then washed with brine, dried and evaporated to obtain 728 mg of oil, which was dissolved in 40 ml of methanol and hydrogenated overnight at 35 psi in a Parr apparatus in the presence of Pd/C 10% (100 mg). The mixture was filtered through a Celite pad and evaporated to provide 435 mg (77%) of methyl 2-amino-5-(methoxycarbonylmethoxy)benzoate.

A mixture of 4-(5,6-dichloro-1H-benzo[d]imidazol-2-yl)benzoic acid (120 mg, 0.391 mmol) and a small trace of DMF in 5 ml of SOCl₂ was stirred at 80° C. for 1.5 h. The mixture was evaporated and the residue was resuspended in dry THF (10 ml). Methyl 2-amino-5-(methoxycarbonylmethoxy)benzoate (103 mg, 0.430 mmol) and DMAP (52.5 mg, 0.430 mmol) were added and the mixture was stirred overnight at room temperature. Water (2 ml) was added and the mixture was stirred 15 min, then the precipitate was recovered by filtration and washed with methanol. 163 mg of methyl 2-(4-(5,6-dichloro-1H-benzo[d]imidazol-2-yl)benzamido)-5-(methoxycarbonylmethoxy)benzoate (0.309 mmol, 79% yield) were obtained.

A mixture of this compound (103 mg, 0.195 mmol) and 6 N NaOH (32.5 μl, 0.195 mmol) in 10 ml of THF and 5 ml of MeOH was stirred at room temperature overnight. The mixture was acidified and the precipitate was collected and washed with water, a small amount of methanol and Et₂O. 80 mg of 2-(4-(4-(5,6-dichloro-1H-benzo[d]imidazol-2-yl)benzamido)-3-(methoxycarbonyl)phenoxy)acetic acid (0.156 mg, 80% yield) were obtained as a yellow solid.

A mixture of this compound (80 mg, 0.156 mmol), (COCl)₂ (44 μl, 0.498 mmol) and a small trace of DMF in 20 ml of DCM and 5 ml of THF was heated to 60° C. for 2 h. The solvent was evaporated and the residue was resuspended in 30 ml of dry THF. 4-Chloroaniline (79 mg, 0.622 mmol) was added and the resulting mixture was stirred at room temperature for three days. The mixture was evaporated and the residue was treated with methanol. A yellow precipitate was recovered by filtration, which was purified twice by stirring in 6 ml of a MeOH/conc. ammonia 95:5 mixture. 50 mg (51.5%) of methyl 5-(4-chlorophenylcarbamoylmethoxy)-2-(4-(5,6-dichloro-1H-benzo[d]imidazol-2-yl)benzamido)benzoate were recovered by filtration as a pale yellow solid.

This compound (50 mg, 0.080 mmol) was dissolved in 20 ml of THF containing 6 N NaOH (26.7 μl, 0.160 mmol) and the reaction mixture was stirred at 65° C. for 6 h. Methanol and water were added to obtain a clear solution, which was then acidified and evaporated under vacuum. The residue was stirred in water and the yellow precipitate was collected and washed with methanol. It was then dissolved in 3 ml of a warm MeOH/conc. ammonia/DMSO mixture, from which the yellow ammonium salt of the title compound (31 mg, 62%) was precipitated by addition of water and methanol and collected by filtration, mp>200° C.

¹H-NMR (300 MHz, DMSO-d₆, 300° K), δ ppm: 11.88 (s, 1H), 10.24 (s, 1H), 8.58 (d, 1H), 8.24-8.45 (m, 2H), 8.01-8.24 (m, 2H), 7.92 (s, 2H), 7.66-7.73 (m, 2H), 7.66 (d, 1H), 7.34-7.46 (m, 3H), 4.78 (br.s, 2H); MS: m/z=608.9 [MH]⁺

### Determination of IC₅₀ for Human Neurotrypsin

Assessment of the potency of compounds of examples 1-311 in inhibiting the enzymatic activity of human neurotrypsin was performed according to the method described in patent application WO 2006/1103261

### Determination of the IC₅₀ for Various Proteases

A 6 mM solution of the test compound in DMSO was prepared. In a V-bottom micro plate the 6 mM compound solution was diluted 6 times in DMSO to obtain a concentration row with the following concentrations: 6 mM, 1.8 mM, 540 μM, 162 μM, 48.8 μM, 14.4 μM, and 4 μM. From this DMSO master plate 1:20 dilutions in 10 mM MOPS pH 8.3 were prepared to obtain solutions of 300, 90, 27, 8.1, 2.44, 0.72 and 0.22 μM in MOPS buffer and 5% DMSO.

100 μl of this solution were pipetted into a flat bottom 96 well microplate and placed into a Tecan infinite 200 plate reader. Before measurement, 50 μl of protease solution and, subsequently 50 μl substrate solution were added by the machine. The liberation of free para nitroanilide was detected at 405 nm. The protease solution was freshly prepared by diluting to the desired concentration from a stock solution in 60 mM MOPS; 600 mM NaCl; 20 mM CaCl₂; 10% DMSO; 0.4% PEG 6000 pH 8.3. The substrate solution was prepared by dissolving the solid substrate in an adequate amount of water to get the desired concentrations. The used protease and substrate solutions are given below:

| Protease | concentration | | Substrate | concentration |
|---|---|---|---|---|
| Trypsin | 2 | nM | S-2222 | 0.5 mM |
| Thrombin | 6 | nM | S-2266 | 0.5 mM |
| Plasmin | 8 | nM | S-2288 | 0.5 mM |
| Kallikrein | 2 | nM | S-2288 | 0.5 mM |
| tPA (2 chain) | 40 | nM | S-2288 | 0.5 mM |
| uPA | 40 | nM | S-2288 | 0.5 mM |
| Factor Xa | 5 | nM | S-2288 | 0.5 mM |

All enzymes except trypsin were from human origin (American diagnostic). Trypsin is hog trypsin (Sigma Aldrich). Substrates were purchased from Chromogenix. S-2222 is N-benzoyl-L-isoleucyl-L-glutamyl-glycyl-L-arginine-p-nitroanilide hydrochloride and its methyl ester PhCO-Ile-Glu(OR)-Gly-Arg-pNA HCl (50% where R is H and 50% where R is CH₃). S-2266 is H-D-valyl-L-leucyl-L-arginine-p-nitroanilide dihydrochloride (H-D-Val-Leu-Arg-pNA 2HCl). S-2288 is H-D-isoleucyl-L-prolyl-L-arginine-p-nitroanilide dihydrochloride (H-D-Ile-Pro-Arg-pNA 2HCl). The reactions were performed for 20-30 min and the initial velocities were plotted against the concentrations of the test compound.

To obtain the IC₅₀, a curve with the equation

$$y = \frac{100}{1 + {( \frac{{IC}_{50}}{[ I ]} )}^{h}}$$

was fit to the data points, where “I” is the concentration of the test compound and h represents the Hill coefficient.

The resulting IC₅₀ values for Example 119 are summarized below:

| | Mean ± |
|---|---|
| IC₅₀ (μM) | SD |
| Neurotrypsin | 0.51 ± 0.16 |
| Trypsin | >150 |
| Thrombin | 37.7 ± 25.2 |
| Plasmin | 2.4 ± 1.6 |
| Kallikrein | 27.1 ± 9.2 |
| tPA | 47.1 ± 22.4 |
| uPA | >40 |
| Factor Xa | 39.8 ± 33.3 |

### Test of In Vivo Efficacy of Example 119, Mono-Lysine Salt, on Body Weight and Plasma Levels of C-Terminal Agrin Fragment (CAF)

### Experimental Animals

Adult (P50) C57/BL6 mice from Charles River were used for the experiments. All animal experiments are performed in accordance with international guidelines for proper conduct of animal experiment under jurisdiction of the Swiss law (Permit number ZH-18/2008). For every mouse in the experiment the well-being during the interventions is documented in a score sheet.

The mice are housed under optimal hygienic conditions (OHB) with controlled air-conditioning and lighting (temperature: 20-24° C., humidity: 55-65%, day/night cycle: 12 h/12 h, lights on 7 a.m.). Individually ventilated cages of type II, L (530 cm² floor space) with standard litter are used. A maximum of 6 mice is kept in each cage. Mice are fed with standard rodent chow (Extrudat, KLIBA NAFAG) ad libitum and have free access to drinking water. Animal colony is managed and maintained at BioSupport, Schlieren.

### Compound Formulation

Example 119, mono-lysine salt, was prepared for the dosing as suspension in 20% DMSO, 80% PBS with a final concentration of 2.5 mg/ml active compound (corresponding to 3.13 mg/ml of the lysine salt). First, compound of Example 119, mono-lysine salt, was dissolved in DMSO at a concentration of 15.7 mg/ml. Then sterile PBS was added to get a final concentration of 3.13 mg/ml and the suspension was thoroughly vortexed. The suspension was kept at room temperature in the dark until used.

### Treatment Schedule

5 adult mice were treated with 10 ml/kg of a 3.13 mg/ml suspension of Example 119, mono-lysine salt. This corresponds to a dose of 25 mg/kg active ingredient. As vehicle control 5 mice were treated with 10 ml/kg of 20% DMSO in PBS. Each mouse received three intraperitoneal injections. Between dose 1 and dose 2 there were 8 h, between dose 2 and dose 3 16 h. 5 h after the last injection the mice were euthanatized with CO₂.

### Sample Collection and Preparation

Before the first injection the mice were weighted and ˜100 μl of blood was collected from the lateral tail vein. Serum was prepared with centrifugation of blood samples in Microvette 500 Z-Gel tubes (10'000 g, 20° C., 5 min). Supernatant was transferred to an Eppendorf reaction tube and centrifuged again (21'000 g, 20° C., 5 min). Supernatant was transferred to screw top reaction tubes and the serum was stored at −20° C. until analysis. Before scarification mice were weighted again. Post-treatment blood was collected by cardiac puncture right after euthanasia. Serum was prepared as described above for pre-treatment samples.

### Effect of Compound on Body Weight

For every animal the weight loss caused by treatment was computed as ratio of post-treatment/pre-treatment weight. The values were averaged for compound of Example 119, mono-lysine salt and the vehicle treated mice, respectively. Sample means are indicated with standard deviations.

Weight loss in both groups of mice is below the 10% threshold. Example 119, mono-lysine salt does not produce any increase in weight loss compared to the vehicle control.

| | | Average weight loss | |
|---|---|---|---|
| Treatment | n | (% of T = 0 weigth) | SD |
| Vehicle | 5 | 5.67 | 2.50 |
| Example 119, mono-lysine salt | 5 | 5.52 | 1.42 |

### Analysis of Serum CAF Levels by Western Blotting

For the western blot analysis the blood samples from the two groups (vehicle and Example 119, mono-lysine salt) and two sampling times (0 and 1) were pooled (10 μl of each individual sample) resulting in following four pools: Vehicle-0, Vehicle-1, Ex. 119-0, Ex. 119-1. 50 μl of PBS was added to each pool and the diluted samples were centrifuged with 100 kDa size cut-off filters (Microcon, 14'000 g, RT, 30 min). Filtrates were mixed with 4×Lämmli buffer and loaded with a Hamilton syringe onto a 4-12% gradient NUPAGE gel for electrophoresis. Pooled sera from wild type mice and pooled sera from neurotrypsin knockout mice that had been processed as described above were loaded as controls. After electrophoresis the proteins were transferred onto a PVDF membrane with semi-dry blotting. The membrane was then stained with affinity purified (against recombinant C-terminal agrin fragment) polyclonal agrin antibodies. A secondary antibody conjugated with HRP together with the luminol/peroxide chemiluminescence system (ChemiGlow West, Alpha Innotech) was used to detect the CAF signal.

After staining for CAF the membrane was washed thoroughly and reused for a staining for endogenous mouse antibodies that served as loading control. A rabbit anti-mouse antibody conjugated with HRP together with the chemiluminiscence system described above was used. After imaging with a Stella imaging system (Raytest) the intensity of the CAF bands was measured with AIDA picture analysis software (Raytest) and normalized to the loading levels determined from mouse antibody light chains. The background present in the KO sample was then subtracted from every sample. The western blot was repeated once, the values for the CAF level were averaged and then normalized to the vehicle level. The normalized means with the standard deviation are depicted below.

| | CAF levels | | | |
|---|---|---|---|---|
| | Average | | Average | |
| Treatment | (AU) | SD | (% of vehicle) | SD |
| Vehicle | 0.68 | 0.03 | 100.00 | 4.37 |
| Example 119, mono-lysine salt, | 0.38 | 0.20 | 56.15 | 29.03 |

Treatment with Example 119, mono-lysine salt, led to reduction of CAF levels to 56.2%±29.0% of vehicle control.

For selected examples IC₅₀ was measured with respect of neurotrypsin, and the results collected in the following Table:

| Example N^{o} | Neurotrypsin IC₅₀, μM |
|---|---|
| 1 | 8.5 |
| 5 | 1.9 |
| 9 | 25.7 |
| 16 | 8.0 |
| 18 | 8.3 |
| 23 | 2.6 |
| 24 | 4.0 |
| 25 | >150 |
| 26 | 3.2 |
| 27 | 5.0 |
| 28 | 11.3 |
| 32 | 13.6 |
| 33 | 4.5 |
| 34 | 14.1 |
| 35 | 3.2 |
| 36 | 3.5 |
| 37 | 1.6 |
| 38 | 4.8 |
| 40 | 0.8 |
| 42 | 4.4 |
| 43 | 3.9 |
| 44 | 1.6 |
| 45 | 1.5 |
| 46 | 3.4 |
| 47 | 3.3 |
| 48 | 3.4 |
| 49 | 6.6 |
| 51 | 0.5 |
| 52 | 15.4 |
| 59 | 9.0 |
| 62 | 7.5 |
| 63 | 129.5 |
| 64 | 23.3 |
| 65 | 7.3 |
| 67 | 10.7 |
| 69 | 3.1 |
| 73 | 4.4 |
| 74 | 26.1 |
| 75 | 5.6 |
| 78 | 19.2 |
| 82 | 17.8 |
| 84 | 4.8 |
| 85 | 2.3 |
| 87 | 4.3 |
| 88 | 24.8 |
| 90 | 4.9 |
| 91 | 10.0 |
| 97 | 10.3 |
| 99 | 6.9 |
| 101 | 9.0 |
| 103 | 4.1 |
| 107 | 4.8 |
| 108 | 3.9 |
| 110 | 3.4 |
| 112 | 5.0 |
| 113 | 2.7 |
| 114 | 3.0 |
| 115 | 3.1 |
| 116 | 2.4 |
| 117 | 2.9 |
| 118 | 2.2 |
| 119 | 0.4 |
| 120 | 4.1 |
| 121 | 4.9 |
| 122 | 9.4 |
| 123 | 3.5 |
| 124 | 3.9 |
| 125 | 3.5 |
| 126 | 1.2 |
| 129 | 1.0 |
| 130 | 7.6 |
| 131 | 0.7 |
| 132 | 5.3 |
| 133 | 1.9 |
| 134 | 1.9 |
| 135 | 0.9 |
| 137 | 2.4 |
| 138 | 6.6 |
| 139 | 7.5 |
| 140 | 0.9 |
| 141 | 1.9 |
| 142 | 2.2 |
| 143 | 3.9 |
| 144 | 22.1 |
| 148 | 4.3 |
| 149 | 16.5 |
| 151 | 2.7 |
| 156 | 4.7 |
| 157 | 3.8 |
| 158 | 3.8 |
| 159 | 6.5 |
| 160 | 3.9 |
| 161 | 3.0 |
| 162 | 4.5 |
| 163 | 2.6 |
| 164 | 3.5 |
| 165 | 8.9 |
| 188 | 6.8 |
| 194 | 5.9 |
| 196 | 10.2 |
| 218 | 40.8 |
| 227 | 9.7 |
| 228 | 6.6 |
| 229 | 3.7 |
| 246 | 98.4 |
| 248 | >150 |
| 249 | 2.8 |
| 250 | 2.4 |
| 255 | 3.1 |
| 257 | 3.8 |
| 258 | 9.4 |
| 260 | 11.0 |
| 261 | 5.5 |
| 262 | 7.0 |
| 263 | 4.0 |
| 266 | 3.7 |
| 267 | 0.5 |
| 270 | 22.9 |
| 271 | 14.2 |
| 273 | 7.6 |
| 274 | 6.6 |
| 275 | 3.0 |
| 276 | 6.3 |
| 277 | 3.5 |
| 278 | 19.4 |
| 279 | 5.4 |
| 280 | 5.0 |
| 282 | 11.3 |
| 283 | 3.4 |
| 286 | 10.4 |
| 287 | 4.3 |
| 288 | 5.1 |
| 290 | 4.1 |
| 291 | 4.0 |
| 293 | 8.7 |
| 301 | 8.7 |
| 302 | 4.4 |
| 303 | 5.5 |
| 304 | 2.5 |
| 305 | 1.2 |
| 306 | 0.5 |
| 311 | 0.5 |

1. A compound of formula (I) 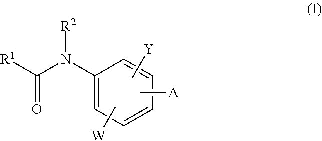
wherein
A is —CONR³R⁴, —NR⁵COR⁶, —NHR⁷, —OR⁸, —SR⁹, —CH₂NR¹⁰R¹¹, —(CH₂)₂—R¹², —CH═CH—R¹², —C≡C—R¹², optionally substituted phenyl, optionally substituted thiophenyl, or optionally substituted 1,2,3-triazol-4-yl;
W is hydrogen, hydroxy or carboxymethoxy;
Y is carboxy, methoxycarbonyl or 2H-tetrazol-5-yl;
R¹ is cycloalkyl, cycloalkenyl, aryl, arylmethyl, heteroaryl, or heteroarylmethyl;
R² is hydrogen or methyl;
R³ is alkyl, optionally substituted amino-, hydroxy-, carbamimidoyl-, or cycloalkyl-lower alkyl;
aryl-lower alkyl, heteroaryl-lower alkyl, cycloalkyl, bicycloalkyl, tricycloalkyl, heterocyclyl, aryl, or heteroaryl;
R⁴ is hydrogen, lower alkyl, carboxy-, lower alkoxycarbonyl-, dimethylcarbamoyl-, hydroxy- or lower alkoxy-lower alkyl;
or R³ and R⁴ together with the nitrogen atom, to which they are bound, are optionally substituted pyrrolidino, optionally substituted piperidino, morpholino, or optionally substituted piperazino;
R⁵ is hydrogen or methyl;
R⁶ is aryl, heteroaryl, optionally substituted alkylamino, arylamino, optionally substituted pyrrolidino, optionally substituted piperidino, morpholino, or optionally substituted piperazino, with the proviso that R⁶ cannot be 2-thiophenyl if R¹ is 2-thiophenyl;
or R⁵ and R⁶ together with the nitrogen atom and the carbonyl group, to which they are bound, are optionally substituted 2-oxopyrrolidino, optionally substituted 2-oxopiperidino, or optionally substituted 2-oxo-oxazolidin-3-yl;
R⁷ is cycloalkyl, cycloalkenyl, aryl, aryl-lower alkyl, optionally substituted alkylsulfonyl, or arylsulfonyl;
R⁸ is phenyl if Y is carboxy and R¹ is optionally substituted benzimidazolyl-phenyl or chloro-substituted benzothiophenyl, aryl-lower alkyl with the exclusion of ortho-methoxybenzyl, optionally substituted benzocycloalkyl or benzocycloalkenyl, optionally substituted alkyl-, dialkyl- or aryl-carbamoylmethyl, or heterocyclylcarbonylmethyl, wherein heterocyclyl is bound to carbonyl through a nitrogen atom;
R⁹ is aryl;
R¹⁰ is arylcarbonyl, heteroarylcarbonyl or optionally substituted alkylcarbonyl;
R¹¹ is hydrogen or methyl;
or R¹⁰ and R¹¹ together with the nitrogen atom, to which they are bound, are optionally substituted 2-oxopyrrolidino, optionally substituted 2-oxopiperidino, or optionally substituted 2-oxo-oxazolidin-3-yl; and
R¹² is aryl or aryl-lower alkyl if R¹ is optionally substituted benzimidazolyl-phenyl or chloro-substituted benzothiophenyl;
and salts thereof.

2. The compound of claim 1 according to formula (I) wherein
A is —NHR⁷, —OR⁸ or —SR⁹;
W is hydrogen;
Y is carboxy, methoxycarbonyl or 2H-tetrazol-5-yl;
R¹ is optionally substituted phenyl, optionally substituted thiophenyl or benzothiophenyl, or optionally substituted 1,3-thiazol-2-yl;
R² is hydrogen; and
R⁷ is C₃-C₇-cycloalkyl, optionally substituted phenyl, optionally substituted benzyl or phenethyl, optionally substituted benzo-C₅- or C₆-cycloalkyl or -cycloalkenyl, optionally substituted alkylsulfonyl, or optionally substituted phenylsulfonyl;
R⁸ is optionally para- or meta-substituted benzyl, optionally substituted phenethyl, optionally substituted benzo-C₅- or C₆-cycloalkyl or -cycloalkenyl, optionally substituted alkylcarbamoylmethyl, dimethylcarbamoylmethyl, optionally substituted phenylcarbamoylmethyl, pyrrolidinocarbonylmethyl, piperidinocarbonylmethyl, morpholinocarbonylmethyl, or piperazinocarbonylmethyl; and
R⁹ is optionally substituted phenyl;
or A is —OR⁸; W is hydrogen; Y is carboxy; R¹ is optionally substituted benzimidazolyl-phenyl or chloro-substituted benzothiophenyl; R² is hydrogen; and R⁸ is phenyl;
and pharmaceutically acceptable salts thereof.

3. The compound of claim 1 according to formula (I) wherein
A is —NHR⁷, —OR⁸ or —SR⁹;
W is hydrogen;
Y is carboxy, methoxycarbonyl or 2H-tetrazol-5-yl;
R¹ is optionally substituted phenyl, optionally substituted thiophenyl or benzothiophenyl, or optionally substituted 1,3-thiazol-2-yl;
R² is hydrogen;
R⁷ is optionally substituted benzyl or phenethyl, optionally substituted phenyl, indanyl, 1,2,3,4-tetrahydronaphthalenyl, or optionally substituted phenylsulfonyl;
R⁸ is optionally para- or meta-substituted benzyl or optionally substituted phenylcarbamoylmethyl; and
R⁹ is optionally substituted phenyl;
or A is —OR⁸; W is hydrogen; Y is carboxy; R¹ is chloro-substituted benzothiophenyl; R² is hydrogen; and R⁸ is phenyl;
and A and Y are in position 2 and 4, 2 and 5, 3 and 4, 3 and 5, 3 and 6, 4 and 2, and 4 and 3 of the phenyl ring, respectively;
and pharmaceutically acceptable salts thereof.

4. The compound of claim 1 according to formula (I) wherein
A is —OR⁸; W is hydrogen; Y is carboxy or methoxycarbonyl; R¹ is 4-(1H-benzimidazol-2-yl)phenyl, wherein benzimidazolyl is optionally substituted at the benzo residue by dichloro, or optionally substituted benzo[b]thiophen-2-yl; R² is hydrogen; and
R⁸ is p-chlorobenzyl or p-chlorophenylcarbamoylmethyl;
or A is —OR⁸; W is hydrogen; Y is carboxy; R¹ is 3-chloro-benzo[b]thiophen-2-yl; R² is hydrogen; and R⁸ is phenyl;
and pharmaceutically acceptable salts thereof.

5. The compound of claim 1 according to formula (I) wherein
A is —OR⁸; W is hydrogen; Y is carboxy or methoxycarbonyl; R¹ is 5,6-dichloro-1H-benzimidazol-2-yl, benzo[b]thiophen-2-yl or 3-chloro-benzo[b]thiophen-2-yl; R² is hydrogen;
and R⁸ is p-chlorophenylcarbamoylmethyl;
and pharmaceutically acceptable salts thereof.

6. The compound of claim 1 according to formula (I) wherein
A is —CONR³R⁴;
W is hydrogen, hydroxy or carboxymethoxy;
Y is carboxy, methoxycarbonyl or 2H-tetrazol-5-yl;
R¹ is C₃-C₇-cycloalkyl, optionally substituted phenyl, optionally substituted benzo-C₅- or C₆-cycloalkyl or -cycloalkenyl, optionally substituted thiophenyl or benzothiophenyl, optionally substituted indol-2-yl, optionally substituted 1H-benz[d]imidazol-2-yl, optionally substituted 1,3-thiazol-2-yl, or thiophenylmethyl;
R² is hydrogen or methyl;
R³ is alkyl, methylamino-lower alkyl, carbamimidoyl-lower alkyl; C₅- or C₆-cycloalkylmethyl, optionally substituted benzyl, optionally substituted phenylethyl, optionally substituted thiophenylmethyl, C₃-C₇-cycloalkyl, bicyclo[2.2.1]heptyl, adamantyl, optionally substituted benzo-C₅- or -C₆-cycloalkyl or -cycloalkenyl, optionally substituted phenyl, 2-oxo-pyrrolidino or -piperidino; optionally substituted pyridyl, optionally substituted thiophenyl or benzothiophenyl, 1-methyl-1H-pyrazol-3-yl, pyridazin-4-yl, isoxazol-3-yl, or optionally substituted 1,3-thiazol-2-yl;
R⁴ is hydrogen, lower alkyl, carboxymethyl, lower alkoxycarbonylmethyl, dimethyl-carbamoylmethyl, hydroxy-lower alkyl or methoxy-lower alkyl;
or R³ and R⁴ together with the nitrogen atom, to which they are bound, are optionally substituted pyrrolidino, optionally substituted piperidino, tetrahydro-quinolyl or -isoquinolyl, morpholino, or optionally substituted piperazino;
and pharmaceutically acceptable salts thereof.

7. The compound of claim 1 according to formula (I) wherein
A is —CONR³R⁴;
W is hydrogen, hydroxy or carboxymethoxy;
Y is carboxy, methoxycarbonyl or 2H-tetrazol-5-yl;
R¹ is C₃-C₇-cycloalkyl;
optionally substituted phenyl with one to three substituents, wherein the substituents are selected from the group consisting of lower alkyl, hydroxy, lower alkoxy, halo, cyano, halobenzyl, thiophen-2-yl, and 1H-benzimidazol-2-yl optionally substituted at nitrogen by methyl or carboxymethyl and at the benzo residue by carboxy, chloro or dichloro;
2-indanyl or 2-indenyl, optionally substituted by chloro and/or phenyl;
2- or 3-thiophenyl, optionally substituted by lower alkyl, propen-1-yl, vinyl, halo, cyano, phenyl, halophenyl, methoxyphenyl, ethylenedioxyphenyl, or 4-(4-methylpiperazin-1-ylmethyl);
benzo[b]thiophen-2- or -3-yl, optionally substituted by halo, dihalo or ethylenedioxy;
1H-indol-2-yl, optionally substituted by chloro and/or phenyl;
1H-benz[d]imidazol-2-yl, optionally substituted by halo, dihalo, carboxy or methoxycarbonyl;
1,3-thiazol-2-yl, optionally substituted by lower alkyl, halo, or acetoxy, or with an annullated cyclopenta, benzo, or halobenzo ring;
or 2-thiophenylmethyl;
R² is hydrogen or methyl;
R³ is alkyl; methylamino-lower alkyl, carbamimidoyl-lower alkyl; cyclohexylmethyl, optionally halogenated or carbamimidoylated benzyl, optionally halogenated phenylethyl;
thiophenylmethyl, optionally substituted by halo or chlorophenyl, benzo[b]thiophenylmethyl;
C₃-C₇-cycloalkyl, bicyclo[2.2.1]heptyl, adamantyl, indanyl, tetrahydronaphthalenyl;
optionally substituted phenyl with one to three substituents, wherein the substituents are selected from the group consisting of halo, cyano, lower alkyl, hydroxy-lower alkyl, phenyl-hydroxy-lower alkyl, optionally halogenated benzyl, methylamino-lower alkyl, dimethylamino-lower alkyl, carbamidoyl-lower alkyl, hydroxy, lower alkoxy, hydroxy-lower alkoxy, phenoxy, benzyloxy, pyridoxy, phenyl, carboxy, phenylcarbonyl, carbamimidoyl, methylsulfonyl, N,N-dimethylsulfamoyl, N-carbamimidoylsulfamoyl, and 5-oxo-2,5-dihydro-1,2,4-oxadiazol-3-yl;
2-oxo-pyrrolidino; optionally halogenated pyridyl;
2- or 3-thiophenyl, optionally substituted by chloro, cyano or vinyl;
1-methyl-M-pyrazol-3-yl, pyridazin-4-yl, isoxazol-3-yl, 1,3-thiazol-2-yl, optionally halogenated 1,3-thiazol-2-yl or benzo[d]-1,3-thiazol-2-yl;
R⁴ is hydrogen, lower alkyl, carboxymethyl, ethoxycarbonylmethyl, dimethylcarbamoylmethyl, hydroxy-lower alkyl or methoxy-lower alkyl;
or R³ and R⁴ together with the nitrogen atom, to which they are bound, are pyrrolidino, optionally substituted by keto, phenyl, chlorophenyl or phenyoxy; piperidino, optionally substituted by phenoxy, optionally substituted phenyl wherein the substituents on phenyl are fluoro, chloro, hydroxy, methoxy, trifluoromethyl or methyl, hydroxy, optionally substituted benzyl wherein the substituents on benzyl are fluoro, chloro or methoxy; tetrahydro-quinolyl or -isoquinolyl; morpholino; or piperazino, optionally substituted by keto, 4-benzyl or 4-tert-butyl;
and A and Y are in position 2 and 3, 2 and 4, 2 and 5, 2 and 6, 3 and 2, 3 and 4, 3 and 5, 3 and 6, 4 and 2, and 4 and 3 of the phenyl ring, respectively;
and pharmaceutically acceptable salts thereof.

8. The compound of claim 1 according to formula (I) wherein
A is —NR⁵COR⁶;
W is hydrogen;
Y is carboxy, methoxycarbonyl or 2H-tetrazol-5-yl;
R¹ is optionally substituted C₃-C₇-cycloalkyl, optionally substituted phenyl, optionally substituted benzo-C₅- or C₆-cycloalkyl or -cycloalkenyl, optionally substituted thiophenyl or benzothiophenyl, optionally substituted 1H-benz[d]imidazol-2-yl, optionally substituted 1,3-thiazol-2-yl;
R² is hydrogen or methyl;
R⁵ is hydrogen; and
R⁶ is optionally substituted phenyl, optionally substituted thiophenyl or benzothiophenyl, optionally substituted 1,3-thiazol-2-yl, optionally substituted alkyl- or phenyl- or benzylamino, optionally substituted pyrrolidino, optionally substituted piperidino, or morpholino; with the proviso that R⁶ cannot be 2-thiophenyl if R¹ is 2-thiophenyl; or
R⁵ and R⁶ together with the nitrogen atom and the carbonyl group, to which they are bound, are optionally substituted 2-oxopyrrolidino, optionally substituted 2-oxopiperidino, or optionally substituted 2-oxo-oxazolidin-3-yl;
and pharmaceutically acceptable salts thereof.

9. The compound of claim 1 according to formula (I) wherein
A is —CH₂NR¹⁰R¹¹;
W is hydrogen;
Y is carboxy, methoxycarbonyl or 2H-tetrazol-5-yl;
R¹ is optionally substituted phenyl, optionally substituted thiophenyl, optionally substituted benzothiophenyl, or optionally substituted 1,3-thiazol-2-yl;
R² is hydrogen;
R¹⁰ is arylcarbonyl, heteroarylcarbonyl or optionally substituted alkylcarbonyl;
R¹¹ is hydrogen or methyl; or
R¹⁰ and R¹¹ with the nitrogen atom, to which they are bound, are optionally substituted 2-oxopyrrolidino, optionally substituted 2-oxopiperidino or optionally substituted 2-oxo-oxazolidin-3-yl;
and pharmaceutically acceptable salts thereof.

10. The compound of claim 1 according to formula (I) wherein
A is —(CH₂)₂—R¹², —CH═CH—R¹² or —C≡C—R¹²;
W is hydrogen;
Y is carboxy, methoxycarbonyl or 2H-tetrazol-5-yl;
R¹ is optionally substituted benzimidazolyl-phenyl or chloro-substituted benzothiophenyl
R² is hydrogen; and
R¹² is aryl or aryl-lower alkyl;
and pharmaceutically acceptable salts thereof.

11. The compound of claim 1 according to formula (I) wherein
A is phenyl, halo-, methoxy- or cyanophenyl, thiophenyl, or halo- or carbamoyl-thiophenyl;
W is hydrogen;
Y is carboxy, methoxycarbonyl or 2H-tetrazol-5-yl;
R¹ is optionally substituted benzimidazolyl-phenyl or chloro-substituted benzothiophenyl; and
R² is hydrogen;
and pharmaceutically acceptable salts thereof.

12. The compound of claim 1 according to formula (I) wherein
A is optionally substituted 1,2,3-triazol-4-yl;
W is hydrogen;
Y is carboxy, methoxycarbonyl or 2H-tetrazol-5-yl;
R¹ is optionally substituted benzimidazolyl-phenyl, optionally substituted thiophenyl, or optionally substituted benzothiophenyl; and
R² is hydrogen;
and pharmaceutically acceptable salts thereof.

13-14. (canceled)

15. A pharmaceutical composition comprising a compound according to claim 1.

16. A method of treatment and/or prophylaxis of diseases caused by deficiency of synapses, comprising administering to a patient in need thereof a therapeutically effective amount of a compound according to claim 1.

17. A method of treatment and/or prophylaxis according to claim 16, wherein the diseases caused by deficiency of synapses are skeletal muscle atrophy, schizophrenia, Alzheimer's disease, and cognitive disturbance.
